(19) United States
(12) Patent Application Publication
(10) Pub. No.: US 2007/0202508 A1
(43) Pub. Date: Aug. 30, 2007

(54) Novel thermophilic proteins and the nucleic acids encoding them

(76) Inventors: Arcady Mushegian, Leawood, KS (US); Jing Liu, Salt Lake City, UT (US); Tatyana Naryshkina, East Brunswick, NJ (US); Konstantin V. Saverinov, Piscataway, NJ (US)

Correspondence Address: POLSINELLI SHALTON FLANIGAN SUELTHAUS PC, 700 W. 47TH STREET, SUITE 1000, KANSAS CITY, MO, 64112-1802, US

(21) Appl. No.: 11/361,092

(22) Filed: Feb. 24, 2006

(51) Int. Cl. C12Q 1/68; C07H 21/04; C12P 21/06; C12P 19/34; C12N 9/02; C12N 9/10; C12N 9/12; C12N 9/22; C12N 9/64

(52) U.S. Cl. 435/6; 435/69.1; 435/193; 435/199; 435/194; 435/189; 435/226; 435/320.1; 435/91.2; 435/252.3; 536/23.2

(57) ABSTRACT

The disclosed invention relates to the fields of molecular biology and biochemistry. Thermophilic proteins and the nucleic acids encoding them are disclosed. The thermophilic proteins are from, or derived from, a bacteriophage, YS40, that infects the thermophilic bacterium Thermus thermophilus. These proteins have enhances stability, particularly at high temperatures.

### FIELD OF THE INVENTION

The disclosed invention relates to the fields of molecular biology and biochemistry. Thermophilic proteins and the nucleic acids encoding them are disclosed. The thermophilic proteins are from, or derived from, a bacteriophage, YS40, that infects the thermophilic bacterium Thermus thermophilus. These proteins have enhanced stability, particularly at high temperatures.

### BACKGROUND OF THE INVENTION

In the last decade, bacteriophage (phage) genome sequencing projects have deposited more than 200 complete phage genome sequences in the public databases. While the hosts of these phages are phylogenetically quite diverse, only 10 completely sequenced phages are known to infect thermophilic microorganisms. Most of these thermophilic phages were isolated from a small number of archaeal species (Palm, P., et al. 1991; Wiedenheft, B., et al. 2004; Arnold, H. P., et al. 2000). The only sequenced genome of a phage from a thermophilic bacterium is RM 378 that infects Rhodothermus marinus (Hjorleifsdottir, S., et al. Patent: WO 0075335-A 14 Dec. 2000).

Bacteriophages may be the most abundant living entities on Earth, represented by about 10³¹ individuals, as indicated by random sampling and sequencing of DNA from environmental sources (Hendrix RW. Bacteriophage genomics. Curr Opin Microbiol. October 2003; 6(5):506-511). It has been proposed that the origin of dsDNA bacteriophages is as ancient as DNA replication itself (Filee J, Forterre P, Sen-Lin T, Laurent J. Evolution of DNA polymerase families: evidences for multiple gene exchange between cellular and viral proteins. J Mol Evol. June 2002; 54(6):763-773), and the analysis of the currently known bacteriophages may provide clues to early evolution of cellular and viral genomes. Phage genomes thus present a relatively unexplored source of genetic variation and enzymatic activities that may be of considerable commercial import.

### SUMMARY OF THE INVENTION

Bacteriophage YS40 infects the thermophilic bacterium Thermus thermophilus H8. Analysis of the YS40 genome revealed a dsDNA molecule of 152,372 bp with no terminal repeats or redundancies that contains 169 putative open reading frames, which express polypeptides longer than 50 amino acids, and three tRNA genes. The ability of YS40 to infect and propagate in T. thermophilus at permissive temperatures from about 56 to about 78° C. suggests that proteins encoded in the YS40 genome may have enhanced stability, particularly at higher temperatures. In addition to greater stability, proteins of YS40 may also possess novel enzymatic characteristics with commercial applicability.

Accordingly, the present invention provides isolated proteins that include a thermophilic amino acid sequence at least 75%, or even at least 80%, 85%, 90%, 95%, 96%, 97%, 98% or even at least 99% identical to a YS40 amino acid sequence encoded by at least 25, or at least 30, 35, 40, 45, 50, 60, 70, 75, 80, 90, or even at least 100 contiguous codons from SEQ ID NO: 1-170; preferably from SEQ ID NO: 2, 4-64, 70-149, 151 or 153-170, or conservatively modified variants of the same.

In certain aspects, the thermophilic amino acid sequence is identical to the YS40 amino acid sequence. In some aspects, the YS40 coding sequence is to a YS40 structural protein expressed by a nucleotide sequence selected from SEQ ID NOs: 1, 3, 65, 69, 71, 151 or 152. The thermophilic amino acid sequence confers to the protein, at a permissible temperature of at least 36° C., more preferably at least 45° C., 55° C., 65° C., or even 75° C., an enzymatic activity. Exemplary enzymatic activities of proteins of the present invention include, but are not limited to, decarboxylase, nuclease, synthase, recombinase, helicase, dehydrogenase, reductase, nucleotide primase, kinase, protease, nucleotidyltransferase, nucleic acid polymerase, deaminase, acyltransferase, terminase, helicase, glycosyltransferase and peptidase activities. For enzymes, the YS40 coding sequence is selected from SEQ ID NOs: 5, 8, 9, 12-14, 17, 18, 23-27, 29, 33, 38, 41, 42, 52, 57, 59, 60, 62, 71, 79, 114, 144 or 161. Where the enzyme is a DNA polymerase, the YS40 coding sequence preferably is SEQ ID NO: 33.

The invention also contemplates nucleic acid embodiments where the nucleic acids encode proteins of the invention. It is desirable that the encoded thermophilic amino acid sequence of the encoded protein is identical to the YS40 amino acid sequence. The YS40 amino acid sequence may be encoded by at least about 25, 50, 75 or 100 contiguous codons of the YS40 coding sequence, with the YS40 coding sequence being from SEQ ID NO: 1, 3, 65, 69, 71, 151 or 152. In some aspects the thermophilic amino acid sequence confers an enzyme activity to the encoded protein at a permissible temperature of at least 36° C., more preferably at least 45° C., 55° C., 65° C., most preferably at least 75° C. The enzyme activity may be a decarboxylase, nuclease, synthase, recombinase, helicase, dehydrogenase, reductase, nucleotide primase, kinase, protease, nucleotidyltransferase, nucleic acid polymerase, deaminase, acyltransferase, terminase, helicase, glycosyltransferase or peptidase, depending upon the YS40 coding sequence selected. The YS40 coding sequence may be from SEQ ID NO: 2, 4-64, 70-149, 151 or 153-170, more preferably from SEQ ID NO: 5, 8, 9, 12-14, 17, 18, 23-27, 29, 33, 38, 41, 42, 52, 57, 59, 60, 62, 71, 79, 114, 144 or 161. Ideally, the protein encoded by the nucleic acid is a DNA polymerase at a permissive temperature, and the YS40 coding sequence selected is SEQ ID NO: 33.

Nucleic acids of the present invention may include a YS40 nucleotide from SEQ ID NO: 1-170. The YS40 nucleotide sequence may encode a YS40 structural protein that does not take a random coil structure at a permissible temperature of at least 36° C., 45° C., 55° C., 65° C., or even at least 75° C., or a YS40 enzyme. YS40 enzymes may display decarboxylase, nuclease, synthase, recombinase, helicase, dehydrogenase, reductase, nucleotide primase, kinase, protease, nucleotidyltransferase, nucleic acid polymerase, deaminase, acyltransferase, terminase, helicase, glycosyltransferase or peptidase activities when analyzed, at a permissible temperature of at least 36° C., 45° C., 55° C., 65° C., or even at least 75° C. Such nucleic acids may optionally be operably linked to a regulatory sequence. Such nucleic acids may also be used to transform a cell, and such recombinant cell types form part of the present invention.

Other embodiments of the invention include recombinant vectors. The recombinant vectors include a nucleic acid encoding a protein of the invention, as discussed above, operably linked to a promoter. Introduction of the vector into an expression system produces a protein having, at a permissible temperature of at least 36° C., or even at least 45° C., 55° C., 65° C., or even at least 75° C., and enzymatic activity. These proteins may be characterized as being decarboxylase, nuclease, synthase, recombinase, helicase, dehydrogenase, reductase, nucleotide primase, kinase, protease, nucleotidyltransferase, nucleic acid polymerase, deaminase, acyltransferase, terminase, helicase, glycosyltransferase, peptidase, or combinations thereof. The promoter is preferably inducible or constitutive, and ideally is a strong promoter. In some embodiments the YS40 coding sequence is selected from SEQ ID NO: 2, 4-64, 70-149, 151 or 153-170, or from SEQ ID NO: 5, 8, 9, 12-14, 17, 18, 23-27, 29, 33, 38, 41, 42, 52, 57, 59, 60, 62, 71, 79, 114, 144 or 161, or the enzymatic activity is a DNA polymerase at the permissible temperature and the YS40 coding sequence is SEQ ID NO: 33.

The present invention also includes protein expression systems. These embodiments include a recombinant vector, as discussed immediately above, and produce the recombinant protein encoded by the vector when incubated under permissible conditions, including a permissible temperature. Protein expression systems of the present invention may be cell-based, or cell-free in nature.

A further embodiment of the present invention are vectors that include no more than about 99.9% of the nucleotide sequence of SEQ ID NO: 171 and a non-YS40 nucleotide sequence of at least 20 contiguous nucleotides. The non-YS40 nucleotide sequence is inserted into the vector sequence whereby it is flanked on the 3′ end and the 5′ end by at least 10 contiguous nucleotides of YS40 genomic sequence. The non-YS40 nucleotide sequence may optionally be operably linked to a regulatory sequence, such as a promoter. Recombinant cell systems incorporating such a vector are also contemplated as part of the present invention. Preferably the cells in such recombinant systems are Thermus thermophilus transformed with the vector.

The present invention also includes method embodiments for amplifying nucleic acids. These methods involve contacting a nucleic acid with a PCR reagent mixture including a protein of the present invention, as described above, where the protein has an enzymatic activity necessary for DNA amplification when incubated at a permissive temperature under permissible conditions. Variants on these embodiments include amplification methods where whole cells are the starting material. In these variants, the reaction mix includes at least one protein of the present invention that possesses an enzymatic activity that facilitates entry of PCR reagents into the cell. Such enzymes usually lyse the cells, but do not have to, in order to form part of the present invention.

Method embodiments for decomposing a biodegradable material are also contemplated. These methods involve contacting the biodegradable material with at least one protein of the present invention as described above, that has an enzymatic activity necessary for decomposing the biodegradable material when incubated at a permissible temperature. Exemplary enzymatic activities suitable for this purpose include, but are not limited to, amylase, cellulase, nuclease, lipase, deaminase and peptidase.

Finally, the invention also includes kit that are suitable for amplifying a nucleic acid. The kits include a reagent that has at least one protein of the present invention as described above and a buffer solution. Proteins of the invention suitable for inclusion in kit embodiments have an enzymatic activity necessary for DNA amplification or DNA entry into the cell when incubated at a permissible temperature. Kits may optionally include primers suitable for hybridization with the nucleic acid being amplified, and/or control nucleic acids and primers for quantifying the reaction.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

“Open Reading Frame”, or “ORF,” refers to a series of at least 25 contiguous codons, preferably beginning with the codon “ATG.”

“Permissible temperature” refers to a temperature at which a cell may grow and divide, or a protein is capable of retaining its tertiary structure and any innate enzyme activity, or enzymatic activity, the molecule may possess.

“Modulate” refers to the property of being able to quantitatively increase or decrease one or more chemical or physical characteristics of a molecule or process by at least 10% of the initial baseline characteristic in response to an environmental or metabolic change. Modulate may also refer to the ability to qualitatively alter a chemical or physical characteristic of a molecule or process in response to an environmental or metabolic change. Methods for determining modulation of chemical or physical characteristics of a molecule are well known in the art and include, but are not limited to, enzyme assays and spectroscopic analysis.

The terms “enzyme activity” and “enzymatic activity” are used interchangeably herein.

A reference to “displaying (an enzyme activity or enzymatic activity)” refers to a molecular characteristic where a biomolecule such as a protein or nucleic acid catalyzes a chemical reaction. Exemplary enzyme or enzymatic activities displayed by YS40 proteins include, but are not limited to, decarboxylase, nuclease, synthase, recombinase, helicase, dehydrogenase, reductase, nucleotide primase, kinase, protease, nucleotidyltransferase, nucleic acid polymerase, deaminase, acyltransferase, terminase, helicase, glycosyltransferase and peptidase.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers, those containing modified residues, and non-naturally occurring amino acid polymer.

The term “amino acid” refers to naturally occurring and synthetic amino acids, as well as amino acid analogs and amino acid mimetics that function similarly to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, e.g., an α carbon that is bound to a hydrogen, a carboxyl group, an amino group, and an R group, e.g., homoserine, norleucine, methionine sulfoxide, methionine methyl sulfonium. Such analogs may have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions similarly to a naturally occurring amino acid.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical or associated, e.g., naturally contiguous, sequences. Because of the degeneracy of the genetic code, a large number of functionally identical nucleic acids encode most proteins. For instance, the codons GCA, GCC, GCG and GCU all encode the amino acid alanine. Thus, at every position where an alanine is specified by a codon, the codon can be altered to another of the corresponding codons described without altering the encoded polypeptide. Such nucleic acid variations are “silent variations,” which are one species of conservatively modified variations. Every nucleic acid sequence herein which encodes a polypeptide also describes silent variations of the nucleic acid. One of skill in the art will recognize that in certain contexts each codon in a nucleic acid (except AUG, which is ordinarily the only codon for methionine, and TGG, which is ordinarily the only codon for tryptophan) can be modified to yield a functionally identical molecule. Accordingly, often silent variations of a nucleic acid that encodes a polypeptide is implicit in a described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing fluctionally similar amino acids are well known in the art. Such conservatively modified variants are in addition to and do not exclude polymorphic variants, interspecies homologs, and alleles of the invention. Typically conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

“Homologous,” in relation to two or more peptides, refers to two or more sequences or subsequences that have a specified percentage of amino acid residues that are the same (i.e., about 60% identity, preferably about 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site http://www.ncbi.nlm.nih.gov/BLAST/ or the like). The definition also includes sequences that have deletions and/or additions, as well as those that have substitutions, as well as naturally occurring, e.g., polymorphic or allelic variants, and man-made variants. As described below, the preferred algorithms can account for gaps and the like. Preferably, identity exists over a region that is at least about 25 amino acids in length, or more preferably over a region that is 50-100 amino acids in length. “Identical” may be used interchangably with “homologous”.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of one of the number of contiguous positions selected from the group consisting typically of from about 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

Examples of algorithms that are suitable for determining percent sequence identity and sequence similarity include the BLAST and BLAST 2.0 algorithms, which are described in Altschul et al., Nuc. Acids Res. 25:3389-3402 (1977) and Altschul et al., J. Mol. Biol. 215:403-410 (1990). BLAST and BLAST 2.0 are used, with the parameters described herein, to determine percent sequence identity for the nucleic acids and proteins of the invention. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, e.g., for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a peptide is considered similar to a reference sequence if the smallest sum probability in a comparison of the test peptide to the reference peptide is less than about 0.2, more preferably less than about 0.01, and most preferably less than about 0.001. Log values may be large negative numbers, e.g., 5, 10, 20, 30, 40, 40, 70, 90, 110, 150, 170, etc.

The terms “sequence similarity”, “sequence identity”, or “percent identity” in the context of two or more nucleic acids or polypeptide sequences, refer to two or more sequences or subsequences that are, when optimally aligned with appropriate nucleotide insertions or deletions, the same or have a specified percentage of amino acid residues or nucleotides that are the same (i.e., 50% identity, 65%, 70%, 75%, 80%, 85%, 90%, 91%, 92%, 93%, 94%, 95%, 96%, 97%, 98%, 99% or higher identity to an amino acid sequence such as SEQ ID NO:2, or a nucleotide sequence such as SEQ ID NO:1), when compared and aligned for maximum correspondence over a comparison window, or designated region as measured using one of the following sequence comparison algorithms or by manual alignment and visual inspection. This definition also refers to the compliment of a test sequence. Preferably, the identity exists over a region that is at least about 25 amino acids or nucleotides in length, or or even over a region that is about 50-100 amino acids or nucleotides in length. These relationships hold, notwithstanding evolutionary origin (Reeck et al., Cell, 50:667 (1987)). When the sequence identity of a pair of polynucleotides or polypeptides is greater or equal to 65%, the sequences are said to be “substantially identical.”

Alternatively, substantial identity will exist when a nucleic acid will hybridize under selective hybridization conditions, to a strand or its complement. Typically, selective hybridization will occur when there is at least about 55% homology over a stretch of at least about 14 nucleotides, more typically at least about 65%, 75%, 85% and or even at least about 90%. See, Kanehisa, Nuc. Acids Res., 12:203-213 (1984), which is incorporated herein by reference. The length of homology comparison, as described, may be over longer stretches, and in certain embodiments will be over a stretch of at least about 17 nucleotides, generally at least about 20 nucleotides, ordinarily at least about 24 nucleotides, usually at least about 28 nucleotides, typically at least about 32 nucleotides, more typically at least about 40 nucleotides, 50 nucleotides, and or even at least about 75 to 100 or more nucleotides.

Macromolecular structures such as polypeptide structures can be described in terms of various levels of organization. For a general discussion of this organization, see, e.g., Alberts et al., Molecular Biology of the Cell (3^{rd} ed., 1994) and Cantor and Schimmel, Biophysical Chemistry Part I. The Conformation of Biological Macromolecules (1980). “Primary structure” refers to the amino acid sequence of a particular peptide. “Secondary structure” refers to locally ordered, three-dimensional structures within a polypeptide. These structures are commonly known as domains. Domains are portions of a polypeptide that form a compact unit of the polypeptide and are typically about 5 to 350 amino acids long. Typical domains are made up of organized sections of peptide such as stretches of β strands (that can interact to form β sheets) and α helices. “Tertiary structure” refers to the complete three-dimensional structure of a polypeptide monomer. “Quaternary structure” refers to the three dimensional structure formed by the non-covalent association of independent tertiary units. A “random coil structure,” when referring to the structure of a protein or peptide indicates a lack of higher level (secondary or tertiary) structure, or a relatively disorganized structural sequence between secondary structural motifs, such as β-sheets and α-helices.

A “label” or a “detectable moiety” is a composition detectable by spectroscopic, photochemical, biochemical, immunochemical, chemical, or other physical means. For example, useful labels include fluorescent dyes, electron-dense reagents, enzymes (e.g., as commonly used in an ELISA), biotin, digoxigenin, or haptens and proteins or other entities which can be made detectable, e.g., by incorporating a radiolabel into the peptide or used to detect antibodies specifically reactive with the peptide. The radioisotope may be, for example, ³H, ¹⁴C, ³²P, ³⁵S, or ¹²⁵I. The labels may be incorporated into the antibodies at any position. Any method known in the art for conjugating the antibody to the label may be employed, including those methods described by Hunter et al., Nature, 144:945 (1962); David et al., Biochemistry, 13:1014 (1974); Pain et al., J. Immunol. Meth., 40:219 (1981); and Nygren, J. Histochem. and Cytochem., 30:407 (1982). The lifetime of radiolabeled peptides or radiolabeled antibody compositions may extended by the addition of substances that stablize the radiolabeled peptide or antibody and protect it from degradation. Any substance or combination of substances that stablize the radiolabeled peptide or antibody may be used including those substances disclosed in U.S. Pat. No. 5,961,955.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, e.g., recombinant cells express genes that are not found within the native (non-recombinant) form of the cell or express native genes that are otherwise abnormally expressed, under expressed or not expressed at all. By the term “recombinant nucleic acid” herein is meant nucleic acid, originally formed in vitro, in general, by the manipulation of nucleic acid, e.g., using polymerases and endonucleases, in a form not normally found in nature. In this manner, operably linkage of different sequences is achieved. Thus an isolated nucleic acid, in a linear form, or an expression vector formed in vitro by ligating DNA molecules that are not normally joined, are both considered recombinant for the purposes of this invention. It is understood that once a recombinant nucleic acid is made and reintroduced into a host cell or organism, it will replicate non-recombinantly, i.e., using the in vivo cellular machinery of the host cell rather than in vitro manipulations; however, such nucleic acids, once produced recombinantly, although subsequently replicated non-recombinantly, are still considered recombinant for the purposes of the invention. Similarly, a “recombinant protein” is a protein made using recombinant techniques, i.e., through the expression of a recombinant nucleic acid as depicted above.

The term “operably linked” refers to a linkage of polynucleotide elements in a functional relationship. With regard to the present invention, the term “operably linked” refers to a functional linkage between a nucleic acid expression control sequence (such as a promoter, or an array of transcription factor binding sites) and a second nucleic acid sequence, wherein the expression control sequence directs transcription of the nucleic acid corresponding to the second sequence. Thus, a nucleic acid is “operably linked” when it is placed into a functional relationship with another nucleic acid sequence.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and o-phosphoserine. “Amino acid analog” refers to compounds that have the same basic chemical structure as a naturally occurring amino acid, i.e., a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that function in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The term “amino acid sequence” refers to the positional relationship of amino acid residues as they exist in a given polypeptide or protein.

The term “coding sequence”, in relation to nucleic acid sequences, refers to a plurality of contiguous sets of three nucleotides, termed codons, each codon corresponding to an amino acid as translated by biochemical factors according to the universal genetic code, the entire sequence coding for an expressed protein, or an antisense strand that inhibits expression of a protein. A “genetic coding sequence” is a coding sequence where the contiguous codons are intermittently interrupted by non-coding intervening sequences, or “introns.” During mRNA processing intron sequences are removed, restoring the contiguous codon sequence encoding the protein or anti-sense strand.

The term “contiguous” in the context of polynucleotide or polypeptide sequences, refers to an uninterrupted sequence of bases or amino acids, each base or amino acid being immediately adjacent to its neighbors in the sequence.

The terms “expression vector” and “expression cassette” include any type of genetic construct containing a nucleic acid capable of being transcribed in a cell. The expression vectors of the invention generally supply sequence elements directing translation of the coding sequence into a protein of the present invention, as provided by the invention itself, although vectors used for the amplification of nucleotide sequences (both coding and non-coding) are also encompassed by the definition. In addition to the coding sequence, expression vectors will generally include restriction enzyme cleavage sites and the other initial, terminal and intermediate DNA sequences that are usually employed in vectors to facilitate their construction and use. The expression vector can be part of a plasmid, virus, or nucleic acid fragment.

The term “fusion gene” refers to the combination of one or more heterologous coding sequences joined in frame to form a single translational/transcriptional unit. Typically the heterologous coding sequences are joined end-to-end. The definition however includes fusion genes where one sequence, or fragment thereof, intervenes in another heterologous sequence.

The term “heterologous” when used with reference to portions of a nucleic acid or protein indicates that the molecule comprises two or more subsequences that are not found in the same relationship to each other in nature. For instance, a heterologous nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

The terms “primers” or “primer pairs” refer to oligonucleotide probes capable of recognizing and hybridizing to specific nucleotide sequences found in a target gene or sequence to be amplified by polymerase chain reaction (PCR). The degree of complementarity required between the primers and the target sequence determines the specificity, or stringency of conditions required for hybridization of the sequences. A temperature of about 36° C. is typical for low stringency amplification, although annealing temperatures may vary between about 32° C. and about 48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of about 90° C.-95° C. for 30 sec-2 min., an annealing phase lasting 30 sec.-2 min., and an extension phase of about 72° C. for 1-2 min. Protocols and guidelines for low and high stringency amplification reactions are provided, e.g., in Innis et al., PCR Protocols, A Guide to Methods and Applications, Academic Press, Inc. N.Y. (1990)).

The term “Regulatory sequences” refers to those sequences, both 5′ and 3′ to a structural gene, that are required for the transcription and translation of the structural gene in the target host organism. Regulatory sequences include a promoter, ribosome binding site, optional inducible elements and sequence elements required for efficient 3′ processing, including polyadenylation. When the structural gene has been isolated from genomic DNA, regulatory sequences also include those intronic sequences required to remove of the introns as part of mRNA formation in the target host.

The term “recombinant” when used with reference, e.g., to a cell, or nucleic acid, protein, or vector, indicates that the cell, nucleic acid, protein or vector, has been modified by the introduction of a heterologous nucleic acid or protein, or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under-expressed or not expressed at all.

DNA regions are “operably linked” when they are functionally related to each other. For example, DNA for a signal peptide (secretory leader) is operably linked to DNA for a polypeptide if it is expressed as a precursor which participates in the secretion of the polypeptide; a promoter is operably linked to a coding sequence if it controls the transcription of the sequence; or a ribosome-binding site is operably linked to a coding sequence if it is positioned so as to permit translation. Generally, operably linked means contiguous and in reading frame.

### BRIEF DESCRIPTION OF DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 shows single and multiround transcription at the T7A1-tR′ and galP-tR′ promoters. Two transcriptionally competent open model promoters from different classes, −10/−35 class (T7A1) and extended—10 class (galP1), attached to a rho-independent terminator were used to qualitatively visualize efficiency of run-off transcription. Reaction 1, in 20 μl of transcription buffer (30 mM tris-HCl, pH 8.0, 10 mM MgC12, 40 mM KCl, 1 mM β-mercaptoethanol), contained core enzyme, sigma (σ) and Ys18. Reaction 1 was incubated at 65° C. (for T. th) and 37° C. (for E. coli) for 10 minutes, followed by the addition of promoter DNA fragments. Reaction 2, in 10 μl of transcription buffer, contained core enzyme and sigma, and, in parallel, Ys18 and a promoter DNA fragment. Reaction 2 was incubated at 65° C. (for T. th) and 37° C. (for E. coli) for 10 minutes and then mixed together. For both reactions 1 and 2, after 10 minutes of incubation at the same temperatures, 200 μM ATP, CTP, UTP, 20 μM GTP and 10 μCi of [α-³²P] GTP were added, the reactions were incubated for the next 10 minutes, and terminated by an equal volume of 9 M urea loading buffer. In E. coli, if during the transcription reaction heparin is not added with the nucleotides, the RNA polymerase is able to initiate transcription from the promoter repeatedly (multi-round transcription). If heparin is added, the RNA polymerase is only able to transcribe once (single-round transcription). The open complexes formed by T. th RNA polymerase at the promoters used are very sensitive to heparin so single-round transcription cannot be performed.

FIG. 1A shows multi-round transcriptional inhibition by Ys18 of both T7A1-tR′ and galP-tR′ with the T. th RNA polymerase core and σ^{A}. With both promoters, increasing amounts of Ys18 (triangle) repressed transcription in a dose dependent manner (run-off bands) when the components were mixed as in Reaction 1 (order 1). When the components were mixed as in Reaction 2, Ys18 did not significantly affect transcription of promoter T7A1-tR′, but repressed transcription of promoter galP-tR′. The similar change in band intensity of run-off bands and terminator tR′ bands indicated that Ys18 did not affect elongation or termination.

FIG. 1B shows single-round (+heparin) and multi-round (−heparin) transcriptional inhibition by Ys18 of both T7A1-tR′ and galP-tR′ with the E. coli RNA polymerase core and σ⁷⁰. With both promoters, increasing amounts of Ys18 (triangle) repressed transcription in a dose dependent manner (run-off bands). In the presence of heparin, when the components were mixed as in Reaction 1 (order 1), Ys18 was not as active in repressing transcription as when the components were mixed as in Reaction 2 (order2). In the absence of heparin, Ys18 was more active in repressing transcription when the components were mixed as in Reaction 2 (order 2) with the T7A1-tR′ promoter. There was only a slight difference in repressing activity of Ys18 in the absence of heparin between the two reaction mixtures. The similar change in band intensity of run-off bands and terminator tR′ bands indicated that Ys18 did not affect elongation or termination.

FIG. 1C shows the relative transcriptional activity of the run-off assay in graphical form. Transcriptional activity without Ys18 present is 100% (dark bar). The addition of Ys18 in incremental amounts represses transcriptional activity in a dose dependent manner (light bar). The amount of transcriptional repression by Ys18 presences differs with amount of Ys18 added, promoter type, RNA polymerase core, reaction mixture and presence or absence of heparin.

FIG. 2 shows native binding experiments with histidine-tagged phage protein Ys18 and primary sigma factors from T. thermophilus and E. coli. Reactions, containing corresponding proteins in 20 μl of binding buffer (20 mM tris HCl, pH8.0, 0.5 M NaCl, 2 mM imidazole, 5% v/v glycerol), were preincubated for 10′ at 65° C. (for T. th σ^{A}) and 37° C. (E. coli σ⁷⁰). The binding mixtures were then added to Ni—NTA agarose beads equilibrated in the binding buffer. Reactions were incubated for 10′ at room temperature. The agarose beads were pelleted by quick centrifugation and the unbound proteins were withdrawn. The beads were washed 3 times with the binding buffer containing 20 mM imidazole, and the bound proteins were eluted with the binding buffer containing 200 mM imidazole. Fractions were resolved by SDS-PAGE and stained by Coomassie (L=proteins loaded, U=proteins unbound, B=proteins bound to Ni—NTA agarose).

FIG. 2A shows Ys18_{HIS} bound to the primary sigma factor from T. thermophilus (σ^{A}). With both Ys18_{HIS} and σ^{A} present in the sample (+lane, L), σ^{A} was detected in the unbound (+lane, U) and the bound fractions (+lane, B). In the absence of Ys18_{HIS} (−lane, L), σ^{A} was exclusively observed in the unbound fraction (−lane, U). σ^{A} cannot bind to the Ni—NTA agarose beads without Ys18_{HIS}, indicating Ys18_{HIS} was capable of binding to σ^{A}.

FIG. 2B shows Ys18_{HIS} bound to the primary sigma factor from E. coli (σ⁷⁰). When both Ys18_{HIS} and σ⁷⁰ were present in the sample (+lane, L), σ⁷⁰ was detected in the unbound (+lane, U) and the bound fractions (+lane, B). In the absence of Ys18_{HIS} (−lane, L), σ⁷⁰ was exclusively observed in the unbound fraction (−lane, U). σ⁷⁰ cannot bind to the Ni—NTA agarose beads without Ys18_{HIS}, indicating Ys18_{HIS} was capable of binding to σ⁷⁰.

FIG. 2C shows Ys18_{HIS} bound to the primary sigma factor from E. coli lacking region 4 (σ⁷⁰₁₋₅₄₉). When both Ys18_{HIS} and σ⁷⁰₁₋₅₄₉ were present in the sample (+lane, L), σ⁷⁰₁₋₅₄₉ was detected in the unbound (+lane, U) and the bound fractions (+lane, B). In the absence of Ys18_{HIS} (−lane, L), σ⁷⁰₁₋₅₄₉ was exclusively observed in the unbound fraction (−lane, U). σ⁷⁰₁₋₅₄₉ cannot bind to the Ni—NTA agarose beads without Ys18_{HIS}, indicating Ys18_{HIS} was capable of binding to σ⁷⁰₁₋₅₄₉ in a region other than region 4.

### DETAILED DESCRIPTION

I. Introduction

The present invention provides novel proteins from the bacteriophage YS40 . These novel proteins retain their functionality at mesophilic or thermophilic temperatures, and consequently allow biosynthetic and/or biodegradative processes to proceed at higher temperatures.

The YS40 bacteriophage infects Thermus thermophilus HB8, and grows over the temperature range of about 56 to about 78° C. The bacteriophage has a large genome (165 Kbp, ˜150 genes) containing multiple DNA polymerase genes. The phage reproduces above 70° C., and the thermophilic enzymes have an extrinsic structural stability. Most of the YS40 proteins have a strong similarity to prokaryotic enzymes, including the length of their amino acid sequences, and the potential to encode most of the proteins required for its own replisome.

YS40 encodes its own A-type DNA polymerase (encoded by SEQ ID NO: 134), which has a conserved region in its C-terminus including 3 motifs with invariant residues ranging from amino acid residues 825-1102. Like the Klenlow fragment from E. coli DNA pol I, the YS40 A-type DNA polymerase has no N-terminal 5′-3′ exonuclease domain. Other proteins encoded by YS40 include gp166 (encoded by SEQ ID NO: 166), which is similar to podovirus phi 29 terminal protein. gp166 may be involved in protein-primed DNA replication of the linear phi 29 genome (linked to 5′ ends of both strands via phospodiester bonds). gp106 (encoded by SEQ ID NO: 106) is an S-adenosylmethionine decarboxylase (key enzyme in biosynthesis of spermidine and spermine.).

Thus, the molecules of this invention may find utility in a wide variety of applications including, but not limited to, synthetic nucleic acid synthesis, biodegradative processes and other applications requiring resilient molecules capable of retaining their integrity, including enzyme activity when present, at higher temperatures such as least about 36° C., or even at least about 45° C., 55° C., 65° C., or even about 75° C. The following sections detail embodiments of the present invention, and how they may be used in biometabolic reactions.

II. Identifying Open Reading Frames

Nucleic acids encoding proteins and peptides of the present invention may be identified by screening the YS40 genomic sequence for open reading frames (ORFs) using any method known in the art. Using these methods, nucleic acid coding sequences for proteins of the present invention, as found in wild-type and cultured bacteriophage YS40 strains, may be identified. These coding sequences and/or proteins may be further modified as described herein, to provide additional coding sequences of the invention.

By way of example, the genome sequence of YS40 may be searched for ORFs using the hidden Markov model approach implemented in GeneMark program (See Besemer J. and Borodovsky M (1999), NAR, Vol. 27, No. 19, pp. 3911-3920). Using this technique, 170 open reading frames (ORFs) encoding preferred proteins of the present invention are predicted, as identified in Table 1, below:

TABLE 1
Gene products of phage YS40 and their predicted molecular functions

| | | | The best | | |
|---|---|---|---|---|---|
| | | | database | | |
| ORF | | | match with | Taxonomic | |
| (SEQ | | ORF | validated | origin of the best | Functional annotation/ |
| ID NO.) | ORF strand/position^{a} | length (aa) | similarity | match | characteristics^{b} |
| 1 | —/(7 . . . 1938) | 643 | 34419532 | phage KVP40 | distal tail fiber protein |
| 2 | —/(1941 . . . 4586) | 881 | | | |
| 3 | —/(4573 . . . 7410) | 945 | 48696430 | phage K | portal protein |
| 4 | —/(7412 . . . 8068) | 218 | | | TM, |
| 5 | —/(8096 . . . 8530) | 144 | 19924248 | Methanocaldococcus | S-adenosylmethionine |
| | | | | jannaschii | decarboxylase |
| | | | | | (adoMetDC) |
| 6 | —/(8564 . . . 8788) | 74 | | | |
| 7 | —/(8801 . . . 9412) | 203 | | | |
| 8 | —/(9399 . . . 9941) | 180 | 9631083 | Lymantria dispar | dUTPase |
| | | | | nucleopolyhedro | |
| | | | | virus | |
| 9 | —/(9955 . . . 10782) | 275 | 33357605 | Thermotoga | flavin-dependent |
| | | | | maritima | thymidylate synthase |
| 10 | —/(10816 . . . 11331) | 171 | | | |
| 11 | —/(11310 . . . 11783) | 157 | | | |
| 12 | —/(11776 . . . 12795) | 339 | 23029929 | Microbulbifer | RecA/RadA recombinase |
| | | | | degradans | |
| 13 | —/(12792 . . . 13367) | 191 | 46200225 | Thermus | putative recombination |
| | | | | thermophilus | protein, ERF |
| | | | | HB27 | |
| 14 | —/(13413 . . . 14756) | 447 | 22978288 | Ralstonia | DNA helicase DnaB |
| | | | | metallidurans | |
| 15 | —/(14743 . . . 15036) | 97 | | | |
| 16 | 15124 . . . 15453 | 109 | | | |
| 17 | 15467 . . . 16576 | 369 | 23029305 | Microbulbifer | IMP dehydrogenase/GMP |
| | | | | degradans | reductase |
| 18 | 16640 . . . 17050 | 136 | 23110678 | Novosphingobium | DNA binding HTH- |
| | | | | aromaticivorans | domain protein, |
| | | | | | transcription regulator |
| 19 | —/(17108 . . . 18343) | 411 | | | |
| 20 | —/(18400 . . . 18837) | 145 | | | |
| 21 | —/(18834 . . . 19214) | 126 | | | |
| 22 | —/(19187 . . . 19960) | 257 | | | |
| 23 | —/(19944 . . . 21620) | 558 | 27262500 | Heliobacillus | DNA primase bacterial |
| | | | | mobilis | DnaG type |
| 24 | —/(21669 . . . 22277) | 202 | 37526389 | Photorhabdus | thymidine kinase |
| | | | | luminescens | |
| 25 | —/(22302 . . . 23015) | 237 | 15595102 | Borrelia | ClpP protease |
| | | | | burgdorferi | |
| 26 | —/(22975 . . . 23901) | 308 | 9964625 | phage SIO1 | RecB family exonuclease |
| 27 | —/(23898 . . . 25247) | 449 | 15900485 | Streptococcus | DEAD domain helicase |
| | | | | pneumoniae | |
| 28 | 25396 . . . 26796 | 466 | | | |
| 29 | 26822 . . . 27331 | 169 | 33864277 | Prochlorococcus | nucleotidyltransferase |
| | | | | marinus | |
| 30 | —/(27328 . . . 29085) | 585 | | | |
| 31 | —/(29090 . . . 29803) | 237 | | | |
| 32 | —/(29818 . . . 30291) | 157 | | | |
| 33 | 30387 . . . 32498 | 703 | 29348669 | Bacteroides | DNA polymerase, without |
| | | | | thetaiotaomicron | N-terminal 5-3 |
| | | | | | exonuclease domain |
| 34 | —/(32491 . . . 32781) | 96 | | | 3 TMs |
| 35 | —/(32768 . . . 33034) | 88 | | | 2 TMs |
| 36 | —/(33031 . . . 33309) | 92 | | | |
| 37 | 33381 . . . 33746 | 121 | | | |
| 38 | 33730 . . . 34158 | 142 | 21229604 | Xanthomonas | deoxycytidylate deaminase |
| | | | | campestris | |
| 39 | 34188 . . . 34616 | 142 | | | |
| 40 | 34631 . . . 35155 | 174 | | | |
| 41 | 35201 . . . 37594 | 797 | 23104360 | Azotobacter | ribonucleotide reductase, |
| | | | | vinelandii | alpha subunit, the N- |
| | | | | | terminus |
| 42 | 37607 . . . 38206 | 199 | 20808702 | Thermoanaerobacter | ribonucleotide reductase, |
| | | | | tengcongensis | alpha subunit, the C- |
| | | | | | terminus |
| 43 | 38240 . . . 38446 | 68 | | | |
| 44 | 38459 . . . 38911 | 150 | | | |
| 45 | 38898 . . . 39227 | 109 | | | |
| 46 | 39224 . . . 39439 | 71 | | | |
| 47 | 39441 . . . 39884 | 147 | | | 4 TMs |
| 48 | 39877 . . . 40185 | 102 | | | |
| 49 | 40201 . . . 40548 | 115 | | | |
| 50 | 40558 . . . 41013 | 151 | | | |
| 51 | 41010 . . . 42482 | 490 | | | |
| 52 | 42536 . . . 43408 | 290 | 45914890 | Mesorhizobium | UDP-3-O-[3-hydroxy- |
| | | | | sp. BNC1 | myristory] glucosamine N- |
| | | | | | acyltransferase |
| 53 | 43411 . . . 43938 | 175 | | | |
| 54 | 43940 . . . 44425 | 161 | | | |
| 55 | —/(44426 . . . 45127) | 233 | 23055325 | Geobacter | |
| | | | | metallireducens | |
| 56 | 45187 . . . 46209 | 340 | 51891857 | Symbiobacterium | conserved bacterial protein |
| | | | | thermophilum | |
| 57 | 46199 . . . 47536 | 445 | 42521856 | Bdellovibrio | spore cortex synthesis |
| | | | | bacteriovorus | protein SpoVR |
| 58 | 47564 . . . 49414 | 616 | | | |
| 59 | 49453 . . . 51312 | 619 | 23112542 | Desulfitobacterium | serine kinase |
| | | | | hafniense | |
| 60 | 51410 . . . 51997 | 195 | 29366771 | phage phi-BT1 | dNMP kinase |
| 61 | 52035 . . . 52484 | 149 | | | |
| 62 | —/(52477 . . . 54345) | 622 | 15668504 | Methanocaldococcus | terminase large subunit |
| | | | | jannaschii | |
| 63 | —/(54320 . . . 55108) | 262 | | | |
| 64 | —/(55105 . . . 55485) | 126 | | | |
| 65 | —/(55466 . . . 56017) | 183 | 22855150 | phage B103 | terminal protein |
| 66 | 56049 . . . 56315 | 88 | | | |
| 67 | —/(56362 . . . 57102) | 246 | | | |
| 68 | —/(57104 . . . 57754) | 216 | | | |
| 69 | —/(57775 . . . 59721) | 648 | 22973075 | Chloroflexus | tail sheath protein |
| | | | | aurantiacus | |
| 70 | —/(59782 . . . 60492) | 236 | | | |
| 71 | —/(60495 . . . 61157) | 220 | 48696435 | phage K | Zn ribbon, similar to |
| | | | | | archaeal transcription |
| | | | | | factor IIB |
| 72 | —/(61167 . . . 61682) | 171 | | | |
| 73 | —/(61756 . . . 63168) | 470 | | | |
| 74 | —/(63204 . . . 64838) | 544 | 48696431 | phage K | 3 coiled coil regions |
| 75 | —/(64838 . . . 65098) | 86 | | | |
| 76 | —/(65085 . . . 69662) | 1525 | | | |
| 77 | —/(69684 . . . 74918) | 1744 | | | 3 coiled coil regions, |
| | | | | | unknown |
| 78 | —/(74931 . . . 75296) | 121 | | | |
| 79 | —/(75309 . . . 79883) | 1524 | 40744644 | Aspergillus | DEAD domain helicase |
| | | | | nidulans | |
| 80 | —/(79880 . . . 80743 | 287 | | | |
| 81 | —/(80788 . . . 82740) | 650 | | | |
| 82 | —/(82771 . . . 84609) | 612 | | | |
| 83 | —/(84867 . . . 85094) | 75 | | | |
| 84 | —/(85328 . . . 85558) | 76 | | | |
| 85 | —/(85767 . . . 85919) | 50 | | | |
| 86 | —/(86022 . . . 86273) | 83 | | | |
| 87 | —/(86382 . . . 86618) | 78 | | | |
| 88 | —/(86909 . . . 87154) | 81 | | | |
| 89 | —/(87505 . . . 87990) | 161 | 15805515 | Deinococcus | 2 TMs |
| | | | | radiodurans | |
| 90 | —/(88074 . . . 88529) | 151 | | | |
| 91 | —/(88642 . . . 89250) | 202 | | | |
| 92 | —/(89349 . . . 89783) | 144 | | | |
| 93 | —/(89796 . . . 90221) | 141 | | | |
| 94 | —/(90481 . . . 90927) | 148 | | | |
| 95 | —/(91036 . . . 91212) | 58 | | | |
| 96 | 91231 . . . 91359 | 43 | | | |
| 97 | —/(91417 . . . 91824) | 135 | | | 3 TMs |
| 98 | —/(91835 . . . 92380) | 181 | | | |
| 99 | —/(92503 . . . 93045) | 180 | | | |
| 100 | —/(93045 . . . 93635) | 196 | | | |
| 101 | —/(93619 . . . 94131) | 170 | | | |
| 102 | —/(94337 . . . 94873) | 178 | | | |
| 103 | —/(94885 . . . 95373) | 162 | | | |
| 104 | —/(95510 . . . 96025) | 171 | | | |
| 105 | —/(96096 . . . 96626) | 176 | | | |
| 106 | —/(96833 . . . 97354) | 173 | | | |
| 107 | —/(97575 . . . 99263) | 562 | | | |
| 108 | —/(99280 . . . 100323) | 347 | 15643692 | Thermotoga | ATPase |
| | | | | maritima | |
| 109 | —/(100462 . . . 101157) | 231 | | | |
| 110 | —/(101227 . . . 101973) | 248 | | | |
| 111 | —/(102138 . . . 102530) | 130 | | | |
| 112 | —/(102531 . . . 103076) | 181 | | | |
| 113 | —/(103077 . . . 103616) | 179 | | | |
| 114 | —/(103616 . . . 104107) | 163 | 11992695 | Escherichia coli | glycosyltransferase |
| 115 | —/(104451 . . . 104693) | 80 | | | |
| 116 | —/(104803 . . . 105279) | 158 | | | |
| 117 | —/(105422 . . . 105979) | 185 | | | |
| 118 | —/(105969 . . . 106520) | 183 | | | |
| 119 | —/(106510 . . . 107076) | 188 | | | |
| 120 | —/(107090 . . . 107539) | 149 | | | |
| 121 | —/(107552 . . . 108046) | 164 | | | |
| 122 | —/(108141 . . . 108644) | 167 | | | |
| 123 | —/(108772 . . . 109290) | 172 | | | |
| 124 | —/(109328 . . . 109819) | 163 | | | |
| 125 | —/(109998 . . . 110513) | 171 | 18462664 | Shigella flexneri | |
| 126 | —/(110561 . . . 111145) | 194 | | | |
| 127 | —/(111157 . . . 111654) | 165 | | | |
| 128 | —/(111663 . . . 112133) | 156 | | | |
| 129 | —/(112165 . . . 112677) | 170 | | | |
| 130 | —/(112689 . . . 113195) | 168 | | | |
| 131 | —/(113202 . . . 113630) | 142 | | | |
| 132 | 113852 . . . 114388 | 178 | | | coiled coil |
| 133 | —/(114385 . . . 115032) | 215 | | | |
| 134 | —/(115155 . . . 115724) | 189 | | | |
| 135 | —/(115727 . . . 116299) | 190 | | | |
| 136 | —/(116271 . . . 116693) | 140 | | | |
| 137 | 116815 . . . 117474 | 219 | | | |
| 138 | —/(117442 . . . 118005) | 187 | | | |
| 139 | —/(118395 . . . 119999) | 534 | 19552983 | Corynebacterium | |
| | | | | glutamicum | |
| 140 | —/(120226 . . . 120777) | 183 | | | |
| 141 | 120821 . . . 120994 | 58 | | | |
| 142 | —/(120953 . . . 123997) | 1014 | | | coiled coil |
| 143 | —/(124012 . . . 124536) | 174 | | | |
| 144 | —/(124553 . . . 125593) | 346 | 10956653 | Rhodococcus | M27/M37 peptidase |
| | | | | equi | |
| 145 | —/(125598 . . . 126548) | 316 | | | |
| 146 | —/(126553 . . . 126813) | 86 | | | 3 TMs |
| 147 | 126870 . . . 127055 | 61 | | | |
| 148 | 127065 . . . 127460 | 131 | | | |
| 149 | 127471 . . . 127959 | 162 | | | |
| 150 | 127979 . . . 129967 | 662 | 34762157 | Fusobacterium | baseplate assembly protein |
| | | | | nucleatum | |
| 151 | 129964 . . . 131859 | 631 | | | |
| 152 | 131870 . . . 134260 | 796 | 903862 | phage K3 | wac fibritin neck whisker |
| 153 | 134253 . . . 136364 | 703 | | | |
| 154 | 136388 . . . 137287 | 299 | | | |
| 155 | 137294 . . . 137644 | 116 | | | |
| 156 | 137634 . . . 138497 | 287 | | | |
| 157 | 138469 . . . 139269 | 266 | | | |
| 158 | 139253 . . . 143296 | 1347 | | | |
| 159 | 143322 . . . 143846 | 174 | | | |
| 160 | 144155 . . . 144367 | 70 | | | |
| 161 | —/(144357 . . . 145424) | 355 | 15674141 | Lactococcus | Radical SAM superfamily |
| | | | | lactis | enzyme |
| 162 | —/(145421 . . . 146374) | 317 | | | |
| 163 | —/(146390 . . . 147022) | 210 | | | |
| 164 | 147094 . . . 147639 | 181 | | | |
| 165 | 147677 . . . 148306 | 209 | | | |
| 166 | 148300 . . . 148689 | 129 | | | |
| 167 | 148736 . . . 150229 | 497 | | | |
| 168 | 150256 . . . 151341 | 361 | | | |
| 169 | 151338 . . . 151907 | 189 | | | |
| 170 | 151894 . . . 152157 | 87 | | | |

^{a}position of the ORFs in the phage YS40 genome; “—” indicates a leftwards transcription orientation.
^{b}presence of transmembrane domains (TM) and coiled coil regions are indicated.

Regions between the identified ORFs may be screened for additional genes using the Blastx and tBlastx programs (Schafer et al., 1997), and identified ORF sequences compared with sequences in available databases (e.g., GenBank, GenPept, and the database of unfinished microbial genomes at NCBI) to provide a putative activity or function to the protein encoded by the ORF.

III. YS40 Proteins

Once identified, ORFs may be used in expression systems to produce YS40 proteins of the present invention, or the proteins may be isolated from cultures of Thermus thermophilus infected with bacteriophage YS40 . Alternatively, proteins and peptides of the present invention may be synthesized using solid or liquid phase techniques well known to those of skill in the art. These proteins include a thermophilic amino acid sequence at least 75%, 80%, 85%, 90%, 95%, 96%, 97%, 98%, or even at least 99% homologous to a YS40 amino acid sequence encoded by at least about 25, 35, 45, 50, 65 75 85, 95 or even about 100 contiguous codons of a YS40 coding sequence selected from SEQ ID NO: 1-170. In certain instances, the YS40 coding sequence is selected from SEQ ID NO: 2, 4-64, 70-149, 151 and 153-170 and has an enzyme activity that is a decarboxylase, nuclease, synthase, recombinase, helicase, dehydrogenase, reductase, nucleotide primase, kinase, protease, nucleotidyltransferase, nucleic acid polymerase, deaminase, acyltransferase, terminase, helicase, glycosyltransferase or a peptidase that is active at a permissible temperature of about 36° C., or even about 45° C., 55° C. or 65° C., or is active at about 75° C. Activity at about 75° C. is preferred but not requisite. Alternatively, the YS40 coding sequence may be from SEQ ID NO: 5, 8, 9, 12-14, 17, 18, 23-27, 29, 33, 38, 41, 42, 52, 57, 59, 60, 62, 71, 79, 114, 144 or 161. In certain instances, the YS40 coding sequence is from SEQ ID NO: 33, and is a DNA polymerase.

IV. Purifying Proteins

As noted above, certain proteins of the present invention may be isolated from cultures of Thermus thermophilus infected with bacteriophage YS40 . Proteins of the invention isolated in this manner will typically be encoded by the ORFs SEQ ID NOs: 1-170, more typically by SEQ ID NO: 2, 4-64, 70-149, 151 or 153-170, even more typically by SEQ ID NO: 5, 8, 9, 12-14, 17, 18, 23-27, 29, 33, 38, 41, 42, 52, 57, 59, 60, 62, 71, 79, 114, 144 or 161, and most typically by SEQ ID NO: 33. In certain embodiments of the invention, the proteins contemplated are fragments of one or more of those proteins described above.

Briefly, YS40 proteins of the present invention may be obtained by growing cultures of Thermus thermophilus infected with bacteriophage YS40 at a permissible temperature using media and techniques well known to those of skill in the art. During culture, preferably in the exponential growth phase of the bacteria, the culture is fractionated by separating the bacteria from the culture media using, for example, low speed centrifugation. If a lytic strain of YS40 is used, then the proteins of the invention may be harvested from the supernatant. If a non-lytic strain of YS40 is used, then the proteins of the invention may be harvested from the bacterial cells after lysis using, for example, a french press or other method well known in the art. Whichever approach is used, proteins of the invention may be further purified using any combination of a variety of techniques well known to those of skill in the art. (cf., Colley et al., J. Biol. Chem., 264:17619-17622 (1989), and Guide to Protein Purification, in Vol. 182 of Methods in Enzymology (Deutscher ed., 1990), Morrison, D. A., J. Bact., 132:349-351 (1977), or by Clark-Curtiss et al., Methods in Enzymology, 101:347-362 (1983), eds. R. Wu et al., Academic Press, New York. (for suitable media, see the catalogues of the American Type Culture Collection)). Additional isolation techniques are described in detail in the following sections.

Proteins and peptides of the present invention may be purified to substantial purity by standard techniques, including column chromatography, immunopurification methods, electrophoresis, centrifugation, crystallization, isoelectric focusing and others (see, e.g., Scopes, Protein Purification: Principles and Practice (1982); Ausubel, et al. (1987 and periodic supplements) Current Protocols in Molecular Biology; Deutscher (1990) “Guide to Protein Purification” in Methods in Enzymology vol. 182, and other volumes in this series; and manufacturers' literature on use of protein purification products, e.g., Pharmacia, Piscataway, N.J., or Bio-Rad, Richmond, Calif.; and Sambrook et al., supra).

Standard Purification Techniques

Gel Filtration

The individual molecular weights of proteins of the present invention may be used to isolate it from proteins of greater and lesser size by, for example, using ultrafiltration through membranes of different pore size (for example, Amicon or Millipore membranes). As a first step, the protein mixture is ultrafiltered through a membrane with a pore size that has a lower molecular weight cut-off than the molecular weight of the protein of interest. The retentate of the ultrafiltration is then ultrafiltered against a membrane with a molecular cut-off greater than the molecular weight of the protein of interest. The recombinant protein will pass through the membrane into the filtrate. The filtrate may then be chromatographed as described below.

Exchange Chromatography

Proteins of the present invention may also be separated from other proteins on the basis of size, net surface charge, hydrophobicity, and affinity for ligands. In addition, antibodies raised against proteins may be conjugated to column matrices and the proteins immunopurified. All of these methods are well known in the art. It will be apparent to one of skill that chromatographic techniques may be performed at any scale and using equipment from many different manufacturers (e.g., Pharmacia Biotech).

Tagging Techniques

Purification segments, or “affinity tags” may be fused to appropriate portions of proteins of the present invention to assist in isolation and production. For example, the FLAG sequence, or a functional equivalent, may be fused to the protein via a protease-removable sequence, allowing the FLAG sequence to be recognized by an affinity reagent, and the purified protein subjected to protease digestion to remove the extension. Many other equivalent segments exist, e.g., poly-histidine segments possessing affinity for heavy metal column reagents. See, e.g., Hochuli, Chemische Industrie, 12:69-70 (1989); Hochuli, Genetic Engineering, Principle and Methods, 12:87-98 (1990), Plenum Press, N.Y.; and Crowe, et al. (1992) OIAexpress: The High Level Expression & Protein Purification System, QIAGEN, Inc. Chatsworth, Calif.; which are incorporated herein by reference.

Affinity tags may also be incorporated into protein constructs of the present invention as analytical tools. Affinity tags provide a convenient way of removing the protein construct from a sample at a desired time, or to detect the location of the protein construct in a sample. Many other applications of affinity tagged protein constructs will be readily apparent to one of skill in the art.

His-Tag

Protein constructs of the present invention may also contain a string of histidine residues, incorporated at the amino or carboxyl terminal of the novel protein. The polyhistidine tag allows convenient isolation of the protein in a single step by nickel-chelate chromatography. When a protein that has been “his-tagged” is placed on the nickel column, the histidine residues form a chelate complex with the nickel bound to the column, immobilizing the tagged protein. Contaminating components of the solution comprising the tagged protein may be washed away prior to elution of the tagged protein with a suitable competing chelator, typically imidazole.

The polyhistidine tag may be added to the protein through the use of peptide linkers as described in detail below. Alternatively, the tag may be linked to a protein by appending a nucleic acid encoding the tag onto the coding region of recombinant protein, the resulting construct being incorporated into a suitable expression vector that is subsequently used to transform an appropriate host cell. Protein produced in the transformed host cell may then be purified as noted above.

Epitope Tagging

Epitope tags are another useful sequence that may be included in a protein construct of the present invention. The epitope tag may consist of an amino acid sequence that allows affinity purification of the activated protein (e.g., on immunoaffinity or chelating matrices). Thus, by including an epitope tag on the activation construct, all of the activated proteins from an activation library may be purified. By purifying the activated proteins away from other cellular and media proteins, screening for novel proteins and enzyme activities may be facilitated. In some instances, it may be desirable to remove the epitope tag following purification of the activated protein. This removal may be accomplished by including a protease recognition sequence (e.g., Factor IIa or enterokinase cleavage site) downstream from the epitope tag on the activation construct. Incubation of the purified, activated protein(s) with the appropriate protease will release the epitope tag from the proteins(s).

In libraries in which an epitope tag sequence is located in the protein construct, all of the tagged proteins may be purified away from all other cellular and media components using affinity purification. In addition to purifying the tagged protein, this method also concentrates the protein sample.

V. Recombinant Expression

A preferred method of producing proteins of the present invention is through recombinant expression of the proteins in a heterologous host system. Such systems are preferably cellular in nature, but may be cell-free. Preferable cell-based systems include bacterial hosts, most preferably E. coli hosts. As described below, nucleic acids encoding proteins of the present invention are typically inserted into an expression vector suitable for the chosen host, with the coding sequence of the nucleic acid aligned in-frame and operably linked to suitable control sequences such as a promoter and a transcriptional terminator. The expression vector is then inserted into the host cell, which is then cultured under conditions that allow for the expression of the protein of the invention. After protein expression, the protein is preferably purified using techniques such as the examples provided below.

Proteins expressed in bacteria may form insoluble aggregates (“inclusion bodies”). Several protocols are suitable for purification of Recombinant proteins from inclusion bodies. For example, purification of inclusion bodies typically involves the extraction, separation and/or purification of inclusion bodies by disruption of bacterial cells, e.g., by incubation in a buffer of 50 mM Tris/HCl pH 7.5, 50 mM NaCl, 5 mM MgCl₂, 1 mM DTT, 0.1 mM ATP, and 1 mM PMSF. The cell suspension can be lysed using 2-3 passages through a French Press, homogenized using a Polytron (Brinkman Instruments) or sonicated on ice. Alternate methods of lysing bacteria are apparent to those of skill in the art (see, e.g., Sambrook et al., supra; Ausubel et al., supra).

Thus the present invention contemplates a recombinant cell or other expression system including an isolated nucleic acid that contains a YS40 nucleotide sequence having the nucleotide sequence SEQ ID NO: 1-170. The YS40 nucleotide sequence encodes either a YS40 structural protein that does not take a random coil structure at a permissible temperature of at least 36° C., or a YS40 enzyme that displays decarboxylase, nuclease, synthase, recombinase, helicase, dehydrogenase, reductase, nucleotide primase, kinase, protease, nucleotidyltransferase, nucleic acid polymerase, deaminase, acyltransferase, terminase, helicase, glycosyltransferase or peptidase activity at a permissible temperature of at least 36° C. In certain embodiments the YS40 nucleotide sequence is operably linked to a regulatory element, preferably a promoter, most preferably a constitutive promoter.

Other embodiments of the invention include a recombinant vector comprising an isolated nucleic acid encoding an isolated protein comprising a thermophilic amino acid sequence at least about 75% homologous to a YS40 amino acid sequence encoded by at least about 25 contiguous codons from SEQ ID NO: 1-170. The YS40 amino acid sequence is operably linked to a promoter such that introduction of the vector into an expression system produces a protein having, an enzyme activity selected from the group consisting of decarboxylase, nuclease, synthase, recombinase, helicase, dehydrogenase, reductase, nucleotide primase, kinase, protease, nucleotidyltransferase, nucleic acid polymerase, deaminase, acyltransferase, terminase, helicase, glycosyltransferase and peptidase when assayed at a permissible temperature of at least about 36° C. more typically at least about 45° C., 55° C., or 65° C. and most typically at least about 75° C.

VI. Preparation of Nucleic Acids Encoding YS40 Proteins

Several embodiments of the present invention utilize nucleic acids encoding proteins of the present invention in the production of the proteins. These nucleic acids may be any coding sequence capable of expressing a protein of the present invention, when operably linked to appropriate control sequences, including a promoter. Thus, so long as a protein expressed from the nucleic acid is a protein of the invention described herein, the nucleic acid may include a partial deletion, substitution or insertion of the nucleotide sequence, or may have other nucleotide sequence ligated therewith at the 5′-terminus and/or 3′ terminus thereof.

In general, nucleic acid sequences encoding proteins of the present invention may be isolated from Thermus thermophilus strains infected with bacteriophage YS40 , or may be isolated from phage libraries constructed from the YS40 bacteriophage genome using methods well known by those of skill in the art. Generally, cDNA or genomic libraries are constructed and screened to identify the correct sequence. (For cDNA libraries, see e.g., Gubler & Hoffman, Gene, 25:263-269 (1983); Sambrook et al., 2001, Molecular Cloning: A Laboratory Manual (3^{rd} ed.); Cold Spring Harbor Laboratory, Cold Spring Harbor, N.Y.; Ausubel et al. (eds.), 1993, Current Protocols in Molecular Biology, John Wiley & Sons, NY. For genomic libraries, see Benton & Davis, Science, 196:180-182 (1977); Grunstein et al., Proc. Natl. Acad Sci. USA, 72:3961-3965 (1975); and Gussow, D. and Clackson, T., Nucl. Acids Res., 17:4000 (1989).)

PCR amplification techniques can also be used to identify and isolate nucleic acid sequences encoding proteins of the invention and are discussed generally in PCR Protocols. A Guide to Methods and Applications (Innis et al., eds, 1990).

Nucleic acids encoding proteins of the invention may also be prepared using synthetic techniques. Chemical synthesis of linear oligonucleotides is well known in the art and can be achieved by solution or solid phase techniques. Moreover, linear oligonucleotides of defined sequence can be purchased commercially or can be made by any of several different synthetic procedures including the phosphoramidite, phosphite triester, H-phosphonate and phosphotriester methods, typically by automated synthesis methods. The synthesis method selected can depend on the length of the desired oligonucleotide and such choice is within the skill of the ordinary artisan. For example, the phosphoramidite and phosphite triester method produce oligonucleotides having 175 or more nucleotides while the H-phosphonate method works well for oligonucleotides of less than 100 nucleotides. Oligonucleotides of the present invention can be synthesized chemically according to the solid phase phosphoramidite triester method described by Beaucage and Caruthers (1981), Tetrahedron Letts., 22(20):1859-1862, e.g., using an automated synthesizer, as described in Needham-VanDevanter et al. (1984) Nucleic Acids Res., 12:6159-6168. Oligonucleotides can also be custom made and ordered from a variety of commercial sources known to persons of skill in the art. Purification of oligonucleotides, where necessary, is typically performed by either native acrylamide gel electrophoresis or by anion-exchange HPLC as described in Pearson and Regnier (1983) J. Chrom. 255:137-149. See also Sambrook, J. et al. Molecular Cloning, A Laboratory Manual, 2d Ed. Cold Spring Harbor Laboratory Press, New York, 13.7-13.9 and Hunkapiller, M. W. (1991) Curr. Op. Gen. Devl. 1:88-92.

VII. Expression Systems

Nucleic acids encoding proteins of the present invention may be expressed in a variety of host organisms once they are operably linked in expression vectors suitable for the selected host organism. Suitable expression vectors typically comprise regulatory sequences operable in the host organism. These regulatory sequences are necessarily operably linked to the nucleic acid to control its expression. The expression vector includes a promoter that is either inducible or constitutively drives transcription, and may optionally comprise other regulatory, replication or manipulation sequences to aid in the expression and incorporation of the nucleic acid into the expression vector, as required by the particular application being pursued.

For example, to obtain a high level expression of a protein in a prokaryotic system, it is essential to construct expression vectors that contain, at a minimum; a strong promoter to direct transcription, a ribosome-binding site for translational initiation, a transcription/translation terminator, and unique restriction sites in nonessential regions of the plasmid to allow insertion of foreign nucleic acids. Other factors may also be carried on the expression vector, such as selectable and/or scorable markers, such as those described below. Suitable expression systems for use with the present invention are well known in the art. See, e.g., Pouwels, et al. (1985 and Supplements) Cloning Vectors: A Laboratory Manual, Elsevier, N.Y.; Rodriquez, et al. (eds.) Vectors. A Survey of Molecular Cloning Vectors and Their Uses, Buttersworth, Boston, 1988; Luckow, V. A. and Summers, M. D., Bio/Technology, 6:47-55 (1988); Herskowitz, I. and Hagen, D., Ann. Rev. Genet., 14:399-445 (1980); and Yanofsky, C., J. Bacteriol., 158:1018-1024 (1984).

Exemplary bacterial host organisms suitable for use in the present invention are well known in the art and include gram-positive and gram-negative bacteria such as Escherichia coli (cf. Sambrook et al., supra). E. coli strains are particularly preferred host organisms for expression of proteins of the present invention. Exemplary E. coli strains include BL21 (DE3), BL21-Gold (DE3), BL21 (DE3)-pLysS (Stratagene), MMLV-RT: JM109, DH5.alpha.f′, XL1BLUE STRATAGENE®, San Diego, Calif.), JM105, ER 1458, NM 522, In αf′ (Invitrogen, San Diego, Calif.), TOPP™. strains 1-6 (STRATAGENE®), 1200, MRE 600, Q13, and A19. Some of these strains (1200, MRE 600, Q13, and A19) are mutants that have reduced levels of RNase I (referred to as “RNase I deficient”) compared to wild type strains (Durwald et al., 1968, J. Mol. Biol. 34:331-346; Clark, 1963, Genetics 48:105-120; Gesteland, 1966, J. Mol. Biol. 16:67; Reiner, 1969, J. Bacteriol.97:1522), while others are common laboratory strains. Some of these strains contain the lac I^{q} repressor and required use of isopropylthiogalactoside (IPTG) to induce transcription. The level of RT expression of host cells containing the RT gene was estimated by visualizing the resulting proteins on SDS-polyacrylamide gels and also, in most cases, by enzyme activity assays on crude cell lysates. Of the RNase I deficient strains, E. coli 1200 (Strain 4449, available from the E. coli Genetic Stock Center, Yale University) consistently showed high levels of enzyme expression using these assays; unless indicated otherwise, all experiments described herein were conducted using this strain.

Standard transfection methods are used to introduce expression systems for proteins of the present invention to host organisms. (see, e.g., Morrison, J. Bact., 132:349-351 (1977); Clark-Curtiss & Curtiss, Methods in Enzymology, 101:347-362 (Wu et al., eds, 1983); Sambrook et al., and Ausubel et al., supra.). The proteins can be recovered from the cells or from the culture medium by standard protein purification techniques as described above.

Selectable Marker Genes

Identifying host organisms that have successfully incorporated nucleic acids encoding a protein of the present invention is preferably accomplished through inclusion of a selectable marker gene into the vector or expression system used for producing the protein. Selectable markers allow a transformed cell, tissue or animal to be identified and isolated by selecting or screening the engineered material for traits encoded by the marker genes present on the transforming DNA. For instance, selection may be performed by growing the engineered cells on media containing inhibitory amounts of an antibiotic to which the transforming marker gene construct confers resistance. Further, transformed cells may also be identified by screening for the activities of any visible marker genes (e.g., the β-glucuronidase, green fluorescent protein, luciferase, B or C1 genes) that may be present on the recombinant nucleic acid constructs of the present invention. Such selection and screening methodologies are well known to those skilled in the art.

Physical and biochemical methods may also be used to identify a cell transformant containing the genetic constructs of the present invention. These methods include but are not limited to: 1) Southern analysis or PCR amplification for detecting and determining the structure of the recombinant DNA insert; 2) Northern blot, S-1 RNase protection, primer-extension or reverse transcriptase-PCR amplification for detecting and examining RNA transcripts of the gene constructs; 3) enzymatic assays for detecting enzyme activity, where such gene products are encoded by the gene construct; 4) protein gel electrophoresis, western blot techniques, immunoprecipitation, or enzyme-linked immunoassays, where the gene construct products are proteins; 5) biochemical measurements of compounds produced as a consequence of the expression of the introduced gene constructs. The methods for performing these assays are well known to those skilled in the art

VIII. Chemical Protein Synthesis

Proteins of the present invention may also be synthesized chemically. For chemical synthesis, peptides may be synthesized either in solution, solid phase or a combination of these methods following standard protocols. See, for example, Wilken et al. (Curr. Opin. Biotech. (1998) 9(4):412-426), which reviews chemical protein synthesis techniques. The solution and solid phase synthesis methods are readily automated. A variety of peptide synthesizers are commercially available for batchwise and continuous flow operations as well as for the synthesis of multiple peptides within the same run. Briefly, the solid phase method consists of anchoring the growing peptide chain to an insoluble support or resin. This is accomplished through the use of a chemical handle, which links the support to the first amino acid at the carboxyl terminus of the peptide. Subsequent amino acids are then added in a stepwise fashion one at a time until the peptide segment is fully constructed. Solid phase chemistry has the advantage of permitting removal of excess reagents and soluble reaction by products by filtration and washing. The protecting groups of the fully assembled resin bound peptide chain are removed by standard chemistries suitable for this purpose. Standard chemistries also may be employed to remove the peptide chain from the resin. Cleavable linkers can be employed for this purpose.

Solution phase peptide synthesis generally involves reacting individual protected amino acids in solution to generate protected dipeptide product. After removal of a protection group to expose a reactive group for addition of the next amino acid, a second protected amino acid is reacted to this group to give a protected tripeptide. The process of deprotection/amino acid addition is repeated in a stepwise fashion to yield a protected peptide product. One or more to these protected peptides can be reacted to give the full-length protected peptide. Most or all or the remaining protecting groups are removed to generate an unprotected synthetic peptide segment. Thus, solid phase or solution phase chemistries may be employed to form synthetic peptides comprising one or more functional protein modules.

In general, the method of chemical synthesis employs a combination of chemical synthesis and chemical ligation techniques. By way of example, chemical synthesis approaches described above may be utilized in combination with various chemoselective chemical ligation techniques for producing the proteins of the invention. Chemoselective chemical ligation chemistries that can be utilized in the methods of the invention include native chemical ligation (Dawson et al., Science (1994) 266:77-779; Kent et al., WO 96/34878), extended general chemical ligation (Kent et al., WO 98/28434), oxime-forming chemical ligation (Rose et al., J. Amer. Chem. Soc. (1994) 116:30-33), thioester forming ligation (Schnolzer et al., Science (1992) 256:221-225), thioether forming ligation (Englebretsen et al., Tet. Letts. (1995) 36(48):8871-8874), hydrazone forming ligation (Gaertner et al., Bioconj. Chem. (1994) 5(4):333-338). thaizolidine forming ligation and oxazolidine forming ligation (Zhang et al., Proc. Natl. Acad. Sci. (1998) 95(16):9184-9189; Tam et al., WO 95/00846). The preferred chemical ligation chemistry for synthesis of cross-over proteins according to the method of the invention is native chemical ligation.

Synthesis of proteins by a combination of chemical ligation and chemical synthesis permits facile incorporation of one or more chemical tags. These include synthesis and purification handles, as well as detectable labels and optionally chemical moieties for attaching the protein to a support matrix for screening and diagnostic assays and the like. As can be appreciated, in some instances it may be advantageous to utilize a given chemical tag for more than one purpose, e.g., both as a handle for attaching to support matrix and as a detectable label. Examples of chemical tags include metal binding tags (e.g., his-tags), carbohydrate/substrate binding tags (e.g., cellulose and chitin binding domains), antibodies and antibody fragment tags, isotopic labels, haptens such as biotin and various unnatural amino acids comprising a chromophore, some of which have been discussed supra. A chemical tag also may include a cleavable linker so as to permit separation of the protein from the chemical tag depending on its intended end use.

IX. Thermophilic Applications

A. Nucleic Acid Amplification Techniques

Proteins of the present invention find application in a variety of processes, including biosynthetic and biodegradive processes, particularly those where performance of the process at a mesophilically or thermophilically compatible temperature is beneficial. For example, proteins of the present invention that catalyze reactions of import in nucleic acid synthesis are particularly suited for nucleic acid amplification processes. Methods of “quantitative” nucleic acid amplification are well known to those of skill in the art. For example, quantitative PCR involves simultaneously co-amplifying a known quantity of a control sequence using the same primers. This type of quantitative amplification provides an internal standard that may be used to calibrate the PCR reaction.

One exemplary internal standard is a synthetic AW106 cRNA. The AW106 cRNA is combined with RNA isolated from the sample according to standard techniques known to those of skilled in the art. The RNA is then reverse transcribed using a reverse transcriptase to provide cDNA. The cDNA sequences are then amplified (e.g., by PCR) using labeled primers. The amplification products are separated, typically by electrophoresis, and the amount of radioactivity (proportional to the amount of amplified product) is determined. The amount of MRNA in the sample is then calculated by comparison with the signal produced by the known AW106 RNA standard. Detailed protocols for quantitative PCR are provided in PCR Protocols, A Guide to Methods and Applications, Innis et al., Academic Press, Inc. N.Y., (1990).

Other suitable amplification methods include, but are not limited to polymerase chain reaction (PCR) (Innis, et al., PCR Protocols. A guide to Methods and Application. Academic Press, Inc. San Diego, (1990)), ligase chain reaction (LCR) (see Wu and Wallace, Genomics, 4: 560 (1989), Landegren, et al., Science, 241: 1077 (1988) and Barringer, et al., Gene, 89: 117 (1990), transcription amplification (Kwoh, et al., Proc. Natl. Acad. Sci. USA, 86: 1173 (1989)), and self-sustained sequence replication (Guatelli, et al., Proc. Nat. Acad. Sci. USA, 87: 1874 (1990)).

Methods of in vitro polymerization are well known to those of skill in the art (see, e.g., Sambrook, supra.) and this particular method is described in detail by Van Gelder, et al., Proc. Natl. Acad. Sci. USA, 87: 1663-1667 (1990) who demonstrate that in vitro amplification according to this method preserves the relative frequencies of the various RNA transcripts. Moreover, Eberwine et al. Proc. Natl. Acad. Sci. USA, 89: 3010-3014 provide a protocol that uses two rounds of amplification via in vitro transcription to achieve greater than 106 fold amplification of the original starting material, thereby permitting expression monitoring even where biological samples are limited.

It will be appreciated by one of skill in the art that the direct transcription method described above provides an antisense (aRNA) pool. Where antisense RNA is used as the target nucleic acid, the oligonucleotide probes provided in the array are chosen to be complementary to subsequences of the antisense nucleic acids. Conversely, where the target nucleic acid pool is a pool of sense nucleic acids, the oligonucleotide probes are selected to be complementary to subsequences of the sense nucleic acids. Finally, where the nucleic acid pool is double stranded, the probes may be of either sense as the target nucleic acids include both sense and antisense strands.

The protocols cited above include methods of generating pools of either sense or antisense nucleic acids. Indeed, one approach can be used to generate either sense or antisense nucleic acids as desired. For example, the cDNA can be directionally cloned into a vector (e.g., Stratagene's p Bluscript II KS (+) phagemid) such that it is flanked by the T3 and T7 promoters. In vitro transcription with the T3 polymerase will produce RNA of one sense (the sense depending on the orientation of the insert), while in vitro transcription with the T7 polymerase will produce RNA having the opposite sense. Other suitable cloning systems include phage lambda vectors designed for Cre-loxP plasmid subcloning (see e.g., Palazzolo et al., Gene, 88: 25-36 (1990)).

Exemplary reagent mixtures for use in amplifying nucleic acids according to the methods of the present invention include a recombinant protein that has a thermophilic amino acid sequence at least about 75% homologous to an YS40 amino acid sequence encoded by at least about 25 contiguous codons of SEQ ID NO: 2, 4-64, 70-149, 151 or 153-170. This thermophilic amino acid sequence confers to the recombinant protein an enzyme activity necessary for DNA amplification when incubated at a permissible temperature of at least about 36° C., more typically at least about 55° C. most typically at least about 65° C. Typically the YS40 amino acid sequence is encoded by at least 25 contiguous codons of SEQ ID NO: 5, 8, 9, 12-14, 17, 18, 23-27, 29, 33, 38, 41, 42, 52, 57, 59, 60, 62, 71, 79, 114, 144 or 161. Most typically the YS40 amino acid sequence is encoded by at least about 25 contiguous codons of SEQ ID NO: 33, and the enzyme activity is DNA polymerase.

In some embodiments of the present invention, amplification of nucleic acids is contemplated as taking place directly from whole cells containing the nucleic acid to be amplified. Exemplary proteins of the present invention possessing protease, lipase or other enzymatic activities that degrade biomolecules of a cell may be included in the amplification reaction. These enzymes, together with the elevated temperatures of the reaction, provide a means of breaching the cell membrane and allowing the nucleic acid within the cell to be amplified. Methodology for carrying out such reactions will be obvious to one of skill in the art, and may be adapted to virtually any cell system through routine experimentation.

Methods of the present invention for amplifying nucleic acids from whole cells include subjecting the cell preparation to at least one thermophilic protein that has a recombinant amino acid sequence at least about 75% homologous to an YS40 amino acid sequence, which is encoded by at least 25 contiguous codons from SEQ ID NO: 2, 4-64, 70-149, 151 and 153-170, more typically SEQ ID NOs: 5, 8, 9, 12-14, 17, 18, 23-27, 29, 33, 38, 41, 42, 52, 57, 59, 60, 62, 71, 79, 114, 144 or 161, most typcially SEQ ID NO: 33. In certain instances, the thermophilic protein encoded by SEQ ID NO: 33 is preferred. When incubated at a permissible temperature greater than about 36° C., more preferably greater than about 55° C., and most preferably greater than about 65° C., the cell membrane is breached, allowing the amplification reagents to contact the nucleic acids of the cell, which are subsequently amplified.

B. Biodegredation Techniques

Another preferred application of the proteins of the present invention is the use of the proteins in commercially important biosynthetic or biodegradative processes. For example, the present invention contemplates using proteins described herein in mesophilic and thermophilic processes for the synthesis or degradation of biomaterials. Using protein enzymes of the present invention, these reactions may be carried out at elevated temperatures that are incompatible with growth of bacteria that may normally interfere with such processes, while providing accelerated enzymatic activity resulting from the higher temperature. By way of example, processes in which protein enzymes of the present invention may be used include, but are not limited to, waste water treatment, fermentation processes, composting, paper manufacture, etc. It will be readily appreciated by one of skill in the art that the proteins of the present invention find use in many processes in addition to those listed here, and may be applied to such processes through routine experimentation.

Methods of the present invention suitable for decomposing a biodegradable material involve contacting the biodegradable material with at least one recombinant protein that has a recombinant amino acid sequence that is at least about 75% homologous to an YS40 amino acid sequence encoded by at least 25 contiguous codons of SEQ ID NO.: 2, 4-64, 70-149, 151 or 153-170 of Table 1. The recombinant amino acid sequence confers to the recombinant protein, at a permissible temperature greater than 36° C., more preferably greater than 55° C. and most preferably greater than 65° C., an enzyme activity necessary for decomposing the biodegradable material, which may be a protease, amylase, cellulase, nuclease, lipase, deaminase or a peptidase.

X. Thermus Expression System

In addition to the proteins of the present invention and the nucleic acids encoding them, the present invention also contemplates a Thermus thermophilus expression system for expression of foreign proteins at elevated temperatures. Central to Thermus thermophilus expression systems of the present invention is an expression vector based on the YS40 bacteriophage genome. The expression vector includes no more than 99.9% of the nucleotide sequence of SEQ ID NO: 171 or its complement. Inserted into this vector is a non-YS40 nucleotide sequence of at least 20 contiguous nucleotides. This non-YS40 nucleotide sequence is inserted into the vector sequence such that it is flanked on its 3′ and 5′ ends by at least 10 contiguous nucleotides from the YS40 genome. The non-YS40 nucleotide sequence may include a promoter suitable for expression of the protein encoded by the non-YS40 nucleotide sequence in T. thermophilus, and/or the non-YS40 nucleotide sequence may be operably linked to one or more regulatory sequences of YS40 bacteriophage.

The expression vector containing the non-YS40 nucleotide sequence is then introduced into T. thermophilus using any technique known to those of skill in the art, such as those described above. The transformed T. thermophilus is then cutured at a permissible temperature under suitable conditions allowing expression of the protein encoded by the non-YS40 nucleotide sequence.

Although use of T. thermophilus is a preferred embodiment of the present invention, other cellular hosts are also contemplated, as are cell-free expression systems, such as reticulocyte lysates.

XI. Kits

The present invention also contemplates kit embodiments suitable for amplifying nucleic acid samples. These kits include a reagent containing at least one recombinant protein that has a thermophilic amino acid sequence at least about 75% homologous to an YS40 amino acid sequence encoded by at least about 25 contiguous taken from one of the sequences SEQ ID NO: 1-170. The reagent has an enzyme activity necessary for DNA amplification or DNA entry into the cell, as described above, at a permissible temperature of at least about 36° C., more typically at least about 55° C. most typically at least about 65° C. Kit embodiments also include a buffer solution for diluting the reagent and may optionally include universal primers and/or known calibration nucleic acids known to those of skill in the art.

All publications and patent applications cited in this specification are herein incorporated by reference as if each individual publication or patent application were specifically and individually indicated to be incorporated by reference.

Although the foregoing invention has been described in some detail by way of illustration and example for clarity and understanding, it will be readily apparent to one of ordinary skill in the art in light of the teachings of this invention that certain changes and modifications may be made thereto without departing from the spirit and scope of the appended claims. The following examples are included to demonstrate certain embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLES

### Example 1

This example describes one method of identifying coding sequences of the present invention starting from the genomic sequence of the YS40 thermophilic phage.

The genome sequence of YS40 was searched for open reading frames (ORFs) using the hidden Markov model approach implemented in GeneMark program (See Besemer J. and Borodovsky M (1999)., NAR, Vol. 27, No. 19, pp. 3911-3920). Using this technique, 170 open reading frames (ORFs) encoding preferred proteins of the present invention were predicted (Table 1). Regions between the identified ORFs were then screened for additional genes using the Blastx and tBlastx programs (Schafer et al., 1997) to identify regions having similarity with available entries in GenBank, GenPept, and the database of unfinished microbial genomes at NCBI. This latter search did not identify any additional coding sequences.

The predicted YS40 ORFs have lengths between 43 and 1744 codons. As with most other phages, the genome of YS40 is tightly packed, with little space between ORFs and 46 cases of overlaps (from 1 to 40 bases in length) between the adjoining ORFs. Ninety-five percent of the YS40 genome is occupied by coding sequence, and on the average, there are 1.129 genes per 1 kb. The G+C-content are 32.29% and 33.92% for coding and non-coding regions, respectively. The longest non-coding region is 390 bp and length; it lies between the ORF138 and ORF139 and does not appear to demarcate any functional regions.

Among the 170 predicted ORFs, most initiate translation at the AUG codon, 22 appear to use GUG and 3 use UUG. Among the stop codons, TAA is found in 90 cases, TGA in 66, and TAG in 16 cases.

The two thirds of YS40 genome (114 genes) are transcribed leftwards, and 56 genes are transcribed rightwards. The G+C content is approximately the same for both sets of ORFs. The largest cluster of consecutive genes with the same transcriptional orientation contains 35 ORFs that encodes mostly proteins with unknown function (SEQ ID NO:97 through SEQ ID NO:131).

tRNAs Genes

Similarly to some large tailed dsDNA bacteriophages, such as coliphage T4 (Miller ES, et al. 2003), vibriophage KVP40 (Miller E S, et al. 2003) and phage phiKZ of P. aeruginosa (Mesyanzhinov VV., et al. 2002), YS40 encodes several tRNAs. Using the tRNA scan-SE program (Lowe, T. M. & Eddy, S. R. 1997), three tRNA genes were identified within the YS40 genome in two intergenic regions, with Met^{AUG} (SEQ ID NO:172), Arg^{AGA} (SEQ ID NO:173), and Thr^{ACA} (SEQ ID NO:174) specificities. The first two tRNA genes are located in a non-coding region between ORF71 and ORF72, whereas tRNA-Thr gene overlaps with ORF164. Given the significant difference in G+C content of YS40 and its Thermus host, the phage own tRNAs presumably influence the rate of translation of YS40 proteins.

### Sequence Analysis of Predicted YS40 Proteins

There are 170 potential ORFs in YS40 genome, coding for predicted proteins ranging from 43 to 1744 amino acid residues. These are presented as SEQ ID NO:1 through SEQ ID NO:170. Analysis of intrinsic sequence features indicates that least 7 proteins contain putative transmembrane domains (from one to three), and 4 proteins have coiled coil regions. There is only one predicted non-globular proteins, gp107 in YS40 . And one protein, gp35, that is predicted to have an N-terminal secretion signal peptide, while there are about 10 proteins have such predicted N-terminal signal peptide. All deduced amino acid sequences were compared to proteins in the protein sequence database using the PSI-BLAST program (Altschul S F, Madden T L, Schaffer A A, Zhang J, Zhang Z, Miller W, Lipman D J. Gapped BLAST and PSI-BLAST: a new generation of protein database search programs. Nucleic Acids Res. 1997 September 1;25(17):3389-3402) with a slightly relaxed cutoff for profile inclusion (−h parameter) of 0.02, and the output was analyzed for the presence of conserved sequence motifs, with particular attention paid at the matches to proteins from other bacteriophages (Table 1). The comparison showed that about 25% of the YS40 proteins that are longer than 100 amino acids display sequence similarity to proteins of known function from a diversity of bacteria and bacteriophages (Table 1).

### YS40 Proteins Involved in Nucleotide Metabolism

YS40 encodes a number of enzymes that are involved in nucleotide metabolism. They are gp8, a homolog of mammalian/virus UTPase (EC 3.6.1.23); gp9, related to flavin-dependent thymidylate synthase (EC 2.1.1.148); gp17, a GMP reductase, having sequence similarity to EC 1.7.1.7; gp24, a thymidine kinase, having sequence similarity to EC 2.7.1.21, PF00265; gp38, a deoxycytidylate deaminase, having sequence similarity to PF00383, EC 3.5.4.12; gp60, a dNMP kinase; and the α subunit of ribonucleotide reductase, encoded by two adjoining ORFs, gp41 and gp42.

### YS40 Proteins Involved in DNA Replication and Recombination

YS40 encodes most of the proteins required for its own replisome formation, namely gp27 and gp79, two helicases with DEAD signature in the Walker B motif; gp14, replication initiation helicase DnaB; gp23, bacterial DnaG-family DNA primase; gp26, RecB family exonuclease; gp33, type A DNA polymerase; and gp65, a terminal protein that may be covalently attached to the 5′ of YS40 genome DNA terminus. YS40 also encodes two recombination proteins, gp12, RecA/RadA recombinase; and gp114, recombination protein ERF.

As with most A-type DNA polymerases, gp33 contains conserved nucleotidyltransferase domain and 3′-5′ exonuclease domain. However, like the Klenow fragment of E. coli DNA polymerase I, gp33 lacks the N-terminal 5′-3′ exonuclease domain. Furthermore, in YS40 genome, there are no gene products with detectable sequence similarity to single-stranded DNA binding protein from any known class (Ponomarev VA, et al. Mol Microbiol Biotechnol. 2003), nor to any DNA ligases from other bacteria or bacteriophages.

The protein gp65 is of particular interest in understanding the replication mechanism of YS40 . It shows striking sequence similarity to the C-terminal portion of the podovirus phi29 terminal protein (TP) that is essential for the protein-primed DNA replication of the linear phi29 genome. In phi29, the 5′-terminal dAMP is linked via a phosphoester bond to the hydroxyl group of Ser₂₃₂ of the TP (Hermoso J M, et al. 1985), and this Ser₂₃₂ is absolutely critical for the priming activity of TP (Garmendia C, et al. 1988; Garmendia C, et al. 1990). As shown in figure #, this serine residue is conserved in all TPs from phi29 family phages and YS40 . The sequence similarity between gp65 and phi29 TP strongly suggests that YS40 replicates its genome in a linear form, probably adopting the similar protein-primed replication mode as phi29 family phages.

### Identified YS40 Structural Proteins and DNA Packaging Proteins

The overall architecture of YS40 genome is unique, as compared to other sequenced phage genomes. In particular, the tendency towards tight clustering of gene coding for virion component, that is so prevalent in lambdoid phages and T4-like phage groups, is hardly observed in YS40 genome. For example, gp150, encoding a putative Myovirus-like baseplate assembly protein, is adjacent to gp152 that encodes a putative Myovirus-like wac fibritin neck whisker, but these two structural genes are located far away from other recognized YS40 structural genes, such as the genes coding for gp1 (distal tail fiber protein), gp3 (portal protein), gp62 (terminase large subunit) and gp69 (tail sheath protein).

### YS40 Protein Sequences and Thermophily

YS40 is capable of withstanding temperature as high as 75° C. in its Thermus host. Thus its molecular milieu is extremely resistant to elevated temperatures, such as those desirably employed in bioreactors, including PCR processes. However, only one YS40-encoded protein, gp13 (recombination protein ERF), has the best database match in Thermus bacteria. And for 6 of the 170 YS40 predicted gene products, the best database match is from thermophilic microorganisms, including Thermotoga maritima, Thermoanaerobacter tengcongensis, and Methanocaldococcus jannaschii. Further investigation on the sequence, function and evolution of these groups of thermophilie-affiliated YS40 proteins may give us more clues on the survival strategy of this phage under the extreme temperature. For instance, the best database match of gp5, S-adenosylmethionine decarboxylase (adoMetDC), a key enzyme in the biosynthesis of spermidine and spermine, is from M. jannaschii. The placement of YS40 adoMetDC on a thermophilic species-specific clade, including both bacterial and archaeal species, such as Aquifex aeolicus, Thermoplasma, Picrophilus and Pyrococcus, in the phylogenetic tree built on the basis of multiple sequence alignment of adoMetDC enzymes (data not shown) suggests that thermophilic adoMetDC enzymes are evolutionarily specialized, therefore may be important for the survival of thermophilic microorganisms in extreme high temperature.

### Example 2

This example describes the ability of the phage protein Ys18, encoded by SEQ ID NO: 18, to negatively regulate transcription initiation by binding RNA polymerase sigma factors from T. thermophilus (T. th) and E. coli. Binding experiments and transcription experiments have been used to determine the function of Ys18.

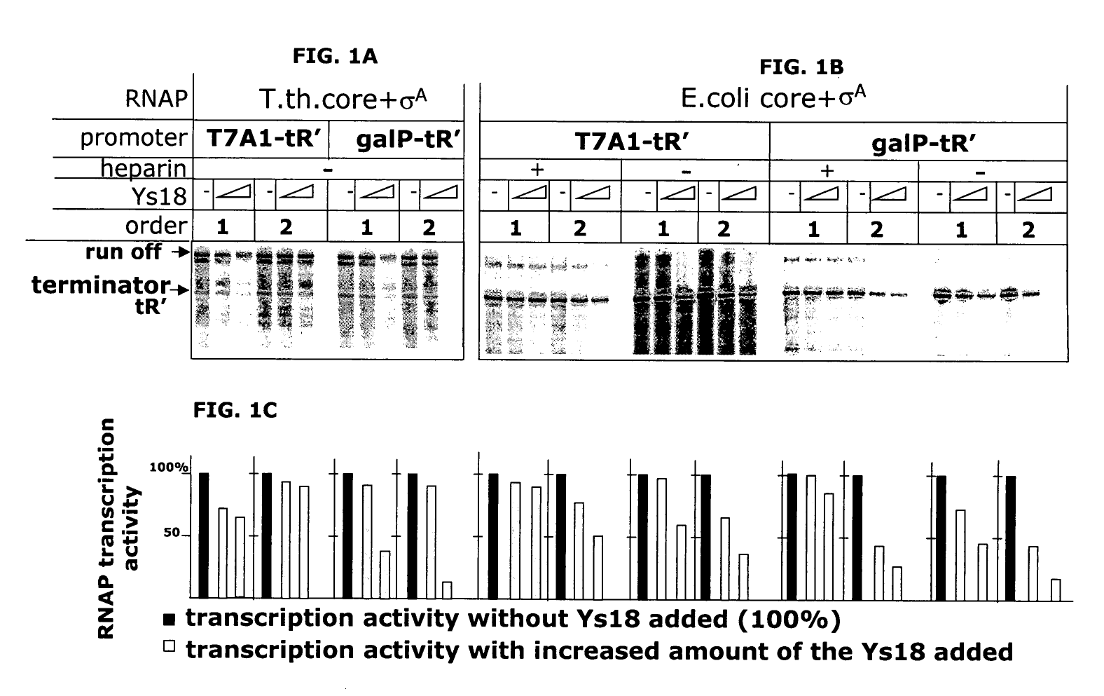

The function of Ys18 was analyzed by using a run-off transcription assay to determine if Ys18 was involved in transcription (FIG. 1). Two transcriptionally competent open model promoters from different classes, −10/−35 class (T7A1) and extended −10 class (galP1), attached to a rho-independent terminator were used to quatitatively visualize efficiency of run-off transcription. Reaction 1, in 20 μl of transcription buffer (30 mM tris-HCl, pH 8.0, 10 mM MgCl2, 40 mM KC1, 1 mM β-mercaptoethanol), contained core enzyme, sigma and Ys18. Reaction 1 was incubated at 65° C. (for T. th) and 37° C. (for E. coli) for 10 minutes, followed by the addition of promoter DNA fragments. Reaction 2, in 10 μl of transcription buffer, contained core enzyme and sigma, and, in parallel, Ys18 and a promoter DNA fragment. Reaction 2 was incubated at 65° C. (for T. th) and 37° C. (for E. coli) for 10 minutes and then mixed together. For both reactions 1 and 2, after 10 minutes of incubation at the same temperatures, 200 μM ATP, CTP, UTP, 20 μM GTP and 10 μCi of [α−³²P] GTP were added, the reactions were incubated for the next 10 minutes, and terminated by an equal volume of 9 M urea loading buffer.

In the presence of Ys18, the T. th RNA polymerase inhibited transcription in a YS18 dose-dependent manner to different degrees with each promoter (FIG. 1A). This data suggests that YS18 inhibited transcription more actively from extended—10 promoter galP1, likely due to the absence of the −35 box stabilizing effect.

Ys18-dependent inhibition of transcription by E. coli RNA polymerase (FIG. 1B) was similar to that of T. thermophilus (FIG. 1A). In the presence of Ys18, the E. coli RNA polymerase inhibited transcription in an YS18 dose-dependent manner to different degrees with each promoter in multi- and single-round transcription (FIG. 1B). The results demonstrate that Ys18 only slightly, if not at all, inhibited single-round transcription, especially at the −10/−35 promoter T7A1. Ys18 inhibited multi-round transcription from extended −10 promoter galP1 more actively.

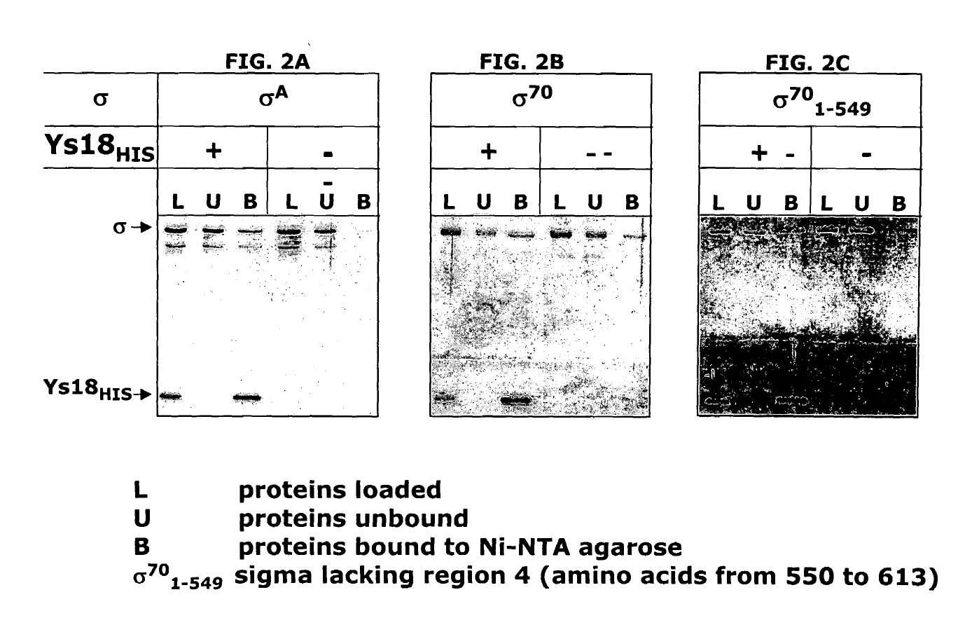

With both E. coli and T. th RNA polymerases, the difference of transcriptional inhibition between the different orders of promoter DNA addition to the transcription reaction (FIG. 1C), indicates that Ys18 interacted not only with RNA polymerase through sigma (σ) but also with promoter DNA. Further the proportional change in terminator bands to run-off bands suggests that Ys18 does not affect termination. Thus the phage protein seems to negatively regulate transcription initiation but not elongation or termination. Taken together, the results obtained in the transcription (FIG. 1) and the Ni—NTA agarose binding (FIG. 2) experiments suggest that Ys18 inhibited transcription initiation through interaction with RNA polymerase sigma subunits.

Binding experiments using Ni—NTA agarose suggest Ys18 associates with RNA polymerase sigma subunits to inhibit transcription. Primary sigma factors were preincubated in the presence and absence of His-tagged Ys18 (YS18_{HIS}) in 20 μl 1 of binding buffer (20 mM tris HCl, pH8.0, 0.5 M NaCl, 2 mM imidazole, 5% v/v glycerol) for 10 minutes at 65° C. (for T. th σ^{A}) and 37° C. (E.coli σ⁷⁰). The binding mixtures were then added to Ni—NTA agarose beads, equilibrated in the binding buffer, which bind His tags. Reactions were incubated for 10 minutes at room temperature. The agarose beads were pelleted by quick centrifugation and the unbound proteins were withdrawn. The beads were washed 3 times with the binding buffer containing 200 mM imidazole. Fractions were then resolved by SDS-PAGE and stained by Coomassie.

Ys18_{HIS} bound to the RNA polymerase sigma factor from T.th (σ^{A}). In the presence of both Ys18_{HIS} and σ^{A} (FIG. 2A), σ^{A} was detected in the unbound and the bound fractions. In the absence of YS18_{HIS} (FIG. 2A), σ^{A} was exclusively observed in the unbound fraction. These results show σ^{A} cannot bind to the Ni—NTA agarose beads without Ys18_{HIS}, indicating Ys18_{HIS} was capable of binding to σ^{A}.

Ys18_{HIS} also bound to the RNA polymerase sigma factor from E. Coli (σ⁷⁰). In the presence of both Ys18_{HIS} and σ⁷⁰ (FIG. 2B), σ⁷⁰ was detected in the unbound and the bound fractions. In the absence of Ys18_{HIS} (FIG. 2B), σ⁷⁰ was exclusively observed in the unbound fraction. Further, Ys18_{HIS} also bound to the primary sigma factor from E. coli lacking region 4 (σ⁷⁰₁₋₅₄₉). When both Ys18_{HIS} and σ⁷⁰₁₋₅₄₉ were present in the sample, σ⁷⁰₁₋₅₄₉ was detected in the unbound and the bound fractions (FIG. 2C). This data suggests Ys18_{HIS} bound to σ⁷⁰ in a region other than region 4.

What is claimed is:

1. An isolated protein, including conservatively modified variants thereof, comprising a thermophilic amino acid sequence at least 75% identical to a YS40 amino acid sequence encoded by at least 25 contiguous codons of a YS40 coding sequence selected from the group consisting of SEQ ID NO: 1-170.

2. The protein of claim 1, wherein the thermophilic amino acid sequence is identical to the YS40 amino acid sequence.

3. The protein of claim 1, wherein the YS40 amino acid sequence is encoded by at least 50 contiguous codons of the YS40 coding sequence.

4. The protein of claim 1, wherein the YS40 amino acid sequence is encoded by at least 100 contiguous codons of the YS40 coding sequence.

5. The protein of claim 1, wherein the YS40 coding sequence is from a YS40 structural protein.

6. The protein of claim 5, wherein the YS40 coding sequence is selected from the group consisting of SEQ ID NO: 1, 3, 65, 69, 71, 151 and 152.

7. The protein of claim 1, wherein the YS40 coding sequence is selected from the group consisting of SEQ ID NO: 2, 4-64, 70-149, 151 and 153-170.

8. The protein of claim 7, wherein the thermophilic amino acid sequence confers to the protein, at a permissible temperature of about 36° C., an enzymatic activity selected from the group consisting of decarboxylase, nuclease, synthase, recombinase, helicase, dehydrogenase, reductase, nucleotide primase, kinase, protease, nucleotidyltransferase, nucleic acid polymerase, deaminase, acyltransferase, terminase, helicase, glycosyltransferase and peptidase.

9. The protein of claim 8, wherein the YS40 coding sequence is selected from the group consisting of SEQ ID NO: 5, 8, 9, 12-14, 17, 18, 23-27, 29, 33, 38, 41, 42, 52, 57, 59, 60, 62, 71, 79, 114, 144 and 161.

10. The protein of claim 8, wherein the YS40 coding sequence is SEQ ID NO: 33, and the enzymatic activity is DNA polymerase.

11. The protein of claim 7, wherein the permissible temperature is at least 45° C.

12. The protein of claim 7, wherein the permissible temperature is at least 55° C.

13. The protein of claim 7, wherein the permissible temperature is at least 65° C.

14. The protein of claim 7, wherein the permissible temperature is at least 75° C.

15. An isolated nucleic acid encoding the protein of claim 1.

16. The nucleic acid of claim 15, wherein the thermophilic amino acid sequence of the encoded protein is identical to the YS40 amino acid sequence.

17. The nucleic acid of claim 15, wherein the YS40 amino acid sequence is encoded by at least 50 contiguous codons of the YS40 coding sequence.

18. The nucleic acid of claim 15, wherein the YS40 amino acid sequence is encoded by at least 100 contiguous codons of the YS40 coding sequence.

19. The nucleic acid of claim 15, wherein the YS40 coding sequence is selected from the group consisting of SEQ ID NO: 1, 3, 65, 69, 71, 151 and 152.

20. The nucleic acid of claim 15, wherein the thermophilic amino acid sequence confers an enzymatic activity to the encoded protein at a permissible temperature of 36° C., the enzymatic activity selected from the group consisting of decarboxylase, nuclease, synthase, recombinase, helicase, dehydrogenase, reductase, nucleotide primase, kinase, protease, nucleotidyltransferase, nucleic acid polymerase, deaminase, acyltransferase, terminase, helicase, glycosyltransferase and peptidase.

21. The nucleic acid of claim 20, wherein the YS40 coding sequence is selected from the group consisting of SEQ ID NO: 2, 4-64, 70-149, 151 and 153-170.

22. The nucleic acid of claim 21, wherein the YS40 coding sequence is selected from the group consisting of SEQ ID NO: 5, 8, 9, 12-14, 17, 18, 23-27, 29, 33, 38, 41, 42, 52, 57, 59, 60, 62, 71, 79, 114, 144 and 161.

23. The nucleic acid of claim 21, wherein the YS40 coding sequence is SEQ ID NO: 33, and the enzymatic activity is DNA polymerase.

24. The nucleic acid of claim 20, wherein the permissible temperature is at least 45° C.

25. The nucleic acid of claim 20, wherein the permissible temperature is at least 55° C.

26. The nucleic acid of claim 20, wherein the permissible temperature is at least 65° C.

27. The nucleic acid of claim 20, wherein the permissible temperature is at least 75° C.

28. A recombinant vector comprising the nucleic acid of claim 15 operably linked to a promoter wherein introduction of the vector into an expression system produces a protein having, at a permissible temperature of 36° C., an enzyme activity selected from the group consisting of decarboxylase, nuclease, synthase, recombinase, helicase, dehydrogenase, reductase, nucleotide primase, kinase, protease, nucleotidyltransferase, nucleic acid polymerase, deaminase, acyltransferase, terminase, helicase, glycosyltransferase and peptidase.

29. The vector of claim 28, wherein the promoter is inducible.

30. The vector of claim 28, wherein the permissible temperature is at least 45° C.

31. The vector of claim 28, wherein the permissible temperature is at least 55° C.

32. The vector of claim 28, wherein the permissible temperature is at least 65° C.

33. The vector of claim 28, wherein the permissible temperature is at least 75° C.

34. The vector of claim 28, wherein the YS40 coding sequence is selected from the group consisting of SEQ ID NO: 2, 4-64, 70-149, 151 and 153-170.

35. The vector of claim 34, wherein the YS40 coding sequence is selected from the group consisting of SEQ ID NO: 5, 8, 9, 12-14, 17, 18, 23-27, 29, 33, 38, 41, 42, 52, 57, 59, 60, 62, 71, 79, 114, 144 and 161.

36. The vector of claim 34, wherein the YS40 coding sequence is SEQ ID NO: 33, and the enzyme activity is DNA polymerase.

37. A protein expression system comprising the vector of claim 28 wherein incubating the expression system under permissible conditions produces the recombinant protein encoded by the vector.

38. The protein expression system of claim 37, further comprising a cell wherein the vector is within the cell.

39. An isolated nucleic acid comprising an YS40 nucleotide sequence selected from the group consisting of SEQ ID NO: 1-170,
wherein the YS40 nucleotide sequence encodes
a YS40 structural protein that does not take a random coil structure at a permissible temperature of 36° C., or
a YS40 enzyme that displays, at a permissible temperature of 36° C., an enzyme activity selected from the group consisting of decarboxylase, nuclease, synthase, recombinase, helicase, dehydrogenase, reductase, nucleotide primase, kinase, protease, nucleotidyltransferase, nucleic acid polymerase, deaminase, acyltransferase, terminase, helicase, glycosyltransferase and peptidase.

40. The nucleic acid of claim 39, further comprising a regulatory element operably linked to the YS40 nucleotide sequence.

41. The nucleic acid of claim 39, wherein the permissible temperature is at least 45° C.

42. The nucleic acid of claim 39, wherein the permissible temperature is at least 55° C.

43. The nucleic acid of claim 39, wherein the permissible temperature is at least 65° C.

44. The nucleic acid of claim 39, wherein the permissible temperature is at least 75° C.

45. A recombinant cell comprising the nucleic acid of claim 39.

46. An isolated nucleic acid comprising:
a) a vector sequence comprising no more than about 99.9% of the nucleotide sequence of SEQ ID NO: 171 and
b) a non-YS40 nucleotide sequence of at least 20 contiguous nucleotides having a 3′ end and a 5′ end,
wherein the non-YS40 nucleotide sequence is inserted into the vector sequence whereby the non-YS40 nucleotide sequence is flanked on the 3′ end and the 5′ end by at least 10 contiguous nucleotides of the vector sequence.

47. The nucleic acid of claim 46, further comprising a regulatory element operably linked to the non-YS40 nucleotide sequence.

48. A recombinant system comprising a cell including the nucleic acid of claim 46.

49. The recombinant system of claim 48, wherein the cell is Thermus thermophilus.

50. A method of amplifying a nucleic acid comprising contacting the nucleic acid with a PCR reagent mixture including a recombinant protein, including conservatively modified variants thereof, comprising a thermophilic amino acid sequence at least 75% identical to an YS40 amino acid sequence encoded by at least 25 contiguous codons of a YS40 coding sequence selected from the group consisting of SEQ ID NO: 2, 4-64, 70-149, 151 and 153-170, wherein the thermophilic amino acid sequence confers to the recombinant protein, at a permissible temperature of 36° C., an enzyme activity necessary for DNA amplification.

51. The method of claim 50, wherein the YS40 coding sequence of the thermophilic amino acid sequence is selected from the group consisting of SEQ ID NO: 5, 8, 9, 12-14, 17, 18, 23-27, 29, 33, 38, 41, 42, 52, 57, 59, 60, 62, 71, 79, 114, 144 and 161.

52. The method of claim 50, wherein the YS40 coding sequence of the YS40 amino acid sequence is SEQ ID NO: 33, and the enzyme activity is DNA polymerase.

53. A method of amplifying a nucleic acid from a whole cell, the method comprising: contacting the cell with at least one recombinant protein comprising a thermophilic amino acid sequence, including conservatively modified variants thereof, that is at least 75% identical to a YS40 amino acid sequence encoded by at least 25 contiguous codons of a YS40 coding sequence selected from the group consisting of SEQ ID NO: 1-170, wherein the thermophilic amino acid sequence confers to the thermophilic protein, at a permissible temperature of 36° C., an enzyme activity necessary for DNA amplification or DNA entry into the cell.

54. The method of claim 53, wherein DNA entry into the cell comprises lysing the cell.

55. A method for decomposing a biodegradable material comprising contacting the biodegradable material with at least one recombinant protein, including conservatively modified variants thereof, comprising a thermophilic amino acid sequence at least 75% identical to an YS40 amino acid sequence encoded by at least 25 contiguous codons of a YS40 coding sequence selected from the group consisting of SEQ ID NO: 2, 4-64, 70-149, 151 and 153-170, wherein the thermophilic amino acid sequence confers to the recombinant protein, at a permissible temperature of 36° C., an enzyme activity necessary for decomposing the biodegradable material selected from the group consisting of protease, amylase, cellulase, nuclease, lipase, deaminase and peptidase.

56. A kit suitable for use in amplifying a nucleic acid, the kit comprising:
a) a reagent comprising at least one recombinant protein, including conservatively modified variants thereof, comprising a thermophilic amino acid sequence at least 75% identical to an YS40 amino acid sequence encoded by at least 25 contiguous codons of a YS40 coding sequence selected from the group consisting of SEQ ID NO: 1-170,
wherein the thermophilic amino acid sequence confers to the recombinant protein, at a permissible temperature of 36° C., an enzyme activity necessary for DNA amplification or DNA entry into the cell; and,
b) a buffer solution.

57. The kit of claim 56, further comprising primers suitable for hybridization in a polymerase chain reaction mixture with the nucleic acid being amplified.


SEQUENCE LISTING (174 sequences)

SEQ ID NO: 1 - DNA, 1932 bp, YS40 BACTERIOPHAGE
  misc_feature: YS40 ORF1
atgcttatagacttcacgaaagaaactttatcgctatatacttcaagtgaagactattttccagtattagttactagggttgttgattataaagaagcagaggaaggaaaaccagatttttatctattaggtgtaagaacagacttaccacaacaaataataacgatacttcatccttttagaaacgaaataccagttgaacctggagattacatacttgtaaagcgtttaagcgctggatattctatttttgttgcaaaattaaataaaaaatttgatgaagaaaacagagaaaacgaaaggtttatatttaagccaacaatacctcaattttctgataaaaaatcttttggtcaatcttacaactcttctcttataaatctaacctatactggttcttacctaaaaaaagaaccagatgcactttacaaaagatttgttgaatttccttcttatgacctttataggtcaagttatgatgtctttaactacgatgaaaaattttgggaaatattaaacgataaaaacgaagacgaaatttcaagaaagtatatttttcacttatcagaaaacccaacttacaaagactacttattagacagaaataaagacgaaaactttccctttagaccagatttagtatttgactttacacatgaatattcgccccaaaataaacaagataagctactaaagttagacaaaaaaataaccctatttacagaagttgatacagataaatatccttttctccaagctaaagaagaggttggcgaaaatttagcgctaacatcttatgatgaaattttgaatacaaaagcagatggttatcttggagaggatatattctatgcgcaagagttcagagaaataaaattaggaagaaataaaattggtctttataagataagcaaaaatgactctttcctacttttaaagacaagcaaggaccaacagtttgcagcaattaattttgaaaataaaagtcaattaagaataagaaacgatgatggttcatctattttgttagaaaaaggtgaaggatttaacagaacttttatctcaacctatccttcactattatttgaacagttttataaggaaagctatcaacatgtccttattttgaatgcggaaaaggagggctttaaaaatttacaaaaatcctcttccggcttagaggattactcgtttggactatttttaagaggaaataaagatggttccttgaaaagaacagattttacttataaaaatgccttaagtggaggtcaatcagtagtttttggtaccaaaagacaaagttctttttcctacattacgttatcttcagattcttataaatcaatttttaacacacatgtagaaaactctacttatgttgacattttagctgactcttccagcggatgttttaaagtagaaaacaacaaaaacggcgtaaaaaagtttacattttgtgcagaccagtttgttattgaagattatttgacagtccataaggacttattagttaacggaaatacacaattgcaaggaaacttgaatgttttaggaacaacatcacttacaactacatatgttaatggaatcctaatagtaagtggagatgtgtggatacaaggcactctgtaccaaacctccgatgctaggtttaagaccaaccttgaaccaattcaagacgctttagaaaaactcgggcaaatcaccggttatacctttgaaatgagaggtaagcgtttggccgggttgattgctcaggaggttcagaatgtcctacctgaggcagttgatgtagatcagaatggttatttacagttaaactacaacgctgttgtagcactcttagttgaagccttaaaagaaaagaaaaagaaaattgatgagttagaagatagaataaaaaagattgaggaatttttgaaggggtaa

SEQ ID NO: 2 - DNA, 2646 bp, YS40 BACTERIOPHAGE
  misc_feature: YS40 ORF2
atggagaactctaatgagtttataaaaatcagagaagatggaaaacaggtggttaaaaaggttccaccaggctatattttgcttcataagcctacaggaatgttttttcccccagaatcttctcccggttggaaaggtgttttgttagatgaagattgggaattagtagatgtttcttttacaaatcaaaacgtcttgctttctccaatgccgcaacagagttccgttaaggaaaacacgaaaaaaacacaaacaaaaagacaaataaatactcaacttaaaagcgagataaaaacaattcaatcagcgttaaagctttcttcatttctagagagtagtgatgaagtagatatagaagatttatgggaatcaagtatagaatttttagaacattcgactgaaaaccttagagagcttgtagagaaagcagttttagaaattagaggaaagacaaatttgagcaaagaagaagtaaagtttttaaattcagctcaagatctgacagattatttaaaaacacttttatcacttgtttctctaaaaggagatttaaggaaatctttgtcttctggtgtaagcttgtcaaatttatcaaaacaaataataaacggcaattctatatataagcgatttaaagatgctttaggaaaacggaaatctgaaggttttttcgtaggaaagtttttatacgacatatatgatgcgctaagacaatttgaattttcaagaagatcgttatcaatgttttatgagttatacgaaatttctgaacaattcttaaaagaaaaggatttgcaaaaaaagaagaaatatcaattaccaatggaacttttactaagccaaattcaaaaaattctaaattctagaaaaggtaattttatatctgttcccatacacgaagagtttgttgacaaaggaatgtttgcgttttttgtttcttatcccgaatttttgtttaacttggttaaaaaggatttgggaataaaggaaaaaattaagagttctgtatatgctggaaacggagtttttaactatgaactagaaaatggaacctatgttgttgttgaggtgtccaaatactttccaggaaaggagaatttatatgttccgaaaactgattataagtataagggtcaacaaaactattttttacctatattgagagtcttaaattacgacaaaatatctgatgagttttcaataaaagcaaggatatattcagatgaggaagtagaaaagaaaagtatagaaaagacttttgtttacatgccgttttctcacggctttgaatcttcgtttgtagttacacattttttggtttttgatgcggacgaaattgataaaattgaggaatacaaaagatttttgaaaaaaacaggaaaaagagccaaaaaaatatcaaacattgtagattctttagacttcaaaactctagtaaaagagagcataaattcagtattatacgatatgatatcaagaccgcccacgcttggattgtcttttttagatttcctgtttttgaagcaattatatcccaaggtttttgaaggcgaacactcctttcaggaagtagataaattaattaaggatttatttggattaaagctcctatttcctaaagatgaacaaaggttaatagatgaagaaatttttcaagctggacctaatagtttttacattttgcgaagaaatttactttcagcatccctaaggtatctttctctaattgattatgaatataacagaaataaaagtggtccagtttttgactttacagtaagagaaaaaagtccaaatgaaattttttctgaggcaaataatttagcgccaaatgatttggatgactttgatcatctttatagaatagctgagttgagctttactttagcaaataagttctttgatgggtcgttaacggatagagatattataaatacgctgctaaatagtggtttaataaagaatttttataaagatcttcattctttgtacatcacttcttcgctaatgattaaagagttaagtcaaatatcttcaaatattagtgtatggatgaaatacaaaggaggagcatttgaaaagatatttttaaaaattttagaaacctacttgaatgacagatacaataacaaagaatactcaacttcatcggtaaaaagaagactagaaatacaagaaataagatataaagcatatgaagttggtactaatagatacattctttacataaaagaggagtatggaggtatcggagatgtaagaatgtttgtttatgacaaatcttcgaaagaagtaattcctataatttatgaattaaaagcaagagataagtatgtagttccacaactaaattccccaaaagagctttatagaacaatacacgatgtgcttttgtcttttgatgaaaaagctacaagctacaaaatttacaaaccgtatatttacggtattggcgtaaaagcaaaacaaggtactagcgatatttatattgttgatatgaataacggagaaaatcttgccgcaaacataaaaaaagtgaaaatcaaaaataaagacgctataataatttatgctcctagagtagcagtaactcctttctccttgataggaaattcagtagttacttccgaggaaacctttaactaa

SEQ ID NO: 3 - DNA, 2838 bp, YS40 BACTERIOPHAGE
  misc_feature: YS40 ORF3
atgaaaagcttggaaaacatcatcaagggatttatagtaaatgcaaacgagcaaaagaggccgagttttagttcaaatataaaggccaatgtagattctctttctaggggaaaagattatccaggatttaagcctttactgacatatagagcgctagcttggaatagcacagtagtttattcgattattatttttagaaaaaatcaggtattaaaaaaggaaaaaatcatagttccttataatcatcaagaacctccttttaagtttaacctgtttgaatactcaccagaaagtttgatgtatcttccttcaattagtgatccagatgctttctttctaataaatcttttcagaaagtatcgttttaacaacgatagcaagcttattaaagttagtgaaataccaaaaaaacttactagtaaagagctggaaatatacaaacatatagaggataaacatgtgaactattatttaaagagaataagagatgctagaaatatattggaatttttagaaagaccagatccttatttttcagaggtaaatagttgggaataccttcttggaatggtgttagatgatatcttgacaatagatagaggagcaatagtaaaaattagggatgaacagggcaatttagttgctataacaccagtagatggcacgacaataaaaccgattttaagtgaagacacaggaattgtggttggctatgtccaagaagtagatggagctattgttgcccactttgataagcgagatgttgttttatttagacaaaaccttacaccagatgtttacatgtatggctactccttgccgccaattgaaattctttataaagtaatactttcagatatttttatagacaaaggaaacctagactattacagaaagggaggaagcataccagaaggtattttagctattgaacctccttcttataaagaaggagatatttatccacaactatctagagaacagctagaatctatacaaagacagcttcaagcaataatgatgggcgattatacgcaagttcctattctctctggtggaaaatttacctggatagactttaaaggaaaaagaagagatatgcagtttaaagaacttgcagaatttgttgctagaaaaatttgcgctgtatatcaagtatctcctcaagatgttggtattcttgaaggttcaaataaagcaacagcagaagtaatggcttcactaaccaaagcaaagggtctagaacctcttatggctactatttctaagggttttgatgaggtagttagtgagtttagaaatgaaaaagacataaaattgtggtttaaagaagacgatttagaaaaagaaagagattggtggaatataattcaaggacaattaaatacaggattcaggtcaataaatgaggcgagaatggaaaagggcttagaacctgtaccttggggagatgtacctttctctggtttaagaaattggaagcctgaggacgaacaggctaaagcgcaacaaggcgctatgcctcctcagcttgcacaggcaatggcagatcagccttcccaacaaggaggaggagtagacgaaaatagtagtgttccctctgagcaaaaaaatgctggattagaagttcttagaaatttatttaaatctttagacgctaacgcctcggaaaatctcaaacaggtaatagaacttactaacgatgataattatctaaaagaaaaagaacttcttacaagagttttgaaatctgtaggtttggactctgtaagtgagtttatagaaaataacagccaaacagatgttgaagtaagcgcaaaagacattttgagttttaagtataattctctagttgaagatgaaacaatctacgcaacagaaaaagacattgttcttaggctattggggaaaaattcattagaccatagagaaacttttggattaatagcaacacactttgattgtcatactgtgcctaaagttgagttttattcagataaagacaaaagatatgtttttgcaaacctttcagtttgttctccgataaattctgtagaaaaatttacgagattagatgaactattaagttgtactacgaaaaagccaaccaagctagagattgtttttgattggttggagtttaagaattttaaagatattgacctattaaaatattctcaacttggcgattcaataaagaagaaaaactatgacagctttgattatttggatgatgtagaaaaattattagtagctccatatgaagagttagaaaatctcgttaatttgctaaagaagaagtttttcgtagaaggagaactatttagtcttgttgacagctacaaagctacacctcactcattgttccttctattttctttgatagaagaacttggtgtagaaaaaattaatagcttagttgcctcttgcaaaggcgttttagaaatgctattgggtgtgcaagtagaaaattttgaaaccaatcatatggttcattggttattgagttttagctacgacaaaatcagagaagtgatcgcatcgctgtcatactttatcgaaacaattagcattgattgcgaatttgaaaaaattcgtttgcttggtactatcggctcattaatttcttctttcggccaaaacaaaaatataaactttgtttttgtagcagaaaactttgatgattatatgaacttagtagaaagtatatctgaagaagttgataatatatttatgaagagctttgtttttaaatctcttctaggtgaacaactagaaaggcaagaagcaagagattatgtaaaagagtttattaagaaaaagtatggagaactctaa

SEQ ID NO: 4 - DNA, 657 bp, YS40 BACTERIOPHAGE
  misc_feature: YS40 ORF4
gtggaaaagcttaaagtagaaataacttttaacaactatcaagaatcagattattcaaggacaaagagatttaaaaatcttagaaaattactttttttatttgcccaatctatatactctttgtacatttcgctttatattcttcttaggtcaagggtcttttttgtcaaagaagaaaacttttctaagtttaaagaaaagataaaatttaataactacacaaactcaggaaactattttgattctactaggttatcagatatttcaaaatcgtttaatctaaaagtgtcttctccttattactacgaagggttttttaagaaaaacacgacaagaagtgggttggtaaattatatactttttgatgcctttttaaaactttgcttagaaaaatattacgatgacgagttttgcgtttctaagttttcagatgtagagataaatatcttggatatggaaaatggaatttttaatgaagattatatagtaaactcaagtaatactatggaaatttttgtttcagtaaaaaacaaagtgagcaatccacaagtaaaaaggatactagaagataaacttttcgtaaaatatattaatgaggtttttagaaacgagatcccctttatacacatatctgggatagctattttgggagattga

SEQ ID NO: 5 - DNA, 435 bp, YS40 BACTERIOPHAGE
  misc_feature: YS40 ORF5
gtgtgggggttaatttttttaacaaaattgtataatttttttgtgaaaaataagcaggttcctactgagggcactcatgtcatattagagttaagagttatggaaggtaaagattttaggggcgataaattgaaatttttcttctacgatttgctaaaagaatcaaacgcaacaataatagattgtaaacactttgtttttcctaacgaagcttcttctggagtttgtcttttgggcgaatctcacctttcttggcactactggatagatgaaggatatgtttctttagatatttacacttgcggaaatagctttaatcatatcaaagctttgtcgattttgaaagaaaactttgttatagatgacataaaaatactaaaaagagggataaacaagcggggaatgtataagataaagataagcgacgagatttaa

SEQ ID NO: 6 - DNA, 225 bp, YS40 BACTERIOPHAGE
  misc_feature: YS40 ORF6
atgcgatacagggtaaaaggagtagacaggctggacgatctgatggtggctgtagaagaggagtttattgccctgcagaagaactttagagagcttacccagaagggcaagaaggtagcaaggcaaaggcttcgcaagaatactctagcgcttgaaaagcttcttaaggagtctagaaagcttctcaaggaaattgaatttgaaccagtaaatactgtagagtaa

SEQ ID NO: 7 - DNA, 612 bp, YS40 BACTERIOPHAGE
  misc_feature: YS40 ORF7
gtggacacatttaagtttaagtacgacaaaaacagcactttgttagctggaaagcttgtagatgctagaaatgatgtacttatggttgaagttgagaaaaatgtttttccagtattagttccaatggacttttctatagttaaggaaaaggttgtaagtagcttgaatcaaaaagcttctttgtacgctttagtgtacgggatgttgtcaaatggtgaaaaggaggactatatttatgcaacatcaatttcattttcttcatatccccaaaaagataaaaggtttgctacaatttctggtactttggttggagtttatgataatggcaatcgtaagaggctactaattgaaactacaagaaaaaagtctactagaatttttgtaacgcttttcaacagcagaggaatacatgtagataaaagtatgattggaaagaaactaatgattgacggcatattggaaaattacaaaaataagttttctataatcgctgattatgttttcgtagaacttgctggtgctgaaggcaaaacagtaaatagcaactcagatgaagagaatttggtgtataatgatgaagcaaaggaactcggagatgagttcctgatttag

SEQ ID NO: 8 - DNA, 543 bp, YS40 BACTERIOPHAGE
  misc_feature: YS40 ORF8
atgctggtaacggaggtggagatgaccaaaatcccctacattaagctaaataaatcggtcaaggatttgaaggttaacattttggaaaatgctggtattgatttgtttcctgatgtttcaactgttgaaattaaagacgaaaaattaattcgtattaaaaaggtttctaatgatgaggtaagtcttcttgttctttatcctggttcttatgtctttgttactactccatataaaattgcattacccaaaggtattcacggagttatatttggtaggagcggcaatttcttcaagtctggagtagatgtgtttcatggggtgattgatagctcatatcgtggtccaataaaagttggactaaaattttatagtacaggaatatttaacattaggaatgatgttgctgtggcacagttggtgttaattggatatgatagtaggtcattttttgaaatggttgaagtaagtgaggaagaatttttaaataaatatgccaacacagaaaggggagaaagggggtttggttctagtggacacatttaa

SEQ ID NO: 9 - DNA, 828 bp, YS40 BACTERIOPHAGE
  misc_feature: YS40 ORF9
atgcaggagggttttggagagttcccaagagaaataaaggtgcttgataagggttttgttcgtcttgttgactacatgggaagcgatcagctaattgttcaggctgctagagtttcttacaactttaaaggaaagtctaaggatgaagcgttaatagattacctaatgcgcaatgaacatatgtcgccctttgaaatggtagaatttgtttttcaccttaaagtacctatctttgtagcaaggcaaatttttagacacagaacagctagctttaatgaaataagtggtagatatgtaaaacttgaagatgagttttatgttcctagtgaatttagaacaaactccaaagcaaacaaacaatcctccgctgaagaaaacaatacagaaaaactacttaagacaataaaagtttttgatgaaacttataaatatgtttacaaaaactaccaagaaatgttagatgagggtttggctagagaagttgcgagaagccttttgcctttaggtacatacacaatgttctttttcaagcaggacttgagaaacctaatgaactttttaagacttagactagacgatcacacgcaaaaagaaactagagaagttgcaaaggcaatagcttcttttgttgctaagattgttcctatcacttggcgagcctttttaagacaccaacttaacgctgtaaagctttctgaagatgatgttaaacacatagactttacaaactttacgataattaataaagaagaattaagtgaaaccaagctagaggagcttgaaagcaaggtaaagaggctgaggaagcttaaggggcttgacatatga

SEQ ID NO: 10 - DNA, 516 bp, YS40 BACTERIOPHAGE
  misc_feature: YS40 ORF10
atggatagccaaagaagactagaaagcccttacaaaacaatttttaaggaaattcctgatgtttataaaatcagcataaaagatgttgcacaaacattaaagataccatttaatgaagaaacatattttttggacttttctgttataaacgatttttctcccgaagaactaaatgagttgttattttctgttctttcttggttagagcatataaattacaaaaagactctaattgaaggaattttggcaataaaagataacgaagaagaatctatgtatgccaatctttttatacaaacaaagacttctttatcatcagaaaagaaaattacaaatactgaagtagaaaagctagttgagataaacgaagaatatagaaacttaaagaaaaaaatcgccttatataaggcttatttaaattacctttcaggtctttatgagattttagaaatgttgcactactcaataaaacataaaatttccataagtgcttcaaacgaaagaaagattttctga

SEQ ID NO: 11 - DNA, 474 bp, YS40 BACTERIOPHAGE
  misc_feature: YS40 ORF11
atgagtgatttttcttattactgtaattcaatgagagaatatcttaatagtgatgtttttgggcaactagaaaaggaatatttagagtatattcataattttgaatgtattgcgtgctatttgggccaaagaagtttttcaaaaacggaaacaattgctcatcatgaaaacctttcctattgttatagaaattataaaagatttacagatttctcaacgataccattgtgtgattatcatcacaaagaaagacacgattttggattttcaatgacatataaaaaatggtttggcaacgaaaatatagtttatttacttgttttcaacttaatcaaagaattctttaataaaaaagaagaccttagaagagaaaaaattgcagacgaaattttaaagagcgtatccctagaggacgtattgacaaacagaaagctttttgataggctactagtatggatagccaaagaagactag

SEQ ID NO: 12 - DNA, 1020 bp, YS40 BACTERIOPHAGE
  misc_feature: YS40 ORF12
gtgaacaccgaaaaaataaagcaaatcatttcttcttatgcgaagaagtttagtaaagaagaaatttatacaggtcaagaattaaagcaaactaaagaggaaatagtttctacaggtatattgacagtagatttggcgctaggaattggtggaattccgatgggaaaaattattgaagtatacggtcaagagtcttctggtaaaactacttttagcctaataactatttctcaaatgcaaaaagcgaataaaatatgtgcttttattgatgccgaaaactcttttgatccgatttgggctgaaactcttggtgtaaatcttgatgaactgcttttaattgaggcaaattccttggaagaaagcctcgaaaaattagagtttctaataaaccaaggagttaaatatgtcgtttacgattcaattgtagctccgcctactgtatctcaaaataacgctgattacggagatagcctggttggtgtgagggctagaattctttcaatggcactttcaaaattaatgcctatcataaggcaaaataaagcaacaataatgtttattaaccaaattcgtgaaaaaataggagtttatggaaatccagaaaccactccaggtggtagagctttaaaatttttctcctctcttagacttaatatgcgcaaaaagccgataaaagacgaagatggagaaattggagatgaagttcatgtaaaagtcgagaaaaacaagttcgctcctccaatgagaaaggctaaattcttgttgttttacgatggaagttatactatagattatccagaaatattgagccaacttgggcttgttaagaaatcaggtgcttggtataaagaacaaataactggagaagaaaaaacatttcacggcgcaattcaattaattgaggaaataaagaataacaaaccctttgcagagcttgttgaccaaaaaattagagaaaagattgcaaatcttaaatctgtaagtttatctcaagaggaggaagaggaggaagatgagtga

SEQ ID NO: 13 - DNA, 576 bp, YS40 BACTERIOPHAGE
  misc_feature: YS40 ORF13
atgactgaaaaggagatcagaattgagttaatgaggccttttccagaccaagcaattcttttccgtgtagataaaaagctaaaaaatggttcttatttgatagttccatatcttgatgttagatacatcattcatcgcttgaacacaatgattcctggtgattgggaactaaaaacagagataactccaataactgtagaaactactgataaaagcggatttttaattgttggacatatggcaaaggctgaacttacaatcatgggtaaaacaatgacaggaactggttcttcttacttggtttttgataatgatttagaaaagttaaaaaaacaatttattaaggcagatcctaagtctgccgaaacagatgcaataagaagagctgctgcaaaccattcgatcggtttgtatatttggtttttcaaaaatcaaatatttgcaacggaagaagaactaaagaatcgcaactccaaaccgatacaagaggctttggcaaaccttaggataatcggcgataagctttacaggcaagcaaaggagtacgctttaggaaaaaggggaggtgctgagtga

SEQ ID NO: 14 - DNA, 1344 bp, YS40 BACTERIOPHAGE
  misc_feature: YS40 ORF14
atggaccaaaatgaactttttgacgaaaatctttatcttattgagttagaaattgtttttagccttattaaacacaaagatttcatacaaaaggttatctttctttcagaagatgattttgtgtatctaggaaaagcttttgagtatgttaaaaaccataatcaagaaccagaagcatctttattgtctgatattgtgcaacttggtctagttaaagcagaagatgttgaactttatgatgaaattactccagatgaaaaggggcttaatcttacagttaaatatgcgaattggctaaaagaaaaaagtgttcttagaaaaatttctcctcagttaaaagaattgtcaataaaaaaatttttatcaacttcagaagtttactcgaaacttaaagaaatccaggaagaaatttctagaagcggaataaaaaatgttgatattttatctgttgaagacatgtctagggaattttttaatgagcttaacagaaaagacacttataggtttggctttaggtttggaaatatgaaccttgacgattatatttatgattttgtacctggaaatgttgttgttataggtgcaagacctggtatcggaaaaacgcttttatctctgcatgtcgcttacaagcaagctttagaaaatatacctgttcatttgatttcaatggaaatgacaaaatttcagatttttggtaggcttatttcaatgatttcaaagatacctgcgtcaaaaatattcaaaaaagaactaaccgatgaggaaaagaaattggcgcaaataacagttgaaaaaatgaacagttggcccctttattttacttctactcatgatggatctatagaaaacatagagtcaattattagaagaagtgtatatgaaaatggcacaaaaattgtcttcattgattacttacaacttatgtcaaacattaagttttctggaaatagacatttagaaattggctcaattgcgcaaagactaaaaacattagctattgaattggatgttgcaattgttgaaatttctcagttgtcaagaagattaaatgaagaacctaatattgacgacctaaaggaaagtggagatattgagcaagccgcttctctaatcgcattaatgtctaacaaaaaagaggaagagattgattcgataacgctaaacaacggagaaaagattgtttcaacaagaaagtatatcaatttttatgttaaaaaacaaagaaatggtccactttttactgcggtcatttcatacgatacaaaaacttttgattttaatgtagaagagattaagtttggtagtactaaaaagacagatgacaaaaaaggcaagtcttga

SEQ ID NO: 15 - DNA, 294 bp, YS40 BACTERIOPHAGE
  misc_feature: YS40 ORF15
atggtggtaagaaaaagaaaagtaattaagatgaatccctataggcaaacatatgatttaacgcaagaagacctagatattgttcaaaatccatacgatacaagcgaagataaatatgtttttgccaaagaagaaacagattataacgaatacaaatcgtcatacataatgagccaagacgagaacatgtgtgtaaaatataatccctatactatgcaatacgaaaaagttccctgtgattgggatttacaaatgaacccctatacggggaggtacgaatatggaccaaaatga

SEQ ID NO: 16 - DNA, 330 bp, YS40 BACTERIOPHAGE
  misc_feature: YS40 ORF16
atggctaagaagttttcaaaagaaccaacagtagcaacactaaaaagtgaaataagaaaaatactagattcttattatccatacaacaattttaaagtttctttaaaaactgctaacgacacgctaataataaacattgatgggtgcaactttgacgaagaatttcgaaacatgttaaataggtttgttgattacaaaattaaacacatttacagaaaaaagactataaaagaagaaaacaaggttgttgtaaaaaggataccttacaaagacttttacataaacataaatgatgattgcatcatgttcagctactatcgctatctgtaa

SEQ ID NO: 17 - DNA, 1110 bp, YS40 BACTERIOPHAGE
  misc_feature: YS40 ORF17
atgcagatgtacaactacacacaagaagacatttccttcattgagttaatagaggatgtaaactttcacacagaaaacttctacggtagctttgaggattacctaatacttccaacatttcttgaaaaggaaatctctagaaaacagacaaatataatgcaagaaattattgatattaaaggaagtaaaaggcttacaaaaccagttatctcttcaccaatgagcttttttggtaagaggtttggtgaaagattagtagagagaaactttatttactcaattccacgaattaataagccaatagaggagagaatagaaatttacaaagaactgaaatctaaatataactctacaattatcttttctgttggggcaaaggaagaaattacaaatgagataatagataattcagacatcttattacttgatatagctcacggtgcctctaagcattctgtaaatttcttatactctctaagcagacttggtattaattctggtgtgatggtaggaaatattgggtcaataaatgggatgttgtatttattgtactttgcggcaaaatttggctttaaaaacatttacatcagaagtggaataggatcaggaagcgcttgcacaacaagactcaatactggagttggatttccacaattagacttaatgaaagatttacgaaactttttaaacaacttaaattcttctagactaggaaaatacatagaaaatctttctgacacaaaagtatatcttgtttctgatggaggaataaaaaattatggagatatagcaaaggctttgatattttcagatttagtaatgggcggaaaaatctttgcttcaagagaagtagatacttttaataacgaaaaggtgttttattatggtatggcatcacaatacgcaaaacaaaataaagaaaatgttgaaggtgaaggttttgaaattaaaaatcctccacatttagaagaaatactactaggaatagaagacggattgaggtctgctcttacttacactaactcaacaaacctagaagaatttagaagaaaggccaaactaataagagtaagctcttctacactaaaagaaacatatgttggattttaa

SEQ ID NO: 18 - DNA, 411 bp, YS40 BACTERIOPHAGE
  misc_feature: YS40 ORF18
atgactagaccatggacagaagctgaaataaatgaactaattgaattagtaaatgctggtagaaagcgtagagaaattgccaaaatactaggacgaacacctaaatcaatagaatctaagcttagagatattcgtaaaaagagtggagagggcaatgaaaaagttttagcaaataataaaatcaatgaaaacaacaattcacaaaaaaatattgaaaagacgaatagcgaaaaagtcgatgcaattgtcctatatagaaagtttctcaggaaatttttgcagaaagatagcttaccagaaagagagtttgatagcttggcaaatgctgtagaagaatattacataaaaacaaaaaatctaagtggcgcaattcttcttcttgaaacaataatggaatctggtaagtactag

SEQ ID NO: 19 - DNA, 1236 bp, YS40 BACTERIOPHAGE
  misc_feature: YS40 ORF19
atgacatctcaagggtattcacttaaatacattggagctaaatttgtcttagtaaatggagaaggaagatactctaaatatgtaagagcttctgatggaaaggaaatttcaatttatttaaaccccgcaatttacggtggcgttagcacagaagtagttccagttttggatggcactactctgtcttttcaggcattaactccctctgagcttgatgataggtatacagtaggcagacagcttagacttgtttttggtggttctactgctactccagtatttgatgcaacaccagacctttctaattttgaacttgtaaaggtttatgaagatgctagagtagttccacttcttctctttagggtaggagattataggttagcttcttttaatacttttgccgtaccttacgaaaccttctttgaggatttggctagtgaaatagcaggaatgactcctccagatgaaacgctttttttaaagtatctcaacaaccccaaagaaatatttaataagtggattactgtagcttctgcttgcgctggttctggtgcgccaacagttagatttgtagataatgatatttaccttgatgttgatttctctaggtatattactcatgattctgtagccctacaaaacgatgagcacctttttggtatgtctaatactttaaacaggggtagcttcttctattctgttctttctaaggttttctgcgataagtatcagattattcctttctacgacactctacccaagccaatgaatggaaactatacagtatttggcagaccagaaagacttgtagattttaataatgtagtttcaggtgcggtaagtaaagttgctgctcttattagcgatactacagttaaaacttcttttgaagatttggctaaaaagatactttctgaagggcttaaagattggttccgtccttcttcttatcgtgttgttgttgcatacgataataatggaaatccaatctatcaaacagtaaatgtattcctactctatcaaggtgcagctaacggcgctgtattttttgactttaatggaaaagctattagcgtacctgtaacatttggtgatacatttgattacacaaccctagaatatcagaaggcttatcctacatcaaagtcctatgtaactggtccttacggtgccattcccgtttatgacgacaacggaaaccttattggttggtactctgacgcttattctaagtaa

SEQ ID NO: 20 - DNA, 438 bp, YS40 BACTERIOPHAGE
  misc_feature: YS40 ORF20
ttgacagcaacaacaagcttagtaaaattcaaagaaaaagcggatagagaagtcttttttgctaaatttgaggaaataaaaaaatttgtttttgccgcattaaagctggctgagttagtaggaaagtcaatgagcgcatcggaaaacgaaaaagaagatgtttacaaaacaatagaaatgattttcggcaacgaagtccttagtatggtgaagaataacccaaatgaagtttataaaaacacttctcaatttctaaatgataaaatttttgaacttttgacctttgctataaataaaggagcagagataaaagtttttaatgtggaagaagcggtaatggaattgctactttctggagaaatggatgatgtggatgatgatttgatggaaaagataatagaagggatttcaattactcaaaaagttggtgtagcataa

SEQ ID NO: 21 - DNA, 381 bp, YS40 BACTERIOPHAGE
  misc_feature: YS40 ORF21
atgcaagaggttaccttatcagatataaagtctgtgattgttgcgatttctaatcacataatgtttttatgcaaaaagaaaataatatccacatgtttctatgaatttgatacctccataattaacattaaaagcaacccagaggatgatatgtttgaatatatagatttgtttttcctaaaaccaataagagataaaataaatcaagctaaaacaataaaagagcacaaggaacttttggaggaattgaagtttgttacagaagagataaaagtaatagtaaatgaatttttcgaactcaaagaaaaggcaagagagcagtataataaagagtattcttttggaattgataagattttgaaaggagggggagcaagttga

SEQ ID NO: 22 - DNA, 774 bp, YS40 BACTERIOPHAGE
  misc_feature: YS40 ORF22
gtgaatttaccttttaaaatatcaccatcgttagtaaaatcctatgtcttttgcccaaggaattttcagataacaaagttttatttgaaagataacaatatacctaagaaaattccaaccagaagtgctgtcttgggtagattttttcacgcatccttttactatatgtacagagatattttgggaaagtttgattctagtgaagatttagatgaattcaaatttcttttagacaaaaaacatgactttgtaaaagtgttgtacgaaaaggatattattgaagaaaaagacaacctttatgagctagaaaagaacttatttataaattcttttgatttcctgcttcaaataagaaattctatacaagtagagaaagttttggtttgggaggagaaaatagaaggaaaaataagagaatttccagagcttgtgtttgaaataataccagactttgttgcactcacaaaaagaggaaattatgttttgatagacataaaaaatagaagaaactcagaaagccaaaaaataccgtacatcatatacagagaaatacttaacgaaaagtttaatgtagagtttatagttctttctatagatatcctgaaaaggaaaatatatagacacgattacaatcttgagtacaaacaatatttgtattcacagcttaatatgattaggaaatcgcttactacaggcatttatccgcaaaaaacaaggaattgcgtagaatgtaaagtgaggagtttatgcaagaggttaccttatcagatataa

SEQ ID NO: 23 - DNA, 1677 bp, YS40 BACTERIOPHAGE
  misc_feature: YS40 ORF23
atgggcattgagcacaggctggatttttttgaaatagctagtaagtacacaaatcttagacatagaggaggaggaaagtattatggcctttgtcctctacatcccgaagatacaccttcttttcatatagacactaaccttggtgttttttactgttttggatgcaatacaggcggaaacatttatcagtttatttccttagtggagcatgtaccgataaatcaagttggagaaattcttagggaaaaatttggcattgatgtaaaaacaaaaggcgaaaagaagtatgaagatgatcatgatctgttttcaaaatttcttaagttgctgtccaagtataacgatacagaaactgtagacgattttaatattaaaaagttaggattaaaaccaaagtatccgatgttttatgtccctaatgaaagattgttagattttacgaatgaagtaaaggaaaagttagacatagagaggttgataacacttggaatttttagcagagatgaagagggaaaactgaaatttaggtacatgagtagaattattattccaatctactccagagttaatagaattgttggtttagttggtagatcgctaaatgatatgaacttaccaaaatacttaaatacaaaattcagtaaaactgagcatctgccctttttaaacgaggctgtaaagatagcaaagaagaaaaacttaaacactatctacatagtagaaggaccatatgatgcgcttgctttaatagaaagagatatacctgccgcctcattgttgggcatacattttacagtagagcaaattaaccttttaaactcattttccgttgtttattttgtatttgacaacgatgatgctggaacctcagcttatttcaatgttgctaaaacaattataaaaaccaatcgcccttctttttctacaatgtttgttttttataaagataaggaagacatagacgaaattttaaaaaagaaatcgtttgaggaatttataaaaacagcagacacaaaagatgtttatgatgtatttatagacgctcacctgagaaatatcgtcaaggatttgcccattaaaaataaggagctaattagagaagaactaataaaaaggctgatgaaattatttttcaactatagagaaaacgagcatgtttataacttaatgctgagaatatgtgaaaggtcaaaatatccttttgatgcgcttattagaagggtagactatgtgataaataaaggtctgtctaaggcgaaaaaagacatagagtctgtagaaatagattatatacctcccaaagaaagaaagcttctcaggggagtgttataccttgtcaatggacatcacgataagctcatgacgctaaagagagagttgcttttatacaagtataaaagtaagactgtaaaatctttggttgaatttttcttagaagaaagaagtgaaattagtgaggaagaatattctttgctcgtagaaactgaacctgaggaggtagatttaatttctttaatctctgagcagacgacagataagaggtcagaaaaattaaaatctatagcgaaaagattgggctttttaagcgtttctagggcattagagattgaaggaaatatagaaccagtagaagaggaaagcagaaaggagggagaagaaagtgaatttaccttttaa

SEQ ID NO: 24 - DNA, 609 bp, YS40 BACTERIOPHAGE
  misc_feature: YS40 ORF24
atgtcagtctttggagtttcttggggaacagtaggatcaggaaaaagtgaaatggtctacatgtcttccattttatcaaaaaggtataatgtaaattatttactaataaggccaacttttatttctaatccaaaagaaattcgttttaaaaatagaaaggtgtattatcaacactcttattatcctcacgaagttttctataaagtgaaaaaagacgaaaggtatgtcatacatgttcagcatttccatctaattgaacaatatgagttaaagcaaatcataaagttctttaaatataaagatgtcatgataaacttcttaggtttaaaatttgattacgaagctaatgtttacgagcaatttgaatttctcattaaagaggctgacatcagcgaagaattagaatcctattgttctgtttgtggaagaagggctaaatataaccaagcagtttatgatgacgagcctttaccaataattccaagtaagcaaacactaatttatttaaattacgaaccaagatgtgaggattgttatatacatcctaaggaagtttctaaagaaatattagaaagaccagatttattttttagtcttaagcagtcctga

SEQ ID NO: 25 - DNA, 714 bp, YS40 BACTERIOPHAGE
  misc_feature: YS40 ORF25
atgaaagtgctacacaaaaagattctgaactgtggttctgaaaaccaaaggcctcaaagtgtaatttatgataactttgttgagattaactttagtgcagacatagatgaattttctgttaatgagtttgttgatagtatttattttgtttctcatgagctttccaagtttgaagtagttaaaggagatgagtttggaattggactaaataagattccaattattgtacagttttcaacctttggcggagatgtgatgcaagctatgagagtaatcaatttaattaaggggtttgaaagagatgtaatttttgtagctaattctattattgcaagtgctggtgtttacatttttctttccgctaaagagtctaataggtttgttacatctaactcgttctttattatgcatgattttcttgtaaatcatcctcaacccattggttcaaaagatattgttgaaaatttggataaacaatacaaagtattgaagactatctttgaaaacgagtttataaagaagattgggctcaaaaagaaggtaattggaaaaggagcgcataaagatatttacatcacaccagaagaagcaataaaatacaatattgctagcgactttttcaaaggaaaacataaagtttttgagaattttggacttagttacgatgaggtaaagtctatgttctataaatttaaaatgactattgacaactga

SEQ ID NO: 26 - DNA, 927 bp, YS40 BACTERIOPHAGE
  misc_feature: YS40 ORF26
atgatatgtttgaaaaacagcacattagggaggatacagatgaccgttgaagaagttttaaagtttaaggaagaaaacaacaagccgccaagagcaaaatctgtttatgacgttcttagattagtggaagaatacttagtctacatgacaaaaaatggaggaagggttaactttaatttttccttagagtattcaaaagaaaaaggaaattatttaagactctcttcttctggtaagtgtcctagggcaatagcttattctgtaatatatcaatttgaagaacatggagaaattaaagacttatctccaagagctgtaagtatttttcaaatgggccatgctttgcacgaattagagcgtgcacttatttctgagattaatgaacttgtaagcgttgaagatacagtatatcttgaggttgacgattataaaataccaggacatatagacggagttctaaaattagaggatcgtgatgtaattatagacataaaaactgtaaacgaaaagacttttaacgaattcaaaaaagagccaagagaagattatgttaagcaacttaacgcttacatgtatgctacaggcataaaagaagcttatctttgggtttacaacaaaaatacaagcaacagaatgattgttccaattctgtactctgaaaaggttgtagaagaagttatagacaactttagaaaggctataagaggtattaaaggtgagctacctgagcgtccttatcaatatagaattgaagaccttggaaatggtttagtagtgaagaccttaccttggcaatgttcttattgcgcttttacacgaatttgttggcccgatttcaaactcttgacagacgggggcaaggtgaggtatattaaggttgaggaggatgaaagtgctacacaaaaagattctgaactgtggttctga

SEQ ID NO: 27 - DNA, 1350 bp, YS40 BACTERIOPHAGE
  misc_feature: YS40 ORF27
atgagcgctttacctaaggctgtaagttttgtagtacacccgaattgtgtgtttattgagtattcctcacctcttgctaagaggtggatttacgaatccttgattgaaaataggttttctccagaaagtatgtttttgtttaaggaggaaaatggtagagataagggacttgttgttgttccttcatctgttgcttacctgatgggaataaggacattttctcaaagttttaaaccaaagaatcgcaagcttaatttgatagaaaaagtagatcctttttattggcagagaagggccatagaaaaaattatttctgagagaaaaagaaatggattaataaaagctcctactggttcaggtaagactgtttttgctttgcttttggctaataggttaggtagtccttctgtaattgtagttgacagagaggtgttaattgaccaatgggtacataagattaatgaagtttttgaaaaatcggaggttatagttttggatagaaaaaatgttgaggatatcttagacaacgaatatgattttgcagttactacagttcagtttcttaattctctaatgaaaaaagatttcaaaaagtattttgatatttttgacaaatcaaactttaatatggtcatttatgatgaagctcacactactagtgcagcaattgctttcggtaaaagcgtagctttatttggaagatttgaatacatatttggcgttactgctacaccttatatttctaactttcctttacacttttacacgataggtagtgtaataatagatgccgaaaaattaggttacaaaaacgacttaaaagacaaactttcaattgaggttgtgaaagattcctttataaacaaattgaagatttggaatggaatggatagaacattattgttagctcaatatcagacgcaactagagtcctcagaaagatttagggatcgatttgttaatatcattaacaagaagttgtctgaaggaagaagagtacttgttgtagtttctcgcttggaaaccatagactaccttgctaaggtttttaaaaatgcgaaaatacttacatcaaaaagaaaggatactcttgatgaaaatgaaaatctaattatagcaacatatggcgttgtgtctaaaggatttgattatccttctttagatacgctgataacttctgttttaatttacggcaaagtttcttctgttcagcttgttggcagaattttaagaaaaaccaaaaacaaaaaagaaccacaagctatttttgtagtggacaaatcaatggagaacattcttgatgttgacattaaaagagaacttgagtttaagatagatgtatattcatga

SEQ ID NO: 28 - DNA, 1401 bp, YS40 BACTERIOPHAGE
  misc_feature: YS40 ORF28
gtgggactactagagaagattacagaagaccttgggtcttttactcacctagttggcgtagataaagtctactctcctttggatacttttggaattagtgaaattaagtctttcttatctggtagtaaattctactcatttagcttactgagtggaaactactttagattaatcgttattgatgttgatcaagaatgggaagaaaataaagtattgctttttcttaactacttgtttgaggtttacaaccttaaagcttacgctgaaaaaagtatcggtggaagaattcatgttttgattcctttcaactctggaataaagtttgatgagagtaaaatattcttgaagttcatagagagtgtagctattggtttttggagggatagcgtagaggtgtttcctaatggtcgttctataacgctgatatttgatgaggtttatggaagaaaaggactcttgttttcaaatagcgattttaagaagaagttaaggttcttttataggtttttcaaaaagccgttttctaacaccaagaaaacctttgaggaaattttaaaaacacacaaggacttttttggcctaaattcttatgtgaaagaggctttaagttcaaagagtgaagagattgatgttgttagcgttggagaggagttagaagatgtttttagagagattgttcaaaagatagcagaaaaggattatgttagaggtagaagaaacgatttgatttttgagttgtctggatttctagcttttatgggcaaaagttttgaggaaataaagaatcttttatggcctttgattgaaagagatgatgaaagagataaaagaattgagcttattattcgtgctttagctagaaagcgtgaaggaaaaaagttgtattttaaacaacttcttacgatgtttcctagtttgggcaactttgttgtgagaaaaagcattgaatatgctgctttaggagatgttgttatgtcaaggatttctaaagaaacaaacgataaaagagttctaaaggttgccaattttatatctaaactgacgagcaaatacggcagtcgtgtggttcttggatacaattctctgtcaaaaaacctaaatatgagcaaaagagatgtttcgtttgcgctaaagatgttgagcaatctaggaatcataagaagaattgcaaaaggcagatacttgaatattgatggagaaaagatttctataggctcaatttatgaggtttgtattttgagtgcagcttcttttgtctacaagatatctaaagatatgaagtttgcttttgaagcaattaagaacaaaattcgtgaagtgggagaattttacttttctagggcgaactttgaatatttaagagaaaataagtttcttctgttgaatttgaatagcgtttaa

SEQ ID NO: 29 - DNA, 510 bp, YS40 BACTERIOPHAGE
  misc_feature: YS40 ORF29
atgaaaaaggcgcttgtagagcatgtaaagaaagaaaaagatgatgaatttcacactcctcgctttgcagttgaaccgcttttaaaatacataccaaaagataaagttatatggtgtccttttgatactgaagaatcaaattatgttaaagtttttatggagaacggttataaggttgtttattcgcatataagtatgggacaagatttctttttctacgaaccagagaattatgatgttatcgtttctaatccaccgtttagtgtaaaaactgaaattttgaagagggcttatagtcttggaaaaccctttgcgtttttactgccaataacttcgctagaaggaaagaaaagaggagaattgtttagaaaatatggacttcagcttattgtttttgataggagaatagaatttatgtcgacaacaaaaactggcgtttggtttaacaccagctatttctgttataaattgcttcctagggatttaatttttgaacagctagaagtttaa

SEQ ID NO: 30 - DNA, 1758 bp, YS40 BACTERIOPHAGE
  misc_feature: YS40 ORF30
atggaactaaagaaactaattaaagaaatcgacaattctcaattagaccctcaattaatcctaaaaaagagagcgttttctatagaacagctaaaagattattttctgaattcactaataaaattaaccgaaaataagaacgcctatttgtttacaaaggaaaaaacaacattcgatatagaaataaatagcattggaaacgattggaactacacgatatatgtagataagaaatttaaaaaacttgtaaaagtaggcaacttagtactagactaccagtttttgggaaaccaagtaaaaatctggataagtaatagtttatataactacctagataaagttcaggtatttgtgagaacagatattgaatatcttcaattcgaaaaacttaaaagtgaaggaataaatgattataacacatggaaatttgaagggccaatatacggaaatccctctgagaattatgtagtaatcaaatttttactaaccaatggttcaaagctttttgtgaatttaccagtaaattacaatctttactatccgtttggtgtagattataaagttgaaatttacaaaataactgatataccatacctagtagacactatatatccttctttctctttagaaaacaatttcttcgtttactccggctcgataagtaaaacaaacgaaaaagaagtattagcgataaaaataaaaatagacgatgaagaagattttatatactggaaaacttatcttcctgtaagctataaagattttccgtttttaagaacatatatttacgatttagcactttctatactaacatttgtttcagagtttaggttaagctcaaaggattatataaaaagtctaattgtaaagatgtttaattcctccaaaaaccttcttaactcaaaaaattctttcaattttagttatccatcgtacggattcttttctgacgaatacataagaaacggtagtgtagcttggcttctagaagcgcttgcaaaggcatacaaatatattcctgaacttcaaacattagaaaataaaaactttataaaatccatagcagattatttgataggagaaatacaatctaacggtttagtaagaggaggttacggaatttatgacgctcaatataactttgacgcttcctatgtcgtaccatggtacagtacagaacacaacatagacagctactttgcgttaaaaagcgtatatgatataacaaacgagtctatttacaacacaaaggcagaacagataaaaaactctatcataaacaacatttatgacggcgaaaaattaacacaaggatatcaagatacagcgggagcattagatatttatacctggggcacgatatttctttataaaatagagtttgatataaacaaagtatttaaaaactaccaatatttagaccaattctttaaaataaggccaacaaacacttccttcataccaaatttatacaaaccttactccaatgaatttggttatccaggcgcaaaaaacctcctctggtttgaaggctattttcaagcgctttacgcaaagtatttagtctttaaaaatgaacttgagttaagggaagataaaaacaaaatccaaaattttattttaaaagaattacttccttatgcagttggttatgacgatatatatgaaataggtgagtatcctagcatagcctcaataagttggttcttgttcttctactacttgtattaccacaacctagagtctttctggtattaa

SEQ ID NO: 31 - DNA, 714 bp, YS40 BACTERIOPHAGE
  misc_feature: YS40 ORF31
atggagcttaaaatattaaaagtaaacaccaaaataaattgggaagaggttttccagggtttcaataaggactttattgagaatgtaaaaaagatggtttcaaatttaaaaagtttaggaacaaaggatatcagattttttaatatcccttcggcatcttcgcttaaagacattagccaaagctttgaaagcaaggttataagcgaaaattctttattttttaaagaaaaaatgaatattacagcttccgatttgaataaaaaatttttaactttatatttacagtttgaaccacagtatatagaagctgtaaatatagacatatacttaaacaaaaagaaaatagggaatgtaagcttttctataaaaaattcagttataaagtacccaaaagttccttcattgagcaaaaaactttcagatttgtttttgcaacccgaatattttgagggggcaaattttataagtggaggcaatggcttagaagaatttttgtttacgataattgagtttcctggcttgcaagtaattgaaccaatagttaagattcttttttcccgccataaaccaatagatgaaagtggagaagtagagattgttggagagctaaagactctaagtaatttcgataattcaaagtttaaatttgctatctcagaagaaccagagtatcacacgccatatgtatatcgcattaacaaatatgtaagctga

SEQ ID NO: 32 - DNA, 474 bp, YS40 BACTERIOPHAGE
  misc_feature: YS40 ORF32
atgacacaatttactatagacttttctaattgcacctgttcatcagaactatttaaggtaaccttcgaacaaaacaaaaagggtatttcggtaaagataaaaaaccaaacttccagcgttttaaatttagttatcgaagcttatctaatctcaacacaaaaatacttagcaaaaagtgtaagcatacaagctaatgaagaatctttgtccttttttcctgtgcctggtgtagatgatgtaatttttgcaatatacgaaaaaactgatgactgcactatggtatttgatgaacaaacaaaggttctaacttttactcctacaaaatctggtgttggtgctttgtgctttttatctggagatgatgtttcattaaaagtattcattaaaggaaatcaggttccagtatccacgtcctttgaagacacctattctgttttcaacattaattgcttctgtgtaaaaatgcaacaataa

SEQ ID NO: 33 - DNA, 2112 bp, YS40 BACTERIOPHAGE
  misc_feature: YS40 ORF33
atggacaaagaccttttgggatacaaaaaaattgttggaattgatatagaaacttgggatggaaaaaaaggaggttcacttgatccttactcaggaaaaatagcgttagttcagattgcctatgacagaaataacattgttttatacagaccttgggaaaagaattttgaggaagcattgaaggtacttagcgataaagatgttttaaaagttggacacaacttaaaatttgatttaaagtttttctacatgaacaacatatatccagaaccaatatttgatacgatgattgcatcacaaatgatttatgctgggttagatgaaccagatgaaatttctgaagttttagaagcacttaaagacgatttgactacagaagaagtagctatatttgatgtgtttggcaagaaaagaacaaaagctaccaagttttctcacagtctgcaggctgtcttaaagagagaactaaacattttcttaccaaaagatgttcaaaacagcaattggggaggtgagcttagtgaagcccaaaaggaatatgcaattaacgatgttaaatatcttaaagatttgtcaatagagttatggaaaaaaataaaggcattgaacattgaaaatgtgtttatgttagaaatgaagtttttgcctgtgttagtaatgttagagttaacaggggtgaaaattgataaagaaggttggaagaaaaaaatggaaaaagaaaatcaagagcttttagagctagaaaaagaacttaaaaaggaaatatatgaaaaatttgtttctaaacaaagtgaaaaaatgatggtcggattgtttgaagatgcagaatatgaaaaaataaatctaaactcacctttacaaatggcaaaaatttttggattgccaaatgtttctaagctaacccttgagaaaattgaagatccgacaataaaaaagtatgtggaatacaaaaagaaagcaaagttagtaacaacctactcaaaagaatacttagaaaaactaacagaaaacaataggttaatgtccgattactcacaaacgaagactgcaactggaagaattagttctagtaaccccaacctccaaaatgttccctcttggtttaagagatacataacagcagaagaaggatatgttcctgtttttgctgattattctcaagttgagctgagaatcttagcttatctatctggagatgaggccatgattgacagctgtaattctaaagacatgcacagcgaaaatgctagaaagatttacaaaattcccccagatcaacctgttccaccagaactaaggaagaaagcaaaaacagtttcattcgccattccttatggttcctcagcttatgggttggttgtgcgtggcatgttctctagtttggcggaagcagaggaagctattgaaggttttttcaatagctttcctaaagttaaagagtttctagaaaaaagcgcaaagagtgcgcaaaacggcattactagagatgcgttgggtagagtaagaaactacaagaaaatctatattagcgaagatgaaaaaggagctttaaagatacttatgtttagattaccttctggaatttctaaagatgagtttataaaggaggttttagagaaaaatagctttaaagacgctgcaaaaatttttggaatggatgaacaaatgctacagctagcaatttctgcttacgaaaaaacaaagaaattggctaagattgcgagagaaggacaaaatcacccgattcaagctacttctgcatcaattacaaaaacagctatggttgatctcttagaatacctgtcaatgacagggtatggatatatgactttaacagttcatgacagcattttctttgaaatcaagaaagaatttatttttgatgcacttccaaaaataaaagaaataatggaacaagctggtccaaaagttgttcccggaattataactccagttgatgttgaggtaggagagaaaatatcggtatattgcaattcttgcggtaaagaaaacgagattaatagatacatggtaaatctaaatgagaagataattgttgattttactaaggatccttatatttgcgatgaatgctttaacagggttatttaa

SEQ ID NO: 34 - DNA, 291 bp, YS40 BACTERIOPHAGE
  misc_feature: YS40 ORF34
atggaactaaatagcgacatagtgaggctctttgttaatttcttgggaatgataataacttcctgggctacattttcactagtagtgttaataggaagcttcataattaaacaaaagccgagttttcgtgatatttcttggggagtagctggatttctgtggattatatctaatctatttgaagacttgattggcagaaaattggatttgttagaagtttttatagtagcatcgtttgaaatatccctaatgcccttctggaatttttggttaagaaactattttaaataa

SEQ ID NO: 35 - DNA, 267 bp, YS40 BACTEROPHAGE
  misc_feature: YS40 ORF35
atgaatcaagaaaacactataataagaataactttattaacaattacctccgttactgcactagcactcttaatacgcagtttattaacgggagaggatatacctcaaggttgggcatttttaataggctcagcctggggtgccctattaggtataggtgaactcgcacacagattaaaaggaaattcctataatgtcaacgaaaaagagaacgatgatgataaaaaagataggacgggggtaaagaacgacaatggaactaaatag

SEQ ID NO: 36 - DNA, 279 bp, YS40 BACTERIOPHAGE
  misc_feature: YS40 ORF36
atggtagctatattaaaaataatctcattcctaaacctagttgttcaatacctccctacagttataaaagtggtccaaaaggtagaaagtctatataaagaaaaagacgggaaagaaaagaagagaattgcaatggatcttcttgatgaagcgctaaacataacttcattaagcgaggaaaagcaaaaggaaattgttaattttgtttctggtcttattgatgctgtagtttccttcttgaaccttaagaatgcgtgggaaaatgaaaagcaaaaatga

SEQ ID NO: 37 - DNA, 366 bp, YS40 BACTEROPHAGE
  misc_feature: YS40 ORF37
atgctgttttatggtaatattgaacaagggagaaacatgctaggagcagtttacatactagaaaagaggggaaggggtttttacaaggttatagagaaggtggttaatgagtttgtcaatgattctgatttagagagtttaacaattagtttggcgaatgatgtttttgcaaaaaatggagcaattggtaaagttgaaccaacaaatgaattttttatgagcggttctgtagaaatcgatggaaaaaaatttaaaacgagaagagtaaaaacttatagagtaaagataaatcactcaaaactgacaaaagatgcggaattttttgttgaatctgttattttaagtgagaatggacaagtggcataa

SEQ ID NO: 38 - DNA, 429 bp, YS40 BACTERIOPHAGE
  misc_feature: YS40 ORF38
atggacaagtggcataaaagattcattgaattagcaaaaacaattgctcaatattcgaaggatccaagaacaaaagtaggtgctgtaattgttgataatgaaagaagaattgtttctatgggatataatggcttcccaagaaaagttttggatttagaagaaagattaaataacagaaaagaaaaactcaaatatgttgttcatgcagagttgaatgctattttaaatgctaaaagagatatagagggaacaacaatttatgtttatccttattttccttgcaatgagtgtgccaaagcaataattcagtctggtataaagaaggtaataactacgggaatagtagatgataattggaaagaatcagttgaagcctcaaaaaacatgtttaatgaagctggtataattgtggaaatctttcaagaataa

SEQ ID NO: 39 - DNA, 429 bp, YS40 BACTERIOPHAGE
  misc_feature: YS40 ORF39
atgtatattgatgttataaaattaatcttgcttatagtttcttttacaaatggtaaaccagctacagaggatatcgcttggcaagtagcttttaaatttacactaccagaaaatacgccaatagttgaagtttcatttgattatgtgcctatttacttttcttctttaaacggaaatctttgcggaatatcttatggagcgtttgtgattattgatccaaacaataaagaaaaaggttgtgaaaatacattggaacacgaattggcgcatatttggcaatataggtcttatgggtttatattacccataacatatccatttatgaaagacttttgggaaactgatgaaccatggcacaaaataccagaaacacacagagttttaaactttccccttataagattttatataccgttaacttttccgtga

SEQ ID NO: 40 - DNA, 525 bp, YS40 BACTERIOPHAGE
  misc_feature: YS40 ORF40
atgaaggatactttcaacatctacatagacgtagccttaagaaaatctggtgttttaataacaaaaggcaaacaaccgatagagcattttttaattaagtatgacaaagaatttaatgtagaaaactttgaagctattgttgcacacaaaaaatttataaaaaagacatttcaaaaaataaaagaaaagtataaaaagtttgataaggcatttttagaagcagatgttcttggttatagaaaagggggttttaagaataaggaattaatgacaatagtaagaatgaattatagcttttctttaagtgaagttttagttaaaccaaaaaatattcactttataacagcgtctgtatggaaaagaaaactactaggaaatgccgctttaaaaaaagcagagtacaaagaaaggttggtaaaattgattgaaaaggaattgcacatagaaattagtgatatcaaaagtatggatatattggatgcatacggcattttcctgtatttctttaaatcaaatgttgtgtag

SEQ ID NO: 41 - DNA, 2394 bp, YS40 BACTERIOPHAGE
  misc_feature: YS40 ORF41
atgactgacctagcaagatacaaggaagagctttacggatctgaagccttaagggttctttttgataagataaagagtaattttgaagaggatgttgatacttttgagaaatttttggaatatggagaaccagaaaatgcgctaacaaaagaagaaattaacaatgtatttactgaacttgaaaaactaggattaattgaagaaattaaagatgagctttcttctttctacaaagtaaagaaagaagatttgattgacgggttttttgttcaaaacatttctagcgaaatctacaaacaaaaataccaactgaaggatggaaaaaacaaaaatgagctagttgatttaacgccataccacacatttgttcgtgtagcactagctgtagcattagttaatgctagatactcaaaaagattagaaaagtatggtttagatgcatttaagacttttgttgacgattttctttccttcttttacattttggcaactggaagatctactggtgcgggtagagtaatgtcaaacgctggttctaagttatataagacttacacgacactaataaactgtacagtaatgaggcaaattcctgatagcatgcatggaataatggaaacactaactgaagctagtcttaccttaaaggctggtgcgggaacaggatacgatttttcctctattcgtcctaagggcgcttttgttcacggtgctggtgcagaaacttctggtgtagtttcatttgctgaaatttttgacaaaatgtgttcaactattatgtctgctggtggaagaagaggagcacaaatgcttgttattgatgtaagacatccagaaagcattgatctatttatggcaaaaagaaatgatggggttcttagatatttcaacatttctcttcttttccccaattcactaatggaaaaagttaaaaatgacgaagaatttgagcaatggttctggacttttgagatgacatttactcctagtgaggttattgaaaaaaatggtaacaatgttttctttattggagataaagaagtagtaattgttgaaaatggcaaacttccttatgagtacgaagaatacgactattttgcttttggtgaagatcatcaagtaatgaaatatgaaaattttggtaaagataagtatagaatttacaaaaaagaagtatttaagagagtaagagctagggaaatttatgatatcgtaatgcaatctacctacgactatgcagaaccaggaattatttttattgataatgtaaacaacaaaaatgttttgaagggaaaggaatacattagggcaacaaatccatgtggagaacagcctttgccaccatacggttcttgcaatttaggttctattttccttcacaggtttgttaaggagccctttaattatgatttgaaaccagaagaaaattttgattttgacaaactttttgaggtttccaggttaatgaatagatttcttgattttgttaatgacataacaaatcttccgctagttcaattaaaagaggccgcttttactaagagaaggcatgggcttggaatttctggtattgcagacacattaataatggttggacttagatacggttctgaggaagctgcaaagtttttagaaaaaataatgtatacgatacaacttgcttctgcagtagaaaatgttagaatggccgatatttatggtcctgctcccgttggcgttacactaaaagaatgggtagatgtgtttggtcatttaaaagagaagcttgaagagatttacggagaagacttttataaactaccacttagatattctcacgcaatctctattgctccaactggaacaatttctttaacattcggaaacaatgtagctaacggaattgagcctacttttgctttgtcttacatgaggaacattagattgcctggcaaaaaaacaaaaactcaagaagaggtctattcatttgctgcaatcctttttaagaattactttggacacacaaattacaatgaatattttgttactacaaacgatattactgtagatgaacatataaggatgcaagctagcgcacagaaatatgttgatagcgcaatttcaaagacctgcaatgttcctactgattatccatttgagatgtttaaagaagcttatattaaggcatatgaattgggccttaaaggatttaccacattcagatttaaccccaagttttctgtaggcgttcttgtaaagaaagatgacttagagaacctgttaattgaatttgaaacagatactggggagaaatatgttgtaaaaggttcagatattgttgtttatgatggtgaagaacatgtggcagccaatctttacgaggcattaaaagaaggcatttacgggcgcatgtaa

SEQ ID NO: 42 - DNA, 600 bp, YS40 BACTERIOPHAGE
  misc_feature: YS40 ORF42
atggtagagataaagggaaagataatcggatttcgtcttctagaaaacaaagaagatgataaacaaaaaccccttattagaaatcaagtggttcatggttatacaataaaaatttcatacaacgatcactctctctatttcaccttaaattttgatgagcaaggaaatcttttagagctttttgttaacagctcctcaacatcgtcttcaactaatgcttatatacaagcgatcgctagaatggtaagcaatcaattaaaatatagaattccaatagaaaagattatcaagcacttagataaccttgattctggagaaacttatttggtaaagtttcctggaatgaagaaatctaaattcattaaaagtattcctgatttaatagcaaaagttttgacatactacaaagatgaagaaacgcttaaaaaaatttcccaaggaaataatttagatgaaaaaccagaagaaaaatataatgatgaaacaaaaattgaagttaagacctcttctataacttgtccctcatgtggaagcaatagcgtaaaagttgaagaaggatgctttacatgcttgtcttgcgggtattctaagtgtggctga

SEQ ID NO: 43 - DNA, 207 bp, YS40 BACTERIOPHAGE
  misc_feature: YS40 ORF43
atgaacctttcctgggtggaagaacttagaaaggaaagagaaatgatcgaaagcgaatatgcctctcttggacaaaagatacaagaactagaagctagaaggaaaatgttaagagaacgggcaatttatttggagggtgttttagatgctgtaaagaagattcaagaagaaacacaagccaacgaaacaagccaagaaggtaactaa

SEQ ID NO: 44 - DNA, 453 bp, YS40 BACTERIOPHAGE
  misc_feature: YS40 ORF44
atgttagtagatcaggatggaaagccactaagcaagaaggagcttaagtatcttcctaattttttgggaattgtttctgttgttacaaagaaaagcgttgtggaaacaaatgaaccacttgaaccatactttataatgtttatgcttgttacgcctcaaaaaatggagctttattctcaggctggttcgttgttgcaggaggtaatacttcacaatcttcttgactcatatgttaagaaggaatacgggaaagaattaaaggaaatggacgaaaaagaagcacaaaaaatgctttccgaatttttgaggaaatatcaagtaaacattcagccacaaccaattcctctggattttgttgttttagaaaacattgacgaagaagaattaaagaagctagtagagcaggaaaaactaaagagcggctttttggtaagcggcaatgaacaaggataa

SEQ ID NO: 45 - DNA, 330 bp, YS40 BACTERIOPHAGE
  misc_feature: YS40 ORF45
atgaacaaggataaagttcttacagtcaactcgctaattgttagggttcaaaagagtggagagaagtatactgatactggattagttgttgtggaagaagaaaaggctgtaaattttggagaagttgtatctttaccctttctaattacagaatctcataaagaatacgaagaacttgtaaaacatatttcggtaggagatactgtaatatttaatgcaaaaatgacatacaatatcattaagcttgaaaatcaaaaaccaaacgaagaatttttaataatatcacttgataacatacacgcaattgtaaagaagaaggaggtgcaatga

SEQ ID NO: 46 - DNA, 216 bp, YS40 BACTERIOPHAGE
  misc_feature: YS40 ORF46
atgacaacaaccttctcttttaaaacgaaaaaaacatgttcttgtaaattttctgacgaatatacacttgtttgcgaaaatgatagctgcaaattgtattccaacggtattttagtaacaaaagataaaaattacgcaagaacactaatagaaagtgttaaggattctttagaagaagaggactatagaaaggcattggagcttttggggggttag

SEQ ID NO: 47 - DNA, 444 bp, YS40 BACTERIOPHAGE
  misc_feature: YS40 ORF47
atggacattgttttgtttttcaaaacctctttactaaccttcttaatttctcatgtttatatacgatacctaaagtatatacccaaatttcgtatctttaagggcatttttcaagaatattactattgttatgcgtgtgtaggtttttgggtagcggcgcttattgttgtgattgttcgtcctgaaataactgataagcttttgtttgcaattttaggaacaattggatttttgtataattttgatacaagcgtccttttttcaagaattagagaaaatggacttttttcttttacccctgttgtaagtgctgtaatccttgctgcttttgtctttttaatttccttgaaaatttctgcaatttctttgctattaacaatatcgctaacaaatgttatttacaaagaactctatgaacgctttttaggaggctccgatgcctaa

SEQ ID NO: 48 - DNA, 309 bp, YS40 BACTERIOPHAGE
  misc_feature: YS40 ORF48
atgcctaaaaaggtaaaaacagaagatattttgtcttctttaatacaaacaaactttacactttccaacaaaaaggatttttttccaacaaccaagaaggaatttttaaaactgaaaaagtttttgggaaagtcattaaacctagaaggaagatatgattttgatgctatatatttgcctataaatgaaattatatctttagtttcacagaaggattattggacaaccaaagaactgaaactacttgatgagcagattaaagagtattctcttagatatttagtaaagactgtagcaaatatgatataa

SEQ ID NO: 49 - DNA, 348 bp, YS40 BACTERIOPHAGE
  misc_feature: YS40 ORF49
atggacagaaacaacacaagacaaaaatttcaagaggtaagacaggcaaaaatgcttgcatttttgtctaatacatgtagtttttgtcaacaactgttaaatgaatatggtaatgttttagatactttaggtgtcgtaagactgtttgttgaagaagatagaattcttttcaacaaagagtatgtatatgtaagagaatggctaaaaaacaacaatcaagaactagcaaagtatccatatataactccggttcttttagttagggatgataatttgaacacagtcggagtttatcttggatatatgagtataaaaacctttttagatcaattaacaaatacgcaataa

SEQ ID NO: 50 - DNA, 456 bp, YS40 BACTERIOPHAGE
  misc_feature: YS40 ORF50
atgagagaagttattttacctgaaataaaaattacctatatacctgatagaaaggtcaggttttatattgattttgaaaagctttttaatcttggccttcataagtcaaaccttgttgtaagtttccaaaacaaaaactttgagttttctgaagaaaatatctttgaaagactactttttaacccccctcctgaggataatattgttgaggaatttcaactaatagttataactttagcaataaaagtcgtagaaaaattaattgttagatttctggaatcaaaaagtgaatacgatgttaactatgaagaattagaatctaaatttaacgaaataaaatcaaaatatgaaaaccttggatttatacttgaatttgaaacgacagcattagttcccttttatctgttcgtgttggatgttttattagatgtatttaaaattggggaggagcaatga

SEQ ID NO: 51 - DNA, 1473 bp, YS40 BACTERIOPHAGE
  misc_feature: YS40 ORF51
atgaaatatctaactatagcaagttctggggcggaagattttatttgtatttctgattctccagtagatcaaacaacgaataatctccctgttaattctaaatgtataagactaagaacaaaagcatcttcaattgaaggattttgccctccattaggacaaaaaataccaaaagaaaattatgttgtgtataacgaatcgtcaaattcaattgattattacgaagcaagtgcgaatacaaattgttctgttggatacacgaaaaaaacaatagctttatatcaaaaacccaaattcagatatttcagagctttaattttatacggatggagcatgaagaattattcgtgttgcggatgggacactagtgtagctccaaataggtccaagcgtggtttagtgccgtttagatatgaagtttggtattttgagtttgattccatagaagacgcatatgcctggaggaatccaaagccctataatctgacaccaatgcacttatctactgagtcaagtatatcttctacaacaaactaccaaagaggggttggttatgcatcatctgcagattattcctttcttggtagttacctacatggaacatatggtcttcaaaatatagatgatgaaaatggaacaaaaacaatttttataaccaccggaggatccacaaattctgtacatatattgtctggcaactacacaaaagtaagaatgtatgaaggggatttttacaattacctttataatcaagcatacaattcttccggttatcaaaacataccactttggcctttaagacggcaattcacaaccagctataatgactatacagatgaagaaaaggaatctatagaaactttttcaaatctttatattgcaggaatagaatatgttcttccagatagatattttagacaaaatccaatatacacaggtggaggcggatattgggtatatcattattctcttactccaagcttagaaaaattagattcaactaatactattggtacagatataatgggttttgctgatttagagataaattggtattttggtagtaaaacattttttacaaagacagacggcgtttacttctccggagctacttcttcatgttcatttccttttgatcctatttttgccaatattatgcaatattatatgtctagaaccctatattataacccatcgggagcttcactttatgcgacaaagaatagcatttctggatatacaacatctccaccagattccaaaccaccagtaatgtttccaataattgtaactatgagaggagatataggaggagatagggtaagacatgcagcaataaaacttggcataagtatcgtttatgctgtgcaatatggtcaaaatgaagataaacaaaacttgccttcatatcaatttcctgacgtttgtccgccaaacaccatacctaatgcaaactggattcttaatctgtaa

SEQ ID NO: 52 - DNA, 873 bp, YS40 BACTERIOPHAGE
  misc_feature: YS40 ORF52
atggcaatatgttatataggacaagaagaagtttatagtacaccaagccataatgacagacttgctattcttgatgcagaaaatgctacatcattaaaggatgatttaaactttattagaagtgtaatttttaatattttggggttagattttggaaatgcggataataaatggtataaagttcctttttataatttgacttttctttttgaaaaatattcttcactagaatcagatttttctgtagaacactacacatcttccgaaaatccaaatttagctgggaagcataaaaatatcacagcaacaggaaacttaaatgtttctggatatggtttaataggacaaaatttatcggttggaagctatttagatgttggtacgactttaacgacaggttctgacgctactataggtggaaatctaacagtaaatcttagtggttcaatattaattgatttgagtgtaggaagagatttaagtgttggacgaaatacccaaataggaaataatttgtctgttaataacaatgcaactattgttaacaatacgaatattggtggaaatttaactgtaggagggtttggaacatttggcagctatataaatgtacaaggtaatgcaacaattcaaggagccgcatctataggacaaactttaactgtaactggtgctacaactcttcaaagcacgcttagtgtttataacaacacaagtatatctggaactttatctgttttaggtaatgcaactttttctgctaacgcaacaatatcaggaaaccttactgtaaatggacatacatctacaggttctttgtatgttggaaagcagtatatagcgttattaggaacagcaaatgtaaccaattaa

SEQ ID NO: 53 - DNA, 528 bp, YS40 BACTERIOPHAGE
  misc_feature: YS40 ORF53
atgagagtcattatagttaataaaaaattagaacaatacctctcaaaatcgtttaagttgagagaagatttaatatttgagtattataggggaaggtggatttactataacgacattattattcctcatatctctaatgacgattttgaaggatttttcttttctggagaaaatattataacgacagacaaagaacttgaggagaataaaatttattttgtaaatgattttgagggtatttttacaaaggaggaggcagctttaacaaagctaagggttgcctacgaactatctccaacagagtactttgttgtgttgaggaacaacaaatttctcggcgttctgtacaaaaacaacctccctttaagtttggtagattacataatgttagaaacgaataaaagaccgaaatttaaagttaatgtagatagatttgaaatgaaaaagaagtttgtacacatactaaaaatgtctaatattgaaattgatgaagaaatgttctttagattagatgaaattatagaatga

SEQ ID NO: 54 - DNA, 486 bp, YS40 BACTERIOPHAGE
  misc_feature: YS40 ORF54
atgccacgctggaatacattggcaataaaaaattttggttttattgaagataatgtattatctgtttttcaggactataaaaatcaatttaacttcaaaaatcaagaacaaaccataccaaatgacattaaactaaattatgaaggggacatagatttttacaacgatgatgttgttctagtatacaatctagaggcatttattcaaagactttattttttccttataacgaaaaagggaaccttacctggaaacaatgaatttggcgcatcgttagaagattacatcggaacaggaagacctaatcttaaccttcttttaagaaccctagaggaagatttaaagacttttgatgaaatagagtttattaatgatataaaggcatctttagtttcaaatgaacagacagaaactatagaaataaatctggacttaaaagttaggggatttaaatacagggcgtttttagacttggagcttatttaa

SEQ ID NO: 55 - DNA, 702 bp, YS40 BACTERIOPHAGE
  misc_feature: YS40 ORF55
atgaagaaaataaaagaagatctaaaagaaaagctactcagacttgtttccctatttgaagtaagtggggattggggtagagtagcaggaaattttgacggtcaaatactttcatatggcccccttcaatggaacataggacaaggaacacttcaaagactattgaaaatgataagcaaagaaaccttagaaaaatacctaggaaaagggtttgtagaggctttatacaacggaacactcaaagacttcattcttaaaaatgttctcgttggaaaaggagtaaaaagagagtggtcaataaagtttgaaaacctagctaaggaaaaagaaactgtagaagcctttaaaaaggctgcaaacaactactttaggagggcgcaacttctttgtgaagcattaaattttgaaagcgaaagagccttagccttgtgttttgatgtggctgttcaaaacggcgctcctagaaaggatcacataaacgaatacaacaagagaataaaagaactaaacccaaaatatgaatgggaaaaactaaaagttttggcttatgttgttgcagatttagctaatcccagatggagaaatgatgttttaagtagaaagctttctattgcaataggtgaaggtgttgttcatggacacaagtttaacctaaagaaagactttgatatagactacaacagaaagtggtacgaggattaa

SEQ ID NO: 56 - DNA, 1023 bp, YS40 BACTERIOPHAGE
  misc_feature: YS40 ORF56
atgaagactagaacaattgacgaaattagatatctaaaaagactagaaaacatcataaaagctagaatgcaagatattgttaatagcaatgatatcattgagtctaccccagaagataaagtaagaatacggatacctataatggacgaaccgtattttaaacctgtgtttcctggtagtggtgctggggcaggatctggatcggggagtgaacctggagagggtagcgaggaaggtgatcacgaaattgaaattgaacttactgttgaagaactttctgaacttctatttgaatacctaggattgcccaaaataaaaccaaaaggctcttctgtagagaaagaagaatatttaattgaaggaatttctaagactggaccaagatctagaatacacaggaggaaaacatattacgaaattatgaagtatggttataaagaagattctataaggtacaaacaccttaggaaaagagaagtaccaatatttgatgcaatagtttattttgcaagagattactctgcatcagtagatgacaagaaaaaatttaaaataaaatcaacagcgttttggataaataacttcattaaatacaattacaagaatgtaacaacaaagtttgctgttcacgacacaaaggctaaatttgtaagcgagcaagatttttttaagttgtctgaaggtggagctacgctttgttcttcagtttttgagttaatatatgaagactacagaaggtattcagtagacgattataatttttacctcttttacttctctgatggcgaaaatctaccagatgacaatcctaaacttagggaattagtagaaaagctatcagaggactttaacttaatagcttatggagaggtaaaaagtacagatgtttattacggatatggatctaaagaaactctggttccattatttatttcagttggaaaagataatgtatttgcagaaaaaatcttttcagtaaaggattttatattgaggctttttggtgagaaaaatgaaaggtag

SEQ ID NO: 57 - DNA, 1338 bp, YS40 BACTERIOPHAGE
  misc_feature: YS40 ORF57
atgaaaggtagaaattacgaaaaagaaaccttagaaattcttgaatgggtaaaagatttgggtttagaaccgaaagacacaatttttgtagatgtagatagagataagctaatttttgcaatatcaaaccactttgttaatggaattccacattggacaaaaggaagaaatttcataaaatacaaagaacatgaattgcaaggaggaaaagtatttgaagttgtttttaatagtacaccagcagtagcctatatgagtctggcgttgacagaagctgatatattgacgattattgctcatgtttacggtcatacacacatttttgcaaataatatttttgaatacaaggatgacaacatattagaaaaacttaactattctctccaagtttacgaagaaatgctttcttattattcttacgatgagctagatgtaatttatgactacgcttatactgcttggaaccttttggttcctgatgaaattcgtgagaataaaatagaaaacacaactaaagaagagtttactgagaataaaatatttaacaaatttttgggaaggaagccaaaagtagaagattttagggaaaagtacaaaaaagagaaagaaacggagtttaaaaagaaatatggaataggttctgtgtatatacacgaatacatactagaacattcacccattcctgattggatgaaaacgcttataaacaatgaaattgatctgcttaaagtaataatgagaaagtcaaaattaaaaattctccacgaggggtttgcttcttgggtagatacaaaatatctgcttgaaaaaatggagggaaaagagttttttagtagcttaatttcatctactcttgttggttatcccagtttgagaaatccttactatattggatataagttgttagaatttgcagagaattttatgaataagaaaataccagaatatgttaaatatgttgatgattttgagcttatagaggaggtgtttaatgaagactttttttattggcttttaaagcagtttcctctagatgaggatgagaagaaaattttgtctgacagagaattgtttgaagaagtttttgagaatcagaaacaaaaacttttatcagaatgtctaaattattatccaatcatttatatagatgactacccagataacattgaggagccgtcatatgattatctaaaagtatcttcgtattcgcaaaacagaaaattaaagcttacttcacaaaccccgttggacactaggtatgcttacgaaacactaaaagttttagaaaaaatttggatgggaccaattgtgttaagatacaaaacgctaaaaaaatag

SEQ ID NO: 58 - DNA, 1851 bp, YS40 BACTERIOPHAGE
  misc_feature: YS40 ORF58
atgaaccttgtggatattataagtttctttggaactacaggatcacaacttccgtttaacataaaagaagaggaaggaaaatattacctaataaacaaccccaataacaaagtaaacaatattccgctttcagatggaatttattttgaagtaattcaaggtacgctagacaatcaaaacgggctaactcaaaatttggttgttaagctaagaaatccaaacaatatacaaatagcttcctcgtatgttgttagactttacaacgcccaaaacggtgctctactatttgaaacttatgtacccacttccttcgatggtttagaagtttctttttcaactaatgtaaatgttccacttattacgattgaagcaaacgggttagtcctctacacaagttatgaatacttaaatggctatattcctttagaccaaagaatttatgcttcttttagagatcctaataacattgaagtttttagggctttttttgttagaaaggtaaacaacaaaagatatttcacttcaaccttcagatctgtttacaacaagtttttgaattcgtcggcaattccttttagatttttagacgcttacctaaacaatacaataataggaaatttcaattatcaaataatcacagaatttaaatttccacagcagtatagatttatttcagaacaagagtcgctttctataagcgcttattcaataatgcttttagctttctataaagattttgttgaaaaactatctggatttttagtagatgaagtagacttttcacaactagataaagattcaagaaactttccttacaaaaagtatttttacaacaaaaacactttgtcttacgaaaaagtatcctcctatgtggacataacagagcttttgcttgcaatactactaggaataaaaatatatggaatgctagattccagcaaacaaataattgataatttggttaatgcgctaaacactttttcaccaactgtttattatccagacgcatcagttggttcaaaggctttaatacctgatatcataaattcaaatgtagaagaacagtattctttgtttacaaatgttttatttgcgcatatattaaatcttttaggaattgataattcgacattaatcgatgaaataaacagagtatttgtcaataatagcaaaaaaacagaaaataggtctttaggcactataaactcttttactcctcttgaagcggcagttattttaaaatttgcaccccaatatcttgatacaagtaacaattatgtttcttcactaatttcttcagcaacctctcatctctattctgtagaatatgtaaactctatagctttaaattctgatgcaaattcagattcttacaacccaatatatagtgaaaatctgtcttcagctttgccattttacgacaaaagaacttatgtaaatctaattataaatagtctgttagataatccgataaacaactttagagctttttcttctgacgaagcaatattggatagctacaaatattctatttacgaaggtttttatatattgccttcacttagagcaaacctgacatatttctttttgagtatggagaatgcatttgaaggtagggtagaagaaaaatctgaagttgtgcttatagatagtgttataagtgttggaacaaatcagttgattgtggaattaatttttaataaaccagtaaaaattttgtcaataatttctaatcaaggagtacctacagcttacgcatcgtcaatgtctgaaagcgctcaccatgtattaacattttctgttattaacacctacttaaatggggttttagaaatttacgttctaagttaa

SEQ ID NO: 59 - DNA, 1860 bp, YS40 BACTERIOPHAGE
  misc_feature: YS40 ORF59
atgaagttaaacaggtttttcaagggtaaaacagcggagagcagaaaactttcatttaaagaatttcttgaggaaaaatttgtaagaaaccccgcaaagtattctagaagtgcacataaatacctttttgatgcaattacatattttgggttcgatgaggaaagaaaaattccaaaaatctttgcagatgagctttttggtgttgacgataaaataagagaattcatgactattttagaagcgggcgcaaatggtcatgatgtaagaagaagaataatcctttttgttggtccagtaggaacggcaaaaagtacctttgtaagcattattaagaaggcactagaaagatactccaaaacagaagaaggagaaatttatgctatagaattttgtccgatacatgaagatccactaaatttgattccagaagatacaagagatgatttttataaggagtttggagtaaaaatagatcaagaactttgcccagtttgcaggcataaattaaaggaactgaaatatgaagatgttagtacagtaccagtagttaggtttactttttctgaagccgaaagaagaggaattgctacatttgttcctgcagattggacctcacaagatgtttctgtacttgttggctcagaaaatttcagagctgtgcaagagttaggtgatccttcacatcctttgggttggaattttaatggttcattatttgttgcaaacagaggaattcatgaatttattgagcttcacaaagcaaaaccagaccttctttttcctttaattactgttgcacaagaaagaaaagtaaaagttgataggtttggtttaatcgatgttgatgaggtcttgattgcgcatacaaactatgcagaatacaataaattcgttaacaaaaaagaaaatgaagcgttaaaagatagaacaagagaaataaattggatttacaatttaagatgggacgatgaagaaaaaatttataaaaaaatgttgggagaaactacatatcatttggctccttggacattaaagttcatttcgggtattgcaattatgtctagattatcaccagatagagataagaagtatgacttgttaacttcattaaaactttacaacggtgattatggtgcaatgtactctgaataccatgtagacgaaatgaaacatcctgacgatggtaagttcggcatttcgccaagggtagcagtagatgcgctaagctttgcttcagtaggaaacacttgtgttactccaattagagcctacgaaaaactagaagaaataattaaatctggaaatgttgacatacctcataaagaatttttggcgctaaaagatatgattttaacagaatacaacaaatttatcaaagatgcagtcttcagagctttcatcggttcttcctttgccaccgaagcgcaaaagtatgtggatcgttatattgaaaacgcccaggcgtttattaatggagaaaaaataaaagatccatttacaggaaattatgtagaacctgatgaaaaatttttaaggtcaattgaagagctagttggaatttctaaagaagccgcaaaagattttagaatgaaaattcttgttaaagcaacaaatttactgagaatgggcaaaaagattacttacgatattcatcctctgctaaaagaagcgattgaaaataaaatctttggcgatagagcaacatttataaggggctcaataacttctgtagtaaaaactgaagaacaggaaaagcacatacaagaagcagttgaagaacttaaaaaggttggattttgcgaagtttgtgcaagagaagctataagctatgtagcatacctacttgatgtagcaagataa

SEQ ID NO: 60 - DNA, 588 bp, YS40 BACTERIOPHAGE
  misc_feature: YS40 ORF60
atgtacctaccgagtttaatagggctagtgggaaaagcgggttcaggaaaagacagattctactattcggttttagcgaaatataattatgtgagaatagcttttggcgatcctgtaaaagttctgtcagtaattcacttactaaaaagttatcctggaattctggaaaaagaagatcaattgctaagagtttttccttctctgtatattgaactattttctaaccaaaaaacctctctttctagaacacttcaacaatatatcggaactgacttagcaagaagatttgatgaaaattattggataaaaatagcagaagaacttataaaggaaaaactagaaaaaggaattcgagtagcagttactgatgtcagatttttgaatgaggcagaaagcattaagaagcttggtggaattttaattaagatagttggtaaaggttcctacgattatggtaatttggaagcaaaacatcaatcagaaagcgaattggaaaaaatagtttgcgattatacagaaaatgagtttacttctctaattaaaaaacaccaaatcctcgcttcggaggtagaagatacaggacagtag

SEQ ID NO: 61 - DNA, 450 bp, YS40 BACTERIOPHAGE
  misc_feature: YS40 ORF61
atggagtgtggtatgatagcttttgagatgagtggcctcgtagctcaacaggaagagcactcgccttgtaagcggggggttgtgggttcaaatcccaccggggcctccagaaagatgaaaaaagtaaaaataataacctcctcagagcctaataaagactttgttaatataaacctcctaaatcgtaatggaattttgaggaaaagcattacctatggagttatgttgagagaaacaaattatgtattttttggagtcaatggagaaacatacttcaaaaactatgttaaagatgtctttataagcggaaaaaataaacttttagaagaactaaaaacaaaggacacaataaatcttgtaactattgatggaaaagaaaatttttacagcaaaatcttaaaagaagttttatctgatatcttaaaagaagaaggttttgaagtcatttaa

SEQ ID NO: 62 - DNA, 1869 bp, YS40 BACTERIOPHAGE
  misc_feature: YS40 ORF62
atgtggattatgccgaagaagagtgaaagtttctcaataattaaagacataaactctctcatagagaaaaaaacttacgaagctaaggcagaacttaatccaaaagatatttatgaaatagaaccagtagacattattacctttgtaacgagtaacgattatttaggcataaatctttatggtctttctgaacctcaaaagaaggttttagaaatagcagacgattttgaaaacggcatcaattatataatcttatgggttggaaagggtggcggaaaggactttattacaagaataatttttatgaggcttgtttatcgtctactttgcatgtattctccacataaatactttgggataccaagttcagaaattatcacttttcttaatgttgcagcatcagctgaccaagctacaagtgtattttttgatccactaaagaactacataagaaacgctggtccaaaagcctttagacagtttggttttaatccagacacagatataaaggaaaaacacattatctttcccaaaaatattcatctagtcagcggccactcagaaagcgattctctggaaggaaaaaacattcttgtagcagtagcagacgaaatagatgctaagtcttttagaaatccagaaaaaatgtggacaatgttaaggtcttcttctaggtcaagatttaatgggaaagaaaaaatttttgctatttcatacatgcgttacagcgaatcaaacggaatgatacagaaactatataaggaacatttggggctacctcattcttttgtggcaaaatatcctacttgggaatttaatccaaatccaatgataacaagagaaacctttgcctctgagtttgaaaaaaatcccgtagaagcagaaacaatttatgcttgtaatccaccagaacagccaatagacgcttggtttacggatttggaaagattaaaaaacgcaatgaaaccttcggaacttcatccactaaagtttcctttgccaccagaagatttttacaataatcctaaacttgaagctttcaccctaaaagaagatggctcatatgaaaaaatagatccctacaatctagaatttaaagactggttttatggaaaagaaggtgttgaatatgttcttgtagccgatcctggtttaggtagagtttctacagagggagatgcttacgcaatagctctcggacatagagaattctattattcaaaggaaggaaaattaattcccaggccagtcgttgaccttgtatttagatttactggatatatgtttgatgaagaggaggttcaaataaacgccgtgcacaatctaattgaaaaacttatagaagaaaggaagtttaacattacacacacattttttgacatatataactcagcttcaacagcgcaatggctaaaaagacgatatccacacatgaaagttacctacaacaaatatgttacctacgaacactatgcattgttaagagaaagaatttttggtgaagcgcccccatcttctgggaagggagataggttagacaatggtggcatacatctgtattatcatccaattttgttttgggagttgtctaatttaattgaagacagagaaaaaaagaaagttgatcacaaaccagatacatcaaaagatatgagtgataccgttgcgatacttacttatcttttggtaaaccttcctctacagactatttcaatagttggcactccgaaagatttagaaaatgacttatttaaagttgataaaaaaagaagcataatagataaatttgataaacttattagggaagaagaagccaaatttgccaaaagggtttttggtgaaattgttgataatgagattcttaaatga

SEQ ID NO: 63 - DNA, 789 bp, YS40 BACTERIOPHAGE
  misc_feature: YS40 ORF63
atgagggacataaacattaaactattaaaagaagcagaaaagaaggatttacaagaagcaattgctttgtttgaatctttagaagaaacacacaaaaaccacaaaactctatcaagaagagatttaatcgccaaagttttagaagattattccataaacaaactgagcataaatgaaatatcaaaccgctatggcgtatccacaagcttaatacacaaacttcttcaagacttttatcttttctactttgacgataagaagttgcaggaactcagctttgtagatccaagctcacattttggcgttttatattctttttttaattcagtaagcgcactatcaaaggaaataacattcaatacaataatctctaaaaaattaagagaaaaaatatcttccctgttagcagaaaaaggaattgatggtgttcttgaaaatagaaagctcctaaacgcttggaaagatagcataaggagatttgaggttctattgaaactttctgttgatcaaacaaatacttatctaaacctaatagaaaaagtgcttgatagacaaagggaagttgcttttgtaaaggcaatgtacgaaatcttatcagaattagatcctcaaacagcaataaagcttcaagaaaggctttatcaagacgagtatgctagggcattaatagaggccaactctttagaagactttattacactcattgttaacacaactgccgtaagaaacagaattaaagaattaaacgaaaaaaacgcactagatgtggattatgccgaagaagagtga

SEQ ID NO: 64 - DNA, 381 bp, YS40 BACTERIOPHAGE
  misc_feature: YS40 ORF64
atgccaagaaaaatttctgaggaaaaacgaaagctattagaggaggtctataaagagtatcttagagataaagaagctggttattatgatctttcagttaaatacaatattccctattccactttgaggagatacatacttcaacgactaaaagaagatgacatagaaagtgagaaagattatgtagaatttccttacgaaggatttgagtgtttaaaggaaggagatactttttcttggagtgaaagctctgaaacgattttcaaggcagtaaagaaaacaagaatcgctggtgaaatagtttggctttttgtgaaagcaaacaaaaaatctgaaagacattttgtgccagaaactttggatagctgcaaggaaccatga

SEQ ID NO: 65 - DNA, 552 bp, YS40 BACTERIOPHAGE
  misc_feature: YS40 ORF65
atgtttgggcttataaagtctttatttttaatctttccaacacttattcaaattttatcactattagcaaatttgttaaaagcgataggggaattaagcaatcaaaaaaattttgaagatgttaaacagagaaaggaggatacagacactccagaaggaaaaacatctctggttcaggaagaaaaagattacgacaaaaattctgtaattaatcaagaaaatttaatagagttagaaaataggtctttttttgtcattagatgcaaaaacaatttttacagtcttgatgagtctttaagaaagcttccaccagaaatatttaaggaaaaatatttggatttttgtgaaaaaggttcaattgatacaccagaaacaaaaagcgaattaacagaagatatagaatcaataacaaaacaaaaaaatgttttaattaacaaaatctactttattctaaacaaatacggtttatcaaaagactttgatatgttacctcaagaaataaaagaattagatccagaggattttaaagaatatgccaagaaaaatttctga

SEQ ID NO: 66 - DNA, 267 bp, YS40 BACTERIOPHAGE
  misc_feature: YS40 ORF66
gtggaaaagctcattcatatttattatatcaaatctgcaatttctgaagacaagaaaataaaatttctcagattaaaagatttatgcgttcaactagacgacatttctataaaatcttgtaataaagatgtattaaataaaatgtttatcaaagaacatatagtgaactcgccttactctgatgaaatgactttgataaaatttgcagaaaaattaactgctttaagaaaaatttatggcgacaacaagatacttgaaataatttaa

SEQ ID NO: 67 - DNA, 741 bp, YS40 BACTERIOPHAGE
  misc_feature: YS40 ORF67
atgggagtacctattaaaacttctcaatttgccgaaatttatttaattggagtaaaaacaacaggtaccaatgaaacaaatgacttactaaataacgacaaagtaactctagttagtgaagatgaacaaaaattttctgttggtgtagaaaaaattggtttagcaactaacttaagaatcaacgagtcaatgggatcaagaacgagaactgttataggaacgccaatacctatttttgttccaggcttctatgatggttcaataacaatggaaagagctacaatacttttgcaatcctttaaaaacggagtaaatataaaccctctcctcgcttacagttccgaaatatatatgcccgaagctagaggaattaagagagttgaacttcctagtgatgtaaatggtactcccttacctaatgattttctttcctcaaacactactttttattatgtaagcggaagagaatttgaatataatgaaaaatacattcctgggtttctttttgttgttgcattgaaagataaggttttaaatgaagttagtagaaatgctggtctttaccttgcaatgttaagagatttttctgtttcgtattcttctgagaacgctattattctagaaaatattactgccttggcaaggccgctatacaatagcggttggtatcaagttcttgcagatacattcaacacaggtgcttccttcggttacatatattctgatccgcacaagctataa

SEQ ID NO: 68 - DNA, 651 bp, YS40 BACTERIOPHAGE
  misc_feature: YS40 ORF68
atgacaccaagatttagtccttacgcaataataaaaacactatccttcaacacattacttcaagcagggcaatcacctgctgatttaggtttagcgctgctaaacattcttaacaattcaaataatatttggggcgcactagaaggaaatttcgccttgtttaaaccaatcggtatcgcaaacaatgtttctatagatgaatcttataactctagaccagtttggggaattggtgagcctacaaacccaattgttgttcccaataactattctgctacaatctctatctctagaatgacattagatactctttctgtaagagatttcacaacactaccagattactggtatgtacctacacttcaaagacgggttgaagaatttttaggaccagttactggtagggatttcttagattatccattctatactttcatatctataacttctgttgaaacacctgatattggtttttctgacatagtacaaccttttcttaatagatctttatatgttttcatgccctctgagtattcaacaagaattagcggcgatgacacaattataatgactgatgtaaggggaacaggaaaactcgttaacctcagaggactaatagaaaacctcgcaaatgctttattaggaggataa

SEQ ID NO: 69 - DNA, 1947 bp, YS40 BACTERIOPHAGE
  misc_feature: YS40 ORF69
atggctatttccgtatattttgatggaaagttaataaagcaacttggcgcatatgtaaaaactgacctaagcgctgttaagcaaatcaacggagtaggaacaggaattgttgctctactaggtttagctgagggaggcgaaacttataaaccctatagactgacttcctttgctgaagctgtcagcatctttaagggtgggccacttcttgaacatattaaggccgcttttattggtggtgctggtgaggttgttgctgtaagaataggcaacccaacaaccgctagtgtttctattcctgtagcacaaaatacttctgatacatcaccagcaaacttaaattttgtttcttatgaagcttctacacgttccaatcagatttatgtttcttttgatttagatgaaaacttcacttctgcaaatgaagctgatgatactataatctttaccatttaccaaaagcatccagatttttcagtaaccagagaaacttttacttttccaagaaagtttacaacacctactgttttagtaaaaagaggaagcactctattttttgtggatagatctatagtaaacgcagcgctagcagcaggaccagctttccaaactgcactaattaatctgcttaaggaacaacttcaacctactgatgttgttcaaatttttgatgctagtgatacaaatccagtagatatacctcttggactttttgtatatgaggtgctttatggtggactctttggttttacaaagtctaggctagtaaaaactagctttggaacagttgatgatcttctctctaacccgctactattcaacctttcagcaacaccattctttgatgggagtgattatcaagactatacatctctttctgatcccgcaaactggttcgctaaagacgcttacacaatcaatcaccttgtagatacaacaataaacccacacatactagccacaagaatctttagcctttctggtggaacaaacggcgacgatggcacaggatattatcaaactgctgtatctaactacataaacatttggtcccaaggattggcaactttagaagaggaagaagttaactttgtcattccagcttacaagtttacaaatgtaacacaacttaacgatcgtttaacaatatttaagggaattgcttctactttcctttctcacgtacaaacaatgtcccaagttaacagacgaaaggctagagttggcgtgtttggtttaccagcgccttctccaaatgaatctgtaactgcatctgagtacctttataacagaaatattttaaatacaatttctgcaatgtttggtggcacagatagagcacaagctgtcgtatttcctttctacagcaacgtatttaacgatgaaggaaaggttgaacttcttggtggagaattctttgcatcttatgttgcaggcatgcacgccaacagagaaccccaggattctattacattcctaccaataagcggcattggtgctgaacctctctacaattggacctatactcaaaaggacgatcttatttctaacagagttttatttgtagaaaaagtaaaaacttcttttggtggcatcgtttatcgtattcatcacaatccaacaacttggcttggtcctgtaactcaaggcttccaagaatttgttcttagaagaattgatgatttcctacaatcgtatgtttacaaaaacctacaagaacaattcattggaagaaaaagctacgggcgcaaaactgaaaacgacatcaaggtatacacagaagcacttctatctaatcttgttggcaaacaaatcgttgcctataaagatgtaaaagttacttctaatgaagacaaaactgtttactatgtagaattcttctatcaaccagtaaccgaaattaagtttatcctagttacaatgaaagtaacattcgatttggagtaa

SEQ ID NO: 70 - DNA, 711 bp, YS40 BACTERIOPHAGE
  misc_feature: YS40 ORF70
atgtcaacctttagcgatttgacaaatgatttagagtttttgagcattgctcaaaactatgttccaaatagcgatttacctataccttcttatcctaatccaaaattagtttttgtagattattttaagaacatttttaatcagctaaacttaagaaacccacttagtataacaacttcttttccatctgctacatacttcacaaccttaaaaaatgccaatactgataatgaatttttatttaaaagaggcataaacatcgccttagataatcaaggaagtaaaagatttataggtaatgttttgaaaaaagacatttatggtttcatagaagaattcaaagtttctttcttcatttggtcttttgatcctattgacagagatgtgttaggggattttgttttgcgccttttgcttgaagcgcaagaatctttttacctcttaaaaagaggaattccagattttgtaattgaaagatataacgatagccaagacgaaaaaatcatagctaatcatccacttttttacagagaggtatatgcaagcggaaaaaggttgatatttggtaaaaaaattccaagagaagaagcttattttggcataataagagatgttcttcctaacgaagattgcgaagattacgaagcaataataatcaatgaggacggctcctttaaaaaggtttgtataaaaatgcccgtagaaacataa

SEQ ID NO: 71 - DNA, 663 bp, YS40 BACTERIOPHAGE
  misc_feature: YS40 ORF71
atgaaattcaaatttaacgaatatccttcaacaaggctatattctatagcgaaaaacatagtagagaaagccttttttatgggcggatataaagttttatattacgaagctgaactttgcaattatcgttttccagatggcaaaacttgttttgataaaagaacgctaaatccttcaataaattgtcctgtttgtggcggtagcggtgttgtttataagaacccaatagaaactatagccatagttatagacaattctgacactccacaaagaaggcgtgaaggcgtattttttacagatacttttagaatggtaactaaaccagaagttccagtaaaacttctaaaacttcaagaagaagggggattgttttttataagagataggtttgatatttattcttccgatggaacgctctacacaagcgtttatgttgattctgagcctaaagatatttggttagctggaatgctatacaaatcttttaaagtagcaactcattatctatctgaaagaattgctcttggcaaagaaaacgctaattttgaaaattcaaattacacagaagtcgtagaatcagttaattttgtaggcgcaaaaaacgaggaaatagaagatattttaaagaacatttttattgacgcaattaacaaggagaactaa

SEQ ID NO: 72 - DNA, 516 bp, YS40 BACTERIOPHAGE
  misc_feature: YS40 ORF72
atgagccatttgaataaagtaaaaactgtaaagcgttatgaagaatatctccaaaaaaacttcaacttcagagagtttaacttgaagtgttccattaatttgggaagaaaaaagatttcaacaaatgtcttctatgtaagtactaatgattttttaaagtttttggacgctgatgaaattatctttgctggtgaaaattctatgataaaaatttctttatttaggtatagcaaaaaatcatttaatatatcaatggaaacactaaaacagctaatacatgaaaaaacaatttacctaacgaatgtatacagaatatacaattacaacttatttcccaaaggaagctattctttttcgcaaacaataattttctttaaaagagaggatttgttaaatttcaggaaaaacgaactctatatatctaagatgggaaatggcatttttgaggaaaagattttaggtgagcttcagcaaatttcttacataagaaatcttgaaaaaattgatgtattctga

SEQ ID NO: 73 - DNA, 1413 bp, YS40 BACTERIOPHAGE
  misc_feature: YS40 ORF73
atgccatacgaacatttaaagcacttggatgaagcaacactaaaagcgcttaatgcagctggtcaagttgcagagtctttagaaagagaagacctagaacctgaagtaactcaactcaatgttcttgacactccgcttactgatcttctttcaaagaacgctgtaaaggcaaaagcttatgaacacgaatacaatgttgtaactgctaggcacgataagattggttatgctgctttcagagaaggcggtcttcctaggactgtagaggtcaatgttgtccgtagacgcattaggccaatgctagttggacacaggattacagttaccgagctagctaccaggactacccaaaatggcgttatgcagattgacgagttggttaagcgtgaaaagatgatcgctgttgccaacgaatttgagtatttggctttctatggagataaccttcttggcgatgatgttcctggtagcccaaataatcttcaacaggatggcatcatcaatataattaagaggggcgctcctcaaaatgttcttgatgctggaggtagacctctttccattgacctgctttgggaagctgaaagccgtgtagtatctacacaagctttcgctaatcctactgctgtattcattagctatgttgataagcttaacttacaagcttctttctaccaaatctctagagtaatgactactgccgatagaagggctggtctgcttggtgcagatgctcagtcctatatcggcgttcgtggcgaacattctctctatccttctcagttcctcggtgatttccacaagtttaaccctgctcgtttcggtgctgaggtaggtgatttcgctgctccttctaactcttggactgttagcactacagataatttcgtaactctgccttacaacagcggtcttggcgatcctgctaatacaacagtttacagctatgctttcaaggcagccaacttctatggagaaagtgctgccaagtatatagatgtctatatcgactctactgaagctggtaaaggtgttcggttccaattccatggtcttgtcaatgtaaagtggcttgatgtttacagaaaggatcctggttctcaagaatacaagttctataagcgtgttaaggtgtctactgtaaacggcgactttacttggatagatgatggtcatgaaacagtaactactccttccggcgtttatagatggaagaaaattcctggaactggtgtagttgtaggaatagatcctaatgtaactactatggctgtctggattggtatggaactttacagactacctcctgcacttactcatgattacgtcatatggaaggtagcttctgtattctctagagccccagaatttaacttcctcatcgtcaacgttggtcaagaaccaattgtataa

SEQ ID NO: 74 - DNA, 1635 bp, YS40 BACTERIOPHAGE
  misc_feature: YS40 ORF74
atgagtaaagctatgaaagaagtgcttaatattccactagaaacaaagtttgaagtaataaaaagcacaaatgtttacacaacgcctagtggagcagaatttttttctgataaggttccagaaataaaacctatcaaaggcgaattttttgtaaaaagaaaaaatcttgaactaaaacctggtgattgggtaatatttggcgtagcttctacgcctagcattgatttagttaatgatgcgattttagatgttaaatacacttttggagattctttagaagaatttgtaaattcaggaagaattttctatgagcatggctataagcacgctggaaatccttcaaagcatccagatatagatgttcctattggaattcccattgcggcagaaatttatgacaacaagctttttgtttggatattgttagataaaaatcatgagcttgcgcaaaaagtttacaagcatcttcaatctgcagatgataggtttactagtaaaattggtttgtctataggagctatacctattggcaaacctcagacaaaggttatcaatggagtatatgtaaatgttcctcccaaaatgcgcttatatgaagtttctattactggtcaacctataaatattgatactttcgttaaagttttaaaaagcctagtttataatggtgtagaaaaagctatggaggaagatactatggcgaaagacctaaaaaacccagaagaagaaaagcaagaaggagttcttgaggccttggaagaggaggttcaagcagacgcttcagaagatgcttctgaagaaaaacaagaaatgaagcaagatgaagctcaggttttagacttaggcgatctacaaggagaagcttctgaagaaccttctgaagaagataaagaagagggcgctttagaaggtcaaaatgaagaaacacaggacaaagttgctttggttcaagaagaagtagaagaacacgaaaaggaagatgaagaaactttcaattacatccttgataagctagactttctagaagaaaagctttctggggttctttctatgctttcttctaaagcgccgcaagaagaaagcgaagatgcagttgtttctagttctctagcagagctaaaatcttttgttgaatctaaattggcttccttagaaaacagagttaattatgttgttgacattgttgaagctagccttgaaaaattcgcagagctaaagtctgttattagcaagcttgaatcttataacgaaaaaattgaaagcgtagaaaaatctctttcttcgtttgaagacctttacaaggaagttaatgaaaaactagaatctacaaccaagtctttaaatagtgtcaatatcgctcctaggcttggcgtaaatactgaagttcaccctcaagcaggaacatttgaaagcaactttcaagaaaagctaaagtctgttctagctaacaaggctaagctgaaaagcttagaagaaaaaataaaagaatacctaacttataagggaacaccaattcagcttaatgagaagaaaatggagctttataactttgcaaaagaagaatttggtttagaaccagaggaatttgagcttatttacagacaatataagaataatcgcaaaattagccaataa

SEQ ID NO: 75 - DNA, 261 bp, YS40 BACTERIOPHAGE
  misc_feature: YS40 ORF75
atgaagctgagtaaacaacttattgcagccatcacaacgctatttatgaaatctagacttcaagaagctgaaaacacagacgacaaagaagaaaaacaaagacttattgatgaagcagtaaaagcttttattgacacaataaaattgtttgacattaaagaagaagaaattttagattattttcaccttgatacctttgaaggctggttttttgagcttcttttgcaagaagctaaaaaattctataacgaaaaactataa

SEQ ID NO: 76 - DNA, 4578 bp, YS40 BACTERIOPHAGE
  misc_feature: YS40 ORF76
atgggcactttttattcaataactggtcctattacaacagaaaaagattatgatgttgtaaaaagagttcctgaaggatttgttctttatcacaaagactatcctactacttttgtattttttgaaggacaagtaattaaacctaagaacggcgttatcgttaatcaacctgattcaaattggatactcgaagaatacgaaaaatatgcgcaaaaatatcaaattgcgcaaaaacaacaagtctatgaaagctacagagaagaaagttttcaggaacctgaaaaagaagaagaaagcagctatttggaagaatttatagaaaagctagacgaaagcatctttgacatagacgctagcgaaggcaatctaattgaattgaacaatattttaggaattgatgaaaattgggaagttcctagtggaactaaacagatatcaggatttttcactttaatgacttctttagcaatcggatttttgctagaaaggggctatttaaataagtacgattttataggtcaagaagatataacatttgtatttaagaaggaaggcgcttttcaggagctaaagaaatttttgtctaactacttaaaagatatttctgatggcgctttaaatgttctcacttatttagttatttcaaaatataacggcgctcaattagatgttgacgctaactggtttttgtccttttataacctctacaaagacaggttaaaaggaatatacttttcaaaagctaacatagacctctacgacatagagagaaatttaatttacgctttatctccaaaagcgctcacagagcttacagtagaagagttaatgtctaaaaaaagcctaataaaagtaatgcttgatgtaaacaattttttgcgaaaagaattttatggatttgacctttattctccagaaaacataaaaaactatctgagttctttttttgaagttcttctagaagtaacacctagactagataaagctatattggctcctcttactattcaaaaagcctttaaggaatttgcaaattctaaagaagaagttataatgtctgcgctcgttttgtttgcgtccttagataaattttcagaagaaagttcttattttaagctaaaagacttgatagtaaaagctattgaaaatctaagttcgttagaagaattatacgactcttctgacgatgttgaaatagcaatacatgaggcaagatataaagaatctttagacaaacttttagacttcgcaactgagcaagtcatttcgtttgtttctaaggagcctttattaaaagaagatgatgtgcatagtctaattctatttttgtctttcttcctgcctagcgaaaatgcttacaaagaaacttttatagaacatgtattttctgaagaaataaacttactttcagaaaatatacctgcatcaacctatctaagaagattttttcttgatctttacagcgatcgcaatgttgcttcagatttagctgatcttcttgcagacgcaaaagctttaactttaatttctccagagctagctaaatatactgacacttctcttttggctagagaaatacagtttaatcctgaagaaataaaagaaatgcttgaagacaacaatctttatagagttttagaaatattttctcaaagaaaaccttcagaagatatacaagagcttaaatttgttgtagacataataaacacactattgagcaggtatcataacaaaagcgattctcttgtaacagaaaaagtttttccaataataaaagcgcttttagaaaaatatgaagattatccagagaagctttttgagatagctgattttataaaaggatttgttgagtttcttacagatatttctacaaaaaatttgtcttatgttgcagaaccaaataatcttagtgcaattagtgcagtagcagactttacagattacatgatttacgcatacgataaaattattgaaaaagaagttaattcttcaggcgaaaagaagttgatgtatagaatagctgctgcaaatcttcttacacacttagcaaatccttcaacactagctgaatattttaagattacttctggtgaacattttgaggcgattctaaattcttatcaaaatattactgatctactcagaagtatttcatctctttcagtagaacaaaaagcagatattctaaaaggtatagaaaatgttgtatcgtcaataaacaaaatgttaaacgacattaacgcaaaaagaagaattacaaaaagaactatcgcatcactagaatcaatgatgtattacttagaatatttcatacgagaagatttaatgtatctttttgaaagcgaaaaaaatgctttagaaacttttagcgaagagataagtaagatacaaaaagaatacaaaaaaatctacacatctttgtttgctatgagaaataatctgattgcaaactccttggttggaatttttgaaaaagaaaaaccaaaaaggtttcctgcattcttaacttctagatacaaagcgattattgttgatgcgatgagaagactatcagaaaactctgacttgatttacgcagaaaaagaagaaaagaacatcttttcactttacagcgctaatgaaactagctttattagtaacgatcaaaaattagactttataagagttgtaaatctattaggaaaacctgtattaaaagtgagaatttcttctcctgagttttcttcaagaaagaattctgaggatttttataatctaattgacattgaagaaatgaaggaatttttctttgaaaaaacgcttgaagaacttttagataaaagaactcctgaaacatatggacttttagaaattcttcctaaggagcttgctttaagaagtttggatcatatatttactcacaagttaaacagtccaaatttcttgactccaagcgatcttcaagcgatgaccttagctttcatcggtagaggaggagcttattttctatcaggagataagagcgctataagcaaggctttagaaggctttgtttcaagcaaaattcatgagctaagaaaggcgctttcatcaaaagaaaagatgaaaaccttagacaaaataaggctgtctttgcaagattttgaagttgttgactattctgctaatgacgacataagaggactagaattgtcagcaaaagttagaatcggaaaaaatgtctataaagtttcagtaagagatttaggttttcatgtatacatgcatgcgcttgttgaatatttctattcaattggaaaaagcgaaaaagaaattattcaggaattggaatcctttaactacttcatagaagaaacaaaaggaaatttaaaagaaagattaactttcaacaaattttttaagtggtttgaaggaaaaatagaaaaaaatcgtttcgtagaacttctaataaaaaatgcaattgaactttcagcagaaggaaaaagctcaatcgctttatctgatgaagaaagtataagaacaactcctccacctccagaatctgtcaatgcgcaaaaccttgtagattccatggtaggatactactctccttttgatacaccaatgtcttctctaatgtttagtaagtttttagacaaagtgaagcctatattaggcgaagattggtttaattttgttaaagaaatatctgaaaagtatatcaatgtcgtcattaatccaaatggttggtattatgcgtcaggaaacacaattaatatcaatcctatagcctacctattaaaacaacatcttcaaaggccgtctattagcatgatgttgagatcgcttgaaagtgcttctttagataaaaaaggaacaattgacgcaattattgaggataaaaaagaaatatcactagagaaaaaagaaaaagataggtatataaaagtagttacgccttttactgctactttgatgcacgaaataggacatgcgttagcgagtgcatatcgtcatataaaagatttggtacaaagaaaagatgacacaacagaaaactactcatcagaagactatgatgtagaaatagacgaatatgatacaatagaaaatattattgaaaaggaaattgaaagaaaaaattttgattctagcgaaatcaaaaagttgtctgaagaaatcccagaatttattgttttgactttccttagacatcaaaggccaagaatagttgagtttacaaaaagagcatcagaagaattaggattagataaaaacattctaggtaaaatccaactaacatcaacaagtttaagtagcgatcttcaagttctttactcgaattactacagctttctaaaagatgcccagaaaaaaatatcagaaatgggcgaagaatttgaatcaaaattagaagaaatgatgaaagagtatgagataatgaacttttattctttctttaatatcattgaagtaccttcaacttctttagaattttacttcctttatggcgaagaagaattcaaaaacaagtatccaacttactacccgataataaaaagactaatagaagattataaactcgctttgaaaaaggagaaagaaaatgaagctgagtaa

SEQ ID NO: 77 - DNA, 5235 bp, YS40 BACTERIOPHAGE
  misc_feature: YS40 ORF77
atgggccttgtatacgattacaataatacctatactgataactcaccttttgataaggtaaaggtagtacctgaaggaaaggttttatataaggagtctaatcctactagaagcgtctattttcaaggacaaaaaactacaaaacatggtatcgcaatagaaactgaaaacggttggcacttagtagatgttgaagaatacttaaacagatttggtaatgcgctagatgaggaacaattaaacactatttcgaagatatacaatatagacatttctcaatacctgcaacaagaagaaatttcaccagaagaaacaaagatagaagaaaaaactactgagcaaaaagaagaagtttctgttgagcgccaagaagaaattaaagaattacaacaagagttagcagaagaaaaaactgaagaagtagtagatgttttagaggaacagcttaaagaaggttcagaagctttagaagaaatatcagaggaactagaaagcaaagaaatttctacaaaagaagctgaggttttagaaaatcaaaggcagctagaaatacaagcgataaaaaatgatcttcttgaagcaatttctcagttagcagaagaaagattaaaagaaggcaatcctctaaagctttcagaagcaaaagatctttatgaaagccttgataaaaaatcaaaagaaatcttagatgagcttaaagaattttttactttagaagacctaatttctgaggcgaataaaaagttaatagaaagcaaatttgatgaatttgtagagcaattctttcctaacgatttagatgaagacaaagaaagcaatctagaaaagttagaaaatgcgttagacaaagtaaaagaagctttatttgaattaagaggactttttaatgaagaagatttaggaaaacttgttaacctcgccaaagaaaaatttgatgaatttaaagaaacctttgctagcgcacaagacttgtctgaagaaattaaagaagagttaattgatgaagcgcttgataaagttttagaaccaaaagaaacttcgcctgaaaaactttatcaagaaagcatagagtatctagacaaaagccaagaagactacaaagagcacattgacaagataacacaatttttcttgggacaatcttttgctgatttcaatttttcttccttaatgaaaaatgtagaatctttaggctttgagcgtacgcttactgctttagcaaatacatttgcaggccttttggctttaaacaacagccttgaacctttagatatctatcaagcactagagttaatgaaagacttttctaaaagaggcaagacttatgaaattcttcaagacctttgcatttcttacagcatggacttagaatactccaaaagaggatatcaatcctttaaagaggaggcaagaagcgcatcaagcctttgtgaaaataagctagggggtttagctttctttattgacgcttttcttccccaatacgaaactcagcttgttcacggctactttagatttgtaccaaatataaacaaagttttacaacacacaacaagtgaattaataaagcttgctgatcaaaaagaccttcctaaacaaatctttaatcctttagtaagatcttcgcttatcaagcgaatgttagcagaaaaagaaaaggagtatgaagaactcttaaatcaagctgaaatacaaagacaaattgaagaacagcaaatcaaagaactatcaaaaagcataagattaccagaaaaactagaagaaatatttgaagatcttccttctgctataagagaacttattaatcactcacctgaaacaaaaataggaataggtgatgtagaagaaaacctagcaaacttagcgctacttgcaggaatgtttgataggtttgatggaactttaaaggtagttttaaatgacgacacagatcgtgcattaagcggcctcttatatcaagcttcaacttacttagatgcagttaaagaaaggtattcaacgcaactgaaagcaaaatcagaaagcgttagagattttgataaacaagtttttgcagaagctttgaaggtattaatagatagctatgtttgcgctgaaggagaatctaaagaagcttgcacgaacagacttaacgcaacacttttaagtaaagtcaaggagtctgttagaaatttagcttttctacctaaagaaggaaaactttttaatgcgcaaaatttaaaaactgatgagtttggaaacatagtctacgaaaatcactcagaaccaaacaaggagtgggaaatagaatcttatttagacagctttgtaaacagatttagagatttagcttcctcaattgttgcttatcatctttttgatcaatataatccaaatgttgaagaactatctaagaaaattagcgagatagagcaagccaaaggaatgcctcttgaaacttatcttgaacaagaaattgaaggcgtagaagacaatgtaagattaaatgaagttttgagagaaatttcaagcatatttactgacataacaacaaaagaagacctagacacacttgtatttggcacagcgccttatgtttacaaaatcaataccttcatgaaagaagtttttggtaaatatgacggcacaaaagagtattctttctccaataagtttgaagacatagtaccttatagtgagtttgaagattatgaagatgcgattttcaacattttaaagacaaaagtaccgaaagaaaatcttttatatctctttgacgacttttcacctaatgtaatacacacacaaactcctccaattgtctttgaagaagaaattcctgctattggcgaagaagagcagccttctttaactgaggaaacatcttcaatacctgaagctcctttggaaaaagtaagttataaagaagttgcctttgaagacctagaaaatcttgagaaaacactagataagggcgaatttttgactctttatgtagacgaagataaagaagttgaaattaaaaagataggcaataagctatcttttgcatttaaatctaacgataaaattataggaactcttcagctagaggatttaaagaagttaggaaaagagtttaaaaaagcagttgatgaagcttttgaaactatttctgaagcaacttcgcaagaaaagcttctaaattcgtctattgaacaacttaacaaggcaataaaagagaaaaagaagtctaaaaagaaagctaaaaagaaaattaaagaaaaagaagaaacaccttctgaagaaacaagagcagaagaagctgttgaaacagcttctcaaactgaggaagttcctgaagctactccttcagttgaagaagcttctgaagaagctagaaaaactgaaacaagagcctctagcaaaaaagctaaatccgacaagacaaaaaagacatcttctgataagcaaagagaaaaagctaaagaattcgctaaagaaaaccttgaacaacaagctagcgaggttgttttagtagaagaaaataaagaagctgttgaagaagttgaaaaaagtctttctgatgaaaaagaagagtactactctagtttaataaatcttttttcttcgccttcaacaatttcttatgttgctgaggcgattcaacaagctgttgattttgatcctgaagtagaaaaggacataaaaggtttaatttactttggcatctttaatcctaaaaacttagacaaagtgtcgccttactttgtaagtatagataacgcatacgcttatctagtagattttgaaaaagcttacaatgaagacgaagattttaaaagcttagttgatgtttcttatgaaattctagatgagtattcatcttcaacaaagaaagaatctttgcgtgcaactgctgaaaaagttttcaagcaatcaaaaaatcctttagctagaagcgttgctcaaacaatacttcaaagaacttctgatgaaagctacattagcgatagaattaaaagaaaaattagaagcgaaacatatgcagaactttttaagaaaatatcaaaatctaacgaatatattgaaatgcctttaactgataagatatcgcttgaagtcatcacagacgaaaagggcaaaccagaagtagctactttcaagtttgcagaaagcgaagaaaacgaacactatttagctttagacgatactgataatcctctgttttcaaacaatcttctcattatttatgcagctttagaaaatcctaatgttgaaggctatcttccttacttttcaagaatgctttttgaaagcgattttgagttttcactagaaaacttttcttttggcaaaaaatctttaggagagagaggattttattatatcagagaaggcgaaaagacaaaggtgtattctaggacttcagatttcttagcagaagcatttgcaattgttgatactttgcgtcctaatacagaagctgaatctatacaagaagaagaaacaactgaagctcaacaagaagagcaacaaaaatctagcgaagcaaaaaaagaaaatcgaaagaaagaaaaaaagaaatcaaaatctaagaaaaaacctaaaggcaaaaaagatgttgtagatgtgaatgtaacagaagaaggaatacaaacaacaattcagacagaagatgagcaaggatttatacaacaagaaaaggttaacatttcacctgataaagcgattgttagtctgacaaatcgtgtgctaaaaaagttcaaccttcctattcttccttatgaaaaagatgaagaactcctcaaagaagctgaaaaaattgcagaaaacattgaaaaagaaaaagctagagtttcagaagacaagaggttaatttactattctgatacaaatgaaattcttgatgcgcttttagaattcgtgcttcaagaaagacctgaagacaaagagcttagagttcttgctaaagatgttataaaaagttattctggaaacaaatttaaagaaaagcttgcgaaatcgctaatacctaaaattgttgctgtgtatgatttaagcgaagaagacttgtcttccttgattgaaggcttagattttcctgacaagatagatgcctatcaatacaaaataaataaaaacaacctatcaacttcagtcgttgaagatttcaagaaaatagtaaaagacgctttgtctagagttggcatagaagcttctgagtccttcattttagacgaacttataaaggcttttgtagagataacaaggtag

SEQ ID NO: 78 - DNA, 366 bp, YS40 BACTERIOPHAGE
  misc_feature: YS40 ORF78
atgacaaacgaagaacaaatgcccaaagaacttcttgaaatgtgcaaagaaatggatactgaaagaagaaaagaatatgtaggaaaatttttatcgctgtttttaccacatttatcttgggaatgggctaatgttttgtatgaaaaaacaccgtctttagaggaagattatagcggtaaaaaattcgctgaatatcttagcggcctaagcgctttggtagaatttgagcaaaatgatgataagtttcagtcttgttatcacttagatttggctatcaaatatctaagcaaatatggcgtagatgaagatccacttactgctttaagagaaatctactgtataccagaagaagaaaaaggcgaatag

SEQ ID NO: 79 - DNA, 4575 bp, YS40 BACTERIOPHAGE
  misc_feature: YS40 ORF79
atgaagaaaattgtatatcatctttctcaaggagataacgaagcagaaattcactttatactagcaaaaaacaaaatacaagattattacattaaggcaatcaatgaaaacattaagtttgcgttagaacagatattggaaagagagattttttattctgtagcgaaaagaaacaaacactttagtattcaagtttttgttggcgtagaattaaaaacagaagaggcgctttttatcttgcctcaaattcttgtttggcacggatttaaaatagaaaaagtagagaaaatttcagacagaacgctaaaatctctctcactattagaagatataaaattggaaacagaagaggaaggtgtgaatatgcaaaattacataaagtctattgaggacattctatttagctttcaggaaagaagcaaagatattcttcttttagctactactaagtctatcaactattctcaattcgctaacgaaaaaggcgttgtagaaaaagttcctgcaggttatattgcttacaatgttgaggatgggcgtgttttagttggaggaaaatttttaggtgtaaaagtagaacaactagaagaaggtaaaccttggatgctcatacctgcaaacaaaaatacagcacaagcgctagctaaggttcaagcaggaaaggctagcaaagaagatgttgctggactaattgaagcttctcttcaaaaaaataaaaaagacgaaggcattattgacgaaattttaaaagaaggtgtttattttagaggaattacagaggctaagaaaaagctcttgaaagacttgcttcaagcagtagattctgatgcaaataagtatctgcaacaagaaaagaaactttctgcacaaaatcaaaaaaacaaccttgattatgtttatgtgtacgatccagagaataagtatcttacaaacctttcaacctatctagctacaaaagatcagttttctggaaaagaggttttcttctctgacaacctcctcaaactccgttctcaaggtgctagtaatgaagcgttgtttactgaattgaaaagattgttgaaaattgatagtgaagaatacgaaagagctcttgataatcttctcaccgaaataatacaaaatccaaaacttttaaacaaaagaggcgctgcacaaagagatatcgatatattagtggcagaaattttaggcagtaatgcaataatacctgctggtgttttagatataccaacaggaaacatcattaagctagatgatgatactgcaaaaatatttggtcttcctattggtaaacaaaaatatcttcatcccctttatcaagaagttagttcttctacgattgaagaggccgcaaaaattcttaaaaaagacatttctgcaagagtttttgaaagaggtaaaagagaaaatattcctagcgctgaagaagatcctaatgttattaacacactatttcctgaggaggaaagagattatcatagaaagctattttctcttttcgttaaagggggcaagtttaaagaagaacttgatgatttgtggtctttattaaacatacaaaaagatgctaaaggaaatctaataagaattgacggcaacaaaaaagaaattttacctgttcctaaaggaaactttggcgaaaagtttcctttaatgtggatgccgctactgcttcagaaatttggcattataggatataattctcctaaacttggtccaatttcctacaacgaaagaataaagttcgcaaattctaaaatttctttgcagccttatcaagttcaagcgcttcttaagtttttacatcctcattatctaagacacacttctaagttagaaaacttacagaaagtaggaatgtttattgcgcacgctactggtctaggaaaaacttttactggacttttagcgtacgcactagcaacaaatctcggacttcatacagtttcttataatagggacaacaaaacaattacttttgaccaaagaccagccttagcagtaatgcctgaatccattgcaaaaggctggattgatgacgctgaaacattgtttggctggaaagtgggtcaagaaatatttgtcatagaaggcaatcctcagcaaagaagagaaaaatggagtcaactgctagatttgtgggccagagcaaacaggggagaaaaagtaaatcttcccaaaatggtaatcatgaagctttctacattccaaaaaatggaatctgctagtgcagaggatgaagctgacaaatatttcttaaaccttctttctggtccttctattatttctcacaaaggacaaagacttgcagttcctttcggaatgtttggaatgttgttagtagacgaagcttctacaattttctcaaaaagttctgctagaagaagacacattgaagatttatcagattctataacttctgacgcaaatcgtggttatacattactacttaacgctactgcaatgtctaacagccctaaagatttttcttcagcaatggaaatgctgttctatcaaggacatttatttaaaagaattcttggagatcctgtaattaaaacacctagtggaccaataatcaatgaagacatgcttaaaactactgcaacttatcttgatgttgttggtgctactaaccttgaacttaaaggaattactcttgatataaaaactgatgatcctttagcatggcctacttcatccgctgatccacaaaaactatcttctgatgatcctttagttagagaaccagaaaaagcaaaacaaaagtttgaagacatgttcttaactgtagcttcaactatttatcaaatagctaagttatataaagaagacagacctgtagatacaacaatttatgatcctgttacacgatcaaaaaaatcagtagattctaagttgttctatgaaatgtatgtgcctggttttgctaccttatttagttatgtacaaggtggtgcttttacctgggaacgtgcattagaatacggttttactagctttgatgaagaaggtccattcataattgttgacgaagaaaataaacctaaaaatgcctcagttagagaaattgctataagaagcttggttgacactttatcgtcagttagtccaaatattttagacagatttggcagcaacaacatcttaaaacacataattcccaaaataaaggaatacaagacagctgaaggtgaaaagaaaacaaagttaaagaaagaaattcttgaattggcaacaaaagttcctgtaatttctttgggcaacacacaatatacatttgctgatttgcttagtttaggatttgaaaatgtagtaactcacggccttggttctgctgaaattgagtttgatgtagatgttaatgcttcaagagaaaatagacaacaaaagtttaaaaagggatttaagattagatttcctggttatgcttggaataaaaaggttgataaagaacaagaaatgtctggaaccttgaaaagaaggacaggcgttagcggcttattggagcaaaggaaacaaaaaacaggaagcgaaagaaagatttctattgaaacgagaaacaaaatgatgaaatacactttctttattacacctatgctttctgacgataaacacctctactatccagtaattgaaaacacagcccaaggtgagtttagaattcgcttagctaagcaagaccttcaaacaggtgtaaaaacacttgttgatagaaacgatcctaaggcagcgccttttttaaatgaagatggaacactaaatgaaaatttccaaattaaacaaagcgaaggtaacgatgttacactagatacaattggcgaacagattggtaaagagcttgataaagcttcttacaacgcaatcgcaactacagaacaatttaaaagagtagtagaagaagctttgaagccattcgcaacatcaagtaaaacttctcaagatttaggccgaaaaattctaattacttctcctagcacagcttttgtaaagtctttagactatgtttttgacaatattagaaaaaatacgcctttaggaaaaatgtttagaaatttgataccgttatttggcgataagggcattgttgaggcgcttaagaggtattacaatggagcagttactggtgaaactgacatggctgatagaatgaaaatcattgaaagtcataacgctaacaacaagccttctataactgttctctcaagagcgttagctagaggcgtaaacatgccttcacaaaaaatcgttgttcatggtgctactttctcttacgaaactattttgcaaatgctaggtagagcttttcgccccggtggaaaacaaaaaggtaaagtagatgcgtcaattattcttcctcaaagtgttgcatctgttttaggattgaagtcgtttgaagttaaaaaactctttgatacacaaaaacttcttgctcaaaatacacctattcttgaacaaggcgaggaagaagaatatgaggctattatgagagaaagggaagaagccttctatagaaaagtagtcgatgctttatctaacatcattactcctgttaagatagatgttaagtag

SEQ ID NO: 80 - DNA, 864 bp, YS40 BACTERIOPHAGE
  misc_feature: YS40 ORF80
gtggcaagaattagtgaagataatcctttaatagcccccaattacccagttcctttttcaaataacctttatgaagtaggacaatattcgcctagcgattccacattatcagcgctaaggctattagaagcaaaagaccaaggtttgttttatgatcctactattgatatttatgatgacgaaggaaacattgatgaaaacaaagttgctaggctaaacaaagaagaacttttgagttttgcggcgctagtagtagataatgtaattcctttttatgcgcttttaaatcttcgtgctgtttttaagcaagtagaagtagtgtttaatctctttaaaatcacaaaagtttattggcataagaaaagtgagattagagaacttgtagactttgatcctgatttagctattataggttctagtttttcccatgacaaaatttatcttcatgtctttcaaccttactacctttacttgactgtacctgtagttagaagagtacctaactttactcagggcatttcagtagcttctttggctactgcaagagaaattgcaaatataacctttcaaaagctttcttcttcttttccagaacaaattgaaagatttatatctttgaggggctggaaaagagaaaaactttctgacgaagatttgaaggttggagagttttttggcctaaatttgagtaaaaatttataccatgaaaacgatgaaattatcagcttagaatctacacttgggccaaaagaagatttcatagaagcttttgttttttacttttttcatagaaactatctgcagcagtttcccaaagtctttgcatacatgagaaaccttgaaaagtttttggaggatttatga

SEQ ID NO: 81 - DNA, 1953 bp, YS40 BACTERIOPHAGE
  misc_feature: YS40 ORF81
atggctacagattgtatacaaaaacaaagtgaacaaaaaaatacagaattaacttattggcgagtttctgaagaagggttttggaacccattttatcagacaggagcgtttttagaaaaaaatggtgaaatttatttattgcaaactgaatacttaaattatctatctaatataggtggcgttaattacagattaaagctatacaaacttattgatggatgtacaacatcagaaggaaatgattgtcttgaagctgttgctgtatacaatcccattaacgatttactgagcgtcataaccaatttttattcccacccatttttttcaaatgaaacaggttttattgtaaagtattactatacaaatgatgacactcaaaagataatatttaaaactgaggatggtttttattatgttgaaaagggccttatttacgctgaaaagcaaggtggttcaacttctatttatttattctacgatgacatttattttatttacaaaataacttcaagtagtattactttagaggggatttatcatcctctttctctgtatccgcaagtacctcacggaaattttatggggattactcacttaaaagataatgtatttttggcttcatatttgcatggtgattataatagtgaaagttttactttagattatacttatcatgttttttctattggtctttcaaatgggcaaccaagtataacaccgattaaggtagaaacaagaacggaaaatcatcttaaagacacttcctatcttttggaaaatttttctattaacttggatgtttatgacaataattactattctgtatatacatattttgttaacaacacatatattcgcaagatatattcaaatgaaacaaagtttttagaaccatattctctttatgtttctacctctgttaacagaaaatctataactctttacttggggtacatcagagttgttagtcttgatgacaattttaatttttctttttcaaattatattccatttgtaagagaaaatccaaaagaattttacaaattttcttatttggcctctcttggttcaaatagtacatacactaataatgtaaatatttatgatgacacattgcactctgagctttattataagaaaaatttgttctttgcaaaagaaaaaaataataagttagttatttttgcatatccaagatttagtgatatttttgacacgtcttcttatggtggatcaacaaattctgttctaaggggaaattcttacttggatttctgtccttccctgcctagtgtgttaatttttacaagagataatagtgagtatattttaaatgcggctaaaaagagcggttgctttacattcatggcaacttcaggaaacatagaaattcacaacagcgaacttccgttatttgaagacaaaaagcagtcttcttctcctggaaacccaccaggtttagttgtaaaacaagaatttaatgaagacatttgctcaattttatcttctaaacatccagatattgattatacagggtgtttgtcttatcatttttacaaatctatatacacaacaagtaatgatttttctgttggttttgtagaattttataaagattgtattgttggaattgtttttactattcctgtagctagaaatcctggtcttcaaggtaatcaaaaccaagaaagatacggaagtattctcgtagatgaattttacattactgttgctttatatgtagatacaaacgatccatcaaacgaagttaaagtaaaaattttagatgctggtaagttatacacaattgattcgtcgaatgaatattatcaagataactacttcaacagaagaaattctattgttttgcgccaaatgtttgccgaatcggattttgtggaaaattttatgtttttgtactttaaagacataaatagtgtatatcctccttaa

SEQ ID NO: 82 - DNA, 1839 bp, YS40 BACTERIOPHAGE
  misc_feature: YS40 ORF82
atgggaatagattgcgcaaacataaacagaactcaagaaccaaactttaacggatttattgatgagttgctaccttactattataatcaaaataatctttattcctcgtttaagtttcctataaacggaagaaactatcttttttattttgcaagaagatatgatgaagtaattcaaaactatgtagttgaggcaaaacttgtagatgaaacaagttgttatgaagatacctgcagcgttgtatcatcaattgacttaacttatgtagatagtgaaatttttaacgctctttcacagtatttatgcagcgttcaaggtggtgagcaaatttacaagcttcttaatatttatcctataaatttttatgatctgcaacaagtaggttctgatgaatatttggcggttattaaagtagaaaattccctgtttagaactacgggttattataacacctacattttatcttccttcgatatctatcttatttttagaatctcttctaattcaatgactttagaaagaatagaaattccaattttggaacatgcagcccttggaggttgtctaccagatagcgcaacatatgaacaaggatttttgaagtataaaggaaatagcttatttttatatgcaggagtaagctctacgtcagatctctaccttatgcctaaacttttgatgtattcattagatacgcaaagtggtcaaaagagtatacttgcacaacatcagttgtcattttctacaaacccttcagattatggaataagcactttggatccatatacatttcaagtaaacagctactttttgcaggctgtgcataattctgatgatttgtttgttctaggaacgtatctttacttttatttttacgatgatactagtgggtttggtgtcgaacttgaaccattactattaaaagttattaaattttccaatcgagatggtttaacaatatctgatttttacactccagccttttatagtgatctgaacactcttcttacaaatctacttaatgtaagtaatacatatgttaaccatatcgattttattaatggtaagtttttcaaaattggcaacaaagttgttctggtgggaatgattgattacttaacgatagaagatttaccagatattggtaaaacaaatgtagcaatgtcgcactatacttttgttagaattccagcaacctttgttatctatgactttgatgaagcaaatctaaaatttgtttttaatgaaaattattttactggtgtaagtgaagacaacaatttggatttttactataagttttttaatagtgctttgccaattttcgaagaaaaaacaacaaatccaacaactccttatggagtatttgtaaataactcagcgaagtcagatatatgtcaaattctatcttacaaatatgcaatctctgattgttcttctctagggttttatttcgtaaacattattggttattttattcagctattaaatgcagttcaaagttttagtgatgggtgtagtttatcgatagtaggtgcttttaggcttttgaatacgcaaaacgatttaaaatctgctccaaacgaatccccaatagaagaatatttagttatcgttttgttcagattttcttttgatgaacaaggtaatcttataaaagaatataaaatactagacgctgcaccgctttatgtagcttctcctagtgatacttatactaatagaggagcttctgtacatattccacaaattttctttgataatcaaaaaagattacactattctgtgtatgtttacagcgaactacctcaaggagatggaggtggcgttgctgcgtaa

SEQ ID NO: 83 - DNA, 228 bp, YS40 BACTERIOPHAGE
  misc_feature: YS40 ORF73
atgaaggtggaggtgagtgggatgaagaagcccattggacaattttgggacgaaaaggggtatgttcttgttcttaaccccatgcttgtcaagaacattctcaagactatcggcataaatcgccatgcagaagcccttatcgttaagaaaaggggtgcagaatacattgaggtttgggaggtgccctttctcccttacgcaacagaatatgcctataggctaaagtaa

SEQ ID NO: 84 - DNA, 231 bp, YS40 BACTERIOPHAGE
  misc_feature: YS40 ORF84
atgatgaaggtggaggtgagtaggatggaaaagaagcttctcagggactttgacgatagctacattttcttgcaagattctcaggatgtgaagtccgtacttgagtatattggtacttgtgagcgcttcccctatcttcttgtaaagcttgattcttctggggccgattacgccgaggtgtggggtacctttgggtggaagctttacgatattgcctatcgcttgaagtag

SEQ ID NO: 85 - DNA, 153 bp, YS40 BACTERIOPHAGE
  misc_feature: YS40 ORF85
atgagcaaagtcatcaggttcattgaaaatcttctacacttcatcaggtacgctgaggaaataaggttgtcaggattcgccctagaagctatggataaaaggggcgggaaaatagtattcctcctcatcggaggagaggtcatagaaggttag

SEQ ID NO: 86 - DNA, 252 bp, YS40 BACTERIOPHAGE
  misc_feature: YS40 ORF86
atgagggtggaggtgatgagcatgactaggcaagagaaagaagcgttgaaagagttccttgaatccctcaatctcggaatctcggtaaaaatcttggaggatgaaaactttggtgaagtccttcttttggacttctccgaaactctaagcaagcttgactttgttcatcactctctcgctgtgtttcctgatagggttttgttaataaggcagaggggcttgcaagctccttatgtgaaggagtttaagtaa

SEQ ID NO: 87 - DNA, 237 bp, YS40 BACTERIOPHAGE
  misc_feature: YS40 ORF87
atggtcggaaaggtactaaacatcttactaaacgggtctttggtcaggaaaaaagtaaaggaagtctatacagacggcgatctcttacacatcatcgctgaagatgagtatggtctaacacacgacatctactttaatcttcacaatccaactggagtggccgtggtagttactgacaaagataaagtacttagtgcatggtactacggggatagcgtgatgtatatccgatcctga

SEQ ID NO: 88 - DNA, 246 bp, YS40 BACTERIOPHAGE
  misc_feature: YS40 ORF88
atgggaaagagcacaaaggctagcatgacgaaaaggagcccgacacctaaaaagatcgtcctcgtttgcgagaagtgtggaaaggagtttgtggctgaagctaagaccaaaccacccaagaggtgcgatcaatgccgcgttactagtgctagtagctggaggcttgttggctttgaggacagcccctttggaaaagtgcgcatagaggaaagaaagggcaagattcgtaagatccttgtagagtaa

SEQ ID NO: 89 - DNA, 486 bp, YS40 BACTERIOPHAGE
  misc_feature: YS40 ORF89
atgaaaaaactttttaccattttcctgtttttgttcaatcctctaatgattgatcaatattttgtgatcaaagaaaaaatcgcccataaaagatatcaaatcgcaaagaatttttacataaaagacaaaagcaaagaagactttaatattattctagccaaaaagctaaatagattatccttctataacttagttcctcagcaaactgatgatgatccatatgttgcctcttgtggcccaatagaaagagttaaagaaacaatcatagcgctatcacaagatttattctttgacaaaaatcgtaaaaaacatctttgcggcaaaaaagtaaaaatcatctcagatttaggaatttttgaaggtattgtctacgacacaatgaactccagatacataaaatctgcagatatagtagttgatacttacgagaaagctattaaactaggaatatcaaagaatgctattttgattgtttacgaagattaa

SEQ ID NO: 90 - DNA, 456 bp, YS40 BACTERIOPHAGE
  misc_feature: YS40 ORF90
atgctcggagagggggagggcttgacacaagacgaaaggtgtggtaggatgaaggtgcctggggtaaatcctcaggcgaggaggtgtaagatggaagaaaaactccagaacaagatcgtggaagaggtcaagaggtaccttgagaagtggggtctgcctaccgatgtaaaaatcggtgggaatgtgaagaagggtgaagatgcgcttagcgttagggcctttgtagaggtttctaaccttaggcgcatctttgtagagtacgctggtgcttcacttaggggggttcctgagggttacaatggttttgctaacttctttgtcgttccgaggggggataggaatgtctacctatatcttgacgatgggctaatcatctctagctttgacggaaaaaaactaatcattgaggtagttcctcaccccaaggtgttgccattcttcaggtatgtcagttaa

SEQ ID NO: 91 - DNA, 609 bp, YS40 BACTERIOPHAGE
  misc_feature: YS40 ORF91
atgaggctagagaaaaaactaattttgctagatagcaaaaaatttgaccaaaacaaagttaagtcaatgatttatgaagcagttgaaaaaatccttgaacaaaacaaagagcttctttttcttagaaaaccaaagttttttagcagatcaatactttttcagttaaaagaaagaagactagataaccccttctctcatctagaagctgtaaataaatctttcttttacctcagaaaaacttttgacatgtttccaaataaagaagatttagaaagcggcttagaagactttttagatttgctttttgaaagcatgtatttaaactatgttaatgatgacgaaaagagatatctcatcaactcaacaaaaccttttctcatgaagataaaaacaacacaacaaaaattttacagcccaataacgagatcctatcaaacaatagaaagcaaaaacattttcttttatccggcaaaagaagaagattttgccaaagaaaaagaaatatactttcctttaatctcccaagtttcactatcaaagaaaacagaaagcattgtcgtttctaggagttatatttgcgttctctttgaaatagaagaaaacttttaa

SEQ ID NO: 92 - DNA, 435 bp, YS40 BACTERIOPHAGE
  misc_feature: YS40 ORF92
atgatttttgaagtaaaggccaaaaaatacttttttgcacaacctcactacagcccgatgatttctctaaagcattatgctcacaaaattacttactacaaagattttgagaaagcgtattcaaaatacaaagagaattttaaaaattacaaacacgctttaaatcgctggaaagaaaaaagaatgtttgctttaaaagaacatgcaagcgtggaggatttcaaaagaagaataacattagaacttcttgacattatacaaatagaagattatgaagatttgattagatattacttaaaagaaaagtctgaaaaggattatgcgcaccgatcatattcaatatacaaaccaatttttggagtttatgataaaaatagttcgctatatttgtgctttggagaagttgagtttgtgatattaagagaagtgttttga

SEQ ID NO: 93 - DNA, 426 bp, YS40 BACTERIOPHAGE
  misc_feature: YS40 ORF93
atgacattggaggtcaagggtaaaaaatatttctatgtaaaaccctactacaatccaaagatacacttaaagagatatacttacaaaatttactattacaaaacctttgaaaatgcttttgaagcctacaaaaaagaatttcgtaattacaaagttgcatttaatcgttgggagggaataaaaatgtttgttttagaggaagaattaaaacctatagaatactttgaaaatttaagaacatcagaagttctttatatgtttccaatggaaacttacgaagatttgataacttcatatttgaatgggaagtttaaaagaaatctctttttgatacataaaccgattttggggtttcacgataaaagtaatctagcttatgtatttttcggagaagttgaatatgtgaaaattcatgaagtgttttga

SEQ ID NO: 94 - DNA, 447 bp, YS40 BACTERIOPHAGE
  misc_feature: YS40 ORF94
atggttgcaagcagcacttacgcaaaactttatattgtaaaaagttcgcactttaagaaaattttagaaaagtatgctcaagatgtaaatgaatttcttaatcctcaaggatacggattaaaggaagaccacaaatatagcggatacaagtatgtaagtacagatattcctgtagatattggtattaaggcgtttttaatgctaaaccaatattttacctttacaccctataacttcaggcttaactatagaaaatccaaaaacatttttgaatttattaaaaacccacatccttttattcacaaaaaggtaatatctgcgtataggaaaggtttagttcctgatacaatcatattgtcttttgccgaaagtacacatagatataatccgaagaagagaatacttgtagacttaatatttttccctgactattttccttacttttga

SEQ ID NO: 95 - DNA, 177 bp, YS40 BACTERIOPHAGE
  misc_feature: YS40 ORF95
gtgtgccaaaagtgccaaaaggagtttgttaccgaaagcaaaacccgtcctcctaaatattgtgaagattgccgcaagaatactacatggaaagtgattggctacgaggatactaaatatggtaaggttagggttgaggaaaggaatggtaaagtgaggaaaatcttggttgaatag

SEQ ID NO: 96 - DNA, 129 bp, YS40 BACTERIOPHAGE
  misc_feature: YS40 ORF96
atgaacattacggaccttcttttcgttcttgcccatcttcttcacctccacccccaagataccacaaagaaagatgattgtcaaggggggattgagaagattgacctcggttgggctggttttgcgtga

SEQ ID NO: 97 - DNA, 408 bp, YS40 BACTERIOPHAGE
  misc_feature: YS40 ORF97
gtgaacaagcatgaagcacaaatacaatcaataagaatgtcaatgtttttctatttaatttataacgcattttcttttattgttgcaaaattattagctttaagcgaaaaggaattttatattttgtttgcgatattcatgatgaattttggcaaaaaagctgaggactcttatttgaaaacttatccagatttcaaagatattccccttttaaggattttaatattagctttcattgtaattcaatttatttttctttttgccacagaaatgataacaaaaaagatttatacaaatgacttagttctttatctcctatttaacacactttttatcatcttcttagagtggttttatgctaaaaagttgagggaactagaagaaaataaagaaactgacaatttatga

SEQ ID NO: 98 - DNA, 546 bp, YS40 BACTERIOPHAGE
  misc_feature: YS40 ORF98
atggaagtaccaccattcttagatgttttacataaacgatatgcagtctttaatgaaaaagactatattcaaaaaatgtctgaaaaattggcttcaatcgttagcaacaaagttgttttcatagatgatttttctcaaatagaaaaagattttaaaatgctaaatgaagggttagggccgcaaatattaaaactaaaaaatcttaaacttgtttcggtagaaagtgtggatttgaaagacatatattggcttctttatcccgctagtgtagcaagaacacttgcagtaaaaggatctgctgattctgagttttttttagaaatttcaagaaatttaaaaagagtagaaaaccatgcatttttctttaagtcctttaaagagctttttaaagcatgtaatgatttgcaaattctacaaagaataccagcgtatctttttgtgccaataaaaatttttaggttagaagtagagtttaaaggtgaaagagaagatattttcagagtgtacgctaacgcttaccttgttaaaaagaacatttctttctga

SEQ ID NO: 99 - DNA, 543 bp, YS40 BACTERIOPHAGE
  misc_feature: YS40 ORF99
atgattagaaacatcgtaaatactgaagtttttgagaaaaaattttctgatgaagttcttgttatcttcagatactacattactcagaatggtaaaaagtttgactttaatgaaaagaaaaggcttccaatgatgatttttgaggaagacaaatatgatatagacacaattcttgatttgggaactttatttgttaaaaatctttatcactatagatccgcaccttttgaatcgctacttgacaaatttgcgccaagaaaaataaaaacttttaaaaacgatatagctttttacatagcagattatgctgttccagcaaatggaataaatggaatttatagcgacaaaataagagttaaagttgtaagcaaaaaatacttttacgctattgacaacatctttgattttaaaaaaggtcaaatctttattcccgattattacataacctttgaaaatacaaaacctgaagaaacctctctaaaaatagaggtttctgcaatgctaggaaagatgtatttgtatagccttaacgagatattttaa

SEQ ID NO: 100 - DNA, 591 bp, YS40 BACTERIOPHAGE
  misc_feature: YS40 ORF100
gtgaagagattttttaagtttcattgctatgattatgtctttaagaagggggggattagaatgaaagcagtagaaggtttcttggagtcaaagtcctttaaaatcttgaagagatataaagcgtatctagaactagactattatcctagcgaaaacaatcgctttgctgacgaaatagtaggagagttttctttgtacaaatctttcattatttctgatttcgtagacaacaaaactttttacagatcaattgagtatttttacaacaacttgtcatgtttatctcttgagagagatttgttgaagactttcagcacaaaggttgtttgtagcaaagctattgaaaaaaagtatttctcggtttttattgatgattatagcgaacatgttattgatgactacacaatgtctattgtttatcttaggccttctcaaatagaagagataaaagatttaaaagaagtaatagaaggcgatactttatttgttgcacaaaggaaaatacttttgaaagatacagatgaactttctggaatgatagaaggattaattggagttttgtatgaatacaaaatagaggaggtgttttaa

SEQ ID NO: 101 - DNA, 513 bp, YS40 BACTERIOPHAGE
  misc_feature: YS40 ORF101
atggtgggggaaagcattattaaagaactagaagattacaaaagcctcaaaaactttttgccttttttagaaatcttagatgccaactactttttctctaatagcggcatatctagagaagtgaagattttgaacaaagaggaatacttgaaaaagatatctaagctaacaactgacaagataggacgcaaatacatttatttttaccaagttcttgaccctgagtatgattgctatattatagctcatagtggtagtgagaaaaaggttatttctaacaaaagagtaattcacacaacttatgtttacgatagaatgaagatttcagaagtgaattacataagagagagaataacagatttagaaatgctttatcctgcagtttacttgcttattgttgattctgttgttgagtcatttagagaaggcagatggtttaaaagggtagaattaattgaaaaatgttttgttggaagaatatatagagtaaagcatcgtgaagagattttttaa

SEQ ID NO: 102 - DNA, 537 bp, YS40 BACTERIOPHAGE
  misc_feature: YS40 ORF102
atgcaggtgaaagttttagaggaatttttaaaatcggctaactttaaagcattaaagatatacaaagcttacttagaacttaattactatccaaacaatagcaatcgttttgcagacgaaatagttgaagaattttctcgttgtaaggcttttattatttctgactatgtaagcaacaaaatcttttacaaggccattgagtattactacaatgttatgtctggtttaacattagaaagagatttatttaaaacttttgatacgaaagttattagtggcaagagaattgccaaaaaatattattcgctttttattgacgaatatgttgattatagcaatgaagttagctcaatagtcgtttcatatctcaaaccttcacaaatagaagaagtaaaaagcttaagagaactagtaggaaactaccaactatttgttgctgaaagagaaatattacttgatgatgttagcgattttcctggaatgatagaaggattggctggagttttatatgaatacaaaatagaggaggtgttttga

SEQ ID NO: 103 - DNA, 489 bp, YS40 BACTERIOPHAGE
  misc_feature: YS40 ORF103
atgaaagaatacattcgcttagtaaacgaaaaaatcaagaagattagtggaatgtttaaagcaagacacttttcaatagtaactatcaaatactattattttgataatgatgtggcttcaacgatttctcacatatacgaaactgatctgggaggaaaaaattttgaacattttttaaatactgcaataaaagcttccttagatcaagacgctgatagaaaagtgaaaaatattaagttttacgatgaagaaaagctattttttgtaactaacgatgaaaacggaaaaatcttatttaaagaaataaacgaggaaaatattttctcattcgtaaagccacatattgaagattattttacagactttatgtttcttcctattatggagtacgatgagttttttgtgttaaatgcaggctttagaaaattgaaagaaacacacaatattttaggtctggggtttttattgacctatgattacatgttctga

SEQ ID NO: 104 - DNA, 516 bp, YS40 BACTERIOPHAGE
  misc_feature: YS40 ORF104
atgaaaaacctaataaaagacttgaatgattttcaagatattaaaccttttcttcctttccttgagataattaaatcacaatactttcgttctgggtggaaagtttctaatgaaataaaaatgatagaaaaagaagaatatattagaattttatctcgcctagtagttgacaaaaaaggcaggaagtatttatatttccaccaaatttttcgccctgaaacgaaatactatctagtttatcacgaatgggaagaaagcaatatattatctaatgttagaaagcgttttccttcttacatatatgatagatttaataattcttctcccattaaaataaaaagcttccaaagagaagtagagtttgttagtaacatttatccaaatgtttacttattggtacttgattacaggattgcgtcttataaagaaaatatgtttacaaaaagaatagaaatagttgaggagtcaactttaggacgaatatataagatcaaatatcgcaaagaagtattctaa

SEQ ID NO: 105 - DNA, 531 bp, YS40 BACTERIOPHAGE
  misc_feature: YS40 ORF105
atgaggaaagaagaggcaaaagatttggttaaagattttgacgacttttctaaatttaagcttattctttcatttgcagaagtttttgaaggaaaatattttgttttaaaggaaaacttatgtccagatatagaaatcattgaaaaagaagaatgcttacagacaatatctgagttaataagcgagaaggaagacaataggtataccttcttctacaaaatcatttacccaaaatcttgttgtttattggaatatactggagaggaaactgaagttattcctagtatttgtagacattactcatatattttttctaatggaataaccaatgacgagatagaaaggtttagaagagtccttttaagaatttctagtgagaattttaaaagcgacaagttgatttactttttagttgacgattttgagattgacttgtatatgaaaaacaaattgacagaagaaataactttcgttaaaagatgtacatttggacgaatttacaaaattaaacatcataaagaagttttttaa

SEQ ID NO: 106 - DNA, 522 bp, YS40 BACTERIOPHAGE
  misc_feature: YS40 ORF106
atggaaagatttttaaaggcagggctatttaaaattcttagcaaaaagaaggcttatatggaagttacttatcatcctagcgaaagaaatatttttgccaaagaagtagtttctagttttgaattttataaaacacttattacttgtgattattctgagaaataccaagttttttataagcgcattggacatttttacaatagattcttatctgttcctgttgaaagaggtgctccaaagaaatttgatgtaagatttttgggtgaggaaaaaataaacaaagagtgttattcaatcttaattgacgattataaagctattgaaaaaagtgtaggtgtatatgttacttattttaaaccacagcaaataacggaaataaaagatgtgaaaaacttgctagatgttaatattttatttgttgcaaaaagaaaagtatttctagatgatgttgatgaatgttcaggaataatagaggggttagtaggcactttgtatacttataagcttaatgaggttttctaa

SEQ ID NO: 107 - DNA, 1689 bp, YS40 BACTERIOPHAGE
  misc_feature: YS40 ORF107
atgactgacatcattaaaaagcacgccaaaaagatagaaaatacagaatatttggatatttttaaagtaatcctctttgcccctaatcgtacaaagaatagagaacctctactttatactctcaatggttacgaggatttattggtttccgatatttatgtttttcctgatgagattaatggtcaaaatgttggaatgtacggattcactactcaggaaaaaaataagcccgtaatataccttaacgaaaaactttttacaaagtatactccagtttataatgcagttggaactaccctatttcacgaaatcttgcaccaaaggctatcgcatattaaacgatttgagcattattttagaataaattcgatgctaactaattttgttttggatttgtttattcaggaaatcgtgtatcagtttatagatgatcctaaggaaatcaaggaaaatagtatatgttttgaaaattttgaaaagaatattcgcaatggcgttaatactttgggaatgctacttaagcctaatcttaaaggcgttaatattgagcaactgaagaaaatgagtgattatgagctaatggagttcttctttgatatttttgaagatatttcacaacaaatatctcagatgattagcgaatccattgataaacacttaaataatcaaaatagtcagcaaaatcaaggtaaacaagataatgataatcagcaaaatcaagaggagaataatcaacagaatcaaggtaataaccagcaaaaccaagggggaaacaatcaacagaatcagggtgtacaaaacaataataaccaggaacaaaataacaatcagcaaggacaaggttcccaagaaaacttctttgatctgctaaacaacatctttaatgatgttgctaatcaaataaggaataaggcaaagaacgatccaaagtttaaagaagatttagcgaatgcttcatcatttggtcccttggataatgcaataaaaaacgaaatcattcgccaacttattggcgaaaacaatgaagttagtgattacgatgatgaaaagaacaggagaatacttgaaggatttttggagaaaaataaaaagcacatcggaaatacaggtggattccttaggaatattcttgacacaaatgaaaagataaagattcctcagtttgtccttcagttatttaatcgtttgtatcgtggaggatattttagacatagaatcgtttacccaaagaaatcctttagaagcaacgatggtctagtgttagggaagaaaaaatactttggccttgaaatgtatgttattgtagatacctcgggctcaatgggtaatgaggatttagagaaatcaataaaaatcttgaggtttttggtagagaacaatgctaatgttcatctctactttaatgatgttgatttccaagagatagaaaatgttaatagcgtatcaaaactccataaatccttaaaaaatggtgtaaaaggaggtggtggtagcgtttttgataaagtttttgaacatgtgtctaaaaagaatattcctggtatagtcattacagatttgcatatctacggattagaaaaaatgcccaaagatgttgttgtaatcgctacagaaggttatgataagcaagcctttaatgacttaatgagcaaaggatatcttgtcgttaggatggaagatttggagtaa

SEQ ID NO: 108 - DNA, 1044 bp, YS40 BACTERIOPHAGE
  misc_feature: YS40 ORF108
atggagagagtaacaaaggtaaaaaacatcgctaaaaaaattcttactagtggattcccggtcctattcctcggagataggggaataggaaaaactaatgtatcttgggaaatcgccagggaaatgggtttggaggtattctatcttaatgtttctcaaatgtttccagaaaatatttcattcccacaagtcagggaagaaatgctacactttgtaacctttgatcttaataataaaatcgttattcttgatgagttgacgaataggaatccagatatgcactcgcttttacaatctttggtacttgacaagagaatcggcgacaaaaagtttgaaaatatttacttcattgctacaggcaataaacctgaacaatcttcagtagccacaataatccctcgtccattagtagagaggtttgtagttattgattttcccattccctcaaaagaggaatgggcttcgtatacgatgagtagaggcgggaatcctttgtttgtatctttcattcttaatagtccagatcatatgttttacgaagagccaaaagatgatgatttaaaccagtatccttctccaaggaataatactcgtactgctattcttctaaaggatttaatcaataacgaaatggtaggaaatgaagattatgttatgtctattgatagcgagcttgatattatcatctctggatcatcgggaaggaaagtatatttagcctttaagaactatattattcaaggtaggtattattcttacaaaaactacgctaagggagattatcctagaaatgacaatgaggtgttatcgttgattgtggatgtcgccgatcttgtaaagaaaggaatgattgatcttaataaatttagcgaaatctttgactacattcacaaaaaccacaagaaatatgatcactatatgttgtcctatactgcattggtcaatggtaacgaatggaaagccgatgtttacgagtatgttcacactcacaaaaatagtaccttgagcaaatacttgcaaaatatctccaaaaagggcgtttag

SEQ ID NO: 109 - DNA, 696 bp, YS40 BACTERIOPHAGE
  misc_feature: YS40 ORF109
atgtctgctgatgctaaactaatcacgattactttatataggccgaaaaacaatctgaagtataagattaaaacaaaaactttatctgtattttcatatgaggattttttaaaagtctttgaaaaaaggttgatatcgctgaaaacagaaatagaagaatcctcaaagttttttgtagttcaatacaaaacttcaactttgaggtttgaggtcaaaagaggatttgaaaagtattacacaactcctaacgattttgatagctttgtagggcaagtagttcacaagtttaaaaaatttttgagcaaaaaaaagatttatcgcttggcatttcaacaaaacccaataaaaaagaaaatcaaagaaaagtatcatcttctatcaatagaactagaaaatgaagacaaactttacaaacttccttcaaactacgatacaatcgtttatttacacttcgcaacaataaaagatgcctattattatcgtagcaatccaagcgtttacagcaatgttttaaaagaagctttttttgaaaacctgatttcagcgccaacagaaataaatgaagaaaaaatactctacaacctttcaaaaacactaaatcacaagttctatctttctggtgaaagaataaatgatagagtgttttatttcaactatcacttttcgctaaacatttttcttgaagaagttttctga

SEQ ID NO: 110 - DNA, 747 bp, YS40 BACTERIOPHAGE
  misc_feature: YS40 ORF110
atgaaagagatgaagatctacaacattaaagagtctgtcaatctcgttgaaaatcttcgtaaaaacttcttctatgttcttagggtaaataatgttaaggattttgaaaatgatttggtcctcattaatctttttagcgatgaatacattttcgcccatgaaactgaaaaactcttcttcaactatgttacttctcgttatggtgttttcaagttctctgaacttgacaacattaatccattgtttgaacctgataatccagtttacatctctaggtactatttccttacaaagaacaatactatctactatgtgaactcttattctggtatgtttaaggaaaatatcatttacaagccacttcttctaaacttttccaataaacatacaaaggggaatattatgaagcgtgatgtctacaaaatcatcaacatccttgaagaaaacaaagatgttgcaaatactttccttgaacaaaatgtagatgataaggatcttgaaaagtttataaatgcttttggtccaagggtcattaaaaaggctcttaatactatagcaaaagaaaacgagatcaatgctgagtttttgaagagccacatcactaatgttagggatatggttcaacttgtggttaatattgtggaaaagaacaatgaatctttgaatgctgttaagggattggtaagcacatctgaagagttcactaaaatcctcgtagataaatacaacatcatgagcttgttctaa

SEQ ID NO: 111 - DNA, 393 bp, YS40 BACTERIOPHAGE
  misc_feature: YS40 ORF111
gtgaagagaaaagaagtatttgtaaaggttgtacttacactacaaaacctttcgcaatgtagaaaatctgatgaagttatttatcgtatgaaagaacatagtagccttattcccttttggcgaaaacttaatagaaaaatcctctctcaagtgaacaagattcacaaggtaaaagtagacatctgtattaatgacgaaaaaatcttctcaaaaaatactgatattcgcaacgaaaagtttctttacaaaatcgttgaaaacaaaaatacagaaaagttagacaatgactaccacatttttgcatatattagtttttactgctttttaaatgaagacctgatgaagtttgacataaagcttgaggatagggtagtatttagagagctattttaa

SEQ ID NO: 112 - DNA, 546 bp, YS40 BACTERIOPHAGE
  misc_feature: YS40 ORF112
gtgaaagacaagctcaaagaggcgtggaaatgggaaatgatagctgatcttgacgaatattatgttggtgcctcacattactttatgaacgaaataggtggtgaagattctaaggaaatctatgatagcttcaaaatgaggatagtttcaaatcctaagtttgaagatttctgtaaggtattagagcatgccgaaaagttatattgctacttgcacacattcttacccaatgagcaaaatatgttctatataaacattgaaacaaatattcgtggaaagcgtttaaatccaaaaagattcaaagctttgcccaatgatgatgaaaaatattcggattaccttcttagaaatattaacgagttctttaaacaagcgttgccggagcttttggaaaataatgttaaaagaccagaaatccacgccgcaatatatagaaaaagaaaagttgatacatataacggatataaagtatattctgactatttttacgagtacattttcctggaaatcatcttgaagcttcgccttaataaggaggtgatttaa

SEQ ID NO: 113 - DNA, 540 bp, YS40 BACTERIOPHAGE
  misc_feature: YS40 ORF113
atgaacaaaatctggaaatgggaaatgataactaaactatcttcttactatgttggcagttctcactatgttgtagatgagtttgatgaagaaaatacaaagaaagtatacgccgagttttcaataaaaatattttcaaatcctaagtttgaagatttatacaaaaacttaaagcatgcagaaagcctttatgcctattttcaaacctttcttcccaaggaggaggacatattgtatattgacattgaaacagagccatattctagaagcacaaagccttatacttcttatgtatcacccagaaggtggaaaagctattcagattttatgatgaacaggataaatcatgtatttaaaaagaacttgccaaagcttttgaaagataacattaagaatgttaaaatatatgcttctatatacaggagaagtaaggtgcagttagaagatgatgacattgaagattatgaggattcgttattttatgagtatgtcctttttgaagtttctttaaatctggaaatgctaagggaggtgatttaa

SEQ ID NO: 114 - DNA, 492 bp, YS40 BACTERIOPHAGE
  misc_feature: YS40 ORF114
gtgcaaaagcttttgtgggattcaatatctgagatagaaatgatgtctgtaaaatgtcatgcctatattatagatgatggcggcgaaatagaaagttttagaatctttgatgagttctcaaaaagaatcgtaaaagacaaaagctttgaaaaactttatgaactccttaaagaaggagaagatttttatttatatcttgtggaggatttaaatggtgatgaaagatttttcattagaacctatttagaaggcatgtatcttttgcaccctgattatgtaaaagaggtaaaagcttgtgtaagttatgcagactatatcgtttctgaaatgaaccaatattttaggaatgttttaaaagaaatcgtttcaaaaaacacaaaaaagattaagatgcttcttggtattgagagaagacacaaaatatctgaaatatatactcaatatgccacatttgaaacagaacttcttcttgcgataaagggggtgttctga

SEQ ID NO: 115 - DNA, 243 bp, YS40 BACTERIOPHAGE
  misc_feature: YS40 ORF115
atgtggactctcaaagaagcgctaggcaaggttgtcaaggaattcttcaagaacatcgatgaatatgatcttttaattaatgacaagtatgttgatgaggaagatttgaataagaagcttaaagatgtatttgaaaagtattttggaatttcagccgaaagcttgataaaaaataatccaaatctaaatgtttgggtttcaaggaattcttggaggcacgaagaacatctaaagattttctaa

SEQ ID NO: 116 - DNA, 477 bp, YS40 BACTERIOPHAGE
  misc_feature: YS40 ORF116
atgacaccttttgcaccaaagaaatatcatttagtattttcagttgtcgcaaataaagaaaaagaagaagtgaagaaaagcatcgcttttctaattgattttagagaagaaaagctttacgatattgaggcgttatttagaaaatactttaaaattctcaaaaactctatctcaaaagaaaatacaaaagcgctaatgaagatttattttgaggctacaggaaaaagaaatataactaagaaagaaagaacattaaagacaacaatgagtaagaaagttatattgaactttgattataagcaagatgaggaatatgtaataaagacttacaagaaactcttaaagtttagcgaatcactttcagaccgcacttttattttggtttttaatattctagcaaacaataaaaacaatagcaaaaatgttaagtatgaattaaacataagaacagaatacgaaaaaagcgaagttttttaa

SEQ ID NO: 117 - DNA, 558 bp, YS40 BACTERIOPHAGE
  misc_feature: YS40 ORF117
atgtgttctgacacacttgacaacaaaaagcaaatcgcatatactcaaaacatgaaaagcacgcaagaaaataaagtaaaaatttattgtcatcctgcctttgaattagtagaagttatcctttcactagaagaaagtaacattaaaaaaataaacaaatcaatcaacttcaaaaccaagtctacaaaacacttgcacacttttcatttgtatagcgatgagatttttagaacctataatcttttcctagatttgtttagtaaattcttgccaacaaaattaattctaagcatctacgaagaaaccttttttgatgtgaactttaaattaaatcagtatgcttcacacatcgaatacaaaactctaggttcaccaaaggaagtaaacgcattacttgaagacttttatcttacaactaaaaaagcacaagatagaatctttgagaataaagatgcaaaaagttttaccttttctctaggaactcacttctttactggtgaaaagacaattgaattttcactaaagactaagtttaggagaacttttccttatgtctaa

SEQ ID NO: 118 - DNA, 552 bp, YS40 BACTERIOPHAGE
  misc_feature: YS40 ORF118
atgtcttttgagttaatcaatacctatccttacgaactagataatttttcgttgtgcttttatatacactacaccaataaagataaatcagaagaagaagtaatagaaagcttttttactttggcagcagaggaagaaaggtttaatagaaatgattttaattacaacgaaacaaaaaatactgtttcgctaatgagaacttatgatgtaataaagctgtttaaaagatttttagcgaacaaagcaagaaatcctcaaggcaaaactacattcttcgcattagtagaaggaattgaagacgaaaacaacgatattcgttcagtagaatacaaacagcaaaaagattatattgaagacctaaatttaagcttgtttgtttcaaaaataaacgaaaacctaaacgaatctacaaaacaagcgattgtacgaaatgctagattaatttttgtatacaactatttgatcataaaatcgtctgtagaggagtcaaaacacttctactatatccttaaagcagccttagttcttaaaaaaacaaaagatgtgttctga

SEQ ID NO: 119 - DNA, 567 bp, YS40 BACTERIOPHAGE
  misc_feature: YS40 ORF119
atgaaagaggaaaaaagcaaagcgattgaaattaataactttgttcctttttcgcttaaaagcgcaaaggttgaacatttctctaagttttattcaagaattaatggaggatatgaaaaaattggcgctattgacaactacatagaactaagagaaaatctcatcgactttgatgaagaaagaaacttgtttattctaaaaggagaagaaaaaattcgtgatatgtttgaaaattttgcgaaagactatttctcaaaagataaaaagtttattaaagaaaggttcttttactttgaaatgacatgtgttgcagaatgcgcataccttgacgaacatgagctgataaagatagaaacgccaataaaagaattttctatttcgctatttagcgaaaacctaaacaaatatatcgcacaaggactgcctaaagtcttttacttcgaagctaaagaatttgaagtgatatcttatctttcttacaaatacaaagcaaaagaagctgctataaactcgactaatttgtttaaattaacgctttcttttgaaggaggcaaagatgtcttttga

SEQ ID NO: 120 - DNA, 450 bp, YS40 BACTERIOPHAGE
  misc_feature: YS40 ORF120
atgttaggtttgcctttcgctaaatttgaaagcgctacaatagcgctaagacatcttaacgaagaaaaagactttgaaaattatgaagaatttcttaacgaatttgaaatcattagaagagaactaagagatgactttataatcgcaaaggcaaaaaaagcaggaatattttctgttgttttgtctttctttatgatttatcctgaacctagcaaaaagcaagaaataattcacgactacgaaaagtcttactctttagaagaaatttttaagtttgacataacgaatgaaataagaaacactttatacgacattttgagcaaaaacaaagactttccaaaagctatattttacatgacgatctacgttgagtttttagataatgagctatcaagaaagaagcgttctgtagacatatcagtaagactagtaagagatgagctgttctga

SEQ ID NO: 121 - DNA, 495 bp, YS40 BACTERIOPHAGE
  misc_feature: YS40 ORF121
atgaaacttgacttctttgtgaaagaagaaattccttacaagcttgaaaaagtaaaaataagcttctttgttattgataagacagacgactacagaagaaattttgaagaaaaagtcgttcttaaaagaacaaaaaatgttcttaagaaaaatgaagaaggtttagtctttactaatagcagaaaagcgttgtcttttaaagacaaatttttcaatctcatgcgagaatttgacatcgaaaaatataaggttgagattagatgctattacaaagaaatgtttcctaaagcgtcttataaagaaaacctcgcattaaagaattacgaagaaatcaaagaatttcttgaaaaagcatcttattcaatgcttcgctttggcgctttagtaacaggaagcgaaagcattttcattgatgtagatatgttttttgaaaattctaatttaatatttcatctcagcaaaaacttcttctatatgcacgaagacttgttttga

SEQ ID NO: 122 - DNA, 504 bp, YS40 BACTERIOPHAGE
  misc_feature: YS40 ORF122
gtgttgactagctatgcgaaagtaaatagcgcaaagttgtctttcgctagaaaagacaaagaaacatctgttgagaactatcatctaaacacttcaaacttttctgaatttatcagcgcttttagactgcttcacaaaaggataataaaagaactaaatatctttaagtctttgaaacaaaaagaagaaatagcaaacttgcattttaatgcttatcatttttattctgtttacttttatgctaaagatttgttgtcaggtcaacaagaaagagatttctatcaggttgagcctgttcactacaacattgagaatttcagttcgctaaatattaagtcaaccttgaaaaagattgttaagaaaactatgaaagaaattcctagcttttatgagatttctttttcgttttacttaatctacgagtcaataaaagacaactacgattcagatgtaaaaacaaatctagaaatgacaattcgtatagcgagaaatgatcctttttaa

SEQ ID NO: 123 - DNA, 519 bp, YS40 BACTERIOPHAGE
  misc_feature: YS40 ORF123
atgaagtttgagctttttgtcaaagaagaaatgccttacaaaatcactagagttagcctttatttttacatcgaaaaagaagtcagttatggaattagctattctagaaaattagttcgaaaagaaaaattaatatttacgccaaacaaaaagaaatttagaaaagataaagaagatctgctgtttattaatagcacaaaagctttagaacttagaaaacattttttcaacgccttagaactttttaatattgagcattacgattttaaagtttctgcattttatgaggatccaaatttcaaagtcttcttaaagcaaaatcacaaactaagcaaactagaagaaagcaaagaaattcttaaaagtttttcttcgctagcgataaaagaagcgcttaaactaaaccttaacaatttcattttgacttcaggaaaggatctttatgttgacatagatgaattttcgttagtgttttcttttaacacatccttaaagtacaagtttttaagcgtgttttag

SEQ ID NO: 124 - DNA, 492 bp, YS40 BACTERIOPHAGE
  misc_feature: YS40 ORF124
gtgaggttagccttcacagaattcaaaatcgcttatgtcaaaacagaatattctaagaagataaaaaaagattttgctttagaagaagccaaaagttttcgcaactatgaagaatttttaatggagcttaaaaaccatattaacaaagtaaaagatgaatatctttttgctaaaggaaataatttagagataaatgccatctctttttctgaccttatctatttaccaatagagaaaggactcctcattgatgacttagacctctttggagaatatatctctggagaatcttacaaactagaggaagtttttgaccttgatataacaaacagcattgaaaatctcttatataaagctttaaaaacctgtggatttttttcaaaggcgcaatttaatttgttgatttatgcagcttattttaaaaataaggaaatttacaaaaaacagtccatatcgtgttcagtagaagttgttagcaattttcctttctag

SEQ ID NO: 125 - DNA, 516 bp, YS40 BACTERIOPHAGE
  misc_feature: YS40 ORF125
atgtttgagagctttcttccttactccttagattttatcacaattctctacaaggaggcaaaaaataaaaaagcattaaaacaatcactatacaaagattattttgatttttacaacttttttgaagaagtcaaatactacttagaggaatttaaaaaagacctatcttcaaagtctggactagtaaaagtagactcatcttttgagttctcctacttaatgttctcaccttacaaactagtttctctgccgatttacatttgtaaagaaaacaacataattgcaaactttaatgaaaacgaattcgcttcaatgataaacctatttggaaaatctatcacaataaaagctttaagagaaggaatggtgagcattacttactactttcgctttaatgcaactatcatcaagtttcctacgcttctacagggactactagcaaacaaagaagttgaaagcacagaaaaactcttcgtagtgaagtttgaatacaagaagggtaatgaggttttctag

SEQ ID NO: 126 - DNA, 585 bp, YS40 BACTERIOPHAGE
  misc_feature: YS40 ORF126
atgaagacgaacatcacactagaaaaagcaacaatacactttgatagcggctcttctctggaaataagaaagctatcaaagctggaaataagaaatacatcaaaaagaaccttgatctatgttgaattagatactcgcaaaaaagaattttgtgaagaaaagctgtcttttatcataaagacattcatcaaaaaaactttatctttagacaaagttctaaacgcaaacttctctcttgacctctatttaacgctttctttcgtcaagatttatcacaaacaaataaagaacatgaaagaaaagaccttgagctacattcctgaaatcaaagaagtaaacctaggaagaaacacaacagttgaaaatgtctataacgctttaattaaagaaaaagaaaacttcttgaataatacagaagaagaattaaacgaaaatcttgaaggtgtcgaaatagtagcatcgtttgctagaggaagatacaaatactttgaacttctttatccttatctagagatagtatttctttattctttcgctgttagtgagtatcttccagattcctataatagtgtaccaacctcttga

SEQ ID NO: 127 - DNA, 498 bp, YS40 BACTERIOPHAGE
  misc_feature: YS40 ORF127
atgaaaatcgaagagcttttttcgctgcaaaaacctaaaagtttgaggtttataaaagaatatttgattaacgatagccttctttcaactaggaaaactgagttttttgaagaagaaagaagcgaattctttgatcaagcgtttactaatttctcaaggagttttgagattttaaaaagatactttaattcgccttttttcaaattaggacattttgcagtcgtatctttgctatcaagcgattattatttcgatatgccttacaaagagctaagaaagcgcttaggaaagaagtttattagcttagaagaatttagtgaacaaaagatttttagtctaatgcagcgtgaaattaataaacgctataaatatgctattaaagaagaaaaaacgctcaatgtcttctttttatctttcgcaaagttaaaaactaataaagcgactatttcacaagatttgcgcatagaagtttcttttgagtttaatgtcttattctga

SEQ ID NO: 128 - DNA, 471 bp, YS40 BACTERIOPHAGE
  misc_feature: YS40 ORF128
atgctagcgccttttgaattcgtgaaagtgaagtttgcctttgaatactttaaaaactcaaaagatgaagaaaataatttttcgtttaaaaagatacttaaagaagaaaaagctaaagacttttataagtaccttaaaggtttcatttcaaaatactatcagcaaacaaaaaattctgaaggcgatgttgtgcaagacattttaaaaatacactatacactaatcacaaatacaaaaaacggaacacctaaatttgaagatttgggcgttaaaaatacttctttcgttaataaaaagtcagtcctaaaatttattgaaggaagcataagtcaaatttacaatgaagcgtttaggaacgaaaaagaaattactgcaatagtaatcttcatagaactaggctatctaacaagaaaccttgataaagtctttctagaagtagaagctactctagaaagaaaagaagtcttttag

SEQ ID NO: 129 - DNA, 513 bp, YS40 BACTERIOPHAGE
  misc_feature: YS40 ORF129
atgaaactgaagctcagcgaaatcgcacttttttacttacaggagtcttttgctgcgtcaaaaggacttttaaaagcagcagaaaataaatacaaatttaccttcaacaaagatcacgaagaaaaagaactagaagaaaaagcaattcaactaatgcttaagtttataaaacaaacaatagaaattgaaaaacttcttgaagtatatgaagacgaagatttctatttagcagtcaatcacaacatgaggaaagaagaccttgatactaaagaaaaacctcttggatacgatataaaaccagaagaagtgcttaagacaatcctcaaagaaagaaacgaattgctaacaaagctgttaaagctaaaaaacatagacaaaaaggatcagattatcatttcaataatcttcactaaaggatcaccttataaggatgatcatgttgcatctctagaggttgtatactactacgacatagtacatcttccagactttagtaatagtgtaccaagttga

SEQ ID NO: 130 - DNA, 507 bp, YS40 BACTERIOPHAGE
  misc_feature: YS40 ORF130
atgcgcattgaagacttgattaaaacacacaaaatatttttcgtagaagtgtcgactgagcacttcgtgaagaaaaaatttgttaaagctagaaagcagaaatattactacaattttccagaagaagaaattgaaagctttaatgctgtatttttgaagcagttaaacatcgtcaaaaggtttgtgttgtcaaagtattttcacaaaggctattttgtagttttttctttaggaagtttttctgaagaatattttgagatttatggcagagaaaaaagtctgctagatagatttcctgaaaatgtttatagaattgaagaatttagcgaagaaaaattttctcgtttagcagaagaatgcttctacaagaaatacagagaagctttgttaaataacgaaaacctatcattagaatctatgttctttgttgcgctaaagacagaaacatctttgatagtaaaaaaattgttcttgaaaatgcactactctttgcacggcgttttttga

SEQ ID NO: 131 - DNA, 429 bp, YS40 BACTERIOPHAGE
  misc_feature: YS40 ORF131
gtgaatgaaaaagacaaacttattaaaacgcttgatagctttgtattgaaatcgctaaagcaggatgtaaagacaatctctatttacatattcgtcaattatagaggctatgttttaggaaaaacaaggttttacttcgaggacataaaaaacattaaacaagacgaaattttgtcagacaagaaaaagaagctttcgttttttctcaaagagaaagcttatgaaatgattgaaagcacagaaaaaacactaaatgaaataagaaaaaatattacaaactttaacgattacacgatatctatagcgccaagaatagtttttgaaataaaacaaagcgaaaaagtttgggttttagaaatagaacacatcttttctttaaagcctccttacacgataaaaataaacgacattgttcttcctagcttttga

SEQ ID NO: 132 - DNA, 537 bp, YS40 BACTERIOPHAGE
  misc_feature: YS40 ORF132
atgcatcctcaggaaggttggctttttgatgagtttgtcttcttgagaagagcgttttctcattgttcatatgttgacagcaataacatagttcgtgattatcaggaagaaattgaaagcttagaaaaactagaggctattgttgaattagcaaaggaaaaattaaaagaatatcataaagaattaaaggagctaattaggtttggattaagaatagaagaaaattttcatttaatcgctatagcaaacttttataagaggtcattaagacacaagtacgatagaaagttaacgcttgacgaagtaatgtctttttctattagagatgcgatagacgaaactatatcgcacttcttgagattttgtgaaattgaagatttatctttgattttgagcttgaagtttccttatttgtctgtttacaagcctatgttaatttctagagcttatgattgttttatgatgattccttatacttcacagctttttcacattagcgctgaatttctcgttgataaaaatttgcttgttttctaa

SEQ ID NO: 133 - DNA, 648 bp, YS40 BACTERIOPHAGE
  misc_feature: YS40 ORF133
gtgtttaaagtgtccttcagcgcctttgacatactaaatttcacgagaggcttcgtctacataagctacctagaaaagaaagcagataagaagttgcctgcaaaaaattcttgcgagtttagcgaagtaaaatttagctacttttttccaaactactgtgatgtgtttgaagaaataaaaaacttctttaatctcgctaaagatagagctaagatgtttaatcgtgaattaaataatataaaacgagtagtaagaaatcaaagcagcaacaacaaattaaatgtcgttacttttgaatctttagaatttagcgctttagccttctatggagaagacaaaggatttgacttgaaaaaagaatacaagctttctgaacttccttccttatctctaaaaaaagagttagaaaaagcaacgatgtattttcttaaaaattttggcttaatgtcaaaaatttcctttcggttcaagtttgattttcgattaaatgttttcgatgaagaaatttcatcagatctttattcgacaaacacaaaacaaatagatactaaaatatttgacaaacacactagagagatagtgaagattttaagcgaaccaaatttgttcgattattctttaatcgctgaaatagaagaatttttttag

SEQ ID NO: 134 - DNA, 570 bp, YS40 BACTERIOPHAGE
  misc_feature: YS40 ORF134
atgaaagaacttatcaaaatcaacaagcaaaccttttttcttaatcaaatcgaaataaactacgagtatttcaatgaaaagaaccttgaagagtttaagtttcattatttagcagaaatatcagcgaaaaataaaaactctttagagaggcttatagaaaaagtattgtcaaaagcaataattaagaaagactttgacctaacgatctcaaaaagcataaatgtctattcactaggaaaaaacaaagaatgcaagctagatttagaaatgtctaagcaaattcaatttgcgaggctagtaatgccaaatgaaaagagaataaaaaatgcgttttatttcttcatcgaggattcaatagaagaactaattgaattctttagtctaagaaaaggaattcctttaactagatttgatcttgttgttagcttatcttctcacgaaagatcaagacacaaaactccctacagttctgtctttaaactgaaatacaaagtcttagatcttgggcagcatttattcaaccttccagacttatacaatagtgtaccaactcctcatcctagcgtttaa

SEQ ID NO: 135 - DNA, 573 bp, YS40 BACTERIOPHAGE
  misc_feature: YS40 ORF135
atgaagaagtctttaaagattacacataagaggaggtctaggatgagaaaagagcttttgttttataacaaagacttaggcgaacaaagatttttaatgaaaaattgtgcagttagcatttatagaagaattgattttagcggccttacaaacgaattattcaaccaaagaaagtttgtttcagaagactttgaagctgcaaaaaaatttctcgctaaaacattcaaagaaatcaaaaaagagaatttagatgtcgtcatcgctaatgatgttgaaattgaagtacaaggacaaaagaaaaagagaataaaaaatttaacaaattttctaggttcaaacaaagaattcattgaagatgtcagcgaagtagacctatcaaaaatgatttctgaaagaatagaaaagataatcgaaaatagcatgtttaaagactgcatatacgaaaacaagaccttgaagatatcttttgcgcaaagaattgtcgcaagctttaaaaataaaacaataccttttcctgaaaagtatgtcatttggtttttagtgtattttgaaagaaaaaaaattgagatctga

SEQ ID NO: 136 - DNA, 423 bp, YS40 BACTERIOPHAGE
  misc_feature: YS40 ORF136
atgaagattgagcgtttacttcgtctaatgaatattttgctagaaagagatgcgaatataccaaaaggcaaagtgaagatgtattgcgttggcgtaaatagaaaagaaattctaattggcgaaaataaatacatcatcagcaagcaagttaaatacgactatcctcacttttcagcgcttcacgctgagttagacttttttcacaaggctagaaaagaaaacttttatcctagtgatatttttgttttcggctacagaaaaaaccttcttaagaatactagaccttgcttgtattgcgcaagcatattaaatgaattagacttcaagcgcattcacttctttgaggaaggaaagattataacgctcactaaagaagagtttgaaagagcgctagatgaagaagtctttaaagattacacataa

SEQ ID NO: 137 - DNA, 660 bp, YS40 ORF137
  misc_feature: YS40 ORF137
atgattgtaaaaggagtcgtagaagacgatgtactgatagttaaaagctacaactattcttacattgaagaggaagaaaagaaagaaaagattagaaagttagacgagtttttagagaacaatgttttaaatgaagaccttgatacaatcattttagaaagaagatttaaagacaaaagaaaaaataaaatactaacaaaaaaagaaggatacaaaactaacatagatgcgtttatcactttaagcgaatacgctaagaacaaacttttgaaaagcaatataaagttgctaacagtagaagctagtaatattgaagcttttaaaataacgagaagaaaaggaaacttttcttcaccaggctggaagcaaattcttactggaatcaataagcctaacgacaaagaaattgaaaaagaaataaacagtcttgtaaataaaggaaaaatcatatacgaagtaaacattgaagatttacttgcgcaaaaagttaaattttacatagacttcattgacgctgtaggaatgctttatgcatacattaaaaaaccagaaatttatgcgttagaagaacaaaatgataacaaagaacctgaagcagacttatacgaagaaattacaaaggagttgtcaaaaaaacttcttggcccaatagtgtcttga

SEQ ID NO: 138 - DNA, 564 bp, YS40 BACTERIOPHAGE
  misc_feature: YS40 ORF138
gtgaacactaaactatcgtttccaattctaagagcaagcgaagcgtttaaaagaaacgctagaagcgtttacttcatcgataggattatttttaacataagtccaatacaaaagaagatactgaaagagttcgcttttgttaagcaatacaagtctataagaaaaatctcagagtttgaaagatatcgctttagcactaactatttagaaactttttctagatatttgtttttttacttcctgcaaaatcctttgcctttgtataaggcgtataaaaaattcattgaagttgaagaagtttttgacttctatcacataaagacaaaggaagactacaagacgataaaaagctacatcgtgaattcttattatctagaagacttaattcttttcgttgcagactttaatgaggacgctaggttcgcttataagtactacaaagaaataaacgcttttgttattagcaatgagataaaaaggaacactaaaaaatttttaatttcattaaacacaaaaagagttttatttcactcaagacactattgggccaagaagtttttttga

SEQ ID NO: 139 - DNA, 1605 bp, YS40 BACTERIOPHAGE
  misc_feature: YS40 ORF139
gtgaagatcatccttcctaaccacattgaaaacaaaaaagttcttcaagcgaagaaaaagtttgacaagatgctttcttttgtagaaaacttcgagaaaagattttctaaagacggcgtaattgtcttttataaagacaacaagtttcttattccgtatccttcgctttatacaaaggctacaaacgctcaaaaggcccaaaaagatttagtctataatttagaaacgaagaggcattataaacttgttgagagttttgaagaaatttcttttgagaaagcgttttttgaatttttgactagtatgaaattttttattcatttgccgcaaatttcacatcttgtaaaagaagcagaaaagaatttttcaattgaggctgttttgaatttgtctaatttagaattttctctcatttacaaaaaccaagaaatttggcgcttgaagtacacaacgctacgagatgtttatctaaagaaaaaggaaaggtttttctttttaattgaggagaaaaagaagggagtagataaaaaagaagaaaaacttaagcagaaaaatgaaatcacattaaaagctagacaaaaaatagaagagtttacatctaaggcgaacttaaagattctatatgacgactacaatataaaatctagagttcctttactttgctcttgtggaaatgtttttgaatattatttaaatgttctacttcaatataacataacttcttgtcctagttgtaggattaagaaaagcacgcttgaagaattttttgaaaacgaagttttaaagaacattcctttttcttatgaatttaggtatagagatgaactagaattagacttttttattccggcaaaaaatctagcaatagaactcaacggagcttattggcactcagaggaaaagaaacctttcaatcacctgcaaaagaaaaagctgctgtttcttgatagaggaattgatgttctgttcttctacgaacctattatttaccaaaaactagatatcgtcaaatcgctaattaaagcgaaactaggactctttgaaagaaaaatatttgctaggaaaacaaaaattgaagaagttgactacactactgcaaacgaattttttgaagaaaatcatatttctggaagcgcaagttttaagtatgcttttggtctgtattatgatgacgaaatcgtagcagcagcttcttttgcaaaacctagatacaacgagaatttctcttgggagctagttaggtttgcctcaaagaaaaacacgattgttattggcgcattaggaaaaatgataaaacacttttcaccaaaaaacttaatctcttatgcagaccttatgtattctagtggcaaggtttacgagaaacttggatttaaaagaattggaataacaaagcctggatacttttatacgaagaacggaagagagctttatcctagagaactttttcaaaaacataaattaaaaggttattacgagcaaaataaatacgaaataaaattctttgacgaaaattcatctgaaagcgaaatcatgaaagctaatggctatatgaaagttcctactgctggaaattttgtttatgttttatttaatcccctataa

SEQ ID NO: 140 - DNA, 552 bp, YS40 BACTERIOPHAGE
  misc_feature: YS40 ORF
atggctttagaagtaggagaagttcaaacaaaaatagagagaagacacaaaagccttcacatagaaagagatttcgcaacagagtctttaagagtaacaattgagtacgctgatggtttttataaagacggtaagtggtatacattagaaacaggtagtataactatagaaggagaacctgtttttatcttgatgcagcttactaataaaaaccttggcgttgatgaatgcactataggtaagatgttagaagctggtatttatggagttattggtgggcaaataaaaactacttttaagctaacgctaatcattaaagaccagcaaggcaatcctgttttcgctagatgttcaatgcaaagacaagaaggagaaaaccttgttgaatacaatgttcaagagggtgaagttattgtaattgaagataaggctagagtaaatatgacattaaaagtgtttgcacctggttataaagaattttcaaagtattatacatatctagtaggagaactcgttgaagaggttgttttggagcctgacacacaggcttga

SEQ ID NO: 141 - DNA, 174 bp, YS40 BACTERIOPHAGE
  misc_feature: YS40 ORF141
atggaaatttactacgagctaaaagataaatttgattttggttatttcattaagataaattttgatgcgagaaatttgttttttgttagcgcaacaaattttgaggaaaaattttgcgaaataaaaaatactctacttcttaatgatgttcaaatttcttataaactcctctaa

SEQ ID NO: 142 - DNA, 3045 bp, YS40 BACTERIOPHAGE
  misc_feature: YS40 ORF142
atgcccgaagacagagatgaaaacacaaaagaaattttaggtgagctaagaaatctaataaaatctaaaggctcaatagaagaacttctaggctcagaaacaggaacattttcgcaaattttatcagcaacactaaaagctgcatctagaaatacacaacagctaattgaaactttacagcaattagaatacagcgtttcatcagttagaagatcaagaggcatatccttgccttatgctagagatttaaatgagctgcaaaattttctttcatcattaaagcagcaaatagaaaacttaaatctcctctctcaagctacaggaagaaattttgttgaagaattatcacaacaacttcctaaagcgcttaatatagcttcagtaaatctaggagaatttagaaaggctgtagaaaaatttgttgctgatttaaaacttcaagatattagaactcaggaaaaatctatacaagacatcgctttaggcgcattttataaaagagctatagaaaagtttcctataactggatactatttagaagcttcaggcgatttagatagagattttctttctaactactttgacctaagacaaacaggtgcgcaaagagcgttctcagaatttagaagtctttttgagctagcgaaatcaacaagaaatcagctttctatatctgaaagaatttttaaaagcgaaagagaaatatttctatctagaacgcttagtgagttttacaacgaatctttaaacgcaataagcatcgctaggtcaaattttattgcgcaattaaacagagtaaatgaaagcttacaaaaagcgcaatcaccagaagaagttgctaggcttcaagaagagagatttaactatctaaaatctcttactcagctatatgcagaagaattaaagattagaaatgaagcgaataaagaactaagagaacttttaaactccttcaaaaatttcagcataagagaattagattatattttttcaacaacaagaaatccttttgtagatatctttgcttcttcgcttagacaaggttttctcataggcacaaatgtatttagcgcagtagataatttattagctagaaactttccttcgttaagcagaactcctataacaactcctcctcctactaatagaactcctacacctgttcctacgaatagacctactactgatagaactcctacaaatttttctagaggtttaagagtaggcgcaacaggtgcgttatttggtataggtttagacgctattctaaatactttagaatctggaaatttaggcgaaggattaagaagaacttttaatatcgaaaatgttttaaaaacaggagcttcttttgcagtaggttcgcttgtaggactttttaatcctatactcggcgtagcagcaggtgtaggaactggtttcttattagataagatttttcctcctaaagagaaagaagaagttaagcaatacatctctgaaaccacaaaaaatttaaatcaattttctatcgcaacaagcgcagcagcagtttctgtaggttctttaggtttagcagcttctttgtctgctaacaatctagtaggaagaaacatactcacatcagcagctagcgtaggtgcaggcgttctagcaggttcagcagcttttcttactgatttttatttgcagagcgtaaacaaagcttatgatactttttatagccgctttacacaaataagacttcttcctcagcaaattcgtgagcaattaggaggagatgtagctaatgtttctaggcaatttatttttggaggaagagtatttcctgctaatcaaggatttacaacagaagaatatctaggtacatcagctaggttgttcgcttctattggcggcttaagtagaagcgtagataatttttcttctgctttattaaacagcgctagagtagctaatctgtttggtacttctattgcagaggctgtaaatcttgtagctacttctagaagagcgttaatagatgaaagaagagctataagaggagcctttttattaggaagtgaatattcagcgtttactaaagcttttagtgaagctttgattagcgcctcaacttctatcgctattaggcaaggtttgaatccttatgattactttaatacttttttaacagcgcaaactgttttgcgaaacttttcaaactatcaaatcgctagattggctgaaagaaatcctgagatagttcaacaaggtgtttctacttttaacgaatttattagaagcggcttaagtggaaatcctatagctttaggtattggtattcgtgcaggtttgacacctttagacttacaaagaggtgctacacctgataatttaattcaggttctaagaagactttctatagaaactggattagttagtcaatttactggaggaagatttacagcgcaagcagaaaactttttaattcctataatgcagcaagtcttaggtcttactaatatttctcctgaggtgtttagaactctaattgcgtctgctattactggcgatattacgagagctaggagaattgtagaaacagagctaggaaaaggaatgccaactacaaagacaccagatgaagtttttgctaggcagttagatgtgttaaattcttctttccaacttcttattgacacttttaaagaaaactctcaagcagttgtaagtctaaataaaaacatcactactttagctgctattacgctaaagcttagtgagcctttaggaaaaggaattattaatttaacttctgaggcgatagaaggaggaactgattttctttacaagtatctcaatttaagcccagagtctaaaaaaataattgacgagatgcgtagaaatattgaaaacattccttcaactaatactgcttctagtgtttctatttcttctactacaacgccttctttacaagtatcttctggagtaagaggaacacatgtgattattgtagaaggcaatatttcttcagttcctttagaggagtttataagaaatttgaacatcattaagaagtag

SEQ ID NO: 143 - DNA, 525 bp, YS40 BACTERIOPHAGE
  misc_feature: YS40 ORF143
atgttttcaagtaatcttatttttccactaaagataagaaactcagaaagaaaaaattctctaggcggccacctagcagaagaaacatataaactcacttttacatttaacgaattagattcaattttgggaaattttcaatataggtttttctatttcgttaacgaattagagagcgaattttatgtttataagataaaaattagtcaatacatagactataaatttcaccaggataaatttgatttgtatctgctaaaaaacataaaatcagaagtagttgttgcaaattttttcggcaaaaataaaattcacttatcttatacaccctatggagaaataattgagtttcaaggtaagagtatagatgagtttatagcgtttaaaggaagttcaatagaaattagagaaaggttatattatcaagacttctatcctttcgcactaaaagtaagtctagttggcgcaatagaaagagtgtttaatttgccagtaatactaaatatcaaagcttatctagcataa

SEQ ID NO: 144 - DNA, 1041 bp, YS40 BACTERIOPHAGE
  misc_feature: YS40 ORF144
atggcgcaaaaggtttactttccaatcaactgctcattatcaccaataaccatagccacaaactatggtgaaccgtatcccaatcaaaaattttttacgcaccaagaacaaaaaattttatttgatataacctctttaatacaatcttacgaattaaactattcttccttcaaacaaaattacccaaatttgccacaagatttgtcatctatagaaaatagaatacttcttttggaattcatagtaaacacaaacccgcaaaaattaaacttcgctagaggaaaaattttatcagacagagcaaagttgattaataaagcaaattttaaatatatttcatttcatccaggcgttgatataaactacggttcaggaaatcaagatttaggacttcctgtatatgcagtaactgacggcattgtgatcaatgctagtagacgattttgtagtgcttcttgcgattgttctggatttgtggcaatagaacatagatgtcaagataaagttttctatgcgctttatggtcatgttgttccagaagtaaacattggaaaaaaagtcaaggcgggagaaagaatagcttcaataggagaatacaaatgcaatagcacatctcatcttcactttgaaactacattaaaaaatatctactcaaattttcccaaaaactatccaagaaacatgtataaagacaaaggcctaaatttagcttatatatcagcaattttgtatgacacactaaaaaatacaacatcattaacgcaatattgtatagattacaagtactacatatcttcttttatggatccaagtgggccctggaattttttcggaaaaaataattcatatgttcaagctacctcggacgaaatcttttatggaggctcttatgcgaagtattatggctacattgagcctttaaattttctaaagagttttggtcaaaataagatatatagcccaatgacacaaagcgtttgtcctagctttagcactagaagaagtttgacaccaatgaaagtttgtttcgctgtgcgataa

SEQ ID NO: 145 - DNA, 951 bp, YS40 BACTERIOPHAGE
  misc_feature: YS40 ORF145
atgcttacagaaaaagtaaaacaagacatatttcaaatattgctagacaaagttgagggtgtattattaccagaaattactcctgaagttcagcagtatttagagagagaaatagaaaggtctgcacaatacatagagaacagattgagaacttctcttacaccagctagatttacaaatcttcctaaaactccaaaagaagcttacgaaaacagagctaaatacaaggatgccataaatgaaaatttctatcaatttcctaaatgggaagatttaacagcggctcaaaaaaatctttatggtaacaatcctagtttttatgttccaggttctcttccttttgagcttttgacaaacgatcaattaaaccaatgggtttcaaatattcgtgttgccccaaataaaattggtgatgcagaaagacaagacggaaaaggcacaccactaattattacttattatgccccaataataactgtttattctttggctttagcgtttacagcgcctcctggatacgaagggaaaaatctcttttttaggttgtataaaccaaatgaattttacttttatagaactggagaaatacacatatttcctgctgtaatggctagaattttatcacaaacaacctctcaagacccactttggggttcgcaatatggtccaatcgcaccaagaattcctcaagtcatagtgattgattacgaatttggctacacagacactccatacgatttactagaagcagtagcgataagagctgcaatgcaattcatcttagccatttctgcaaacctaactgcaggacttaggtctttcggtatagaaggctttaacgcatcctttggaaacaatggtctactttacgaaaatctttataacgaatacaaagaaagattaagagaactattatctccttattacagaccagtaatgactgcttggtaa

SEQ ID NO: 146 - DNA, 261 bp, YS40 BACTERIOPHAGE
  misc_feature: YS40 ORF146
atgctaatagtattaggaacattcatgatttggactgttgctttttatttggctggattaaaagaatggtcaatatttactcttctggttcttacagctctaggtataggagaatacatatctataaagctaactggagttacgctttctgaaagattttatagaaaatcaaaaacaaaacctttggttgctcatgttttggcgctattaatgattttgggcctaatagcaataatttatcatctatacactctaatataa

SEQ ID NO: 147 - DNA, 186 bp, YS40 BACTERIOPHAGE
  misc_feature: YS40 ORF147
gtgcagacacttaaaacatggtatcaaaataagatgtcagtttatagagaaacaaacaaaacaattagagatatacaaaacttgaggacattttactatctaatgaaagcagaacacggagaatatgctataaaggattggaacaccttatttcgaaaatttttaaaagaacaaggacttttgtga

SEQ ID NO: 148 - DNA, 396 bp, YS40 BACTERIOPHAGE
  misc_feature: YS40 ORF148
gtggaggaagttatgaagaaggtaaaagtaaagtttaatccaagaacaactcttagagttgcctacaaaggtcatatttacagacttagcacagaagatgaaggacaatttgttgagttaccaatttcaacaatgggcaagaaggaagcaaaagagcttttagagcatctaaaaaatataggaattttatcctcttacgacacagaagttgttgaagataaggaagaagtcaatgttgtgccaaacgatgatgtcttaatgaaagaaatggataatttaactgaagaacaggatactgaaaaaagagttgaagaggaacaaaatactgaagagcctgcggttgagaaagatacaacagaaaagccaaaaagaaaaagaggaaagaaaagcaaataa

SEQ ID NO: 149 - DNA, 489 bp, YS40 BACTERIOPHAGE
  misc_feature: YS40 ORF149
atgattcaaatatcgctttctcaagcaaacccactaacaacaacattatctggtagtacttttgatgttaataatgtaaattattataatgcaggtgtaataaaagaagaagattcagggagaacaaaaatttttatttatggtgaagatttacaacttgttttaagtccaaagtttatctttctaaaaattagtgcagataaatttttgactcaccaagatttttgctcatcttattctgttgatatttgtgatgggtttactataaattatcttcacggcgctgaacatatttcgttaatttacataaggggaaacattctgaatatattaaggtttttgtttagtttgacgcatccaatattcacactaaacacaacttttaatgaaaactacatacacgcaaaagtggcatttgaagaagggccgataaagataagaacagaaaacattgacgctggtagtccttataaaaaatattgtttttga

SEQ ID NO: 150 - DNA, 1989 bp, YS40 BACTERIOPHAGE
  misc_feature: YS40 ORF150
atgaaaacaaagtttgaaatattaaactttttggaaaacacattcttagcaatacaaaataaaataaatgcgataaagcccggtagcgtaataagatctattttttactctgttgctagcgctatagaagatgtttacacgctcattgatgaggtaaaacaaaattcatacatacatacagctacaggtgaatatttggaccaattaatctatggtttttcaaaattagaaagaataagaggacgaagagcttttggatatgtagttcttagccctattggttttaattttgactccattcaaagcatagacttgttgtctaatatagttttctcaaaatatgatataaaaaataacctagttttttctccccccctctctgccaagtttagtatttcaacagcatatggaatttttaactatcttttactacccccgattaaattatcacttcaatacgaagacgatttttattatgaaataaatcccaacacaggaaagcaaagaatagccgaaatttttgctgaaactataaaagctaactttcttcaaaatccgacaaagaggataaaatacatagttctacctataatttcggaagactttggaaaggaaaagaacttactaccatcatcaatagatgttgttttacaatttggcacagcgaaattttctttaagtaacaatttcgaatacatgcttttaacagatgaaacttctgtagatttagattcagtatcttattatgaagacacacttctcgttttgggcgaatcttcggagattaaaggaggatcttcagaagaaacagacgatgaatacagaaacaggtattggaactttttaaactctttatctaaaggaacaatttcctctattgaaaatgccctttctcaagtaatacagggtgcgaaagtaaaggctattaactctcccaatcctggagtaatagatgtttacatttattcagacactcaaataataagcaattcgcttgttttactttctcaagaaattgtagacgaagttaagcctgctggttctataataaatataaggtttcctaaaactgtttacataaatcttcttgttgacagtaaaaactccattacaaaaaacgacattgaaagtcttagattgaagtttagagattcgttttctgaattaaacattggagattctttttcttacaacaagttttgggaatttttaagggaccaaaactcctcgtttgcaaatccaatgtttggcttatcgttaaatcgtgaactttttgagctttataaaaacaatattaaaaagtttgtttgtttaaactactacgaaagccacacatactttacaacactaccagatttttgtagtggcccatcggagttcacttatagtgattatttgagaattatttcagaaggaaaaatagctttatatatttacgataaaaacaacaattacaagtcttacataacaaacaacaactattcttcacagttaaatatacaagcacctagatatccattaaaaaaagttatttcaagagtgcttggaggagaagaaatcctacatccaagcataagaaacgcaataaaagcaagatgcagaaatatttctgcggacatatgtttaaaagaaatatctagaaatggagatttaaacgcttctgaaatccaaagcagaatagctatacctatttttgatttagacgaggtgtatgcaaactacttcaaaataaagctcatgacactaccagtaaggtcaacaaatgacatagaactttgtgaaaataaaatatcagactattcatactgttccggaaattcttatgaatttaagattttgtatttcttgttaaacgatgttcagtatatagaatataaagacttagacgaaatacaatatgcagatggactaatccgcttacaaacatctagtcaacttaataaagactttatagcaaaatataatgtagctgtaaggagcgaacaatga

SEQ ID NO: 151 - DNA, 1896 bp, YS40 BACTERIOPHAGE
  misc_feature: YS40 ORF151
atgaaaaagtttaggcctagttttgcaaaattcataagattttccgatttgagttcgttaaacgaagaaaacacatcctttttattaaagctaataaagactacttttggcaaaggttattattctgacaaaaacataaaccaggttgtttcctttgatgttcaaactgtaggaataacagaatactttaactttaggataaaaacaaacgaaaccttttcatttttagacgaaaatggattttccgaatctgttggcggaaatttgaatttttatagtgttatttttgacgaaaaaaatcctttaaatggttctaccgaaaaccctattagaataccctatggtttgtttccagtaaatactgttgtagggtacagtaaagaaaacattccaattctttatgtaaatggaacaccacaattcagtagcgatttttcaacatctaaagattcagaatattcaaaaataagcttttctagaattcgtggaggatatatttatccaatacccaaggataaaaacggaaagtttaatacaaaggtggttactggtacaacctttttggactttaaacaggatttttgcgatgatgatttaaattgttatttggattttgggtttttaaatacagtaagcacaaataaatatcttgtactaagagttacatcactaggattggaaacatattatcatatacctaaaaacctaccaatttataacaacaatttatactatttaacttctgcagataaaccaaatgttgttattatcaacttttctgagttcgctttaaacgacaacgatactgtagaattctatttcatagaaaattccccaccatcaataaccacaacaagcgaaacttttataaatcagtacaacataaatgtagacaatatccttgtagacacaaattcgtcagcaattgttttggcactagatgaacaaagcgggacatttataaacctttccaatttaaatgcccagctagcagcaaacatttatacaataaatacttcctacggaaaaatacaagtaaatacttcttatctccctagcccacaaagtaaggtctttgtggtgaactactactatgctcccccaataaagatttacccaaatctagattacctaaataacatatcactctatggtacattcggtagctcaatatcaataaattcaaaaccgaaaacaattcaaatttacgcaaaaaatacacatattcctttttacggaacaacatactatgattcctcttcaaacatagtatctgaaccttatgtatacgatatacttccatacttcttgctttctgagtccacaataaacgattctcaatatacttatataggctctgtaaccgtaaacacagacaacagcacaaacttctctccttctagcaacatacaagttccttaccaaaatcttataaacttagctcaacaaatcaatcctcctcccaaatggaggacattaaatataaataaatcagaattctcatctggtacaaacacaacaatcgttattccaatactgcccgttgtaacagaaaataataaggtaacctcaataaaagcgattcatttaaaacttagagtttatgtatacgatataaacaacggtcaagttgatataccatttgacttaaaactctccttgagagatggtaatactataaaaaacacaataaccttaggatcttctttatctacagcaacctctcaagtgtaccaatatggaaacaataatttgtttagtttaacaagtctaatgtcccataattttaattccaataacgatgtagctccaatttattatttagagttttatgcatcttcaacaaattattttgtcgtttttgattacgcaattatctactttgagatatactaa

SEQ ID NO: 152 - DNA, 2391 bp, YS40 BACTERIOPHAGE
  misc_feature: YS40 ORF152
atggctatagaacacttgaaacaatcagaagtttatacctctgaaacaacaaacgactctgtagacttaactacctttgctcctctaaacatacaagacgacttaaatgtaataagaacgatattaaaaagtattattggtggaacaaattttaaagatattgcaaacatctatacgctgaaaaacctatatacttggagaaacacacattttacttcttttggattaaaagcaacatctatagatgttagtgttgttgctgatgtagacaatttcagatttgttttgtctaataactcctattggcaaaaactaaaaagttttgctcaacaatatttaaatacgcaaaacaaagttctttggtctggtatagcttatacgactactgaattagataacgctataagaacacttccagttattacttctcttcaaacaaatgttagcagtttacaaacatcagttaactctcttcaaactaatcaatccattttctttcaagaaatttatggagcagtagtttcttcttcctctggaaaccagcctatattaaataggatttctgccttagaatcacaagttagcagtttaagtggcttacttaattcaagcaattacattaatgtaataagccaagaaatttatggctattttcctcctccaggtgcgccattaggggcagattctatacttacaagactaaacacagtagaaaatgacatagattctttgcaaaacaatattagtcaagttccacaaattcaaacagatttacaaaatctacaaaacaaagttggttactttgactacaacacttacaccaatacaataatcagcataaatacaaatataaacttcctagattttgtttctcaatttggagtaaaagaaaaaatattattggactttgaaaaattaaaagtattagatttagataggacgattaaaaatttcattgaatctcaaccagctgcaaatattatttcacttttaaacgcattaaaaagtaacgatatagcagaagttttcattccctataggtatatacatttggtaaatagttctaatataaccttgattgacaaaataaaagaattgttagagtccttaaactactccggtaatttagataagtttaccaaaataacaatccttataattttgaaccgttctactacacaagatgaaagtgtgctctactctcaattacctcaacttatcgccaaacttcttccctatgtagatgtagcccttcaaatagaagaaaattttgatgtttggatagattctacttattcatctacaattactgtaccttttaatgcctttgctaatcaagtggaaaaagctattaaatacatatacgaaaaagaaaaatctgtaaaagtgtttacaaataacccaaaatttgtgctgtcaaggttggggctatacaatttagaaattttttctccggttcaattttattggcctgtaaatattaatggattcaatgtaaaaattgataaagttacattgtttgataacgcatttgatgtaagctacttttgtacacaatttcctcccaatatagtggatagttcaacagataatactgtatgtgatttaattttcagtccatcattccaaggagttacatttgatgtatcttttgacacaagtacaaataattacacttcgccatcagacattgaaaagctttttgctgcagcatcagatagtattaaggtgggagttcttacaaattatacatccaattataaagtgttttctcccatttccatagatctttctgatccttccaatattcaagtttacttttctagtgtaaaatcaggattttttaagctctatagaaaactgaagatatttaataaagatactaacaacttcccaacaacagaaactggtaagaacacttcagtaaacgaattttatgattactacttgttagacacaataaattcttctgctataatctacgaatcggggataaaagatgcattaatttttgctacatctttaggagagaattttgaaatagaactatttattaatggttatgatatttacaatcaatcgtacaactacatattaaaactttcctatgatgcatctacgggagatttagctggatacgacagttcaaataacgcaataactgggttttctaagaacataggtactagcggtttaggtatttttaaggtgtttcttataaacaacagtacagatttagatatttatttgggaagtatatctgatattacacctccaacatcccccaaatacacaataacaaacatgaattttgatggatcaatcaatattattttgaactttacaggaactacagacttgaaagtaaaagtactgtcaaacgatgtataa

SEQ ID NO: 153 - DNA, 2112 bp, YS40 BACTERIOPHAGE
  misc_feature: YS40 ORF153
atgtataagttaaagataaacaccgaactaggaacttttgaggggatagacacaaaggaagtttatttgccgatttttaacataattgatgaagataatccttatcaggaacttgaacttttaattgaaactactgaaccgcaaagtttagattatatatacccccacacatcgttattaatgtttaatatttctccttccataactttggaaaacgacttctctccattaacattagaagtttcaattcaaaactcagaaaaccttctcgttattaacgacaaaagagcgtttgatgggtatagtgttgttggagtaatcttttcgtctgattacgatttaattgtaaactttgaaaattctgacatcttaagaagaaaatttattcttgttccatataagttatcagcaaatagcgtttactcaataaaaataaaaatctacagaagcacagaagaagctcaagcttcaattataagaaacaaattttacagatatgaaaaaattatatacgacttttcaaaatcctactctggtgctctggaaacaataaataatatatctctagcagagttttacgaaacctacggaagagaaagaagttttttggtgataaacaaagaagccctgcttaataaagttttttatcccaaagactttttatatacgcatctagcaacaaactctaccattgttcttgatgacggctctttagattcgtttattcttaactacgaaaaaaattttttatccgattacagaattgaggcaagcgctgcctcaaaatttccttatctaaaattctccaacacgataaacacattagtaggaacctatgaaaatatatcaatcaacacagaaaactatctaaaagaagtttctaaccttgtttttgcggaaattttaaaaccttatattctcaactctggatttttcccaataaatactgaacataacataaaattattagcaagtttcttggaaagaattcttcaacaacttattacattaactaccgttaaagaaacaatagtaaactatacgattttttctggagatttgttgccgcaagaaaaacaaaaactttggttttctattttatatcttgagtcggttttctttatagacttcgtaaacagccttttgcataaaggatatataagccaaataccaacagaggttttgtctttaaaggatgttctagattcttattttcatacctttttagatttttcttactttttgaataacctaagcctaatagaaaaagatactttagatacagattttataatgtctttgttctttattgtttatttgtatcatttatcaggaaaaaatatatttttctctggctcatcgttagttaaatatttggaaattttttacgattattatatgaaagtttcagaatcattgttttttgtaataacaaatctaaatccaaatagattcataaacgactcattagtttccataaaaacagtatcaaacaaagctttgttgttgtctttatattcgtctttgtttggtagagaagaattctatatagacctactaaatagactaagcgcctatggtgcaagcaatataaatatcgaagaaaacacagaatactatacaatttcaatagaactttcttctttatcctctggaacaagacagaccttgtatggctcaaataccgttgttttatatccaggaatcggaaatgacgaaaatttttatggtaaaaatgttgttttagcaaacccatctctcaacgaatattacatattcttgcccttctctattttttctacagaaataaaaggaaacaaactgattttaaaggttagtaaaagaaactacatagttaatgaagaacaaaaattagttccaaggtttattacactagatctttctgcaaatatgataaatttatacacaaaaataaatgcgctaattatagataacaactactttatttctagtggaggaaataatcaaattgaggctatcgcaaatacatttgtttatgaatatttgaaattcttactttctggaaatttaaacaacaaagtttctcaaaacacaacaaatataaaaattattttgacaaacaccttttga

SEQ ID NO: 154 - DNA, 900 bp, YS40 BACTERIOPHAGE
  misc_feature: YS40 ORF154
atggccgaaagaacttttaagctacattatccacaaaatacgctaatagatccgatgggatcaattatcattaacgtaaacacaacagtaaacccgcaattcgaatgggcatatataaattctttaaacggcgttggtccagttgacccctcaaaagaagtaaatatttccttattaggttttaaagatacaacatcctacaaaataaggtacttagaaaacataactagacacaacaacacatcactaaccgtaggttcagcggcttgtttaacaagtccagatgctgtatcttgtcagggttatggtccttatatagttttcggtaagcggtattatatagattttatcgttttgtattttgtaaacagaagcgtagtaccgcaagggggcaatgaagccgattattatttatctagcccaatattttcagtagatacttctagatgtacaccagattacggaatctttattgcttcaccaaaacccaaaacaccaaccaatagctctgatccatacgtagaagcaacagctggtactggtgatagaaaaatgtttactacactaccttcttatactaatgaactaggattttttaatcaaagtataaaatacagaataaccgctttaacaagcacaaattccaatagctatatggatgatgttttgttaactccaggaaatattgtgcaagcgttgcaaaatacaattaattatggacaaactagcattgtgtctgtaaaagaagacaatacaccaatagtgttaaggcctcaagctagcaacgctcccttatttaaccctcaatcatatttaccttttatcttgttcagatatataagtgtagatgccccaacaaaaagagtatttctttccttctcttttgctcctaacgcatag

SEQ ID NO: 155 - DNA, 351 bp, YS40 BACTERIOPHAGE
  misc_feature: YS40 ORF155
atgaacaccatctacatagattttataaacagattctttccctctattgattttagaggtgcggtaaacatacttaattctgatttccccgatggaaatttagcggtttacaacataataaatagagagtatccaacaattagagattgtaatagaactccaacaatgatctttcatggaaacaacgagcaaataaattttagaatttcatatggaattgttgtttcttctacaaaagaaataaatgttagtgcagaaggaatgattgatgataagcaactaataaagaagctttttgataatttgcctaactattttgcctctttggaggaacaagaggatgaaaattag

SEQ ID NO: 156 - DNA, 864 bp, YS40 BACTERIOPHAGE
  misc_feature: YS40 ORF156
atgaaaattagcaatatattgattgacaataacaaaacttttcctatagtaatttctccagaagtaaataatgttgaagaaatgagaaataaatactatttttatggactaaataagtattttggtgtgttaccaatttccctttataatgaaaaaggacttacttcactatttgagtttctttcaaacacttcgaaaactttagtagatttagcatattctttggatgaattagaaagcaaatattttaaaagttcaaaaataacaaagggagttattgaagaaacaagaagatttttttatccagtatataacgatgatgtgttttctataaaaccagagctttgggacacgctgtataagtttttatacctgttttacaaatctgttttatatgacggcctaaggatatacatgtcttacactttgacaaaagtaaacaagttcttttcgttttcaatattaaaaacagaatgttctaacagtagtgaagtagaaattaaatccttttttgaaaaaataagaagagccttttataaaactataacaccagatacaaatgatgccttaagaagttatcttctttctttgacatcggacgctttttatatagaagatcctattttgcccaacaaggaaacagacttaaacttttatgacgcttatacaaatgcattgaggggagctacgataaaacagctaaaattagaagaactttaccaaagctttaaccaagagtttgattcgttttatagaggaaagttatctgagattttttcccaaaataatattttttaccaagaacttgttagtattgtaggaggaagtttagcagatgaataccttaagttcctcatctcctaa

SEQ ID NO: 157 - DNA, 801 bp, YS40 BACTERIPOHAGE
  misc_feature: YS40 ORF157
atgaataccttaagttcctcatctcctaagttaatatttagtcctcctataccgggcacttcaaagcctgaagaaaaacaattatcttttagtcctgagcaggtatcaataaaagaaagggcctcgctaaaggaatttaacaacaccaagcttacagctaaagatgttacacaagcctttatactttattgggaaccatattacacaaacggctttattaacattccttaccgtgatgtatatgcgaaatttctaagagaatacggaaacaacatagaaccagaaatagtttacagatatcgtgggctggatgtcctcttaatgaatgaaacagatttcaacaactttgaaaacaatgaaatagctgtgtatttgccaatttcagtaggaagatatttggtattagagaagtttgagcttcctaggacaataataagcgcacaaaaagtttcccaagaaggaaaatttgtttctgtgtttacagaaaactttcccagattttcaatgacactaaaagctgtaatctttggtagcgtgcatataaaggcaaagcagttttttgatatcgtaataaaacatatttcaaacaacaagcccggtagattgattctttatgatatatttggaaatgtaaagatatctcaagctgacatttttctaactttgcagtataacaaaacacctttttatcgccttttacctacaaacgtttcttattcaagaacttcatctgaaaacacatacttaaacatagagattactggtacagtattagaaatacacaatgaaagatatgtttag

SEQ ID NO: 158 - DNA, 4044 bp, YS40 BACTERIPHAGE
  misc_feature: YS40 ORF158
atgaaagatatgtttagttcagaaacttttagaacatatgcagttgtagtaattgaatctctttggccttctagggaagattatagaggagaaggagaaaatatagtttttagtagcggcgctaaaaattatgaattactgaaattttctcctcttcaaagcaaatattctggttattacaaaataataccgttaaatattcttagcgttaacacatcgttttccaaggagggtggaaattttaatgtttcgtttacggctaacaatctactatttctatcagatagagaacttccagaaagattgaaaaaaatactagaatcaacttcaaccagattagaaaacttccctatagaccaatttccctttttagaaaaggttttagttggaaatgacaagttaagggttggttacataacaaactacaatgttatatctcaatttataagagaacttgatactgtttcaatctatttggtgaatataaaagaaagtctttcaaacgatttgcttcctttatatatcgggaaaaaacctggaagaacaaagcaatctgaggcggctcaattagtagacaaaaagaatgaaattctaatttctctcggtatatcaaaaaatgaattggtttttttagatagtgtattgagaagtaaagactttaaaaacatgttattttactacagaaagtttagttatcctctcataattaatagatttttcaaggcgttaaacattcaactgttttctcttaattctgagtttagaaacaaaatttatcaacaagaattgaataattcagaaacagaagacatagaaaatttcataaatttgtttgggcgaacaatttttgattactactttaaagatagaaatgctagggaacagattaagtcttgtattgaatcttattataaagcgaattctgttttaagtaaactaatttcatggactgcctttgaaactttgcgattaaagctaaatgaatctttaggcgtttctccaggactttgcttttctagaaactcaaaggtagaagaagaaagaaaatttaaaacaatactgaacgcaataattattgacaactatttgtatgaatatgctaacaaagtcttaggctccgaaagccaagaagaaatagagtttggtataaactttataaacaacattcttggcaataatttgttcagctttgaagattacttgttaggtatacacagccttttgtctatattcgtaaaagattttaataacacagaatcaaatacggaagaaataaaatttgatgatattcaagaagagctaactattaataattccggtgatgcgcttagccttgtctttaggggacacataacagacatttcaagaagtgtatcaattagtgaaaattcatctgatatagttatttcaataaaaggaagggatcttggttatcctttaaggaaccacgaagtttatgcagattatacttcaataaagtttgcggcacaacctctagaaaactactcggtaaaaattttaactccagtagaagctgtaataaaaataatgaactcttttgccccacaaaaaataaacattctggaactagacagagaaaccttcttaacatccgttatttcttctaggggatttgataccttctataacgctggtacacaaaatatattgataaatcagggaaatgtcgttgcgccgtcttatgatgccaaatttgaagaattgtcaatttttacgccaatacattatgttagtttagattttatacaaatggtaaaaaatgcctttgatactgaaggaattcttcaaaaatttttggcgaatgtgaatgttgatatttctgatgggccattgtttgatgtactaaaaagagttgtatcctcaaactctgcatataggatttatgttgatcacctaggttatttaaaaattgagtatgaaccagcacatatatcttttccattttcactaagtcttaatagggaaatagacgactactacacattctccatagaacactcttcaagcgaaagtaatgtaactactttcgtagaagtgataccaaaatcatttggtacgagcagtccaagctctatgggtattgcatcgttgtatgggagaagcatacctccaacaattcaagagctttattcaacagtagaatacatagatagcaacaaagataagtttgtagaatttctcgctgaggcagtaaagttattggatggcagccttaagaatcacattataaaaagagatggcgaatatgaaagggaggtttttaacaaccttacaaagttaaatacctttataaatcaacaaaacgataacgatttgctttccgcccttaggggtcttactaaaaaggaatctgcacctcagcaaatatcttctgtagatatagttaaaacaaccacctataaggaagatttttgtacgggggaaagaatagctaacacctcttattcacttcaagaaactactaaaactatacaaacacaagttgaagtaccaattagtttgtttaaaccagaagttctatcaaatatattcaatgttggaagaatagacgaaaagttcttctatacaaatgtagactacgaatactataaactttttggaaaatcttttgctacattaaacgaatattatgcagacaaaacttctctattaagtggtatatgtacaaaacttaacgctatagcaagaacaaagtttaacctatcctcttctgctagtgtttgctttgaagaaaacttagatttcttggcgagtttaatggttcttggcgcaaaatttgaactttacggaaaaacagataaagaaaacgctactttaggaaatttaattacaaacataattaactccataagaatcaaatcttctgaaaacgtcttcaagattaacttgtcgaaggttgatgagaataaaaatcaaacaataagttatcttaaagacttttacgcatatttcatacccttttcagatatatctaatgttgtaaaaaacgaaaacaatatactcaaaaatctttcaccagacctgtttatatatggtctaaggagaaaaactttttctgacgcttttatatctgtagtggactcattaagaaatgaagaaagaatttctgccaagcgtgctgaaattataaggaagttaaattctaagccaattaacacggcgaaagtttctgttttgggacacaattactttgttggatatactactctactgataaacgaaaaattacctcctttaaatgatgcctttatttctcagaaaactttaaacgacataaacgatgatgctaaatttatcaaagataaggaaaacctaattgtttacatcaacgaaaaggttttaacaagaagattttcagaaatctacaataaattattgccgcaaatacctggttataatcctccttcaaagattgatgaagaaacagtaataaactactttacagaaggtattaagtttgttttaagttttaactatgagtatgtgccttatcaatattgggtacctctttggggagtttatacaaaagaaagcctggaaaacataaacctcggagattatgtaaaaattttgcttactgagcttttgtatcattccccagaaaaacaacaagattcttctgaatactctgacctgttattcaggtttatatcaaacaaccaacaatatatgcacatatatcaagaatatgtaaagccgcaaaattacttagtagcccaaggacatatagaaaatgttgagcactcttggtctttaggaagtgtatacacaacaagtgtcgggcttaactatttacttccagctatttatacatatcttccgctaggaaaaaagaaaataattataggatatgtagttatagactctcctctttccaagttcaacgaatctggtcagtataacaagttcttgaaagatgaggagtttttcaagtactttagacttttgaaacgactgtatttaaacttctatgaaaatgaattaaatttcgtaagaaataatgcaatactgagtttgctataa

SEQ ID NO: 159 - DNA, 525 bp, YS40 BACTERIOPHAGE
  misc_feature: YS40 ORF159
atgagagaaaaacacaaaacaatgttgaactttgaagagggcgttcaaagaaatgaattttcttttggcgcttataaattagtcgttaaagttccaactctttttgaggagtctaaaatttttcttaagaggtttgagatttttaaaaagtttttgaacgaggatgaagattttagaaaatattttagtgatgtaactgtgaaggaactaaagataaatgatgaaggcaaaccagaaatagttgatgtttcctttgatcccaaaaaacaagaccacattgaacatgtatttaataattacctaggagaggaatatagggaacttatcaatagattagcctatcttgatgtcgttgtagaaaaaatttatagaggagaggaagagcttagtggaaagttttctgatctgttcttttttcaaccaacaagtggtattcatttaagagagatatttaatctctctgaggaaatcatcaattggatcaactcagtaaacgaggttgacccttttttgtcaaaaaactaa

SEQ ID NO: 160 - DNA, 213 bp, YS40 BACTERIOPHAGE
  misc_feature: YS40 ORF160
ttgaagattctaaaagacaaaagccttgggctccacgaagaaatagcagaaaaaggtttatcaagactagatgaaatattaacagaaattgaaaaactagacataaaagtaaaaacacttttagcagaggtttcttctgacaagtctttacaaagcgagttgggagttggaggagaagaattgaaaactatgattgaagaatttatagcctaa

SEQ ID NO: 161 - DNA, 1068 bp, YS40 BACTERIOPHAGE
  misc_feature: YS40 ORF161
atgaacctgcaaaacatcaaaaaccaggcaataagcgttgacctaaaagtaactgactattgtgattatggttgcactttttgctaccaaaactctaagattactggaaaacatgctgattttgaatacataaaaaaagttctagattttcttaatgaaaatgatgtgttagaggtagtagttggtggaggagaaccaacacaatatccctatatatttgatgtgtttcttgaaacaataaagagaaggttgttcttttctctaaccaccaagaatttgaattttgcacaagacatatttagttacagaaatcttcttaggctttttgaagaagttaaaagcaataaagaaaattacttgcagtactacttaaattcaaatggtgcagaattttcgttttccataggtgttggtttcagtccaacaccaggtaaggagtatagtgaagtaaaaattttgttaaataatgtggaaaacttttacaattttagattgtatccgttttcatctaaaatacaaaagattatacgaaaagaaaagaataaaataaaaaattacagtctgctagatttgcttgatttagatagtccagcatttgacataaagtttagaataaaagttcacttaattgaaggaatacacttcaaagatcaacaaagtctgaaaaagatcataagaaaggtatatgaggccaaatacaaaagttttaaatttttatccgaagaatttcctagtatttcaagtattgatttaggaattcttcttttgggctataaaagggttggtaggggaaaatatttaaatgttgattcagatgtagcttctattgctaaatatcttgatagaataaagtttctatattcacccaggaacaataaagatgaaactaaaaacgaaatagttaccatatcagttgatagtgtatttgtagaaaaattgaaagaacacatagaaatatacgaagagtctgtcgtaaaggatggttttagaaccttttatatagacgctgtaaataaaaagttcgccaaaaattcttttacagaagaattgtttgatcttaggctataa

SEQ ID NO: 162 - DNA, 954 bp, YS40 BACTERIOPHAGE
  misc_feature: YS40 ORF162
atgagaaggctaaaagaagtaagacttaccagcatgacaaattcctcttccgcttctatgctagttataccttcagataaagattcttcttttgatgatgtgttttatggcgatttgcttttgttcataaaggaagatgagaaatctagtagcttcatcaaaaagctttcaaactacgaaatatttaagaaagaattaaaaggcgcttcctttaattatgtaaatagacttgaagaagatttcgttgtttatttggtcaatgaaaatgaaaagataaggaactttcttctgcttgttttagcagttgaatcatttattgaaatatctgacttttatcacaagtttaaaaaggtagataagaattctgtgaaaaataggatatacaagatacttagaaaaataggagatgttcttcttcctgaaaatgaatttgtaaatatttatttggaggaaatgaagaaagaaggaaatctatttattgccaagtttgccacactaaattttcctctctacatctacgccaactatttgaaaatactgtttaatcctgtactagatagaaaaacatatgattttgacgaagaagcgattttggacaattatagagaattaaagaaaagagtaaaaacactacttctgatatacgaagaggaagaaacttcatttttgggaaaaggtttaacaaatgaagtggcaattactaaattttatggaaattctaagccatacaaaagaaatagaaaagctatttttaaagacattgttatagacagcatcacagaaagacaaaataacctagaaactaatgtttttcatagggaggttaatgccttaggaaaagcgaagaatgttaatttctcaatatcttccagaaaaggaaatgagctagttttatatgatccgcaagaaaacatttatgcgatttacgaactaggaggtggagatctaagatga

SEQ ID NO: 163 - DNA, 633 bp, YS40 BACTERIOPHAGE
  misc_feature: YS40 ORF163
gtgccaggcgttctgaaagacgatagagaaaaattaaaaaaatttttagaggtagcaatagatgcaattaatgaaaaagatgttaaagttaaaaacacaaattttgggttttataagtttagattagatataacttcaagccacattgtttctacaattagcggaaagaaaaatccgtttttagaattttacgaaagctggatttcagcggtaaaattttcaaacaattttgcaaaagaaaagtttaacaatatacaatccacagattttgttttggcggtagatgtactagattatgatttgttttcattgatagcaaaaaattttatagcaattaagtacttaccagaaaaattagatggaggtaaagatgttgagataattactcttcactctttggtatttttaaatttgcttagcttttcgttgataaatgaggtgtttttgaatataaatctctacatttttggcgaatttagcattaaaagtttaaatgttaaaggaatattcattccaataccctctgacgatgttaaacttactaagcgctttgaagatatataccaagtagtagaatttttgaaggctcacaaagcgataactttagatgctgaaaaggtagagcttttctag

SEQ ID NO: 164 - DNA, 546 bp, YS40 BACTERIOPHAGE
  misc_feature: YS40 ORF164
atgacaaacactttaaacttgctagcaaaggaagttgccaatagattgggatttctttacttgaacacaaaagatttacatcacaaacttgaacctgttaaattttttgtttcaagatatttagaagacagagtaatagatgaagaagatatagagttcttaacttctagctacgaagactttccttcatcaaaccttttgagaactgaaagcacttttttaattcaaagcttttctgatttggctggttttgcagaaaacatagagttgtcattgtctaacattgcctcgccctcatctcttataaggatagatgactattcaaaaataataactgttgatgacattgaaataatgttctatgctatagaggattacgagcatgttcttttgccctattttttgtttcttcttcttgtaggtatcaataatgttgaagacgttgtaggtagtaagttagaaccatcttctataaataaagaagactttagaaagttctcaaaagaaacaaaacaaaaaaccaatcttcttgcaaaattattgtga

SEQ ID NO: 165 - DNA, 630 bp, YS40 BACTERIOPHAGE
  misc_feature: YS40 ORF165
atggaaattatagccaaagaatattacaaattgggagagaaccttctttttgttattaggggatttactaatgatgtaagcctaacaatacgaacaataaaatctggaaaatatatttttcgtaacacttttggttggacaacagccgatacacaaaccccaacaacttttcttttttctgatgcattagacgataacggaactaagtattttattatagaaataccaaaggaaagtttattaaaaaacgacatctacaagttatttgttgatgatggtaacacacaaacagaaaagtttgtttttgttggagattctttcacttccccccccaaaataaatgtttacggcaatttatatgatgcacttggtagaccagtttcatctacaacgcttacatttagcgttcttaatcctgttggttactttgataactcaccaattaccagtatgaatgcttctacaacaacagatgaaaatggatacttttcgatactattgaacaaaaattacaactatgtagttgttgttccagaactaaattaccgaaaagttgtaaagctttctgagcttcctgatagtgttacttctgtagaacttgtctttggagaggtaagcaatctatgctaa

SEQ ID NO: 166 - DNA, 390 bp, YS40 BACTERIOPHAGE
  misc_feature: YS40 ORF166
atgctaaaggtgttaaaaattgagttatctaacctgtttacaaaacctttggagggggcaaaaatagaaatttatccctatccaacaaaggtctattatttgcaagacaacagaagaatctatttgcctagcgttattgcttatctaacagcaaaccaatggggaaacgctgaaactcagttagtacccaacgatttgcttagaccttctcccaacttttatgtagtaaaagttatgtataaaggaagaatttactatttcgtagcaaaaataactagtgatatgaatgatattgtttatctacatgatgtagttataaataaggaaaaagaatgtgaaaaccagcaaaaccttccttatagggtgcttggcaataattactatatttaa

SEQ ID NO: 167 - DNA, 1494 bp, YS40 BACTERIOPHAGE
  misc_feature: YS40 ORF167
atggctattgaactacctcaatattcagaaactatagaaaccaaacaatttacaactcagtctgaaattatcagacttccgtacgtatttcatgctattggtattacaaacaaaggaagtgtaccagtaaaggtttacttatcagaatctaatccaaacgcttatgttgttgtagacgctaattcttacaaggaggtttctgcatcctctaactatataaagtttgtttacgatactacaggaacatatgacataacatttgaaatctttagccttttttctgagggatttttagcaccaagatttccaaacctttatgttcaagacataaacggaaattatgttttgtacaatcctctaaatactgacggcttctttacagaaacatactcttatggcgactcattagcagttaatagaccttacaactccgaaacgatgttagaagtagactattggtttaatggtcaaaagttagcttctacagacggcacattttctagcgatttctttacaacattaatacaaaactttataacctctgtatttccctattctgatgtaaatgatgttttgcgccctctcccaggtggacaaactacagatatcataaacaaaatcaaagattattggcagcaaatagatagaccaattaacgatgaatatgtaggcggttgggcaaaacttgagtcctttgacagaaaacttagtttacttagctttgttttaagctctggtgataagatagagtttccgtttactgtattgggttttaatatagaaaaccttgatgactcaaatagcgctactgtaaggatttacaacgacagctcttacacagaccaaaccatttctccttcttctagttatattggaaaccttgaaccagcaacaaagtttgaggtattaagtggattagtaagaatttctttcagcggttacgctgttaaaatgctttcttttgctccaataattaatatttactaccctattggatttgatgaaactagcggtttaccaaacgatgcctatacactcttccaaaacaggctaacagaatttgaaacttggctaacaaactctaaattttctggtagagttttcaaatataagtgggtacagcatgtaccagaaacaagaaatgtttgggatgccgactattatcaaataacttttgccgattctaaaattcctcttccctttgatgagttaaggctttacttaagagattggtatacatattctcaacttgaaaaacaaataatttctagagtttgggacgaaaattattctgttttggggcaatatggaaaacctaaagatataaatgaagttattaataacgacaacattatttatgttttgggacaaaatttctctggatatgctgctggtacggcaagaagagatgcaaaaagagaatttttaagaagagtgttgcttgaaggctatctaggaaactacgccggaactttaacttattccataaattaa

SEQ ID NO: 168 - DNA, 1086 bp, YS40 BACTERIOPHAGE
  misc_feature: YS40 ORF168
atgcctaaggaatatgaacattgcaaagaaagttatctaaaaaagggagtttcagaaaaaaaggcgaaagcaatttgcgctggaatgtattataatagacatggtataacagtaaacgaagccaaaaagagggggttaaaaagcatgaaacttttatatttaggtagaacaaaagaggctctaaaatcattaattttagctaatggagaagaactaaaagactttgttatcgtaaaagtgcccgattctctaaggaagaatgaagtagttaaatctatctatccaaaggtagaaaatgaagagtatgtttatgtggctgttcataagtctgttgttgatgtaactgttgaaggaaaggttgttctttctcttgaaaagctcctttctaaagttgaagaaaacgaaacttctaagcaaaacgaggacaatgttgctcatgtggaagctggtaaggaagttgttaaggaaggaaagctagaaaaaagctctggagaaagcgggcaaactggttctctaaaagaagaaattcgtgaagaggtcgtagtaactaaatctaagcacagcgtagaagaagatgatagtaatgttccttttctagatgatgttattgaaaaactaaagagaaagaatattctcacaaaagaagaaagggctgcgctagttaacgctcttagagtcattgaggaatttatgcaggacaagcttactgacgaacaaaagaccaatctttctactgcaattagcgatgtagatatggcgctttcagaagaaagtaataagagtgaagacaaatcagaagacaaagcattagaagctacctctgctacagtagaggcactagaaaaagaggatcttgaatctgaggttgttcctgtaacaaaaagcctttctgaacacgaaaagtttttgagcttaaagtctattgatggcacaaatcctgtttttgaaagcaagcatgatgctaatagatatgctagattgtacaaccttcacaatgaagataagaaggctgtcgtacttccagcttcaatgaacgataagaggaagtacggtactaaagctaaatatgtagttgtttttgaatga

SEQ ID NO: 169 - DNA, 570 bp, YS40 BACTERIOPHAGE
  misc_feature: YS40 ORF169
atgatacaatttaaaaaaactaagcagggcctgttttggtcctgctttctttgttataatagcaatatgactgagcaagaacttaatcaagttatttttaaaaaatttgaagaactaagggatgctataaaaaccgaattttacaactctatagatgaaaaatatgtagagcaaataagaacaaacaaaactttttataattcagaagaattagctaatcttgtcagcacaaaactaaaaaatcaatttgaacaaatttttcaggaagcagaaaacgaattcaagtcttttaaattctatctatttgatgatgcggttgggagagaggacttgaattttaatcctgggctttgtaaaacatctaaagttcatataattttagaaggccaatttaaaacagtagattgcggctttttagtattagaaaactctcaaatccttaacaaatttattgaatctttcataagaagaacctttggcgcactaatcgtaagaaggctaaatgaaatttctgcaaaaatagtggaagaaaatgctataatatctaccgatagttatgattcagcttaa

SEQ ID NO: 170 - DNA, 264 bp, YS40 BACTERIOPHAGE
  misc_feature: YS40 ORF170
atgattcagcttaagttactagatcaagactttgaagtagaggtagacgacagatatctaattctttactcaaatgacgatttttgctcttctccaaaaacaatgagatttgttggatttgaaatttacacatttgacaatgttttttgcggaataaagattagattagaaaaattagatgacagaaagctagaatcaataattcttgttgatgaatataattacgaaatagatttatcttcatttgtttccagttacatttag

SEQ ID NO: 171 - DNA, 152372 bp, YS40 BACTERIOPHAGE
  misc_feature: The Entire YS40 Genome
ttaaatttaccccttcaaaaattcctcaatcttttttattctatcttctaactcatcaattttctttttcttttcttttaaggcttcaactaagagtgctacaacagcgttgtagtttaactgtaaataaccattctgatctacatcaactgcctcaggtaggacattctgaacctcctgagcaatcaacccggccaaacgcttacctctcatttcaaaggtataaccggtgatttgcccgagtttttctaaagcgtcttgaattggttcaaggttggtcttaaacctagcatcggaggtttggtacagagtgccttgtatccacacatctccacttactattaggattccattaacatatgtagttgtaagtgatgttgttcctaaaacattcaagtttccttgcaattgtgtatttccgttaactaataagtccttatggactgtcaaataatcttcaataacaaactggtctgcacaaaatgtaaacttttttacgccgtttttgttgttttctactttaaaacatccgctggaagagtcagctaaaatgtcaacataagtagagttttctacatgtgtgttaaaaattgatttataagaatctgaagataacgtaatgtaggaaaaagaactttgtcttttggtaccaaaaactactgattgacctccacttaaggcatttttataagtaaaatctgttcttttcaaggaaccatctttatttcctcttaaaaatagtccaaacgagtaatcctctaagccggaagaggatttttgtaaatttttaaagccctccttttccgcattcaaaataaggacatgttgatagctttccttataaaactgttcaaataatagtgaaggataggttgagataaaagttctgttaaatccttcacctttttctaacaaaatagatgaaccatcatcgtttcttattcttaattgacttttattttcaaaattaattgctgcaaactgttggtccttgcttgtctttaaaagtaggaaagagtcatttttgcttatcttataaagaccaattttatttcttcctaattttatttctctgaactcttgcgcatagaatatatcctctccaagataaccatctgcttttgtattcaaaatttcatcataagatgttagcgctaaattttcgccaacctcttctttagcttggagaaaaggatatttatctgtatcaacttctgtaaatagggttatttttttgtctaactttagtagcttatcttgtttattttggggcgaatattcatgtgtaaagtcaaatactaaatctggtctaaagggaaagttttcgtctttatttctgtctaataagtagtctttgtaagttgggttttctgataagtgaaaaatatactttcttgaaatttcgtcttcgtttttatcgtttaatatttcccaaaatttttcatcgtagttaaagacatcataacttgacctataaaggtcataagaaggaaattcaacaaatcttttgtaaagtgcatctggttctttttttaggtaagaaccagtataggttagatttataagagaagagttgtaagattgaccaaaagattttttatcagaaaattgaggtattgttggcttaaatataaacctttcgttttctctgttttcttcatcaaattttttatttaattttgcaacaaaaatagaatatccagcgcttaaacgctttacaagtatgtaatctccaggttcaactggtatttcgtttctaaaaggatgaagtatcgttattatttgttgtggtaagtctgttcttacacctaatagataaaaatctggttttccttcctctgcttctttataatcaacaaccctagtaactaatactggaaaatagtcttcacttgaagtatatagcgataaagtttctttcgtgaagtctataagcatatttagttaaaggtttcctcggaagtaactactgaatttcctatcaaggagaaaggagttactgctactctaggagcataaattattatagcgtctttatttttgattttcactttttttatgtttgcggcaagattttctccgttattcatatcaacaatataaatatcgctagtaccttgttttgcttttacgccaataccgtaaatatacggtttgtaaattttgtagcttgtagctttttcatcaaaagacaaaagcacatcgtgtattgttctataaagctcttttggggaatttagttgtggaactacatacttatctcttgcttttaattcataaattataggaattacttctttcgaagatttgtcataaacaaacattcttacatctccgatacctccatactcctcttttatgtaaagaatgtatctattagtaccaacttcatatgctttatatcttatttcttgtatttctagtcttctttttaccgatgaagttgagtattctttgttattgtatctgtcattcaagtaggtttctaaaatttttaaaaatatcttttcaaatgctcctcctttgtatttcatccatacactaatatttgaagatatttgacttaactctttaatcattagcgaagaagtgatgtacaaagaatgaagatctttataaaaattctttattaaaccactatttagcagcgtatttataatatctctatccgttaacgacccatcaaagaacttatttgctaaagtaaagctcaactcagctattctataaagatgatcaaagtcatccaaatcatttggcgctaaattatttgcctcagaaaaaatttcatttggacttttttctcttactgtaaagtcaaaaactggaccacttttatttctgttatattcataatcaattagagaaagataccttagggatgctgaaagtaaatttcttcgcaaaatgtaaaaactattaggtccagcttgaaaaatttcttcatctattaacctttgttcatctttaggaaataggagctttaatccaaataaatccttaattaatttatctacttcctgaaaggagtgttcgccttcaaaaaccttgggatataattgcttcaaaaacaggaaatctaaaaaagacaatccaagcgtgggcggtcttgatatcatatcgtataatactgaatttatgctctcttttactagagttttgaagtctaaagaatctacaatgtttgatatttttttggctctttttcctgtttttttcaaaaatcttttgtattcctcaattttatcaatttcgtccgcatcaaaaaccaaaaaatgtgtaactacaaacgaagattcaaagccgtgagaaaacggcatgtaaacaaaagtcttttctatacttttcttttctacttcctcatctgaatatatccttgcttttattgaaaactcatcagatattttgtcgtaatttaagactctcaatataggtaaaaaatagttttgttgacccttatacttataatcagttttcggaacatataaattctcctttcctggaaagtatttggacacctcaacaacaacataggttccattttctagttcatagttaaaaactccgtttccagcatatacagaactcttaattttttcctttattcccaaatcctttttaaccaagttaaacaaaaattcgggataagaaacaaaaaacgcaaacattcctttgtcaacaaactcttcgtgtatgggaacagatataaaattaccttttctagaatttagaattttttgaatttggcttagtaaaagttccattggtaattgatatttcttctttttttgcaaatccttttcttttaagaattgttcagaaatttcgtataactcataaaacattgataacgatcttcttgaaaattcaaattgtcttagcgcatcatatatgtcgtataaaaactttcctacgaaaaaaccttcagatttccgttttcctaaagcatctttaaatcgcttatatatagaattgccgtttattatttgttttgataaatttgacaagcttacaccagaagacaaagatttccttaaatctccttttagagaaacaagtgataaaagtgtttttaaataatctgtcagatcttgagctgaatttaaaaactttacttcttctttgctcaaatttgtctttcctctaatttctaaaactgctttctctacaagctctctaaggttttcagtcgaatgttctaaaaattctatacttgattcccataaatcttctatatctacttcatcactactctctagaaatgaagaaagctttaacgctgattgaattgtttttatctcgcttttaagttgagtatttatttgtctttttgtttgtgtttttttcgtgttttccttaacggaactctgttgcggcattggagaaagcaagacgttttgatttgtaaaagaaacatctactaattcccaatcttcatctaacaaaacacctttccaaccgggagaagattctgggggaaaaaacattcctgtaggcttatgaagcaaaatatagcctggtggaacctttttaaccacctgttttccatcttctctgatttttataaactcattagagttctccatactttttcttaataaactcttttacataatctcttgcttcttgcctttctagttgttcacctagaagagatttaaaaacaaagctcttcataaatatattatcaacttcttcagatatactttctactaagttcatataatcatcaaagttttctgctacaaaaacaaagtttatatttttgttttggccgaaagaagaaattaatgagccgatagtaccaagcaaacgaattttttcaaattcgcaatcaatgctaattgtttcgataaagtatgacagcgatgcgatcacttctctgattttgtcgtagctaaaactcaataaccaatgaaccatatgattggtttcaaaattttctacttgcacacccaatagcatttctaaaacgcctttgcaagaggcaactaagctattaattttttctacaccaagttcttctatcaaagaaaatagaaggaacaatgagtgaggtgtagctttgtagctgtcaacaagactaaatagttctccttctacgaaaaacttcttctttagcaaattaacgagattttctaactcttcatatggagctactaataatttttctacatcatccaaataatcaaagctgtcatagtttttcttctttattgaatcgccaagttgagaatattttaataggtcaatatctttaaaattcttaaactccaaccaatcaaaaacaatctctagcttggttggctttttcgtagtacaacttaatagttcatctaatctcgtaaatttttctacagaatttatcggagaacaaactgaaaggtttgcaaaaacatatcttttgtctttatctgaataaaactcaactttaggcacagtatgacaatcaaagtgtgttgctattaatccaaaagtttctctatggtctaatgaatttttccccaatagcctaagaacaatgtctttttctgttgcgtagattgtttcatcttcaactagagaattatacttaaaactcaaaatgtcttttgcgcttacttcaacatctgtttggctgttattttctataaactcacttacagagtccaaacctacagatttcaaaactcttgtaagaagttctttttcttttagataattatcatcgttagtaagttctattacctgtttgagattttccgaggcgttagcgtctaaagatttaaataaatttctaagaacttctaatccagcatttttttgctcagagggaacactactattttcgtctactcctcctccttgttgggaaggctgatctgccattgcctgtgcaagctgaggaggcatagcgccttgttgcgctttagcctgttcgtcctcaggcttccaatttcttaaaccagagaaaggtacatctccccaaggtacaggttctaagcccttttccattctcgcctcatttattgacctgaatcctgtatttaattgtccttgaattatattccaccaatctctttctttttctaaatcgtcttctttaaaccacaattttatgtctttttcatttctaaactcactaactacctcatcaaaacccttagaaatagtagccataagaggttctagaccctttgctttggttagtgaagccattacttctgctgttgctttatttgaaccttcaagaataccaacatcttgaggagatacttgatatacagcgcaaatttttctagcaacaaattctgcaagttctttaaactgcatatctcttctttttcctttaaagtctatccaggtaaattttccaccagagagaataggaacttgcgtataatcgcccatcattattgcttgaagctgtctttgtatagattctagctgttctctagatagttgtggataaatatctccttctttataagaaggaggttcaatagctaaaataccttctggtatgcttcctccctttctgtaatagtctaggtttcctttgtctataaaaatatctgaaagtattactttataaagaatttcaattggcggcaaggagtagccatacatgtaaacatctggtgtaaggttttgtctaaataaaacaacatctcgcttatcaaagtgggcaacaatagctccatctacttcttggacatagccaaccacaattcctgtgtcttcacttaaaatcggttttattgtcgtgccatctactggtgttatagcaactaaattgccctgttcatccctaatttttactattgctcctctatctattgtcaagatatcatctaacaccattccaagaaggtattcccaactatttacctctgaaaaataaggatctggtctttctaaaaattccaatatatttctagcatctcttattctctttaaataatagttcacatgtttatcctctatatgtttgtatatttccagctctttactagtaagtttttttggtatttcactaactttaataagcttgctatcgttgttaaaacgatactttctgaaaagatttattagaaagaaagcatctggatcactaattgaaggaagatacatcaaactttctggtgagtattcaaacaggttaaacttaaaaggaggttcttgatgattataaggaactatgattttttccttttttaatacctgattttttctaaaaataataatcgaataaactactgtgctattccaagctagcgctctatatgtcagtaaaggcttaaatcctggataatcttttcccctagaaagagaatctacattggcctttatatttgaactaaaactcggcctcttttgctcgtttgcatttactataaatcccttgatgatgttttccaagcttttcatatcaatctcccaaaatagctatcccagatatgtgtataaaggggatctcgtttctaaaaacctcattaatatattttacgaaaagtttatcttctagtatcctttttacttgtggattgctcactttgttttttactgaaacaaaaatttccatagtattacttgagtttactatataatcttcattaaaaattccattttccatatccaagatatttatctctacatctgaaaacttagaaacgcaaaactcgtcatcgtaatatttttctaagcaaagttttaaaaaggcatcaaaaagtatataatttaccaacccacttcttgtcgtgtttttcttaaaaaacccttcgtagtaataaggagaagacacttttagattaaacgattttgaaatatctgataacctagtagaatcaaaatagtttcctgagtttgtgtagttattaaattttatcttttctttaaacttagaaaagttttcttctttgacaaaaaagacccttgacctaagaagaatataaagcgaaatgtacaaagagtatatagattgggcaaataaaaaaagtaattttctaagatttttaaatctctttgtccttgaataatctgattcttgatagttgttaaaagttatttctactttaagcttttccactaaaactattttaacagaacaaaccgtttaaatctcgtcgcttatctttatcttatacattccccgcttgtttatccctctttttagtatttttatgtcatctataacaaagttttctttcaaaatcgacaaagctttgatatgattaaagctatttccgcaagtgtaaatatctaaagaaacatatccttcatctatccagtagtgccaagaaaggtgagattcgcccaaaagacaaactccagaagaagcttcgttaggaaaaacaaagtgtttacaatctattattgttgcgtttgattcttttagcaaatcgtagaagaaaaatttcaatttatcgcccctaaaatctttaccttccataactcttaactctaatatgacatgagtgccctcagtaggaacctgcttatttttcacaaaaaaattatacaattttgttaaaaaaattaacccccacacaggtttgtgtggaggttaatttgtaaacttactttactctacagtatttactggttcaaattcaatttccttgagaagctttctagactccttaagaagcttttcaagcgctagagtattcttgcgaagcctttgccttgctaccttcttgcccttctgggtaagctctctaaagttcttctgcagggcaataaactcctcttctacagccaccatcagatcgtccagcctgtctactccttttaccctgtatcgcatgtttcttcctccctaaatcaggaactcatctccgagttcctttgcttcatcattatacaccaaattctcttcatctgagttgctatttactgttttgccttcagcaccagcaagttctacgaaaacataatcagcgattatagaaaacttatttttgtaattttccaatatgccgtcaatcattagtttctttccaatcatacttttatctacatgtattcctctgctgttgaaaagcgttacaaaaattctagtagacttttttcttgtagtttcaattagtagcctcttacgattgccattatcataaactccaaccaaagtaccagaaattgtagcaaaccttttatctttttggggatatgaagaaaatgaaattgatgttgcataaatatagtcctccttttcaccatttgacaacatcccgtacactaaagcgtacaaagaagctttttgattcaagctacttacaaccttttccttaactatagaaaagtccattggaactaatactggaaaaacatttttctcaacttcaaccataagtacatcatttctagcatctacaagctttccagctaacaaagtgctgtttttgtcgtacttaaacttaaatgtgtccactagaaccaaaccccctttctcccctttctgtgttggcatatttatttaaaaattcttcctcacttacttcaaccatttcaaaaaatgacctactatcatatccaattaacaccaactgtgccacagcaacatcattcctaatgttaaatattcctgtactataaaattttagtccaacttttattggaccacgatatgagctatcaatcaccccatgaaacacatctactccagacttgaagaaattgccgctcctaccaaatataactccgtgaatacctttgggtaatgcaattttatatggagtagtaacaaagacataagaaccaggataaagaacaagaagacttacctcatcattagaaacctttttaatacgaattaatttttcgtctttaatttcaacagttgaaacatcaggaaacaaatcaataccagcattttccaaaatgttaaccttcaaatccttgaccgatttatttagcttaatgtaggggattttggtcatctccacctccgttaccagcatagcaccaaacggctcatatgtcaagccccttaagcttcctcagcctctttaccttgctttcaagctcctctagcttggtttcacttaattcttctttattaattatcgtaaagtttgtaaagtctatgtgtttaacatcatcttcagaaagctttacagcgttaagttggtgtcttaaaaaggctcgccaagtgataggaacaatcttagcaacaaaagaagctattgcctttgcaacttctctagtttctttttgcgtgtgatcgtctagtctaagtcttaaaaagttcattaggtttctcaagtcctgcttgaaaaagaacattgtgtatgtacctaaaggcaaaaggcttctcgcaacttctctagccaaaccctcatctaacatttcttggtagtttttgtaaacatatttataagtttcatcaaaaacttttattgtcttaagtagtttttctgtattgttttcttcagcggaggattgtttgtttgctttggagtttgttctaaattcactaggaacataaaactcatcttcaagttttacatatctaccacttatttcattaaagctagctgttctgtgtctaaaaatttgccttgctacaaagataggtactttaaggtgaaaaacaaattctaccatttcaaagggcgacatatgttcattgcgcattaggtaatctattaacgcttcatccttagactttcctttaaagttgtaagaaactctagcagcctgaacaattagctgatcgcttcccatgtagtcaacaagacgaacaaaacccttatcaagcacctttatttctcttgggaactctccaaaaccctcctgcatctctctcctcccaaagccgttgctactagtgtatcagaaaatctttctttcgtttgaagcacttatggaaattttatgttttattgagtagtgcaacatttctaaaatctcataaagacctgaaaggtaatttaaataagccttatataaggcgatttttttctttaagtttctatattcttcgtttatctcaactagcttttctacttcagtatttgtaattttcttttctgatgataaagaagtctttgtttgtataaaaagattggcatacatagattcttcttcgttatcttttattgccaaaattccttcaattagagtctttttgtaatttatatgctctaaccaagaaagaacagaaaataacaactcatttagttcttcgggagaaaaatcgtttataacagaaaagtccaaaaaatatgtttcttcattaaatggtatctttaatgtttgtgcaacatcttttatgctgattttataaacatcaggaatttccttaaaaattgttttgtaagggctttctagtcttctttggctatccatactagtagcctatcaaaaagctttctgtttgtcaatacgtcctctagggatacgctctttaaaatttcgtctgcaattttttctcttctaaggtcttcttttttattaaagaattctttgattaagttgaaaacaagtaaataaactatattttcgttgccaaaccattttttatatgtcattgaaaatccaaaatcgtgtctttctttgtgatgataatcacacaatggtatcgttgagaaatctgtaaatcttttataatttctataacaataggaaaggttttcatgatgagcaattgtttccgtttttgaaaaacttctttggcccaaatagcacgcaatacattcaaaattatgaatatactctaaatattccttttctagttgcccaaaaacatcactattaagatattctctcattgaattacagtaataagaaaaatcactcatcttcctcctcttcctcctcttgagataaacttacagatttaagatttgcaatcttttctctaattttttggtcaacaagctctgcaaagggtttgttattctttatttcctcaattaattgaattgcgccgtgaaatgttttttcttctccagttatttgttctttataccaagcacctgatttcttaacaagcccaagttggctcaatatttctggataatctatagtataacttccatcgtaaaacaacaagaatttagcctttctcattggaggagcgaacttgtttttctcgacttttacatgaacttcatctccaatttctccatcttcgtcttttatcggctttttgcgcatattaagtctaagagaggagaaaaattttaaagctctaccacctggagtggtttctggatttccataaactcctattttttcacgaatttggttaataaacattattgttgctttattttgccttatgataggcattaattttgaaagtgccattgaaagaattctagccctcacaccaaccaggctatctccgtaatcagcgttattttgagatacagtaggcggagctacaattgaatcgtaaacgacatatttaactccttggtttattagaaactctaatttttcgaggctttcttccaaggaatttgcctcaattaaaagcagttcatcaagatttacaccaagagtttcagcccaaatcggatcaaaagagttttcggcatcaataaaagcacatattttattcgctttttgcatttgagaaatagttattaggctaaaagtagttttaccagaagactcttgaccgtatacttcaataatttttcccatcggaattccaccaattcctagcgccaaatctactgtcaatatacctgtagaaactatttcctctttagtttgctttaattcttgacctgtataaatttcttctttactaaacttcttcgcataagaagaaatgatttgctttattttttcggtgttcactcagcacctcccctttttcctaaagcgtactcctttgcttgcctgtaaagcttatcgccgattatcctaaggtttgccaaagcctcttgtatcggtttggagttgcgattctttagttcttcttccgttgcaaatatttgatttttgaaaaaccaaatatacaaaccgatcgaatggtttgcagcagctcttcttattgcatctgtttcggcagacttaggatctgccttaataaattgtttttttaacttttctaaatcattatcaaaaaccaagtaagaagaaccagttcctgtcattgttttacccatgattgtaagttcagcctttgccatatgtccaacaattaaaaatccgcttttatcagtagtttctacagttattggagttatctctgtttttagttcccaatcaccaggaatcattgtgttcaagcgatgaatgatgtatctaacatcaagatatggaactatcaaataagaaccattttttagctttttatctacacggaaaagaattgcttggtctggaaaaggcctcattaactcaattctgatctccttttcagtcatgtcctacctcgctcttatcagattacaacaaactcctgccccttgtcaagacttgccttttttgtcatctgtctttttagtactaccaaacttaatctcttctacattaaaatcaaaagtttttgtatcgtatgaaatgaccgcagtaaaaagtggaccatttctttgttttttaacataaaaattgatatactttcttgttgaaacaatcttttctccgttgtttagcgttatcgaatcaatctcttcctcttttttgttagacattaatgcgattagagaagcggcttgctcaatatctccactttcctttaggtcgtcaatattaggttcttcatttaatcttcttgacaactgagaaatttcaacaattgcaacatccaattcaatagctaatgtttttagtctttgcgcaattgagccaatttctaaatgtctatttccagaaaacttaatgtttgacataagttgtaagtaatcaatgaagacaatttttgtgccattttcatatacacttcttctaataattgactctatgttttctatagatccatcatgagtagaagtaaaataaaggggccaactgttcattttttcaactgttatttgcgccaatttcttttcctcatcggttagttcttttttgaatatttttgacgcaggtatctttgaaatcattgaaataagcctaccaaaaatctgaaattttgtcatttccattgaaatcaaatgaacaggtatattttctaaagcttgcttgtaagcgacatgcagagataaaagcgtttttccgataccaggtcttgcacctataacaacaacatttccaggtacaaaatcataaatataatcgtcaaggttcatatttccaaacctaaagccaaacctataagtgtcttttctgttaagctcattaaaaaattccctagacatgtcttcaacagataaaatatcaacattttttattccgcttctagaaatttcttcctggatttctttaagtttcgagtaaacttctgaagttgataaaaatttttttattgacaattcttttaactgaggagaaatttttctaagaacacttttttcttttagccaattcgcatatttaactgtaagattaagccccttttcatctggagtaatttcatcataaagttcaacatcttctgctttaactagaccaagttgcacaatatcagacaataaagatgcttctggttcttgattatggtttttaacatactcaaaagcttttcctagatacacaaaatcatcttctgaaagaaagataaccttttgtatgaaatctttgtgtttaataaggctaaaaacaatttctaactcaataagataaagattttcgtcaaaaagttcattttggtccatattcgtacctccccgtataggggttcatttgtaaatcccaatcacagggaactttttcgtattgcatagtatagggattatattttacacacatgttctcgtcttggctcattatgtatgacgatttgtattcgttataatctgtttcttctttggcaaaaacatatttatcttcgcttgtatcgtatggattttgaacaatatctaggtcttcttgcgttaaatcatatgtttgcctatagggattcatcttaattacttttctttttcttaccaccatggcgcttctcccttcctaggaggagcttaccacaactttggggtgtctgtcaacaggtagcagaattggtgataaaataattctgctatggctaagaagttttcaaaagaaccaacagtagcaacactaaaaagtgaaataagaaaaatactagattcttattatccatacaacaattttaaagtttctttaaaaactgctaacgacacgctaataataaacattgatgggtgcaactttgacgaagaatttcgaaacatgttaaataggtttgttgattacaaaattaaacacatttacagaaaaaagactataaaagaagaaaacaaggttgttgtaaaaaggataccttacaaagacttttacataaacataaatgatgattgcatcatgttcagctactatcgctatctgtaaaatataaaaggtgatgcagatgtacaactacacacaagaagacatttccttcattgagttaatagaggatgtaaactttcacacagaaaacttctacggtagctttgaggattacctaatacttccaacatttcttgaaaaggaaatctctagaaaacagacaaatataatgcaagaaattattgatattaaaggaagtaaaaggcttacaaaaccagttatctcttcaccaatgagcttttttggtaagaggtttggtgaaagattagtagagagaaactttatttactcaattccacgaattaataagccaatagaggagagaatagaaatttacaaagaactgaaatctaaatataactctacaattatcttttctgttggggcaaaggaagaaattacaaatgagataatagataattcagacatcttattacttgatatagctcacggtgcctctaagcattctgtaaatttcttatactctctaagcagacttggtattaattctggtgtgatggtaggaaatattgggtcaataaatgggatgttgtatttattgtactttgcggcaaaatttggctttaaaaacatttacatcagaagtggaataggatcaggaagcgcttgcacaacaagactcaatactggagttggatttccacaattagacttaatgaaagatttacgaaactttttaaacaacttaaattcttctagactaggaaaatacatagaaaatctttctgacacaaaagtatatcttgtttctgatggaggaataaaaaattatggagatatagcaaaggctttgatattttcagatttagtaatgggcggaaaaatctttgcttcaagagaagtagatacttttaataacgaaaaggtgttttattatggtatggcatcacaatacgcaaaacaaaataaagaaaatgttgaaggtgaaggttttgaaattaaaaatcctccacatttagaagaaatactactaggaatagaagacggattgaggtctgctcttacttacactaactcaacaaacctagaagaatttagaagaaaggccaaactaataagagtaagctcttctacactaaaagaaacatatgttggattttaatcttggtgtttgctctctagggggcttgacaattcttgtcaatttgtttatgctagtcatgctatgactagaccatggacagaagctgaaataaatgaactaattgaattagtaaatgctggtagaaagcgtagagaaattgccaaaatactaggacgaacacctaaatcaatagaatctaagcttagagatattcgtaaaaagagtggagagggcaatgaaaaagttttagcaaataataaaatcaatgaaaacaacaattcacaaaaaaatattgaaaagacgaatagcgaaaaagtcgatgcaattgtcctatatagaaagtttctcaggaaatttttgcagaaagatagcttaccagaaagagagtttgatagcttggcaaatgctgtagaagaatattacataaaaacaaaaaatctaagtggcgcaattcttcttcttgaaacaataatggaatctggtaagtactagcaaaaataaataaataacgacagcctgttttggctgtcgttatttatttttgttctgttacttagaataagcgtcagagtaccaaccaataaggtttccgttgtcgtcataaacgggaatggcaccgtaaggaccagttacataggactttgatgtaggataagccttctgatattctagggttgtgtaatcaaatgtatcaccaaatgttacaggtacgctaatagcttttccattaaagtcaaaaaatacagcgccgttagctgcaccttgatagagtaggaatacatttactgtttgatagattggatttccattattatcgtatgcaacaacaacacgataagaagaaggacggaaccaatctttaagcccttcagaaagtatctttttagccaaatcttcaaaagaagttttaactgtagtatcgctaataagagcagcaactttacttaccgcacctgaaactacattattaaaatctacaagtctttctggtctgccaaatactgtatagtttccattcattggcttgggtagagtgtcgtagaaaggaataatctgatacttatcgcagaaaaccttagaaagaacagaatagaagaagctacccctgtttaaagtattagacataccaaaaaggtgctcatcgttttgtagggctacagaatcatgagtaatatacctagagaaatcaacatcaaggtaaatatcattatctacaaatctaactgttggcgcaccagaaccagcgcaagcagaagctacagtaatccacttattaaatatttctttggggttgttgagatactttaaaaaaagcgtttcatctggaggagtcattcctgctatttcactagccaaatcctcaaagaaggtttcgtaaggtacggcaaaagtattaaaagaagctaacctataatctcctaccctaaagagaagaagtggaactactctagcatcttcataaacctttacaagttcaaaattagaaaggtctggtgttgcatcaaatactggagtagcagtagaaccaccaaaaacaagtctaagctgtctgcctactgtatacctatcatcaagctcagagggagttaatgcctgaaaagacagagtagtgccatccaaaactggaactacttctgtgctaacgccaccgtaaattgcggggtttaaataaattgaaatttcctttccatcagaagctcttacatatttagagtatcttccttctccatttactaagacaaatttagctccaatgtatttaagtgaatacccttgagatgtcatgtttatattacctcctttttagattatatattttcctttttgataaaatcagcattttatgctacaccaactttttgagtaattgaaatcccttctattatcttttccatcaaatcatcatccacatcatccatttctccagaaagtagcaattccattaccgcttcttccacattaaaaacttttatctctgctcctttatttatagcaaaggtcaaaagttcaaaaattttatcatttagaaattgagaagtgtttttataaacttcatttgggttattcttcaccatactaaggacttcgttgccgaaaatcatttctattgttttgtaaacatcttctttttcgttttccgatgcgctcattgactttcctactaactcagccagctttaatgcggcaaaaacaaatttttttatttcctcaaatttagcaaaaaagacttctctatccgctttttctttgaattttactaagcttgttgttgctgtcaacttgctccccctcctttcaaaatcttatcaattccaaaagaatactctttattatactgctctcttgccttttctttgagttcgaaaaattcatttactattacttttatctcttctgtaacaaacttcaattcctccaaaagttccttgtgctcttttattgttttagcttgatttattttatctcttattggttttaggaaaaacaaatctatatattcaaacatatcatcctctgggttgcttttaatgttaattatggaggtatcaaattcatagaaacatgtggatattattttctttttgcataaaaacattatgtgattagaaatcgcaacaatcacagactttatatctgataaggtaacctcttgcataaactcctcactttacattctacgcaattccttgttttttgcggataaatgcctgtagtaagcgatttcctaatcatattaagctgtgaatacaaatattgtttgtactcaagattgtaatcgtgtctatatattttccttttcaggatatctatagaaagaactataaactctacattaaacttttcgttaagtatttctctgtatatgatgtacggtattttttggctttctgagtttcttctattttttatgtctatcaaaacataatttcctctttttgtgagtgcaacaaagtctggtattatttcaaacacaagctctggaaattctcttatttttccttctattttctcctcccaaaccaaaactttctctacttgtatagaatttcttatttgaagcaggaaatcaaaagaatttataaataagttcttttctagctcataaaggttgtctttttcttcaataatatccttttcgtacaacacttttacaaagtcatgttttttgtctaaaagaaatttgaattcatctaaatcttcactagaatcaaactttcccaaaatatctctgtacatatagtaaaaggatgcgtgaaaaaatctacccaagacagcacttctggttggaattttcttaggtatattgttatctttcaaataaaactttgttatctgaaaattccttgggcaaaagacataggattttactaacgatggtgatattttaaaaggtaaattcactttcttctccctcctttctgctttcctcttctactggttctatatttccttcaatctctaatgccctagaaacgcttaaaaagcccaatcttttcgctatagattttaatttttctgacctcttatctgtcgtctgctcagagattaaagaaattaaatctacctcctcaggttcagtttctacgagcaaagaatattcttcctcactaatttcacttctttcttctaagaaaaattcaaccaaagattttacagtcttacttttatacttgtataaaagcaactctctctttagcgtcatgagcttatcgtgatgtccattgacaaggtataacactcccctgagaagctttctttctttgggaggtatataatctatttctacagactctatgtcttttttcgccttagacagacctttatttatcacatagtctacccttctaataagcgcatcaaaaggatattttgacctttcacatattctcagcattaagttataaacatgctcgttttctctatagttgaaaaataatttcatcagcctttttattagttcttctctaattagctccttatttttaatgggcaaatccttgacgatatttctcaggtgagcgtctataaatacatcataaacatcttttgtgtctgctgtttttataaattcctcaaacgatttcttttttaaaatttcgtctatgtcttccttatctttataaaaaacaaacattgtagaaaaagaagggcgattggtttttataattgttttagcaacattgaaataagctgaggttccagcatcatcgttgtcaaatacaaaataaacaacggaaaatgagtttaaaaggttaatttgctctactgtaaaatgtatgcccaacaatgaggcggcaggtatatctctttctattaaagcaagcgcatcatatggtccttctactatgtagatagtgtttaagtttttcttctttgctatctttacagcctcgtttaaaaagggcagatgctcagttttactgaattttgtatttaagtattttggtaagttcatatcatttagcgatctaccaactaaaccaacaattctattaactctggagtagattggaataataattctactcatgtacctaaatttcagttttccctcttcatctctgctaaaaattccaagtgttatcaacctctctatgtctaacttttcctttacttcattcgtaaaatctaacaatctttcattagggacataaaacatcggatactttggttttaatcctaactttttaatattaaaatcgtctacagtttctgtatcgttatacttggacagcaacttaagaaattttgaaaacagatcatgatcatcttcatacttcttttcgccttttgtttttacatcaatgccaaatttttccctaagaatttctccaacttgatttatcggtacatgctccactaaggaaataaactgataaatgtttccgcctgtattgcatccaaaacagtaaaaaacaccaaggttagtgtctatatgaaaagaaggtgtatcttcgggatgtagaggacaaaggccataatactttcctcctcctctatgtctaagatttgtgtacttactagctatttcaaaaaaatccagcctgtgctcaatgcccatggcttcatgctagcatagagaacttagaatgtcaagacacaaaggctttcaggactgcttaagactaaaaaataaatctggtctttctaatatttctttagaaacttccttaggatgtatataacaatcctcacatcttggttcgtaatttaaataaattagtgtttgcttacttggaattattggtaaaggctcgtcatcataaactgcttggttatatttagcccttcttccacaaacagaacaataggattctaattcttcgctgatgtcagcctctttaatgagaaattcaaattgctcgtaaacattagcttcgtaatcaaattttaaacctaagaagtttatcatgacatctttatatttaaagaactttatgatttgctttaactcatattgttcaattagatggaaatgctgaacatgtatgacatacctttcgtcttttttcactttatagaaaacttcgtgaggataataagagtgttgataatacacctttctatttttaaaacgaatttcttttggattagaaataaaagttggccttattagtaaataatttacattatacctttttgataaaatggaagacatgtagaccatttcactttttcctgatcctactgttccccaagaaactccaaagactgacatagttcttatataacacaaaagtcttcagttgtcaatagtcattttaaatttatagaacatagactttacctcatcgtaactaagtccaaaattctcaaaaactttatgttttcctttgaaaaagtcgctagcaatattgtattttattgcttcttctggtgtgatgtaaatatctttatgcgctccttttccaattaccttctttttgagcccaatcttctttataaactcgttttcaaagatagtcttcaatactttgtattgtttatccaaattttcaacaatatcttttgaaccaatgggttgaggatgatttacaagaaaatcatgcataataaagaacgagttagatgtaacaaacctattagactctttagcggaaagaaaaatgtaaacaccagcacttgcaataatagaattagctacaaaaattacatctctttcaaaccccttaattaaattgattactctcatagcttgcatcacatctccgccaaaggttgaaaactgtacaataattggaatcttatttagtccaattccaaactcatctcctttaactacttcaaacttggaaagctcatgagaaacaaaataaatactatcaacaaactcattaacagaaaattcatctatgtctgcactaaagttaatctcaacaaagttatcataaattacactttgaggcctttggttttcagaaccacagttcagaatctttttgtgtagcactttcatcctcctcaaccttaatatacctcaccttgcccccgtctgtcaagagtttgaaatcgggccaacaaattcgtgtaaaagcgcaataagaacattgccaaggtaaggtcttcactactaaaccatttccaaggtcttcaattctatattgataaggacgctcaggtagctcacctttaatacctcttatagcctttctaaagttgtctataacttcttctacaaccttttcagagtacagaattggaacaatcattctgttgcttgtatttttgttgtaaacccaaagataagcttcttttatgcctgtagcatacatgtaagcgttaagttgcttaacataatcttctcttggctcttttttgaattcgttaaaagtcttttcgtttacagtttttatgtctataattacatcacgatcctctaattttagaactccgtctatatgtcctggtattttataatcgtcaacctcaagatatactgtatcttcaacgcttacaagttcattaatctcagaaataagtgcacgctctaattcgtgcaaagcatggcccatttgaaaaatacttacagctcttggagataagtctttaatttctccatgttcttcaaattgatatattacagaataagctattgccctaggacacttaccagaagaagagagtcttaaataatttcctttttcttttgaatactctaaggaaaaattaaagttaacccttcctccattttttgtcatgtagactaagtattcttccactaatctaagaacgtcataaacagattttgctcttggcggcttgttgttttcttccttaaactttaaaacttcttcaacggtcatctgtatcctccctaatgtgctgtttttcaaacatatcatgaatatacatctatcttaaactcaagttctcttttaatgtcaacatcaagaatgttctccattgatttgtccactacaaaaatagcttgtggttcttttttgtttttggtttttcttaaaattctgccaacaagctgaacagaagaaactttgccgtaaattaaaacagaagttatcagcgtatctaaagaaggataatcaaatcctttagacacaacgccatatgttgctataattagattttcattttcatcaagagtatcctttctttttgatgtaagtattttcgcatttttaaaaaccttagcaaggtagtctatggtttccaagcgagaaactacaacaagtactcttcttccttcagacaacttcttgttaatgatattaacaaatcgatccctaaatctttctgaggactctagttgcgtctgatattgagctaacaataatgttctatccattccattccaaatcttcaatttgtttataaaggaatctttcacaacctcaattgaaagtttgtcttttaagtcgtttttgtaacctaatttttcggcatctattattacactacctatcgtgtaaaagtgtaaaggaaagttagaaatataaggtgtagcagtaacgccaaatatgtattcaaatcttccaaataaagctacgcttttaccgaaagcaattgctgcactagtagtgtgagcttcatcataaatgaccatattaaagtttgatttgtcaaaaatatcaaaatactttttgaaatcttttttcattagagaattaagaaactgaactgtagtaactgcaaaatcatattcgttgtctaagatatcctcaacattttttctatccaaaactataacctccgatttttcaaaaacttcattaatcttatgtacccattggtcaattaacacctctctgtcaactacaattacagaaggactacctaacctattagccaaaagcaaagcaaaaacagtcttacctgaaccagtaggagcttttattaatccatttctttttctctcagaaataattttttctatggcccttctctgccaataaaaaggatctactttttctatcaaattaagcttgcgattctttggtttaaaactttgagaaaatgtccttattcccatcaggtaagcaacagatgaaggaacaacaacaagtcccttatctctaccattttcctccttaaacaaaaacatactttctggagaaaacctattttcaatcaaggattcgtaaatccacctcttagcaagaggtgaggaatactcaataaacacacaattcgggtgtactacaaaacttacagccttaggtaaagcgctcatgcttcaatcgtaccccaagaacaaagaaaagtcaagaccctagaaaagacacctcaaaaaagaccctacagaagggttggtacactattaacagagtctggaaagtctagtattgacacaccaaacaggcttgtggtatagtcctattgtgggactactagagaagattacagaagaccttgggtcttttactcacctagttggcgtagataaagtctactctcctttggatacttttggaattagtgaaattaagtctttcttatctggtagtaaattctactcatttagcttactgagtggaaactactttagattaatcgttattgatgttgatcaagaatgggaagaaaataaagtattgctttttcttaactacttgtttgaggtttacaaccttaaagcttacgctgaaaaaagtatcggtggaagaattcatgttttgattcctttcaactctggaataaagtttgatgagagtaaaatattcttgaagttcatagagagtgtagctattggtttttggagggatagcgtagaggtgtttcctaatggtcgttctataacgctgatatttgatgaggtttatggaagaaaaggactcttgttttcaaatagcgattttaagaagaagttaaggttcttttataggtttttcaaaaagccgttttctaacaccaagaaaacctttgaggaaattttaaaaacacacaaggacttttttggcctaaattcttatgtgaaagaggctttaagttcaaagagtgaagagattgatgttgttagcgttggagaggagttagaagatgtttttagagagattgttcaaaagatagcagaaaaggattatgttagaggtagaagaaacgatttgatttttgagttgtctggatttctagcttttatgggcaaaagttttgaggaaataaagaatcttttatggcctttgattgaaagagatgatgaaagagataaaagaattgagcttattattcgtgctttagctagaaagcgtgaaggaaaaaagttgtattttaaacaacttcttacgatgtttcctagtttgggcaactttgttgtgagaaaaagcattgaatatgctgctttaggagatgttgttatgtcaaggatttctaaagaaacaaacgataaaagagttctaaaggttgccaattttatatctaaactgacgagcaaatacggcagtcgtgtggttcttggatacaattctctgtcaaaaaacctaaatatgagcaaaagagatgtttcgtttgcgctaaagatgttgagcaatctaggaatcataagaagaattgcaaaaggcagatacttgaatattgatggagaaaagatttctataggctcaatttatgaggtttgtattttgagtgcagcttcttttgtctacaagatatctaaagatatgaagtttgcttttgaagcaattaagaacaaaattcgtgaagtgggagaattttacttttctagggcgaactttgaatatttaagagaaaataagtttcttctgttgaatttgaatagcgtttaagacagcagggtagggaggaaggcacatgaaaaaggcgcttgtagagcatgtaaagaaagaaaaagatgatgaatttcacactcctcgctttgcagttgaaccgcttttaaaatacataccaaaagataaagttatatggtgtccttttgatactgaagaatcaaattatgttaaagtttttatggagaacggttataaggttgtttattcgcatataagtatgggacaagatttctttttctacgaaccagagaattatgatgttatcgtttctaatccaccgtttagtgtaaaaactgaaattttgaagagggcttatagtcttggaaaaccctttgcgtttttactgccaataacttcgctagaaggaaagaaaagaggagaattgtttagaaaatatggacttcagcttattgtttttgataggagaatagaatttatgtcgacaacaaaaactggcgtttggtttaacaccagctatttctgttataaattgcttcctagggatttaatttttgaacagctagaagtttaataccagaaagactctaggttgtggtaatacaagtagtagaagaacaagaaccaacttattgaggctatgctaggatactcacctatttcatatatatcgtcataaccaactgcataaggaagtaattcttttaaaataaaattttggattttgtttttatcttcccttaactcaagttcatttttaaagactaaatactttgcgtaaagcgcttgaaaatagccttcaaaccagaggaggttttttgcgcctggataaccaaattcattggagtaaggtttgtataaatttggtatgaaggaagtgtttgttggccttattttaaagaattggtctaaatattggtagtttttaaatactttgtttatatcaaactctattttataaagaaatatcgtgccccaggtataaatatctaatgctcccgctgtatcttgatatccttgtgttaatttttcgccgtcataaatgttgtttatgatagagttttttatctgttctgcctttgtgttgtaaatagactcgtttgttatatcatatacgctttttaacgcaaagtagctgtctatgttgtgttctgtactgtaccatggtacgacataggaagcgtcaaagttatattgagcgtcataaattccgtaacctcctcttactaaaccgttagattgtatttctcctatcaaataatctgctatggattttataaagtttttattttctaatgtttgaagttcaggaatatatttgtatgcctttgcaagcgcttctagaagccaagctacactaccgtttcttatgtattcgtcagaaaagaatccgtacgatggataactaaaattgaaagaattttttgagttaagaaggtttttggaggaattaaacatctttacaattagactttttatataatcctttgagcttaacctaaactctgaaacaaatgttagtatagaaagtgctaaatcgtaaatatatgttcttaaaaacggaaaatctttatagcttacaggaagataagttttccagtatataaaatcttcttcatcgtctatttttatttttatcgctaatacttctttttcgtttgttttacttatcgagccggagtaaacgaagaaattgttttctaaagagaaagaaggatatatagtgtctactaggtatggtatatcagttattttgtaaatttcaactttataatctacaccaaacggatagtaaagattgtaatttactggtaaattcacaaaaagctttgaaccattggttagtaaaaatttgattactacataattctcagagggatttccgtatattggcccttcaaatttccatgtgttataatcatttattccttcacttttaagtttttcgaattgaagatattcaatatctgttctcacaaatacctgaactttatctaggtagttatataaactattacttatccagatttttacttggtttcccaaaaactggtagtctagtactaagttgcctacttttacaagttttttaaatttcttatctacatatatcgtgtagttccaatcgtttccaatgctatttatttctatatcgaatgttgttttttcctttgtaaacaaataggcgttcttattttcggttaattttattagtgaattcagaaaataatcttttagctgttctatagaaaacgctctcttttttaggattaattgagggtctaattgagaattgtcgatttctttaattagtttctttagttccataatatcagcttacatatttgttaatgcgatatacatatggcgtgtgatactctggttcttctgagatagcaaatttaaactttgaattatcgaaattacttagagtctttagctctccaacaatctctacttctccactttcatctattggtttatggcgggaaaaaagaatcttaactattggttcaattacttgcaagccaggaaactcaattatcgtaaacaaaaattcttctaagccattgcctccacttataaaatttgccccctcaaaatattcgggttgcaaaaacaaatctgaaagttttttgctcaatgaaggaacttttgggtactttataactgaattttttatagaaaagcttacattccctattttctttttgtttaagtatatgtctatatttacagcttctatatactgtggttcaaactgtaaatataaagttaaaaattttttattcaaatcggaagctgtaatattcattttttctttaaaaaataaagaattttcgcttataaccttgctttcaaagctttggctaatgtctttaagcgaagatgccgaagggatattaaaaaatctgatatcctttgttcctaaactttttaaatttgaaaccatcttttttacattctcaataaagtccttattgaaaccctggaaaacctcttcccaatttattttggtgtttacttttaatattttaagctccatatgcccccttaaagttattgttgcatttttacacagaagcaattaatgttgaaaacagaataggtgtcttcaaaggacgtggatactggaacctgatttcctttaatgaatacttttaatgaaacatcatctccagataaaaagcacaaagcaccaacaccagattttgtaggagtaaaagttagaacctttgtttgttcatcaaataccatagtgcagtcatcagttttttcgtatattgcaaaaattacatcatctacaccaggcacaggaaaaaaggacaaagattcttcattagcttgtatgcttacactttttgctaagtatttttgtgttgagattagataagcttcgataactaaatttaaaacgctggaagtttggttttttatctttaccgaaataccctttttgttttgttcgaaggttaccttaaatagttctgatgaacaggtgcaattagaaaagtctatagtaaattgtgtcattgtgttggcaagtgttacaggcacggtttaattgtatacaatggaaaaggctttttcacttgacacgaaagagctgaggtgctaccatgagtagcatggacaaagaccttttgggatacaaaaaaattgttggaattgatatagaaacttgggatggaaaaaaaggaggttcacttgatccttactcaggaaaaatagcgttagttcagattgcctatgacagaaataacattgttttatacagaccttgggaaaagaattttgaggaagcattgaaggtacttagcgataaagatgttttaaaagttggacacaacttaaaatttgatttaaagtttttctacatgaacaacatatatccagaaccaatatttgatacgatgattgcatcacaaatgatttatgctgggttagatgaaccagatgaaatttctgaagttttagaagcacttaaagacgatttgactacagaagaagtagctatatttgatgtgtttggcaagaaaagaacaaaagctaccaagttttctcacagtctgcaggctgtcttaaagagagaactaaacattttcttaccaaaagatgttcaaaacagcaattggggaggtgagcttagtgaagcccaaaaggaatatgcaattaacgatgttaaatatcttaaagatttgtcaatagagttatggaaaaaaataaaggcattgaacattgaaaatgtgtttatgttagaaatgaagtttttgcctgtgttagtaatgttagagttaacaggggtgaaaattgataaagaaggttggaagaaaaaaatggaaaaagaaaatcaagagcttttagagctagaaaaagaacttaaaaaggaaatatatgaaaaatttgtttctaaacaaagtgaaaaaatgatggtcggattgtttgaagatgcagaatatgaaaaaataaatctaaactcacctttacaaatggcaaaaatttttggattgccaaatgtttctaagctaacccttgagaaaattgaagatccgacaataaaaaagtatgtggaatacaaaaagaaagcaaagttagtaacaacctactcaaaagaatacttagaaaaactaacagaaaacaataggttaatgtccgattactcacaaacgaagactgcaactggaagaattagttctagtaaccccaacctccaaaatgttccctcttggtttaagagatacataacagcagaagaaggatatgttcctgtttttgctgattattctcaagttgagctgagaatcttagcttatctatctggagatgaggccatgattgacagctgtaattctaaagacatgcacagcgaaaatgctagaaagatttacaaaattcccccagatcaacctgttccaccagaactaaggaagaaagcaaaaacagtttcattcgccattccttatggttcctcagcttatgggttggttgtgcgtggcatgttctctagtttggcggaagcagaggaagctattgaaggttttttcaatagctttcctaaagttaaagagtttctagaaaaaagcgcaaagagtgcgcaaaacggcattactagagatgcgttgggtagagtaagaaactacaagaaaatctatattagcgaagatgaaaaaggagctttaaagatacttatgtttagattaccttctggaatttctaaagatgagtttataaaggaggttttagagaaaaatagctttaaagacgctgcaaaaatttttggaatggatgaacaaatgctacagctagcaatttctgcttacgaaaaaacaaagaaattggctaagattgcgagagaaggacaaaatcacccgattcaagctacttctgcatcaattacaaaaacagctatggttgatctcttagaatacctgtcaatgacagggtatggatatatgactttaacagttcatgacagcattttctttgaaatcaagaaagaatttatttttgatgcacttccaaaaataaaagaaataatggaacaagctggtccaaaagttgttcccggaattataactccagttgatgttgaggtaggagagaaaatatcggtatattgcaattcttgcggtaaagaaaacgagattaatagatacatggtaaatctaaatgagaagataattgttgattttactaaggatccttatatttgcgatgaatgctttaacagggttatttaaaatagtttcttaaccaaaaattccagaagggcattagggatatttcaaacgatgctactataaaaacttctaacaaatccaattttctgccaatcaagtcttcaaatagattagatataatccacagaaatccagctactccccaagaaatatcacgaaaactcggcttttgtttaattatgaagcttcctattaacactactagtgaaaatgtagcccaggaagttattatcattcccaagaaattaacaaagagcctcactatgtcgctatttagttccattgtcgttctttacccccgtcctatcttttttatcatcatcgttctctttttcgttgacattataggaatttccttttaatctgtgtgcgagttcacctatacctaatagggcaccccaggctgagcctattaaaaatgcccaaccttgaggtatatcctctcccgttaataaactgcgtattaagagtgctagtgcagtaacggaggtaattgttaataaagttattcttattatagtgttttcttgattcatttttgcttttcattttcccacgcattcttaaggttcaagaaggaaactacagcatcaataagaccagaaacaaaattaacaatttccttttgcttttcctcgcttaatgaagttatgtttagcgcttcatcaagaagatccattgcaattctcttcttttctttcccgtctttttctttatatagactttctaccttttggaccacttttataactgtagggaggtattgaacaactaggtttaggaatgagattatttttaatatagctaccatgattaccacctttatttattttatttaaatgtagagaagagttttatttttcccggtgcgtattgacagctatgctgttttatggtaatattgaacaagggagaaacatgctaggagcagtttacatactagaaaagaggggaaggggtttttacaaggttatagagaaggtggttaatgagtttgtcaatgattctgatttagagagtttaacaattagtttggcgaatgatgtttttgcaaaaaatggagcaattggtaaagttgaaccaacaaatgaattttttatgagcggttctgtagaaatcgatggaaaaaaatttaaaacgagaagagtaaaaacttatagagtaaagataaatcactcaaaactgacaaaagatgcggaattttttgttgaatctgttattttaagtgagaatggacaagtggcataaaagattcattgaattagcaaaaacaattgctcaatattcgaaggatccaagaacaaaagtaggtgctgtaattgttgataatgaaagaagaattgtttctatgggatataatggcttcccaagaaaagttttggatttagaagaaagattaaataacagaaaagaaaaactcaaatatgttgttcatgcagagttgaatgctattttaaatgctaaaagagatatagagggaacaacaatttatgtttatccttattttccttgcaatgagtgtgccaaagcaataattcagtctggtataaagaaggtaataactacgggaatagtagatgataattggaaagaatcagttgaagcctcaaaaaacatgtttaatgaagctggtataattgtggaaatctttcaagaataagatttattttgtttatttaaaataagtgtatgtatattgatgttataaaattaatcttgcttatagtttcttttacaaatggtaaaccagctacagaggatatcgcttggcaagtagcttttaaatttacactaccagaaaatacgccaatagttgaagtttcatttgattatgtgcctatttacttttcttctttaaacggaaatctttgcggaatatcttatggagcgtttgtgattattgatccaaacaataaagaaaaaggttgtgaaaatacattggaacacgaattggcgcatatttggcaatataggtcttatgggtttatattacccataacatatccatttatgaaagacttttgggaaactgatgaaccatggcacaaaataccagaaacacacagagttttaaactttccccttataagattttatataccgttaacttttccgtgataacataaaaacaaatgaaggatactttcaacatctacatagacgtagccttaagaaaatctggtgttttaataacaaaaggcaaacaaccgatagagcattttttaattaagtatgacaaagaatttaatgtagaaaactttgaagctattgttgcacacaaaaaatttataaaaaagacatttcaaaaaataaaagaaaagtataaaaagtttgataaggcatttttagaagcagatgttcttggttatagaaaagggggttttaagaataaggaattaatgacaatagtaagaatgaattatagcttttctttaagtgaagttttagttaaaccaaaaaatattcactttataacagcgtctgtatggaaaagaaaactactaggaaatgccgctttaaaaaaagcagagtacaaagaaaggttggtaaaattgattgaaaaggaattgcacatagaaattagtgatatcaaaagtatggatatattggatgcatacggcattttcctgtatttctttaaatcaaatgttgtgtagactcaagcgttgacttctttctcttcttgtgttatcatagaagccatgactgacctagcaagatacaaggaagagctttacggatctgaagccttaagggttctttttgataagataaagagtaattttgaagaggatgttgatacttttgagaaatttttggaatatggagaaccagaaaatgcgctaacaaaagaagaaattaacaatgtatttactgaacttgaaaaactaggattaattgaagaaattaaagatgagctttcttctttctacaaagtaaagaaagaagatttgattgacgggttttttgttcaaaacatttctagcgaaatctacaaacaaaaataccaactgaaggatggaaaaaacaaaaatgagctagttgatttaacgccataccacacatttgttcgtgtagcactagctgtagcattagttaatgctagatactcaaaaagattagaaaagtatggtttagatgcatttaagacttttgttgacgattttctttccttcttttacattttggcaactggaagatctactggtgcgggtagagtaatgtcaaacgctggttctaagttatataagacttacacgacactaataaactgtacagtaatgaggcaaattcctgatagcatgcatggaataatggaaacactaactgaagctagtcttaccttaaaggctggtgcgggaacaggatacgatttttcctctattcgtcctaagggcgcttttgttcacggtgctggtgcagaaacttctggtgtagtttcatttgctgaaatttttgacaaaatgtgttcaactattatgtctgctggtggaagaagaggagcacaaatgcttgttattgatgtaagacatccagaaagcattgatctatttatggcaaaaagaaatgatggggttcttagatatttcaacatttctcttcttttccccaattcactaatggaaaaagttaaaaatgacgaagaatttgagcaatggttctggacttttgagatgacatttactcctagtgaggttattgaaaaaaatggtaacaatgttttctttattggagataaagaagtagtaattgttgaaaatggcaaacttccttatgagtacgaagaatacgactattttgcttttggtgaagatcatcaagtaatgaaatatgaaaattttggtaaagataagtatagaatttacaaaaaagaagtatttaagagagtaagagctagggaaatttatgatatcgtaatgcaatctacctacgactatgcagaaccaggaattatttttattgataatgtaaacaacaaaaatgttttgaagggaaaggaatacattagggcaacaaatccatgtggagaacagcctttgccaccatacggttcttgcaatttaggttctattttccttcacaggtttgttaaggagccctttaattatgatttgaaaccagaagaaaattttgattttgacaaactttttgaggtttccaggttaatgaatagatttcttgattttgttaatgacataacaaatcttccgctagttcaattaaaagaggccgcttttactaagagaaggcatgggcttggaatttctggtattgcagacacattaataatggttggacttagatacggttctgaggaagctgcaaagtttttagaaaaaataatgtatacgatacaacttgcttctgcagtagaaaatgttagaatggccgatatttatggtcctgctcccgttggcgttacactaaaagaatgggtagatgtgtttggtcatttaaaagagaagcttgaagagatttacggagaagacttttataaactaccacttagatattctcacgcaatctctattgctccaactggaacaatttctttaacattcggaaacaatgtagctaacggaattgagcctacttttgctttgtcttacatgaggaacattagattgcctggcaaaaaaacaaaaactcaagaagaggtctattcatttgctgcaatcctttttaagaattactttggacacacaaattacaatgaatattttgttactacaaacgatattactgtagatgaacatataaggatgcaagctagcgcacagaaatatgttgatagcgcaatttcaaagacctgcaatgttcctactgattatccatttgagatgtttaaagaagcttatattaaggcatatgaattgggccttaaaggatttaccacattcagatttaaccccaagttttctgtaggcgttcttgtaaagaaagatgacttagagaacctgttaattgaatttgaaacagatactggggagaaatatgttgtaaaaggttcagatattgttgtttatgatggtgaagaacatgtggcagccaatctttacgaggcattaaaagaaggcatttacgggcgcatgtaaaaggaggcctacatggtagagataaagggaaagataatcggatttcgtcttctagaaaacaaagaagatgataaacaaaaaccccttattagaaatcaagtggttcatggttatacaataaaaatttcatacaacgatcactctctctatttcaccttaaattttgatgagcaaggaaatcttttagagctttttgttaacagctcctcaacatcgtcttcaactaatgcttatatacaagcgatcgctagaatggtaagcaatcaattaaaatatagaattccaatagaaaagattatcaagcacttagataaccttgattctggagaaacttatttggtaaagtttcctggaatgaagaaatctaaattcattaaaagtattcctgatttaatagcaaaagttttgacatactacaaagatgaagaaacgcttaaaaaaatttcccaaggaaataatttagatgaaaaaccagaagaaaaatataatgatgaaacaaaaattgaagttaagacctcttctataacttgtccctcatgtggaagcaatagcgtaaaagttgaagaaggatgctttacatgcttgtcttgcgggtattctaagtgtggctgaggcttttgtggtatatttaggttggaggtgtacatgaacctttcctgggtggaagaacttagaaaggaaagagaaatgatcgaaagcgaatatgcctctcttggacaaaagatacaagaactagaagctagaaggaaaatgttaagagaacgggcaatttatttggagggtgttttagatgctgtaaagaagattcaagaagaaacacaagccaacgaaacaagccaagaaggtaactaaaggagagaaaacatgttagtagatcaggatggaaagccactaagcaagaaggagcttaagtatcttcctaattttttgggaattgtttctgttgttacaaagaaaagcgttgtggaaacaaatgaaccacttgaaccatactttataatgtttatgcttgttacgcctcaaaaaatggagctttattctcaggctggttcgttgttgcaggaggtaatacttcacaatcttcttgactcatatgttaagaaggaatacgggaaagaattaaaggaaatggacgaaaaagaagcacaaaaaatgctttccgaatttttgaggaaatatcaagtaaacattcagccacaaccaattcctctggattttgttgttttagaaaacattgacgaagaagaattaaagaagctagtagagcaggaaaaactaaagagcggctttttggtaagcggcaatgaacaaggataaagttcttacagtcaactcgctaattgttagggttcaaaagagtggagagaagtatactgatactggattagttgttgtggaagaagaaaaggctgtaaattttggagaagttgtatctttaccctttctaattacagaatctcataaagaatacgaagaacttgtaaaacatatttcggtaggagatactgtaatatttaatgcaaaaatgacatacaatatcattaagcttgaaaatcaaaaaccaaacgaagaatttttaataatatcacttgataacatacacgcaattgtaaagaagaaggaggtgcaatgacaacaaccttctcttttaaaacgaaaaaaacatgttcttgtaaattttctgacgaatatacacttgtttgcgaaaatgatagctgcaaattgtattccaacggtattttagtaacaaaagataaaaattacgcaagaacactaatagaaagtgttaaggattctttagaagaagaggactatagaaaggcattggagcttttggggggttagtatggacattgttttgtttttcaaaacctctttactaaccttcttaatttctcatgtttatatacgatacctaaagtatatacccaaatttcgtatctttaagggcatttttcaagaatattactattgttatgcgtgtgtaggtttttgggtagcggcgcttattgttgtgattgttcgtcctgaaataactgataagcttttgtttgcaattttaggaacaattggatttttgtataattttgatacaagcgtccttttttcaagaattagagaaaatggacttttttcttttacccctgttgtaagtgctgtaatccttgctgcttttgtctttttaatttccttgaaaatttctgcaatttctttgctattaacaatatcgctaacaaatgttatttacaaagaactctatgaacgctttttaggaggctccgatgcctaaaaaggtaaaaacagaagatattttgtcttctttaatacaaacaaactttacactttccaacaaaaaggatttttttccaacaaccaagaaggaatttttaaaactgaaaaagtttttgggaaagtcattaaacctagaaggaagatatgattttgatgctatatatttgcctataaatgaaattatatctttagtttcacagaaggattattggacaaccaaagaactgaaactacttgatgagcagattaaagagtattctcttagatatttagtaaagactgtagcaaatatgatataatctttttgaggatatatggacagaaacaacacaagacaaaaatttcaagaggtaagacaggcaaaaatgcttgcatttttgtctaatacatgtagtttttgtcaacaactgttaaatgaatatggtaatgttttagatactttaggtgtcgtaagactgtttgttgaagaagatagaattcttttcaacaaagagtatgtatatgtaagagaatggctaaaaaacaacaatcaagaactagcaaagtatccatatataactccggttcttttagttagggatgataatttgaacacagtcggagtttatcttggatatatgagtataaaaacctttttagatcaattaacaaatacgcaataaaatgaaagaatgagagaagttattttacctgaaataaaaattacctatatacctgatagaaaggtcaggttttatattgattttgaaaagctttttaatcttggccttcataagtcaaaccttgttgtaagtttccaaaacaaaaactttgagttttctgaagaaaatatctttgaaagactactttttaacccccctcctgaggataatattgttgaggaatttcaactaatagttataactttagcaataaaagtcgtagaaaaattaattgttagatttctggaatcaaaaagtgaatacgatgttaactatgaagaattagaatctaaatttaacgaaataaaatcaaaatatgaaaaccttggatttatacttgaatttgaaacgacagcattagttcccttttatctgttcgtgttggatgttttattagatgtatttaaaattggggaggagcaatgaaatatctaactatagcaagttctggggcggaagattttatttgtatttctgattctccagtagatcaaacaacgaataatctccctgttaattctaaatgtataagactaagaacaaaagcatcttcaattgaaggattttgccctccattaggacaaaaaataccaaaagaaaattatgttgtgtataacgaatcgtcaaattcaattgattattacgaagcaagtgcgaatacaaattgttctgttggatacacgaaaaaaacaatagctttatatcaaaaacccaaattcagatatttcagagctttaattttatacggatggagcatgaagaattattcgtgttgcggatgggacactagtgtagctccaaataggtccaagcgtggtttagtgccgtttagatatgaagtttggtattttgagtttgattccatagaagacgcatatgcctggaggaatccaaagccctataatctgacaccaatgcacttatctactgagtcaagtatatcttctacaacaaactaccaaagaggggttggttatgcatcatctgcagattattcctttcttggtagttacctacatggaacatatggtcttcaaaatatagatgatgaaaatggaacaaaaacaatttttataaccaccggaggatccacaaattctgtacatatattgtctggcaactacacaaaagtaagaatgtatgaaggggatttttacaattacctttataatcaagcatacaattcttccggttatcaaaacataccactttggcctttaagacggcaattcacaaccagctataatgactatacagatgaagaaaaggaatctatagaaactttttcaaatctttatattgcaggaatagaatatgttcttccagatagatattttagacaaaatccaatatacacaggtggaggcggatattgggtatatcattattctcttactccaagcttagaaaaattagattcaactaatactattggtacagatataatgggttttgctgatttagagataaattggtattttggtagtaaaacattttttacaaagacagacggcgtttacttctccggagctacttcttcatgttcatttccttttgatcctatttttgccaatattatgcaatattatatgtctagaaccctatattataacccatcgggagcttcactttatgcgacaaagaatagcatttctggatatacaacatctccaccagattccaaaccaccagtaatgtttccaataattgtaactatgagaggagatataggaggagatagggtaagacatgcagcaataaaacttggcataagtatcgtttatgctgtgcaatatggtcaaaatgaagataaacaaaacttgccttcatatcaatttcctgacgtttgtccgccaaacaccatacctaatgcaaactggattcttaatctgtaagatagttgataaactgtaaacggttttcttaatttttctgataatataaaactatggcaatatgttatataggacaagaagaagtttatagtacaccaagccataatgacagacttgctattcttgatgcagaaaatgctacatcattaaaggatgatttaaactttattagaagtgtaatttttaatattttggggttagattttggaaatgcggataataaatggtataaagttcctttttataatttgacttttctttttgaaaaatattcttcactagaatcagatttttctgtagaacactacacatcttccgaaaatccaaatttagctgggaagcataaaaatatcacagcaacaggaaacttaaatgtttctggatatggtttaataggacaaaatttatcggttggaagctatttagatgttggtacgactttaacgacaggttctgacgctactataggtggaaatctaacagtaaatcttagtggttcaatattaattgatttgagtgtaggaagagatttaagtgttggacgaaatacccaaataggaaataatttgtctgttaataacaatgcaactattgttaacaatacgaatattggtggaaatttaactgtaggagggtttggaacatttggcagctatataaatgtacaaggtaatgcaacaattcaaggagccgcatctataggacaaactttaactgtaactggtgctacaactcttcaaagcacgcttagtgtttataacaacacaagtatatctggaactttatctgttttaggtaatgcaactttttctgctaacgcaacaatatcaggaaaccttactgtaaatggacatacatctacaggttctttgtatgttggaaagcagtatatagcgttattaggaacagcaaatgtaaccaattaaccatgagagtcattatagttaataaaaaattagaacaatacctctcaaaatcgtttaagttgagagaagatttaatatttgagtattataggggaaggtggatttactataacgacattattattcctcatatctctaatgacgattttgaaggatttttcttttctggagaaaatattataacgacagacaaagaacttgaggagaataaaatttattttgtaaatgattttgagggtatttttacaaaggaggaggcagctttaacaaagctaagggttgcctacgaactatctccaacagagtactttgttgtgttgaggaacaacaaatttctcggcgttctgtacaaaaacaacctccctttaagtttggtagattacataatgttagaaacgaataaaagaccgaaatttaaagttaatgtagatagatttgaaatgaaaaagaagtttgtacacatactaaaaatgtctaatattgaaattgatgaagaaatgttctttagattagatgaaattatagaatgatatgccacgctggaatacattggcaataaaaaattttggttttattgaagataatgtattatctgtttttcaggactataaaaatcaatttaacttcaaaaatcaagaacaaaccataccaaatgacattaaactaaattatgaaggggacatagatttttacaacgatgatgttgttctagtatacaatctagaggcatttattcaaagactttattttttccttataacgaaaaagggaaccttacctggaaacaatgaatttggcgcatcgttagaagattacatcggaacaggaagacctaatcttaaccttcttttaagaaccctagaggaagatttaaagacttttgatgaaatagagtttattaatgatataaaggcatctttagtttcaaatgaacagacagaaactatagaaataaatctggacttaaaagttaggggatttaaatacagggcgtttttagacttggagcttatttaattaatcctcgtaccactttctgttgtagtctatatcaaagtctttctttaggttaaacttgtgtccatgaacaacaccttcacctattgcaatagaaagctttctacttaaaacatcatttctccatctgggattagctaaatctgcaacaacataagccaaaacttttagtttttcccattcatattttgggtttagttcttttattctcttgttgtattcgtttatgtgatcctttctaggagcgccgttttgaacagccacatcaaaacacaaggctaaggctctttcgctttcaaaatttaatgcttcacaaagaagttgcgccctcctaaagtagttgtttgcagcctttttaaaggcttctacagtttctttttccttagctaggttttcaaactttattgaccactctctttttactccttttccaacgagaacatttttaagaatgaagtctttgagtgttccgttgtataaagcctctacaaacccttttcctaggtatttttctaaggtttctttgcttatcattttcaatagtctttgaagtgttccttgtcctatgttccattgaagggggccatatgaaagtatttgaccgtcaaaatttcctgctactctaccccaatccccacttacttcaaatagggaaacaagtctgagtagcttttcttttagatcttcttttattttcttcatataatattagtttacagtctttaattgaacgaaaattttgtgataatataaagtcagcaatgaagactagaacaattgacgaaattagatatctaaaaagactagaaaacatcataaaagctagaatgcaagatattgttaatagcaatgatatcattgagtctaccccagaagataaagtaagaatacggatacctataatggacgaaccgtattttaaacctgtgtttcctggtagtggtgctggggcaggatctggatcggggagtgaacctggagagggtagcgaggaaggtgatcacgaaattgaaattgaacttactgttgaagaactttctgaacttctatttgaatacctaggattgcccaaaataaaaccaaaaggctcttctgtagagaaagaagaatatttaattgaaggaatttctaagactggaccaagatctagaatacacaggaggaaaacatattacgaaattatgaagtatggttataaagaagattctataaggtacaaacaccttaggaaaagagaagtaccaatatttgatgcaatagtttattttgcaagagattactctgcatcagtagatgacaagaaaaaatttaaaataaaatcaacagcgttttggataaataacttcattaaatacaattacaagaatgtaacaacaaagtttgctgttcacgacacaaaggctaaatttgtaagcgagcaagatttttttaagttgtctgaaggtggagctacgctttgttcttcagtttttgagttaatatatgaagactacagaaggtattcagtagacgattataatttttacctcttttacttctctgatggcgaaaatctaccagatgacaatcctaaacttagggaattagtagaaaagctatcagaggactttaacttaatagcttatggagaggtaaaaagtacagatgtttattacggatatggatctaaagaaactctggttccattatttatttcagttggaaaagataatgtatttgcagaaaaaatcttttcagtaaaggattttatattgaggctttttggtgagaaaaatgaaaggtagaaattacgaaaaagaaaccttagaaattcttgaatgggtaaaagatttgggtttagaaccgaaagacacaatttttgtagatgtagatagagataagctaatttttgcaatatcaaaccactttgttaatggaattccacattggacaaaaggaagaaatttcataaaatacaaagaacatgaattgcaaggaggaaaagtatttgaagttgtttttaatagtacaccagcagtagcctatatgagtctggcgttgacagaagctgatatattgacgattattgctcatgtttacggtcatacacacatttttgcaaataatatttttgaatacaaggatgacaacatattagaaaaacttaactattctctccaagtttacgaagaaatgctttcttattattcttacgatgagctagatgtaatttatgactacgcttatactgcttggaaccttttggttcctgatgaaattcgtgagaataaaatagaaaacacaactaaagaagagtttactgagaataaaatatttaacaaatttttgggaaggaagccaaaagtagaagattttagggaaaagtacaaaaaagagaaagaaacggagtttaaaaagaaatatggaataggttctgtgtatatacacgaatacatactagaacattcacccattcctgattggatgaaaacgcttataaacaatgaaattgatctgcttaaagtaataatgagaaagtcaaaattaaaaattctccacgaggggtttgcttcttgggtagatacaaaatatctgcttgaaaaaatggagggaaaagagttttttagtagcttaatttcatctactcttgttggttatcccagtttgagaaatccttactatattggatataagttgttagaatttgcagagaattttatgaataagaaaataccagaatatgttaaatatgttgatgattttgagcttatagaggaggtgtttaatgaagactttttttattggcttttaaagcagtttcctctagatgaggatgagaagaaaattttgtctgacagagaattgtttgaagaagtttttgagaatcagaaacaaaaacttttatcagaatgtctaaattattatccaatcatttatatagatgactacccagataacattgaggagccgtcatatgattatctaaaagtatcttcgtattcgcaaaacagaaaattaaagcttacttcacaaaccccgttggacactaggtatgcttacgaaacactaaaagttttagaaaaaatttggatgggaccaattgtgttaagatacaaaacgctaaaaaaataggcgaaagtgccgggatataattagattatgaaccttgtggatattataagtttctttggaactacaggatcacaacttccgtttaacataaaagaagaggaaggaaaatattacctaataaacaaccccaataacaaagtaaacaatattccgctttcagatggaatttattttgaagtaattcaaggtacgctagacaatcaaaacgggctaactcaaaatttggttgttaagctaagaaatccaaacaatatacaaatagcttcctcgtatgttgttagactttacaacgcccaaaacggtgctctactatttgaaacttatgtacccacttccttcgatggtttagaagtttctttttcaactaatgtaaatgttccacttattacgattgaagcaaacgggttagtcctctacacaagttatgaatacttaaatggctatattcctttagaccaaagaatttatgcttcttttagagatcctaataacattgaagtttttagggctttttttgttagaaaggtaaacaacaaaagatatttcacttcaaccttcagatctgtttacaacaagtttttgaattcgtcggcaattccttttagatttttagacgcttacctaaacaatacaataataggaaatttcaattatcaaataatcacagaatttaaatttccacagcagtatagatttatttcagaacaagagtcgctttctataagcgcttattcaataatgcttttagctttctataaagattttgttgaaaaactatctggatttttagtagatgaagtagacttttcacaactagataaagattcaagaaactttccttacaaaaagtatttttacaacaaaaacactttgtcttacgaaaaagtatcctcctatgtggacataacagagcttttgcttgcaatactactaggaataaaaatatatggaatgctagattccagcaaacaaataattgataatttggttaatgcgctaaacactttttcaccaactgtttattatccagacgcatcagttggttcaaaggctttaatacctgatatcataaattcaaatgtagaagaacagtattctttgtttacaaatgttttatttgcgcatatattaaatcttttaggaattgataattcgacattaatcgatgaaataaacagagtatttgtcaataatagcaaaaaaacagaaaataggtctttaggcactataaactcttttactcctcttgaagcggcagttattttaaaatttgcaccccaatatcttgatacaagtaacaattatgtttcttcactaatttcttcagcaacctctcatctctattctgtagaatatgtaaactctatagctttaaattctgatgcaaattcagattcttacaacccaatatatagtgaaaatctgtcttcagctttgccattttacgacaaaagaacttatgtaaatctaattataaatagtctgttagataatccgataaacaactttagagctttttcttctgacgaagcaatattggatagctacaaatattctatttacgaaggtttttatatattgccttcacttagagcaaacctgacatatttctttttgagtatggagaatgcatttgaaggtagggtagaagaaaaatctgaagttgtgcttatagatagtgttataagtgttggaacaaatcagttgattgtggaattaatttttaataaaccagtaaaaattttgtcaataatttctaatcaaggagtacctacagcttacgcatcgtcaatgtctgaaagcgctcaccatgtattaacattttctgttattaacacctacttaaatggggttttagaaatttacgttctaagttaacctaaagaacgggaagaaagtgctataatatactcaagatgaagttaaacaggtttttcaagggtaaaacagcggagagcagaaaactttcatttaaagaatttcttgaggaaaaatttgtaagaaaccccgcaaagtattctagaagtgcacataaatacctttttgatgcaattacatattttgggttcgatgaggaaagaaaaattccaaaaatctttgcagatgagctttttggtgttgacgataaaataagagaattcatgactattttagaagcgggcgcaaatggtcatgatgtaagaagaagaataatcctttttgttggtccagtaggaacggcaaaaagtacctttgtaagcattattaagaaggcactagaaagatactccaaaacagaagaaggagaaatttatgctatagaattttgtccgatacatgaagatccactaaatttgattccagaagatacaagagatgatttttataaggagtttggagtaaaaatagatcaagaactttgcccagtttgcaggcataaattaaaggaactgaaatatgaagatgttagtacagtaccagtagttaggtttactttttctgaagccgaaagaagaggaattgctacatttgttcctgcagattggacctcacaagatgtttctgtacttgttggctcagaaaatttcagagctgtgcaagagttaggtgatccttcacatcctttgggttggaattttaatggttcattatttgttgcaaacagaggaattcatgaatttattgagcttcacaaagcaaaaccagaccttctttttcctttaattactgttgcacaagaaagaaaagtaaaagttgataggtttggtttaatcgatgttgatgaggtcttgattgcgcatacaaactatgcagaatacaataaattcgttaacaaaaaagaaaatgaagcgttaaaagatagaacaagagaaataaattggatttacaatttaagatgggacgatgaagaaaaaatttataaaaaaatgttgggagaaactacatatcatttggctccttggacattaaagttcatttcgggtattgcaattatgtctagattatcaccagatagagataagaagtatgacttgttaacttcattaaaactttacaacggtgattatggtgcaatgtactctgaataccatgtagacgaaatgaaacatcctgacgatggtaagttcggcatttcgccaagggtagcagtagatgcgctaagctttgcttcagtaggaaacacttgtgttactccaattagagcctacgaaaaactagaagaaataattaaatctggaaatgttgacatacctcataaagaatttttggcgctaaaagatatgattttaacagaatacaacaaatttatcaaagatgcagtcttcagagctttcatcggttcttcctttgccaccgaagcgcaaaagtatgtggatcgttatattgaaaacgcccaggcgtttattaatggagaaaaaataaaagatccatttacaggaaattatgtagaacctgatgaaaaatttttaaggtcaattgaagagctagttggaatttctaaagaagccgcaaaagattttagaatgaaaattcttgttaaagcaacaaatttactgagaatgggcaaaaagattacttacgatattcatcctctgctaaaagaagcgattgaaaataaaatctttggcgatagagcaacatttataaggggctcaataacttctgtagtaaaaactgaagaacaggaaaagcacatacaagaagcagttgaagaacttaaaaaggttggattttgcgaagtttgtgcaagagaagctataagctatgtagcatacctacttgatgtagcaagataaaaaagaagaatttgaaatttaaactaacttttccttccctaacaatgggaaggacgaccttgctttccaaaactttttgtgttaagataggtctagtatgtacctaccgagtttaatagggctagtgggaaaagcgggttcaggaaaagacagattctactattcggttttagcgaaatataattatgtgagaatagcttttggcgatcctgtaaaagttctgtcagtaattcacttactaaaaagttatcctggaattctggaaaaagaagatcaattgctaagagtttttccttctctgtatattgaactattttctaaccaaaaaacctctctttctagaacacttcaacaatatatcggaactgacttagcaagaagatttgatgaaaattattggataaaaatagcagaagaacttataaaggaaaaactagaaaaaggaattcgagtagcagttactgatgtcagatttttgaatgaggcagaaagcattaagaagcttggtggaattttaattaagatagttggtaaaggttcctacgattatggtaatttggaagcaaaacatcaatcagaaagcgaattggaaaaaatagtttgcgattatacagaaaatgagtttacttctctaattaaaaaacaccaaatcctcgcttcggaggtagaagatacaggacagtagaattactgtcccgtattttttagacttgactcaaaacatggagtgtggtatgatagcttttgagatgagtggcctcgtagctcaacaggaagagcactcgccttgtaagcggggggttgtgggttcaaatcccaccggggcctccagaaagatgaaaaaagtaaaaataataacctcctcagagcctaataaagactttgttaatataaacctcctaaatcgtaatggaattttgaggaaaagcattacctatggagttatgttgagagaaacaaattatgtattttttggagtcaatggagaaacatacttcaaaaactatgttaaagatgtctttataagcggaaaaaataaacttttagaagaactaaaaacaaaggacacaataaatcttgtaactattgatggaaaagaaaatttttacagcaaaatcttaaaagaagttttatctgatatcttaaaagaagaaggttttgaagtcatttaagaatctcattatcaacaatttcaccaaaaacccttttggcaaatttggcttcttcttccctaataagtttatcaaatttatctattatgcttctttttttatcaactttaaataagtcattttctaaatctttcggagtgccaactattgaaatagtctgtagaggaaggtttaccaaaagataagtaagtatcgcaacggtatcactcatatcttttgatgtatctggtttgtgatcaactttctttttttctctgtcttcaattaaattagacaactcccaaaacaaaattggatgataatacagatgtatgccaccattgtctaacctatctcccttcccagaagatgggggcgcttcaccaaaaattctttctcttaacaatgcatagtgttcgtaggtaacatatttgttgtaggtaactttcatgtgtggatatcgtctttttagccattgcgctgttgaagctgagttatatatgtcaaaaaatgtgtgtgtaatgttaaacttcctttcttctataagtttttcaattagattgtgcacggcgtttatttgaacctcctcttcatcaaacatatatccagtaaatctaaatacaaggtcaacgactggcctgggaattaattttccttcctttgaataatagaattctctatgtccgagagctattgcgtaagcatctccctctgtagaaactctacctaaaccaggatcggctacaagaacatattcaacaccttcttttccataaaaccagtctttaaattctagattgtagggatctattttttcatatgagccatcttcttttagggtgaaagcttcaagtttaggattattgtaaaaatcttctggtggcaaaggaaactttagtggatgaagttccgaaggtttcattgcgttttttaatctttccaaatccgtaaaccaagcgtctattggctgttctggtggattacaagcataaattgtttctgcttctacgggatttttttcaaactcagaggcaaaggtttctcttgttatcattggatttggattaaattcccaagtaggatattttgccacaaaagaatgaggtagccccaaatgttccttatatagtttctgtatcattccgtttgattcgctgtaacgcatgtatgaaatagcaaaaattttttctttcccattaaatcttgacctagaagaagaccttaacattgtccacattttttctggatttctaaaagacttagcatctatttcgtctgctactgctacaagaatgttttttccttccagagaatcgctttctgagtggccgctgactagatgaatatttttgggaaagataatgtgtttttcctttatatctgtgtctggattaaaaccaaactgtctaaaggcttttggaccagcgtttcttatgtagttctttagtggatcaaaaaatacacttgtagcttggtcagctgatgctgcaacattaagaaaagtgataatttctgaacttggtatcccaaagtatttatgtggagaatacatgcaaagtagacgataaacaagcctcataaaaattattcttgtaataaagtcctttccgccaccctttccaacccataagattatataattgatgccgttttcaaaatcgtctgctatttctaaaaccttcttttgaggttcagaaagaccataaagatttatgcctaaataatcgttactcgttacaaaggtaataatgtctactggttctatttcataaatatcttttggattaagttctgccttagcttcgtaagtttttttctctatgagagagtttatgtctttaattattgagaaactttcactcttcttcggcataatccacatctagtgcgtttttttcgtttaattctttaattctgtttcttacggcagttgtgttaacaatgagtgtaataaagtcttctaaagagttggcctctattaatgccctagcatactcgtcttgataaagcctttcttgaagctttattgctgtttgaggatctaattctgataagatttcgtacattgcctttacaaaagcaacttccctttgtctatcaagcactttttctattaggtttagataagtatttgtttgatcaacagaaagtttcaatagaacctcaaatctccttatgctatctttccaagcgtttaggagctttctattttcaagaacaccatcaattcctttttctgctaacagggaagatattttttctcttaattttttagagattattgtattgaatgttatttcctttgatagtgcgcttactgaattaaaaaaagaatataaaacgccaaaatgtgagcttggatctacaaagctgagttcctgcaacttcttatcgtcaaagtagaaaagataaaagtcttgaagaagtttgtgtattaagcttgtggatacgccatagcggtttgatatttcatttatgctcagtttgtttatggaataatcttctaaaactttggcgattaaatctcttcttgatagagttttgtggtttttgtgtgtttcttctaaagattcaaacaaagcaattgcttcttgtaaatccttcttttctgcttcttttaatagtttaatgtttatgtccctcatggttccttgcagctatccaaagtttctggcacaaaatgtctttcagattttttgtttgctttcacaaaaagccaaactatttcaccagcgattcttgttttctttactgccttgaaaatcgtttcagagctttcactccaagaaaaagtatctccttcctttaaacactcaaatccttcgtaaggaaattctacataatctttctcactttctatgtcatcttcttttagtcgttgaagtatgtatctcctcaaagtggaatagggaatattgtatttaactgaaagatcataataaccagcttctttatctctaagatactctttatagacctcctctaatagctttcgtttttcctcagaaatttttcttggcatattctttaaaatcctctggatctaattcttttatttcttgaggtaacatatcaaagtcttttgataaaccgtatttgtttagaataaagtagattttgttaattaaaacatttttttgttttgttattgattctatatcttctgttaattcgctttttgtttctggtgtatcaattgaacctttttcacaaaaatccaaatatttttccttaaatatttctggtggaagctttcttaaagactcatcaagactgtaaaaattgtttttgcatctaatgacaaaaaaagacctattttctaactctattaaattttcttgattaattacagaatttttgtcgtaatctttttcttcctgaaccagagatgtttttccttctggagtgtctgtatcctcctttctctgtttaacatcttcaaaatttttttgattgcttaattcccctatcgcttttaacaaatttgctaatagtgataaaatttgaataagtgttggaaagattaaaaataaagactttataagcccaaacatagttcactcacaaaaatgataaaatgtatgtgtggaaaagctcattcatatttattatatcaaatctgcaatttctgaagacaagaaaataaaatttctcagattaaaagatttatgcgttcaactagacgacatttctataaaatcttgtaataaagatgtattaaataaaatgtttatcaaagaacatatagtgaactcgccttactctgatgaaatgactttgataaaatttgcagaaaaattaactgctttaagaaaaatttatggcgacaacaagatacttgaaataatttaaccccccccacgaaaaataagcttttaattgcgtggggggttttcgtttatagcttgtgcggatcagaatatatgtaaccgaaggaagcacctgtgttgaatgtatctgcaagaacttgataccaaccgctattgtatagcggccttgccaaggcagtaatattttctagaataatagcgttctcagaagaatacgaaacagaaaaatctcttaacattgcaaggtaaagaccagcatttctactaacttcatttaaaaccttatctttcaatgcaacaacaaaaagaaacccaggaatgtatttttcattatattcaaattctcttccgcttacataataaaaagtagtgtttgaggaaagaaaatcattaggtaagggagtaccatttacatcactaggaagttcaactctcttaattcctctagcttcgggcatatatatttcggaactgtaagcgaggagagggtttatatttactccgtttttaaaggattgcaaaagtattgtagctctttccattgttattgaaccatcatagaagcctggaacaaaaataggtattggcgttcctataacagttctcgttcttgatcccattgactcgttgattcttaagttagttgctaaaccaattttttctacaccaacagaaaatttttgttcatcttcactaactagagttactttgtcgttatttagtaagtcatttgtttcattggtacctgttgtttttactccaattaaataaatttcggcaaattgagaagttttaataggtactcccattttatcctcctaataaagcatttgcgaggttttctattagtcctctgaggttaacgagttttcctgttccccttacatcagtcattataattgtgtcatcgccgctaattcttgttgaatactcagagggcatgaaaacatataaagatctattaagaaaaggttgtactatgtcagaaaaaccaatatcaggtgtttcaacagaagttatagatatgaaagtatagaatggataatctaagaaatccctaccagtaactggtcctaaaaattcttcaacccgtctttgaagtgtaggtacataccagtaatctggtagtgttgtgaaatctcttacagaaagagtatctaatgtcattctagagatagagattgtagcagaatagttattgggaacaacaattgggtttgtaggctcaccaattccccaaactggtctagagttataagattcatctatagaaacattgtttgcgataccgattggtttaaacaaggcgaaatttccttctagtgcgccccaaatattatttgaattgttaagaatgtttagcagcgctaaacctaaatcagcaggtgattgccctgcttgaagtaatgtgttgaaggatagtgtttttattattgcgtaaggactaaatcttggtgtcattttttatcctccaacttagtttactccaaatcgaatgttactttcattgtaactaggataaacttaatttcggttactggttgatagaagaattctacatagtaaacagttttgtcttcattagaagtaacttttacatctttataggcaacgatttgtttgccaacaagattagatagaagtgcttctgtgtataccttgatgtcgttttcagttttgcgcccgtagctttttcttccaatgaattgttcttgtaggtttttgtaaacatacgattgtaggaaatcatcaattcttctaagaacaaattcttggaagccttgagttacaggaccaagccaagttgttggattgtgatgaatacgataaacgatgccaccaaaagaagtttttactttttctacaaataaaactctgttagaaataagatcgtccttttgagtataggtccaattgtagagaggttcagcaccaatgccgcttattggtaggaatgtaatagaatcctggggttctctgttggcgtgcatgcctgcaacataagatgcaaagaattctccaccaagaagttcaacctttccttcatcgttaaatacgttgctgtagaaaggaaatacgacagcttgtgctctatctgtgccaccaaacattgcagaaattgtatttaaaatatttctgttataaaggtactcagatgcagttacagattcatttggagaaggcgctggtaaaccaaacacgccaactctagcctttcgtctgttaacttgggacattgtttgtacgtgagaaaggaaagtagaagcaattcccttaaatattgttaaacgatcgttaagttgtgttacatttgtaaacttgtaagctggaatgacaaagttaacttcttcctcttctaaagttgccaatccttgggaccaaatgtttatgtagttagatacagcagtttgataatatcctgtgccatcgtcgccgtttgttccaccagaaaggctaaagattcttgtggctagtatgtgtgggtttattgttgtatctacaaggtgattgattgtgtaagcgtctttagcgaaccagtttgcgggatcagaaagagatgtatagtcttgataatcactcccatcaaagaatggtgttgctgaaaggttgaatagtagcgggttagagagaagatcatcaactgttccaaagctagtttttactagcctagactttgtaaaaccaaagagtccaccataaagcacctcatatacaaaaagtccaagaggtatatctactggatttgtatcactagcatcaaaaatttgaacaacatcagtaggttgaagttgttccttaagcagattaattagtgcagtttggaaagctggtcctgctgctagcgctgcgtttactatagatctatccacaaaaaatagagtgcttcctctttttactaaaacagtaggtgttgtaaactttcttggaaaagtaaaagtttctctggttactgaaaaatctggatgcttttggtaaatggtaaagattatagtatcatcagcttcatttgcagaagtgaagttttcatctaaatcaaaagaaacataaatctgattggaacgtgtagaagcttcataagaaacaaaatttaagtttgctggtgatgtatcagaagtattttgtgctacaggaatagaaacactagcggttgttgggttgcctattcttacagcaacaacctcaccagcaccaccaataaaagcggccttaatatgttcaagaagtggcccacccttaaagatgctgacagcttcagcaaaggaagtcagtctatagggtttataagtttcgcctccctcagctaaacctagtagagcaacaattcctgttcctactccgttgatttgcttaacagcgcttaggtcagtttttacatatgcgccaagttgctttattaactttccatcaaaatatacggaaatagccatgttttttacctccttcccccattttacagaagctattttgtttttttaggaactaaaaatttatgtttctacgggcatttttatacaaacctttttaaaggagccgtcctcattgattattattgcttcgtaatcttcgcaatcttcgttaggaagaacatctcttattatgccaaaataagcttcttctcttggaatttttttaccaaatatcaacctttttccgcttgcatatacctctctgtaaaaaagtggatgattagctatgattttttcgtcttggctatcgttatatctttcaattacaaaatctggaattcctctttttaagaggtaaaaagattcttgcgcttcaagcaaaaggcgcaaaacaaaatcccctaacacatctctgtcaataggatcaaaagaccaaatgaagaaagaaactttgaattcttctatgaaaccataaatgtcttttttcaaaacattacctataaatcttttacttccttgattatctaaggcgatgtttatgcctcttttaaataaaaattcattatcagtattggcattttttaaggttgtgaagtatgtagcagatggaaaagaagttgttatactaagtgggtttcttaagtttagctgattaaaaatgttcttaaaataatctacaaaaactaattttggattaggataagaaggtataggtaaatcgctatttggaacatagttttgagcaatgctcaaaaactctaaatcatttgtcaaatcgctaaaggttgacataattagttctccttgttaattgcgtcaataaaaatgttctttaaaatatcttctatttcctcgttttttgcgcctacaaaattaactgattctacgacttctgtgtaatttgaattttcaaaattagcgttttctttgccaagagcaattctttcagatagataatgagttgctactttaaaagatttgtatagcattccagctaaccaaatatctttaggctcagaatcaacataaacgcttgtgtagagcgttccatcggaagaataaatatcaaacctatctcttataaaaaacaatcccccttcttcttgaagttttagaagttttactggaacttctggtttagttaccattctaaaagtatctgtaaaaaatacgccttcacgccttctttgtggagtgtcagaattgtctataactatggctatagtttctattgggttcttataaacaacaccgctaccgccacaaacaggacaatttattgaaggatttagcgttcttttatcaaaacaagttttgccatctggaaaacgataattgcaaagttcagcttcgtaatataaaactttatatccgcccataaaaaaggctttctctactatgtttttcgctatagaatatagccttgttgaaggatattcgttaaatttgaatttcattttctttattcagaatacatcaattttttcaagatttcttatgtaagaaatttgctgaagctcacctaaaatcttttcctcaaaaatgccatttcccatcttagatatatagagttcgtttttcctgaaatttaacaaatcctctcttttaaagaaaattattgtttgcgaaaaagaatagcttcctttgggaaataagttgtaattgtatattctgtatacattcgttaggtaaattgttttttcatgtattagctgttttagtgtttccattgatatattaaatgattttttgctatacctaaataaagaaatttttatcatagaattttcaccagcaaagataatttcatcagcgtccaaaaactttaaaaaatcattagtacttacatagaagacatttgttgaaatcttttttcttcccaaattaatggaacacttcaagttaaactctctgaagttgaagtttttttggagatattcttcataacgctttacagtttttactttattcaaatggctcatgctagctacattataacaaaaaagaaaatgccccccaacaaatgggaggcatttttttagcttacttcaatctttatacaattggttcttgaccaacgttgacgatgaggaagttaaattctggggctctagagaatacagaagctaccttccatatgacgtaatcatgagtaagtgcaggaggtagtctgtaaagttccataccaatccagacagccatagtagttacattaggatctattcctacaactacaccagttccaggaattttcttccatctataaacgccggaaggagtagttactgtttcatgaccatcatctatccaagtaaagtcgccgtttacagtagacaccttaacacgcttatagaacttgtattcttgagaaccaggatcctttctgtaaacatcaagccactttacattgacaagaccatggaattggaaccgaacacctttaccagcttcagtagagtcgatatagacatctatatacttggcagcactttctccatagaagttggctgccttgaaagcatagctgtaaactgttgtattagcaggatcgccaagaccgctgttgtaaggcagagttacgaaattatctgtagtgctaacagtccaagagttagaaggagcagcgaaatcacctacctcagcaccgaaacgagcagggttaaacttgtggaaatcaccgaggaactgagaaggatagagagaatgttcgccacgaacgccgatataggactgagcatctgcaccaagcagaccagcccttctatcggcagtagtcattactctagagatttggtagaaagaagcttgtaagttaagcttatcaacatagctaatgaatacagcagtaggattagcgaaagcttgtgtagatactacacggctttcagcttcccaaagcaggtcaatggaaagaggtctacctccagcatcaagaacattttgaggagcgcccctcttaattatattgatgatgccatcctgttgaagattatttgggctaccaggaacatcatcgccaagaaggttatctccatagaaagccaaatactcaaattcgttggcaacagcgatcatcttttcacgcttaaccaactcgtcaatctgcataacgccattttgggtagtcctggtagctagctcggtaactgtaatcctgtgtccaactagcattggcctaatgcgtctacggacaacattgacctctacagtcctaggaagaccgccttctctgaaagcagcataaccaatcttatcgtgcctagcagttacaacattgtattcgtgttcataagcttttgcctttacagcgttctttgaaagaagatcagtaagcggagtgtcaagaacattgagttgagttacttcaggttctaggtcttctctttctaaagactctgcaacttgaccagctgcattaagcgcttttagtgttgcttcatccaagtgctttaaatgttcgtatggcatatttgttttacctcctcttcaaatcatataatagcttattggctaattttgcgattattcttatattgtctgtaaataagctcaaattcctctggttctaaaccaaattcttcttttgcaaagttataaagctccattttcttctcattaagctgaattggtgttcccttataagttaggtattcttttattttttcttctaagcttttcagcttagccttgttagctagaacagactttagcttttcttgaaagttgctttcaaatgttcctgcttgagggtgaacttcagtatttacgccaagcctaggagcgatattgacactatttaaagacttggttgtagattctagtttttcattaacttccttgtaaaggtcttcaaacgaagaaagagatttttctacgctttcaattttttcgttataagattcaagcttgctaataacagactttagctctgcgaatttttcaaggctagcttcaacaatgtcaacaacataattaactctgttttctaaggaagccaatttagattcaacaaaagattttagctctgctagagaactagaaacaactgcatcttcgctttcttcttgcggcgctttagaagaaagcatagaaagaaccccagaaagcttttcttctagaaagtctagcttatcaaggatgtaattgaaagtttcttcatcttccttttcgtgttcttctacttcttcttgaaccaaagcaactttgtcctgtgtttcttcattttgaccttctaaagcgccctcttctttatcttcttcagaaggttcttcagaagcttctccttgtagatcgcctaagtctaaaacctgagcttcatcttgcttcatttcttgtttttcttcagaagcatcttctgaagcgtctgcttgaacctcctcttccaaggcctcaagaactccttcttgcttttcttcttctgggttttttaggtctttcgccatagtatcttcctccatagctttttctacaccattataaactaggctttttaaaactttaacgaaagtatcaatatttataggttgaccagtaatagaaacttcatataagcgcattttgggaggaacatttacatatactccattgataacctttgtctgaggtttgccaataggtatagctcctatagacaaaccaattttactagtaaacctatcatctgcagattgaagatgcttgtaaactttttgcgcaagctcatgatttttatctaacaatatccaaacaaaaagcttgttgtcataaatttctgccgcaatgggaattccaataggaacatctatatctggatgctttgaaggatttccagcgtgcttatagccatgctcatagaaaattcttcctgaatttacaaattcttctaaagaatctccaaaagtgtatttaacatctaaaatcgcatcattaactaaatcaatgctaggcgtagaagctacgccaaatattacccaatcaccaggttttagttcaagattttttctttttacaaaaaattcgcctttgataggttttatttctggaaccttatcagaaaaaaattctgctccactaggcgttgtgtaaacatttgtgctttttattacttcaaactttgtttctagtggaatattaagcacttctttcatagctttactcattatagtttttcgttatagaattttttagcttcttgcaaaagaagctcaaaaaaccagccttcaaaggtatcaaggtgaaaataatctaaaatttcttcttctttaatgtcaaacaattttattgtgtcaataaaagcttttactgcttcatcaataagtctttgtttttcttctttgtcgtctgtgttttcagcttcttgaagtctagatttcataaatagcgttgtgatggctgcaataagttgtttactcagcttcattttctttctcctttttcaaagcgagtttataatcttctattagtctttttattatcgggtagtaagttggatacttgtttttgaattcttcttcgccataaaggaagtaaaattctaaagaagttgaaggtacttcaatgatattaaagaaagaataaaagttcattatctcatactctttcatcatttcttctaattttgattcaaattcttcgcccatttctgatatttttttctgggcatcttttagaaagctgtagtaattcgagtaaagaacttgaagatcgctacttaaacttgttgatgttagttggattttacctagaatgtttttatctaatcctaattcttctgatgctctttttgtaaactcaactattcttggcctttgatgtctaaggaaagtcaaaacaataaattctgggatttcttcagacaactttttgatttcgctagaatcaaaattttttctttcaatttccttttcaataatattttctattgtatcatattcgtctatttctacatcatagtcttctgatgagtagttttctgttgtgtcatcttttctttgtaccaaatcttttatatgacgatatgcactcgctaacgcatgtcctatttcgtgcatcaaagtagcagtaaaaggcgtaactacttttatatacctatctttttcttttttctctagtgatatttcttttttatcctcaataattgcgtcaattgttccttttttatctaaagaagcactttcaagcgatctcaacatcatgctaatagacggcctttgaagatgttgttttaataggtaggctataggattgatattaattgtgtttcctgacgcataataccaaccatttggattaatgacgacattgatatacttttcagatatttctttaacaaaattaaaccaatcttcgcctaatataggcttcactttgtctaaaaacttactaaacattagagaagacattggtgtatcaaaaggagagtagtatcctaccatggaatctacaaggttttgcgcattgacagattctggaggtggaggagttgttcttatactttcttcatcagataaagcgattgagctttttccttctgctgaaagttcaattgcattttttattagaagttctacgaaacgatttttttctatttttccttcaaaccacttaaaaaatttgttgaaagttaatctttcttttaaatttccttttgtttcttctatgaagtagttaaaggattccaattcctgaataatttctttttcgctttttccaattgaatagaaatattcaacaagcgcatgcatgtatacatgaaaacctaaatctcttactgaaactttatagacattttttccgattctaacttttgctgacaattctagtcctcttatgtcgtcattagcagaatagtcaacaacttcaaaatcttgcaaagacagccttattttgtctaaggttttcatcttttcttttgatgaaagcgcctttcttagctcatgaattttgcttgaaacaaagccttctaaagccttgcttatagcgctcttatctcctgatagaaaataagctcctcctctaccgatgaaagctaaggtcatcgcttgaagatcgcttggagtcaagaaatttggactgtttaacttgtgagtaaatatatgatccaaacttcttaaagcaagctccttaggaagaatttctaaaagtccatatgtttcaggagttcttttatctaaaagttcttcaagcgttttttcaaagaaaaattccttcatttcttcaatgtcaattagattataaaaatcctcagaattctttcttgaagaaaactcaggagaagaaattctcacttttaatacaggttttcctaatagatttacaactcttataaagtctaatttttgatcgttactaataaagctagtttcattagcgctgtaaagtgaaaagatgttcttttcttctttttctgcgtaaatcaagtcagagttttctgatagtcttctcatcgcatcaacaataatcgctttgtatctagaagttaagaatgcaggaaacctttttggtttttctttttcaaaaattccaaccaaggagtttgcaatcagattatttctcatagcaaacaaagatgtgtagatttttttgtattctttttgtatcttacttatctcttcgctaaaagtttctaaagcatttttttcgctttcaaaaagatacattaaatcttctcgtatgaaatattctaagtaatacatcattgattctagtgatgcgatagttctttttgtaattcttctttttgcgttaatgtcgtttaacattttgtttattgacgatacaacattttctataccttttagaatatctgctttttgttctactgaaagagatgaaatacttctgagtagatcagtaatattttgataagaatttagaatcgcctcaaaatgttcaccagaagtaatcttaaaatattcagctagtgttgaaggatttgctaagtgtgtaagaagatttgcagcagctattctatacatcaacttcttttcgcctgaagaattaacttctttttcaataattttatcgtatgcgtaaatcatgtaatctgtaaagtctgctactgcactaattgcactaagattatttggttctgcaacataagacaaattttttgtagaaatatctgtaagaaactcaacaaatccttttataaaatcagctatctcaaaaagcttctctggataatcttcatatttttctaaaagcgcttttattattggaaaaactttttctgttacaagagaatcgcttttgttatgatacctgctcaatagtgtgtttattatgtctacaacaaatttaagctcttgtatatcttctgaaggttttctttgagaaaatatttctaaaactctataaagattgttgtcttcaagcatttcttttatttcttcaggattaaactgtatttctctagccaaaagagaagtgtcagtatatttagctagctctggagaaattaaagttaaagcttttgcgtctgcaagaagatcagctaaatctgaagcaacattgcgatcgctgtaaagatcaagaaaaaatcttcttagataggttgatgcaggtatattttctgaaagtaagtttatttcttcagaaaatacatgttctataaaagtttctttgtaagcattttcgctaggcaggaagaaagacaaaaatagaattagactatgcacatcatcttcttttaataaaggctccttagaaacaaacgaaatgacttgctcagttgcgaagtctaaaagtttgtctaaagattctttatatcttgcctcatgtattgctatttcaacatcgtcagaagagtcgtataattcttctaacgaacttagattttcaatagcttttactatcaagtcttttagcttaaaataagaactttcttctgaaaatttatctaaggacgcaaacaaaacgagcgcagacattataacttcttctttagaatttgcaaattccttaaaggctttttgaatagtaagaggagccaatatagctttatctagtctaggtgttacttctagaagaacttcaaaaaaagaactcagatagttttttatgttttctggagaataaaggtcaaatccataaaattcttttcgcaaaaaattgtttacatcaagcattacttttattaggctttttttagacattaactcttctactgtaagctctgtgagcgcttttggagataaagcgtaaattaaatttctctctatgtcgtagaggtctatgttagcttttgaaaagtatattccttttaacctgtctttgtagaggttataaaaggacaaaaaccagttagcgtcaacatctaattgagcgccgttatattttgaaataactaaataagtgagaacatttaaagcgccatcagaaatatcttttaagtagttagacaaaaatttctttagctcctgaaaagcgccttccttcttaaatacaaatgttatatcttcttgacctataaaatcgtacttatttaaatagcccctttctagcaaaaatccgattgctaaagaagtcattaaagtgaaaaatcctgatatctgtttagttccactaggaacttcccaattttcatcaattcctaaaatattgttcaattcaattagattgccttcgctagcgtctatgtcaaagatgctttcgtctagcttttctataaattcttccaaatagctgctttcttcttctttttcaggttcctgaaaactttcttctctgtagctttcatagacttgttgtttttgcgcaatttgatatttttgcgcatatttttcgtattcttcgagtatccaatttgaatcaggttgattaacgataacgccgttcttaggtttaattacttgtccttcaaaaaatacaaaagtagtaggatagtctttgtgataaagaacaaatccttcaggaactctttttacaacatcataatctttttctgttgtaataggaccagttattgaataaaaagtgcccatattcaaaatttattttatcatctaccttgttatctctacaaaagcctttataagttcgtctaaaatgaaggactcagaagcttctatgccaactctagacaaagcgtcttttactattttcttgaaatcttcaacgactgaagttgataggttgtttttatttattttgtattgataggcatctatcttgtcaggaaaatctaagccttcaatcaaggaagacaagtcttcttcgcttaaatcatacacagcaacaattttaggtattagcgatttcgcaagcttttctttaaatttgtttccagaataactttttataacatctttagcaagaactctaagctctttgtcttcaggtctttcttgaagcacgaattctaaaagcgcatcaagaatttcatttgtatcagaatagtaaattaacctcttgtcttctgaaactctagctttttctttttcaatgttttctgcaattttttcagcttctttgaggagttcttcatctttttcataaggaagaataggaaggttgaacttttttagcacacgatttgtcagactaacaatcgctttatcaggtgaaatgttaaccttttcttgttgtataaatccttgctcatcttctgtctgaattgttgtttgtattccttcttctgttacattcacatctacaacatcttttttgcctttaggttttttcttagattttgatttctttttttctttctttcgattttctttttttgcttcgctagatttttgttgctcttcttgttgagcttcagttgtttcttcttcttgtatagattcagcttctgtattaggacgcaaagtatcaacaattgcaaatgcttctgctaagaaatctgaagtcctagaatacacctttgtcttttcgccttctctgatataataaaatcctctctctcctaaagattttttgccaaaagaaaagttttctagtgaaaactcaaaatcgctttcaaaaagcattcttgaaaagtaaggaagatagccttcaacattaggattttctaaagctgcataaataatgagaagattgtttgaaaacagaggattatcagtatcgtctaaagctaaatagtgttcgttttcttcgctttctgcaaacttgaaagtagctacttctggtttgcccttttcgtctgtgatgacttcaagcgatatcttatcagttaaaggcatttcaatatattcgttagattttgatattttcttaaaaagttctgcatatgtttcgcttctaatttttcttttaattctatcgctaatgtagctttcatcagaagttctttgaagtattgtttgagcaacgcttctagctaaaggattttttgattgcttgaaaactttttcagcagttgcacgcaaagattctttctttgttgaagatgaatactcatctagaatttcataagaaacatcaactaagcttttaaaatcttcgtcttcattgtaagctttttcaaaatctactagataagcgtatgcgttatctatacttacaaagtaaggcgacactttgtctaagtttttaggattaaagatgccaaagtaaattaaaccttttatgtccttttctacttcaggatcaaaatcaacagcttgttgaatcgcctcagcaacataagaaattgttgaaggcgaagaaaaaagatttattaaactagagtagtactcttctttttcatcagaaagacttttttcaacttcttcaacagcttctttattttcttctactaaaacaacctcgctagcttgttgttcaaggttttctttagcgaattctttagctttttctctttgcttatcagaagatgtcttttttgtcttgtcggatttagcttttttgctagaggctcttgtttcagtttttctagcttcttcagaagcttcttcaactgaaggagtagcttcaggaacttcctcagtttgagaagctgtttcaacagcttcttctgctcttgtttcttcagaaggtgtttcttctttttctttaattttctttttagctttctttttagacttctttttctcttttattgccttgttaagttgttcaatagacgaatttagaagcttttcttgcgaagttgcttcagaaatagtttcaaaagcttcatcaactgcttttttaaactcttttcctaacttctttaaatcctctagctgaagagttcctataattttatcgttagatttaaatgcaaaagatagcttattgcctatctttttaatttcaacttctttatcttcgtctacataaagagtcaaaaattcgcccttatctagtgttttctcaagattttctaggtcttcaaaggcaacttctttataacttactttttccaaaggagcttcaggtattgaagatgtttcctcagttaaagaaggctgctcttcttcgccaatagcaggaatttcttcttcaaagacaattggaggagtttgtgtgtgtattacattaggtgaaaagtcgtcaaagagatataaaagattttctttcggtacttttgtctttaaaatgttgaaaatcgcatcttcataatcttcaaactcactataaggtactatgtcttcaaacttattggagaaagaatactcttttgtgccgtcatatttaccaaaaacttctttcatgaaggtattgattttgtaaacataaggcgctgtgccaaatacaagtgtgtctaggtcttcttttgttgttatgtcagtaaatatgcttgaaatttctctcaaaacttcatttaatcttacattgtcttctacgccttcaatttcttgttcaagataagtttcaagaggcattcctttggcttgctctatctcgctaattttcttagatagttcttcaacatttggattatattgatcaaaaagatgataagcaacaattgaggaagctaaatctctaaatctgtttacaaagctgtctaaataagattctatttcccactccttgtttggttctgagtgattttcgtagactatgtttccaaactcatcagtttttaaattttgcgcattaaaaagttttccttctttaggtagaaaagctaaatttctaacagactccttgactttacttaaaagtgttgcgttaagtctgttcgtgcaagcttctttagattctccttcagcgcaaacatagctatctattaataccttcaaagcttctgcaaaaacttgtttatcaaaatctctaacgctttctgattttgctttcagttgcgttgaatacctttctttaactgcatctaagtaagttgaagcttgatataagaggccgcttaatgcacgatctgtgtcgtcatttaaaactacctttaaagttccatcaaacctatcaaacattcctgcaagtagcgctaagtttgctaggttttcttctacatcacctattcctatttttgtttcaggtgagtgattaataagttctcttatagcagaaggaagatcttcaaatatttcttctagtttttctggtaatcttatgctttttgatagttctttgatttgctgttcttcaatttgtctttgtatttcagcttgatttaagagttcttcatactccttttctttttctgctaacattcgcttgataagcgaagatcttactaaaggattaaagatttgtttaggaaggtctttttgatcagcaagctttattaattcacttgttgtgtgttgtaaaactttgtttatatttggtacaaatctaaagtagccgtgaacaagctgagtttcgtattggggaagaaaagcgtcaataaagaaagctaaaccccctagcttattttcacaaaggcttgatgcgcttcttgcctcctctttaaaggattgatatcctcttttggagtattctaagtccatgctgtaagaaatgcaaaggtcttgaagaatttcataagtcttgcctcttttagaaaagtctttcattaactctagtgcttgatagatatctaaaggttcaaggctgttgtttaaagccaaaaggcctgcaaatgtatttgctaaagcagtaagcgtacgctcaaagcctaaagattctacatttttcattaaggaagaaaaattgaaatcagcaaaagattgtcccaagaaaaattgtgttatcttgtcaatgtgctctttgtagtcttcttggcttttgtctagatactctatgctttcttgataaagtttttcaggcgaagtttcttttggttctaaaactttatcaagcgcttcatcaattaactcttctttaatttcttcagacaagtcttgtgcgctagcaaaggtttctttaaattcatcaaatttttctttggcgaggttaacaagttttcctaaatcttcttcattaaaaagtcctcttaattcaaataaagcttcttttactttgtctaacgcattttctaacttttctagattgctttctttgtcttcatctaaatcgttaggaaagaattgctctacaaattcatcaaatttgctttctattaactttttattcgcctcagaaattaggtcttctaaagtaaaaaattctttaagctcatctaagatttcttttgattttttatcaaggctttcataaagatcttttgcttctgaaagctttagaggattgccttcttttaatctttcttctgctaactgagaaattgcttcaagaagatcattttttatcgcttgtatttctagctgcctttgattttctaaaacctcagcttcttttgtagaaatttctttgctttctagttcctctgatatttcttctaaagcttctgaaccttctttaagctgttcctctaaaacatctactacttcttcagttttttcttctgctaactcttgttgtaattctttaatttcttcttggcgctcaacagaaacttcttctttttgctcagtagttttttcttctatctttgtttcttctggtgaaatttcttcttgttgcaggtattgagaaatgtctatattgtatatcttcgaaatagtgtttaattgttcctcatctagcgcattaccaaatctgtttaagtattcttcaacatctactaagtgccaaccgttttcagtttctattgcgataccatgttttgtagttttttgtccttgaaaatagacgcttctagtaggattagactccttatataaaacctttccttcaggtactacctttaccttatcaaaaggtgagttatcagtataggtattattgtaatcgtatacaaggcccatatctgtattctactattcgcctttttcttcttctggtatacagtagatttctcttaaagcagtaagtggatcttcatctacgccatatttgcttagatatttgatagccaaatctaagtgataacaagactgaaacttatcatcattttgctcaaattctaccaaagcgcttaggccgctaagatattcagcgaattttttaccgctataatcttcctctaaagacggtgttttttcatacaaaacattagcccattcccaagataaatgtggtaaaaacagcgataaaaattttcctacatattcttttcttctttcagtatccatttctttgcacatttcaagaagttctttgggcatttgttcttcgtttgtcatgttatcctccaactacttaacatctatcttaacaggagtaatgatgttagataaagcatcgactacttttctatagaaggcttcttccctttctctcataatagcctcatattcttcttcctcgccttgttcaagaataggtgtattttgagcaagaagtttttgtgtatcaaagagttttttaacttcaaacgacttcaatcctaaaacagatgcaacactttgaggaagaataattgacgcatctactttacctttttgttttccaccggggcgaaaagctctacctagcatttgcaaaatagtttcgtaagagaaagtagcaccatgaacaacgattttttgtgaaggcatgtttacgcctctagctaacgctcttgagagaacagttatagaaggcttgttgttagcgttatgactttcaatgattttcattctatcagccatgtcagtttcaccagtaactgctccattgtaatacctcttaagcgcctcaacaatgcccttatcgccaaataacggtatcaaatttctaaacatttttcctaaaggcgtattttttctaatattgtcaaaaacatagtctaaagactttacaaaagctgtgctaggagaagtaattagaatttttcggcctaaatcttgagaagttttacttgatgttgcgaatggcttcaaagcttcttctactactcttttaaattgttctgtagttgcgattgcgttgtaagaagctttatcaagctctttaccaatctgttcgccaattgtatctagtgtaacatcgttaccttcgctttgtttaatttggaaattttcatttagtgttccatcttcatttaaaaaaggcgctgccttaggatcgtttctatcaacaagtgtttttacacctgtttgaaggtcttgcttagctaagcgaattctaaactcaccttgggctgtgttttcaattactggatagtagaggtgtttatcgtcagaaagcataggtgtaataaagaaagtgtatttcatcattttgtttctcgtttcaatagaaatctttctttcgcttcctgttttttgtttcctttgctccaataagccgctaacgcctgtccttcttttcaaggttccagacatttcttgttctttatcaacctttttattccaagcataaccaggaaatctaatcttaaatccctttttaaacttttgttgtctattttctcttgaagcattaacatctacatcaaactcaatttcagcagaaccaaggccgtgagttactacattttcaaatcctaaactaagcaaatcagcaaatgtatattgtgtgttgcccaaagaaattacaggaacttttgttgccaattcaagaatttctttctttaactttgttttcttttcaccttcagctgtcttgtattcctttattttgggaattatgtgttttaagatgttgttgctgccaaatctgtctaaaatatttggactaactgacgataaagtgtcaaccaagcttcttatagcaatttctctaactgaggcatttttaggtttattttcttcgtcaacaattatgaatggaccttcttcatcaaagctagtaaaaccgtattctaatgcacgttcccaggtaaaagcaccaccttgtacataactaaataaggtagcaaaaccaggcacatacatttcatagaacaacttagaatctactgatttttttgatcgtgtaacaggatcataaattgttgtatctacaggtctgtcttctttatataacttagctatttgataaatagttgaagctacagttaagaacatgtcttcaaacttttgttttgctttttctggttctctaactaaaggatcatcagaagatagtttttgtggatcagcggatgaagtaggccatgctaaaggatcatcagtttttatatcaagagtaattcctttaagttcaaggttagtagcaccaacaacatcaagataagttgcagtagttttaagcatgtcttcattgattattggtccactaggtgttttaattacaggatctccaagaattcttttaaataaatgtccttgatagaacagcatttccattgctgaagaaaaatctttagggctgttagacattgcagtagcgttaagtagtaatgtataaccacgatttgcgtcagaagttatagaatctgataaatcttcaatgtgtcttcttctagcagaactttttgagaaaattgtagaagcttcgtctactaacaacattccaaacattccgaaaggaactgcaagtctttgtcctttgtgagaaataatagaaggaccagaaagaaggtttaagaaatatttgtcagcttcatcctctgcactagcagattccattttttggaatgtagaaagcttcatgattaccattttgggaagatttactttttctcccctgtttgctctggcccacaaatctagcagttgactccatttttctcttctttgctgaggattgccttctatgacaaatatttcttgacccactttccagccaaacaatgtttcagcgtcatcaatccagccttttgcaatggattcaggcattactgctaaggctggtctttggtcaaaagtaattgttttgttgtccctattataagaaactgtatgaagtccgagatttgttgctagtgcgtacgctaaaagtccagtaaaagtttttcctagaccagtagcgtgcgcaataaacattcctactttctgtaagttttctaacttagaagtgtgtcttagataatgaggatgtaaaaacttaagaagcgcttgaacttgataaggctgcaaagaaattttagaatttgcgaactttattctttcgttgtaggaaattggaccaagtttaggagaattatatcctataatgccaaatttctgaagcagtagcggcatccacattaaaggaaacttttcgccaaagtttcctttaggaacaggtaaaatttcttttttgttgccgtcaattcttattagatttcctttagcatctttttgtatgtttaataaagaccacaaatcatcaagttcttctttaaacttgccccctttaacgaaaagagaaaatagctttctatgataatctctttcctcctcaggaaatagtgtgttaataacattaggatcttcttcagcgctaggaatattttctcttttacctctttcaaaaactcttgcagaaatgtcttttttaagaatttttgcggcctcttcaatcgtagaagaactaacttcttgataaaggggatgaagatatttttgtttaccaataggaagaccaaatatttttgcagtatcatcatctagcttaatgatgtttcctgttggtatatctaaaacaccagcaggtattattgcattactgcctaaaatttctgccactaatatatcgatatctctttgtgcagcgcctcttttgtttaaaagttttggattttgtattatttcggtgagaagattatcaagagctctttcgtattcttcactatcaattttcaacaatcttttcaattcagtaaacaacgcttcattactagcaccttgagaacggagtttgaggaggttgtcagagaagaaaacctcttttccagaaaactgatcttttgtagctagataggttgaaaggtttgtaagatacttattctctggatcgtacacataaacataatcaaggttgtttttttgattttgtgcagaaagtttcttttcttgttgcagatacttatttgcatcagaatctactgcttgaagcaagtctttcaagagctttttcttagcctctgtaattcctctaaaataaacaccttcttttaaaatttcgtcaataatgccttcgtcttttttatttttttgaagagaagcttcaattagtccagcaacatcttctttgctagcctttcctgcttgaaccttagctagcgcttgtgctgtatttttgtttgcaggtatgagcatccaaggtttaccttcttctagttgttctacttttacacctaaaaattttcctccaactaaaacacgcccatcctcaacattgtaagcaatataacctgcaggaactttttctacaacgcctttttcgttagcgaattgagaatagttgatagacttagtagtagctaaaagaagaatatctttgcttctttcctgaaagctaaatagaatgtcctcaatagactttatgtaattttgcatattcacaccttcctcttctgtttccaattttatatcttctaatagtgagagagattttagcgttctgtctgaaattttctctactttttctattttaaatccgtgccaaacaagaatttgaggcaagataaaaagcgcctcttctgtttttaattctacgccaacaaaaacttgaatactaaagtgtttgtttcttttcgctacagaataaaaaatctctctttccaatatctgttctaacgcaaacttaatgttttcattgattgccttaatgtaataatcttgtattttgttttttgctagtataaagtgaatttctgcttcgttatctccttgagaaagatgatatacaattttcttcataaatcctccaaaaacttttcaaggtttctcatgtatgcaaagactttgggaaactgctgcagatagtttctatgaaaaaagtaaaaaacaaaagcttctatgaaatcttcttttggcccaagtgtagattctaagctgataatttcatcgttttcatggtataaatttttactcaaatttaggccaaaaaactctccaaccttcaaatcttcgtcagaaagtttttctcttttccagcccctcaaagatataaatctttcaatttgttctggaaaagaagaagaaagcttttgaaaggttatatttgcaatttctcttgcagtagccaaagaagctactgaaatgccctgagtaaagttaggtactcttctaactacaggtacagtcaagtaaaggtagtaaggttgaaagacatgaagataaattttgtcatgggaaaaactagaacctataatagctaaatcaggatcaaagtctacaagttctctaatctcacttttcttatgccaataaacttttgtgattttaaagagattaaacactacttctacttgcttaaaaacagcacgaagatttaaaagcgcataaaaaggaattacattatctactactagcgccgcaaaactcaaaagttcttctttgtttagcctagcaactttgttttcatcaatgtttccttcgtcatcataaatatcaatagtaggatcataaaacaaaccttggtcttttgcttctaatagccttagcgctgataatgtggaatcgctaggcgaatattgtcctacttcataaaggttatttgaaaaaggaactgggtaattgggggctattaaaggattatcttcactaattcttgccacaagactattttaacacaagttaaaaaggtttattttgcaagaaattaaggaggatatacactatttatgtctttaaagtacaaaaacataaaattttccacaaaatccgattcggcaaacatttggcgcaaaacaatagaatttcttctgttgaagtagttatcttgataatattcattcgacgaatcaattgtgtataacttaccagcatctaaaatttttactttaacttcgtttgatggatcgtttgtatctacatataaagcaacagtaatgtaaaattcatctacgagaatacttccgtatctttcttggttttgattaccttgaagaccaggatttctagctacaggaatagtaaaaacaattccaacaatacaatctttataaaattctacaaaaccaacagaaaaatcattacttgttgtgtatatagatttgtaaaaatgataagacaaacaccctgtataatcaatatctggatgtttagaagataaaattgagcaaatgtcttcattaaattcttgttttacaactaaacctggtgggtttccaggagaagaagactgctttttgtcttcaaataacggaagttcgctgttgtgaatttctatgtttcctgaagttgccatgaatgtaaagcaaccgctctttttagccgcatttaaaatatactcactattatctcttgtaaaaattaacacactaggcagggaaggacagaaatccaagtaagaatttccccttagaacagaatttgttgatccaccataagaagacgtgtcaaaaatatcactaaatcttggatatgcaaaaataactaacttattatttttttcttttgcaaagaacaaatttttcttataataaagctcagagtgcaatgtgtcatcataaatatttacattattagtgtatgtactatttgaaccaagagaggccaaataagaaaatttgtaaaattcttttggattttctcttacaaatggaatataatttgaaaaagaaaaattaaaattgtcatcaagactaacaactctgatgtaccccaagtaaagagttatagattttctgttaacagaggtagaaacataaagagaatatggttctaaaaactttgtttcatttgaatatatcttgcgaatatatgtgttgttaacaaaatatgtatatacagaatagtaattattgtcataaacatccaagttaatagaaaaattttccaaaagataggaagtgtctttaagatgattttccgttcttgtttctaccttaatcggtgttatacttggttgcccatttgaaagaccaatagaaaaaacatgataagtataatctaaagtaaaactttcactattataatcaccatgcaaatatgaagccaaaaatacattatcttttaagtgagtaatccccataaaatttccgtgaggtacttgcggatacagagaaagaggatgataaatcccctctaaagtaatactacttgaagttattttgtaaataaaataaatgtcatcgtagaataaataaatagaagttgaaccaccttgcttttcagcgtaaataaggcccttttcaacataataaaaaccatcctcagttttaaatattatcttttgagtgtcatcatttgtatagtaatactttacaataaaacctgtttcatttgaaaaaaatgggtgggaataaaaattggttatgacgctcagtaaatcgttaatgggattgtatacagcaacagcttcaagacaatcatttccttctgatgttgtacatccatcaataagtttgtatagctttaatctgtaattaacgccacctatattagatagataatttaagtattcagtttgcaataaataaatttcaccatttttttctaaaaacgctcctgtctgataaaatgggttccaaaacccttcttcagaaactcgccaataagttaattctgtatttttttgttcactttgtttttgtatacaatctgtagccatatcggtttttattatacaaggaaaatattgttacgcagcaacgccacctccatctccttgaggtagttcgctgtaaacatacacagaatagtgtaatcttttttgattatcaaagaaaatttgtggaatatgtacagaagctcctctattagtataagtatcactaggagaagctacataaagcggtgcagcgtctagtattttatattcttttataagattaccttgttcatcaaaagaaaatctgaacaaaacgataactaaatattcttctattggggattcgtttggagcagattttaaatcgttttgcgtattcaaaagcctaaaagcacctactatcgataaactacacccatcactaaaactttgaactgcatttaatagctgaataaaataaccaataatgtttacgaaataaaaccctagagaagaacaatcagagattgcatatttgtaagatagaatttgacatatatctgacttcgctgagttatttacaaatactccataaggagttgttggatttgttgttttttcttcgaaaattggcaaagcactattaaaaaacttatagtaaaaatccaaattgttgtcttcacttacaccagtaaaataattttcattaaaaacaaattttagatttgcttcatcaaagtcatagataacaaaggttgctggaattctaacaaaagtatagtgcgacattgctacatttgttttaccaatatctggtaaatcttctatcgttaagtaatcaatcattcccaccagaacaactttgttgccaattttgaaaaacttaccattaataaaatcgatatggttaacatatgtattacttacattaagtagatttgtaagaagagtgttcagatcactataaaaggctggagtgtaaaaatcagatattgttaaaccatctcgattggaaaatttaataacttttaatagtaatggttcaagttcgacaccaaacccactagtatcatcgtaaaaataaaagtaaagatacgttcctagaacaaacaaatcatcagaattatgcacagcctgcaaaaagtagctgtttacttgaaatgtatatggatccaaagtgcttattccataatctgaagggtttgtagaaaatgacaactgatgttgtgcaagtatactcttttgaccactttgcgtatctaatgaatacatcaaaagtttaggcataaggtagagatctgacgtagagcttactcctgcatataaaaataagctatttcctttatacttcaaaaatccttgttcatatgttgcgctatctggtagacaacctccaagggctgcatgttccaaaattggaatttctattctttctaaagtcattgaattagaagagattctaaaaataagatagatatcgaaggaagataaaatgtaggtgttataataacccgtagttctaaacagggaattttctactttaataaccgccaaatattcatcagaacctacttgttgcagatcataaaaatttataggataaatattaagaagcttgtaaatttgctcaccaccttgaacgctgcataaatactgtgaaagagcgttaaaaatttcactatctacataagttaagtcaattgatgatacaacgctgcaggtatcttcataacaacttgtttcatctacaagttttgcctcaactacatagttttgaattacttcatcatatcttcttgcaaaataaaaaagatagtttcttccgtttataggaaacttaaacgaggaataaagattattttgattataatagtaaggtagcaactcatcaataaatccgttaaagtttggttcttgagttctgtttatgtttgcgcaatctattcccataagtactattttaatcagaatgttttagcaataatttaaatttgaaaaagcgtttacttacatttcgctctcactacatatataaacttattcctatttcgcactttatccaaaacaaaatccgtttcaagttattgccaaaaaacgcaatatatatggatagcgtttagctcttttttgtatggagtttgtgataagaatgtttctcaaaaaataattaggggggcttgtttggccccccacacccctttcctcccttactttagcctataggcatattctgttgcgtaagggagaaagggcacctcccaaacctcaatgtattctgcaccccttttcttaacgataagggcttctgcatggcgatttatgccgatagtcttgagaatgttcttgacaagcatggggttaagaacaagaacataccccttttcgtcccaaaattgtccaatgggcttcttcatcccactcacctccaccttcataataccctacttaatcctttttgtcaagccccaacacttaatcaccatgcgtatcatgcgtgtattcggcatatagggagggcgcataagcttgagtgagagtgtgttaggaaaatttacacgctgaaacgctaatgcacaaaatgtaacttttttatttttcccctagcgtttaattttgttttaaaaaaagattaaggggggccaagttagcccccccactacgcctatcgctacttcaagcgataggcaatatcgtaaagcttccacccaaaggtaccccacacctcggcgtaatcggccccagaagaatcaagctttacaagaagataggggaagcgctcacaagtaccaatatactcaagtacggacttcacatcctgagaatcttgcaagaaaatgtagctatcgtcaaagtccctgagaagcttcttttccatcctactcacctccaccttcatcataacccattaatgactatttgtcaagcctatctgccaatagcgtgcacctaacgttgaggtaagtatgaagtttgtgttggggaaaattgcttaaagcgtaaacgcataaacgctaaacgcaaagccacagaattttgtctgttggagatattaatggatagcgtttaattgtttttgtatgccccccttcaacgggggggcgtgcaagctaaccttctatgacctctcctccgatgaggaggaatactattttcccgccccttttatccatagcttctagggcgaatcctgacaaccttatttcctcagcgtacctgatgaagtgtagaagattttcaatgaacctgatgactttgctcatggttcacctcccaacactaagatacctcttttagtacattttgtcaaggggggagtaaaaagaaaaacctcccctttacggggaggttaaggttaggggggcttacttaaactccttcacataaggagcttgcaagcccctctgccttattaacaaaaccctatcaggaaacacagcgagagagtgatgaacaaagtcaagcttgcttagagtttcggagaagtccaaaagaaggacttcaccaaagttttcatcctccaagatttttaccgagattccgagattgagggattcaaggaactctttcaacgcttctttctcttgcctagtcatgctcatcacctccaccctcatcttagcaccagaaaataagtgcgtcaagaggcattagcgtttacttagcgttttaaaaaagaaaaacctccccgaaaggggaggctacacttcctctaccatttccactcaggatcggatatacatcacgctatccccgtagtaccatgcactaagtactttatctttgtcagtaactaccacggccactccagttggattgtgaagattaaagtagatgtcgtgtgttagaccatactcatcttcagcgatgatgtgtaagagatcgccgtctgtatagacttcctttacttttttcctgaccaaagacccgtttagtaagatgtttagtacctttccgaccatcttcttcacctccttgagccttcctcaggctcacatctatcttaatacctcagcacctcaaagtcaaggctccatagcgtgtacattatctatttctaggtgcgtatgcttgcgatttgcgatttatttacgctaaaagcgtaatacacgctaaaagcaaaataaaaactatctccaaaatactttttccacttagctttatcaaaacactataggttatcttttgcgtttacttagcacatcttaaaagaaaagacctcccctttcggggaggtatccttgtagggattttactctacaaggatcttacgaatcttgccctttctttcctctatgcgcacttttccaaaggggctgtcctcaaagccaacaagcctccagctactagcactagtaacgcggcattgatcgcacctcttgggtggtttggtcttagcttcagccacaaactcctttccacacttctcgcaaacgaggacgatctttttaggtgtcgggctccttttcgtcatgctagcctttgtgctctttcccatcttttacctcctttgtccccttgcggggacaccttcactatacctcatatccccctttttgtcaagacctatcttgtggggtttattagggggggcgctcaaagcgtgtagatttgaagattgtcgtgagattttgaagatagttaaatgtaaacgctaaagcagagattagggagagttttatgaaaataaacgctaggataataaaagttttgattttgagatttttatttttggggtttttgtttttgagattttttgatcattgggtttgttttgggcgcttgttttaaaatctattgaaagcgtttacttgtttttgcgcaaaataaaaaacaaaacattttcttttaatcttcgtaaacaatcaaaatagcattctttgatattcctagtttaatagctttctcgtaagtatcaactactatatctgcagattttatgtatctggagttcattgtgtcgtagacaataccttcaaaaattcctaaatctgagatgatttttacttttttgccgcaaagatgttttttacgatttttgtcaaagaataaatcttgtgatagcgctatgattgtttctttaactctttctattgggccacaagaggcaacatatggatcatcatcagtttgctgaggaactaagttatagaaggataatctatttagctttttggctagaataatattaaagtcttctttgcttttgtcttttatgtaaaaattctttgcgatttgatatcttttatgggcgattttttctttgatcacaaaatattgatcaatcattagaggattgaacaaaaacaggaaaatggtaaaaagttttttcatcttgttcctcctttgcgtttagttaccgagcctaaagaaaaaacctccccgaaaggggaggttttcttcttcaggtatgcaggttaactgacatacctgaagaatggcaacaccttggggtgaggaactacctcaatgattagtttttttccgtcaaagctagagatgattagcccatcgtcaagatataggtagacattcctatcccccctcggaacgacaaagaagttagcaaaaccattgtaaccctcaggaacccccctaagtgaagcaccagcgtactctacaaagatgcgcctaaggttagaaacctctacaaaggccctaacgctaagcgcatcttcacccttcttcacattcccaccgatttttacatcggtaggcagaccccacttctcaaggtacctcttgacctcttccacgatcttgttctggagtttttcttccatcttacacctcctcgcctgaggatttaccccaggcaccttcatcctaccacacctttcgtcttgtgtcaagccctccccctctccgagcattgacttgagattttcgtgcgcttttgcgagaaagctaaaatgtaaacgctttgggagaaatgaacgctgaaacgcttaagcaaagaatggtgtgtcttgttttgtggtaatattaaaagttttcttctatttcaaagagaacgcaaatataactcctagaaacgacaatgctttctgttttctttgatagtgaaacttgggagattaaaggaaagtatatttctttttctttggcaaaatcttcttcttttgccggataaaagaaaatgtttttgctttctattgtttgataggatctcgttattgggctgtaaaatttttgttgtgttgtttttatcttcatgagaaaaggttttgttgagttgatgagatatctcttttcgtcatcattaacatagtttaaatacatgctttcaaaaagcaaatctaaaaagtcttctaagccgctttctaaatcttctttatttggaaacatgtcaaaagtttttctgaggtaaaagaaagatttatttacagcttctagatgagagaaggggttatctagtcttctttcttttaactgaaaaagtattgatctgctaaaaaactttggttttctaagaaaaagaagctctttgttttgttcaaggattttttcaactgcttcataaatcattgacttaactttgttttggtcaaattttttgctatctagcaaaattagttttttctctagcctcatgtttttaggttagcaaagcgctaaagcgttgtcaagcgttatatatttgagatgacagcgtttacttattttttcgttagaaaaaactaaaaattttctcaaaacacttctcttaatatcacaaactcaacttctccaaagcacaaatatagcgaactatttttatcataaactccaaaaattggtttgtatattgaatatgatcggtgcgcataatccttttcagacttttcttttaagtaatatctaatcaaatcttcataatcttctatttgtataatgtcaagaagttctaatgttattcttcttttgaaatcctccacgcttgcatgttcttttaaagcaaacattcttttttctttccagcgatttaaagcgtgtttgtaatttttaaaattctctttgtattttgaatacgctttctcaaaatctttgtagtaagtaattttgtgagcataatgctttagagaaatcatcgggctgtagtgaggttgtgcaaaaaagtattttttggcctttacttcaaaaatcataattaaaccccttcaaaacacttcatgaattttcacatattcaacttctccgaaaaatacataagctagattacttttatcgtgaaaccccaaaatcggtttatgtatcaaaaagagatttcttttaaacttcccattcaaatatgaagttatcaaatcttcgtaagtttccattggaaacatataaagaacttctgatgttcttaaattttcaaagtattctataggttttaattcttcctctaaaacaaacatttttattccctcccaacgattaaatgcaactttgtaattacgaaattcttttttgtaggcttcaaaagcattttcaaaggttttgtaatagtaaattttgtaagtatatctctttaagtgtatctttggattgtagtagggttttacatagaaatattttttacccttgacctccaatgtcatgactcacctcctagcatcaggctaacacaaggggtgctaaaaggcaagaagggctttggtgtggaaaagggtattaaaatagggggcccaaaaatatgtggatagcgtttacttagttttggcaaaaataaaaacaaaaaataatttcctagaaaatctctagtggaaaaatttcaaaaacttctatatctccgaacaaaagatttctctatgtactattggtctagttttcattacaaacggaatgtttatgttcacctcaaaagtaaggaaaatagtcagggaaaaatattaagtctacaagtattctcttcttcggattatatctatgtgtactttcggcaaaagacaatatgattgtatcaggaactaaacctttcctatacgcagatattacctttttgtgaataaaaggatgtgggtttttaataaattcaaaaatgtttttggattttctatagttaagcctgaagttatagggtgtaaaggtaaaatattggtttagcattaaaaacgccttaataccaatatctacaggaatatctgtacttacatacttgtatccgctatatttgtggtcttcctttaatccgtatccttgaggattaagaaattcatttacatcttgagcatacttttctaaaattttcttaaagtgcgaactttttacaatataaagttttgcgtaagtgctgcttgcaaccatgcttgtaccctatcacacaccaggggaatagtcaaggggggcttagcgtttacttaattttgggtaaaagaaaaacctcccctttcggggaggcttgtattcttcttgctattcaaccaagattttcctcactttaccattcctttcctcaaccctaaccttaccatatttagtatcctcgtagccaatcactttccatgtagtattcttgcggcaatcttcacaatatttaggaggacgggttttgctttcggtaacaaactccttttggcacttttggcacacaatcaagaccttctttacatgaacattacggaccttcttttcgttcttgcccatcttcttcacctccacccccaagataccacaaagaaagatgattgtcaaggggggattgagaagattgacctcggttgggctggttttgcgtgaacttcagactgcaaagttgcgaagtttggaaggggattgttaaaaataaacgcttaatcataaattgtcagtttctttattttcttctagttccctcaactttttagcataaaaccactctaagaagatgataaaaagtgtgttaaataggagataaagaactaagtcatttgtataaatcttttttgttatcatttctgtggcaaaaagaaaaataaattgaattacaatgaaagctaatattaaaatccttaaaaggggaatatctttgaaatctggataagttttcaaataagagtcctcagcttttttgccaaaattcatcatgaatatcgcaaacaaaatataaaattccttttcgcttaaagctaataattttgcaacaataaaagaaaatgcgttataaattaaatagaaaaacattgacattcttattgattgtatttgtgcttcatgcttgttcacttttcacctctcagaaagaaatgttctttttaacaaggtaagcgttagcgtacactctgaaaatatcttctctttcacctttaaactctacttctaacctaaaaatttttattggcacaaaaagatacgctggtattctttgtagaatttgcaaatcattacatgctttaaaaagctctttaaaggacttaaagaaaaatgcatggttttctactctttttaaatttcttgaaatttctaaaaaaaactcagaatcagcagatccttttactgcaagtgttcttgctacactagcgggataaagaagccaatatatgtctttcaaatccacactttctaccgaaacaagtttaagattttttagttttaatatttgcggccctaacccttcatttagcattttaaaatctttttctatttgagaaaaatcatctatgaaaacaactttgttgctaacgattgaagccaatttttcagacattttttgaatatagtctttttcattaaagactgcatatcgtttatgtaaaacatctaagaatggtggtacttccatttaggttcacctccccccaagagggagtgtaacacaagcctaggcgcttgtcaagagcgcttgataaatgtgtatatattttgatagcgtttatttgttttatgacaaaacaaaaaaattccttaaaatatctcgttaaggctatacaaatacatctttcctagcattgcagaaacctctatttttagagaggtttcttcaggttttgtattttcaaaggttatgtaataatcgggaataaagatttgaccttttttaaaatcaaagatgttgtcaatagcgtaaaagtattttttgcttacaactttaactcttattttgtcgctataaattccatttattccatttgctggaacagcataatctgctatgtaaaaagctatatcgtttttaaaagtttttatttttcttggcgcaaatttgtcaagtagcgattcaaaaggtgcggatctatagtgataaagatttttaacaaataaagttcccaaatcaagaattgtgtctatatcatatttgtcttcctcaaaaatcatcattggaagccttttcttttcattaaagtcaaactttttaccattctgagtaatgtagtatctgaagataacaagaacttcatcagaaaattttttctcaaaaacttcagtatttacgatgtttctaatcattaaaacacctcctctattttgtattcatacaaaactccaattaatccttctatcattccagaaagttcatctgtatctttcaaaagtattttcctttgtgcaacaaataaagtatcgccttctattacttcttttaaatcttttatctcttctatttgagaaggcctaagataaacaatagacattgtgtagtcatcaataacatgttcgctataatcatcaataaaaaccgagaaatactttttttcaatagctttgctacaaacaacctttgtgctgaaagtcttcaacaaatctctctcaagagataaacatgacaagttgttgtaaaaatactcaattgatctgtaaaaagttttgttgtctacgaaatcagaaataatgaaagatttgtacaaagaaaactctcctactatttcgtcagcaaagcgattgttttcgctaggataatagtctagttctagatacgctttatatctcttcaagattttaaaggactttgactccaagaaaccttctactgctttcattctaatcccccccttcttaaagacataatcatagcaatgaaacttaaaaaatctcttcacgatgctttactctatatattcttccaacaaaacatttttcaattaattctacccttttaaaccatctgccttctctaaatgactcaacaacagaatcaacaataagcaagtaaactgcaggataaagcatttctaaatctgttattctctctcttatgtaattcacttctgaaatcttcattctatcgtaaacataagttgtgtgaattactcttttgttagaaataacctttttctcactaccactatgagctataatatagcaatcatactcagggtcaagaacttggtaaaaataaatgtatttgcgtcctatcttgtcagttgttagcttagatatctttttcaagtattcctctttgttcaaaatcttcacttctctagatatgccgctattagagaaaaagtagttggcatctaagatttctaaaaaaggcaaaaagtttttgaggcttttgtaatcttctagttctttaataatgctttcccccaccatgacacctcccctcttaaaggctactgctaaggccctataaagtcaagaagagagaggcaaggctttatgaagcacaaaagctaggggggggaagatatatttaggatagcgtttacttgtttttaacgaaacaaaaatcaaaaagcttcatttggagtgtggaaatgtctataaaacgggggactatttactatgtctttaatcatcaaaacacctcctctattttgtattcatataaaactccagccaatccttctatcattccaggaaaatcgctaacatcatcaagtaatatttctctttcagcaacaaatagttggtagtttcctactagttctcttaagctttttacttcttctatttgtgaaggtttgagatatgaaacgactattgagctaacttcattgctataatcaacatattcgtcaataaaaagcgaataatattttttggcaattctcttgccactaataactttcgtatcaaaagttttaaataaatctctttctaatgttaaaccagacataacattgtagtaatactcaatggccttgtaaaagattttgttgcttacatagtcagaaataataaaagccttacaacgagaaaattcttcaactatttcgtctgcaaaacgattgctattgtttggatagtaattaagttctaagtaagctttgtatatctttaatgctttaaagttagccgattttaaaaattcctctaaaactttcacctgcatcacctcctttctcagaacatgtaatcataggtcaataaaaaccccagacctaaaatattgtgtgtttctttcaattttctaaagcctgcatttaacacaaaaaactcatcgtactccataataggaagaaacataaagtctgtaaaataatcttcaatatgtggctttacgaatgagaaaatattttcctcgtttatttctttaaataagatttttccgttttcatcgttagttacaaaaaatagcttttcttcatcgtaaaacttaatatttttcacttttctatcagcgtcttgatctaaggaagcttttattgcagtatttaaaaaatgttcaaaattttttcctcccagatcagtttcgtatatgtgagaaatcgttgaagccacatcattatcaaaataatagtatttgatagttactattgaaaagtgtcttgctttaaacattccactaatcttcttgattttttcgtttactaagcgaatgtattctttcatctcttcaccccctcgccttagtttaaaggacaaaacgccttaggtcaagtagcgtgcaagcgctcagttgtctatatatagaggatagcgtttatctaacctttcacaaaaaagaaaaaaataaaaaacaataatcttagaatacttctttgcgatatttgatcttatatattcgtcctaaagttgactcctcaactatttctattctttttgtaaacatattttctttataagacgcaatcctgtaatcaagtaccaataagtaaacatttggataaatgttactaacaaactctacttctctttggaagctttttattttaatgggagaagaattattaaatctatcatatatgtaagaaggaaaacgctttctaacattagataatatattgctttcttcccattcgtgataaactagatagtatttcgtttcagggcgaaaaatttggtggaaatataaatacttcctgccttttttgtcaactactaggcgagataaaattctaatatattcttctttttctatcatttttatttcattagaaactttccacccagaacgaaagtattgtgatttaattatctcaaggaaaggaagaaaaggtttaatatcttgaaaatcattcaagtcttttattaggtttttcatctctttacctcccctcctccctaaagcttatacctaaagcgccgtggagtcaagggaaggcgcttaactcttaaaaaacttctttatgatgtttaattttgtaaattcgtccaaatgtacatcttttaacgaaagttatttcttctgtcaatttgtttttcatatacaagtcaatctcaaaatcgtcaactaaaaagtaaatcaacttgtcgcttttaaaattctcactagaaattcttaaaaggactcttctaaacctttctatctcgtcattggttattccattagaaaaaatatatgagtaatgtctacaaatactaggaataacttcagtttcctctccagtatattccaataaacaacaagattttgggtaaatgattttgtagaagaaggtatacctattgtcttccttctcgcttattaactcagatattgtctgtaagcattcttctttttcaatgatttctatatctggacataagttttcctttaaaacaaaatattttccttcaaaaacttctgcaaatgaaagaataagcttaaatttagaaaagtcgtcaaaatctttaaccaaatcttttgcctcttctttcctcatccttactcgccccctccttacttgcccctccttgacctatgacacaataaaagcttacccctaaagccccataaggtcaaggaggaggatgaagctttaggagaaggtaagcccaaggctttagggggacataaaactggcgagaagacatttatttaggatagcgtttacttatttttgacgaaacaaaaaatcaaaaagcttcattagaaaacctcattaagcttataagtatacaaagtgcctactaacccctctattattcctgaacattcatcaacatcatctagaaatacttttctttttgcaacaaataaaatattaacatctagcaagtttttcacatcttttatttccgttatttgctgtggtttaaaataagtaacatatacacctacacttttttcaatagctttataatcgtcaattaagattgaataacactctttgtttattttttcctcacccaaaaatcttacatcaaatttctttggagcacctctttcaacaggaacagataagaatctattgtaaaaatgtccaatgcgcttataaaaaacttggtatttctcagaataatcacaagtaataagtgttttataaaattcaaaactagaaactacttctttggcaaaaatatttctttcgctaggatgataagtaacttccatataagccttctttttgctaagaattttaaatagccctgcctttaaaaatctttccatcccaccacctccccttaccttaaaggttactactaaagccttataaggtcaagggggaataggaaaaggccttgtggggattgagagagggaacaagactttacgaggaaaagactagcagggaaatatatatttaggatagcgtttacttgatttctctttaagttttgagaaataaaaaatcattggaaaagaaagatttgtgagaatattaaccctattactccaaatcttccatcctaacgacaagatatcctttgctcattaagtcattaaaggcttgcttatcataaccttctgtagcgattacaacaacatctttgggcattttttctaatccgtagatatgcaaatctgtaatgactataccaggaatattctttttagacacatgttcaaaaactttatcaaaaacgctaccaccacctccttttacaccattttttaaggatttatggagttttgatacgctattaacattttctatctcttggaaatcaacatcattaaagtagagatgaacattagcattgttctctaccaaaaacctcaagatttttattgatttctctaaatcctcattacccattgagcccgaggtatctacaataacatacatttcaaggccaaagtattttttcttccctaacactagaccatcgttgcttctaaaggatttctttgggtaaacgattctatgtctaaaatatcctccacgatacaaacgattaaataactgaaggacaaactgaggaatctttatcttttcatttgtgtcaagaatattcctaaggaatccacctgtatttccgatgtgctttttatttttctccaaaaatccttcaagtattctcctgttcttttcatcatcgtaatcactaacttcattgttttcgccaataagttggcgaatgatttcgttttttattgcattatccaagggaccaaatgatgaagcattcgctaaatcttctttaaactttggatcgttctttgccttattccttatttgattagcaacatcattaaagatgttgtttagcagatcaaagaagttttcttgggaaccttgtccttgctgattgttattttgttcctggttattattgttttgtacaccctgattctgttgattgtttcccccttggttttgctggttattaccttgattctgttgattattctcctcttgattttgctgattatcattatcttgtttaccttgattttgctgactattttgattatttaagtgtttatcaatggattcgctaatcatctgagatatttgttgtgaaatatcttcaaaaatatcaaagaagaactccattagctcataatcactcattttcttcagttgctcaatattaacgcctttaagattaggcttaagtagcattcccaaagtattaacgccattgcgaatattcttttcaaaattttcaaaacatatactattttccttgatttccttaggatcatctataaactgatacacgatttcctgaataaacaaatccaaaacaaaattagttagcatcgaatttattctaaaataatgctcaaatcgtttaatatgcgatagcctttggtgcaagatttcgtgaaatagggtagttccaactgcattataaactggagtatactttgtaaaaagtttttcgttaaggtatattacgggcttatttttttcctgagtagtgaatccgtacattccaacattttgaccattaatctcatcaggaaaaacataaatatcggaaaccaataaatcctcgtaaccattgagagtataaagtagaggttctctattctttgtacgattaggggcaaagaggattactttaaaaatatccaaatattctgtattttctatctttttggcgtgctttttaatgatgtcagtcatcttctcacctccccttctaaacgccctttttggagatattttgcaagtatttgctcaaggtactatttttgtgagtgtgaacatactcgtaaacatcggctttccattcgttaccattgaccaatgcagtataggacaacatatagtgatcatatttcttgtggtttttgtgaatgtagtcaaagatttcgctaaatttattaagatcaatcattcctttctttacaagatcggcgacatccacaatcaacgataacacctcattgtcatttctaggataatctcccttagcgtagtttttgtaagaataatacctaccttgaataatatagttcttaaaggctaaatatactttccttcccgatgatccagagatgataatatcaagctcgctatcaatagacataacataatcttcatttcctaccatttcgttattgattaaatcctttagaagaatagcagtacgagtattattccttggagaaggatactggtttaaatcatcatcttttggctcttcgtaaaacatatgatctggactattaagaatgaaagatacaaacaaaggattcccgcctctactcatcgtatacgaagcccattcctcttttgagggaatgggaaaatcaataactacaaacctctctactaatggacgagggattattgtggctactgaagattgttcaggtttattgcctgtagcaatgaagtaaatattttcaaactttttgtcgccgattctcttgtcaagtaccaaagattgtaaaagcgagtgcatatctggattcctattcgtcaactcatcaagaataacgattttattattaagatcaaaggttacaaagtgtagcatttcttccctgacttgtgggaatgaaatattttctggaaacatttgagaaacattaagatagaatacctccaaacccatttccctggcgatttcccaagatacattagtttttcctattcccctatctccgaggaataggaccgggaatccactagtaagaatttttttagcgatgttttttacctttgttactctctccatcttttacctcctttgcccctcaagggggcgcttttagcttaacacaagccgctacgcaagtcaagcgcctaggcaagacaaaagaagcaaagcgcctaggcgcaacaaagagaataagatagcgtttacttatttttctcagaaaacttcttcaagaaaaatgtttagcgaaaagtgatagttgaaataaaacactctatcatttattctttcaccagaaagatagaacttgtgatttagtgtttttgaaaggttgtagagtattttttcttcatttatttctgttggcgctgaaatcaggttttcaaaaaaagcttcttttaaaacattgctgtaaacgcttggattgctacgataataataggcatcttttattgttgcgaagtgtaaataaacgattgtatcgtagtttgaaggaagtttgtaaagtttgtcttcattttctagttctattgatagaagatgatacttttctttgattttcttttttattgggttttgttgaaatgccaagcgataaatcttttttttgctcaaaaattttttaaacttgtgaactacttgccctacaaagctatcaaaatcgttaggagttgtgtaatacttttcaaatcctcttttgacctcaaacctcaaagttgaagttttgtattgaactacaaaaaactttgaggattcttctatttctgttttcagcgatatcaaccttttttcaaagacttttaaaaaatcctcatatgaaaatacagataaagtttttgttttaatcttatacttcagattgtttttcggcctatataaagtaatcgtgattagtttagcatcagcagacatatctcatttttgcaaaacaaaaagaaaacttcactaccccaaaacgaggtagtgaagtctaaatattgcttagaacaagctcatgatgttgtatttatctacgaggattttagtgaactcttcagatgtgcttaccaatcccttaacagcattcaaagattcattgttcttttccacaatattaaccacaagttgaaccatatccctaacattagtgatgtggctcttcaaaaactcagcattgatctcgttttcttttgctatagtattaagagcctttttaatgacccttggaccaaaagcatttataaacttttcaagatccttatcatctacattttgttcaaggaaagtatttgcaacatctttgttttcttcaaggatgttgatgattttgtagacatcacgcttcataatattcccctttgtatgtttattggaaaagtttagaagaagtggcttgtaaatgatattttccttaaacataccagaataagagttcacatagtagatagtattgttctttgtaaggaaatagtacctagagatgtaaactggattatcaggttcaaacaatggattaatgttgtcaagttcagagaacttgaaaacaccataacgagaagtaacatagttgaagaagagtttttcagtttcatgggcgaaaatgtattcatcgctaaaaagattaatgaggaccaaatcattttcaaaatccttaacattatttaccctaagaacatagaagaagtttttacgaagattttcaacgagattgacagactctttaatgttgtagatcttcatctctttcatctttacctccttgcctacccaaggcggggtaggctatgcctagcctatcactactgaactatttagtcaagggggcctggtaagcagaaggagaagcgcactaaggaataggatagcgtttacttattttttgcggattaaaaaaataatcaaaaacatttcgcttaaaatagctctctaaatactaccctatcctcaagctttatgtcaaacttcatcaggtcttcatttaaaaagcagtaaaaactaatatatgcaaaaatgtggtagtcattgtctaacttttctgtatttttgttttcaacgattttgtaaagaaacttttcgttgcgaatatcagtattttttgagaagattttttcgtcattaatacagatgtctacttttaccttgtgaatcttgttcacttgagagaggatttttctattaagttttcgccaaaagggaataaggctactatgttctttcatacgataaataacttcatcagattttctacattgcgaaaggttttgtagtgtaagtacaacctttacaaatacttcttttctcttcacttaaatcacctccttattaaggcgaagcttcaagatgatttccaggaaaatgtactcgtaaaaatagtcagaatatactttatatccgttatatgtatcaacttttctttttctatatattgcggcgtggatttctggtcttttaacattattttccaaaagctccggcaacgcttgtttaaagaactcgttaatatttctaagaaggtaatccgaatatttttcatcatcattgggcaaagctttgaatctttttggatttaaacgctttccacgaatatttgtttcaatgtttatatagaacatattttgctcattgggtaagaatgtgtgcaagtagcaatataacttttcggcatgctctaataccttacagaaatcttcaaacttaggatttgaaactatcctcattttgaagctatcatagatttccttagaatcttcaccacctatttcgttcataaagtaatgtgaggcaccaacataatattcgtcaagatcagctatcatttcccatttccacgcctctttgagcttgtctttcacttaaatcacctcccttagcatttccagatttaaagaaacttcaaaaaggacatactcataaaataacgaatcctcataatcttcaatgtcatcatcttctaactgcaccttacttctcctgtatatagaagcatatattttaacattcttaatgttatctttcaaaagctttggcaagttctttttaaatacatgatttatcctgttcatcataaaatctgaatagcttttccaccttctgggtgatacataagaagtataaggctttgtgcttctagaatatggctctgtttcaatgtcaatatacaatatgtcctcctccttgggaagaaaggtttgaaaataggcataaaggctttctgcatgctttaagtttttgtataaatcttcaaacttaggatttgaaaatatttttattgaaaactcggcgtatactttctttgtattttcttcatcaaactcatctacaacatagtgagaactgccaacatagtaagaagatagtttagttatcatttcccatttccagattttgttcatcagaacaccccctttatcgcaagaagaagttctgtttcaaatgtggcatattgagtatatatttcagatattttgtgtcttctctcaataccaagaagcatcttaatcttttttgtgttttttgaaacgatttcttttaaaacattcctaaaatattggttcatttcagaaacgatatagtctgcataacttacacaagcttttacctcttttacataatcagggtgcaaaagatacatgccttctaaataggttctaatgaaaaatctttcatcaccatttaaatcctccacaagatataaataaaaatcttctccttctttaaggagttcataaagtttttcaaagcttttgtcttttacgattctttttgagaactcatcaaagattctaaaactttctatttcgccgccatcatctataatataggcatgacattttacagacatcatttctatctcagatattgaatcccacaaaagcttttgcacggctcacctcccaggtctaggataaggctcaagaggagtccagtcaagcccactctaggaaaagatttgattttgcatttcaaactttttatttttcgctaaacacaaaatgaacttctacgctaacatttttattactgcaccttatttttgtaaactacataaaaaaatagctattcttctagtatctttggtttggcttgtgttaaggtttaggattgcgtttatttttttagcaaaaatctttaagctttctcacaaaagttcaatttatgagtaaacgcttaatcatagtttgcgaaaaaaaaaataaaaaatcattagaagaaggatttcttatagtttagaaaatctttagatgttcttcgtgcctccaagaattccttgaaacccaaacatttagatttggattattttttatcaagctttcggctgaaattccaaaatacttttcaaatacatctttaagcttcttattcaaatcttcctcatcaacatacttgtcattaattaaaagatcatattcatcgatgttcttgaagaattccttgacaaccttgcctagcgcttctttgagagtccacattcctatcacctccgatgctcatcctagcaaaggtgtgtccttttgtcaagctctcctcttcaatcaagcgcactcctcttcaaaggtattgcgaatttttttctagcgcttaaaaaacttcgcttttttcgtattctgttcttatgtttaattcatacttaacatttttgctattgtttttattgtttgctagaatattaaaaaccaaaataaaagtgcggtctgaaagtgattcgctaaactttaagagtttcttgtaagtctttattacatattcctcatcttgcttataatcaaagttcaatataactttcttactcattgttgtctttaatgttctttctttcttagttatatttctttttcctgtagcctcaaaataaatcttcattagcgcttttgtattttcttttgagatagagtttttgagaattttaaagtattttctaaataacgcctcaatatcgtaaagcttttcttctctaaaatcaattagaaaagcgatgcttttcttcacttcttctttttctttatttgcgacaactgaaaatactaaatgatatttctttggtgcaaaaggtgtcatgtcttcaagctaacacaatagcgaaacgctgtcaagcgttggtacactaataataaagtctggaaagtctagacgatagacaccttggtacactattaccaaagtctggaggatagagatactagctgaaatattgacatagttagacataaggaaaagttctcctaaacttagtctttagtgaaaattcaattgtcttttcaccagtaaagaagtgagttcctagagaaaaggtaaaactttttgcatctttattctcaaagattctatcttgtgcttttttagttgtaagataaaagtcttcaagtaatgcgtttacttcctttggtgaacctagagttttgtattcgatgtgtgaagcatactgatttaatttaaagttcacatcaaaaaaggtttcttcgtagatgcttagaattaattttgttggcaagaatttactaaacaaatctaggaaaagattataggttctaaaaatctcatcgctatacaaatgaaaagtgtgcaagtgttttgtagacttggttttgaagttgattgatttgtttatttttttaatgttactttcttctagtgaaaggataacttctactaattcaaaggcaggatgacaataaatttttactttattttcttgcgtgcttttcatgttttgagtatatgcgatttgctttttgttgtcaagtgtgtcagaacacatcttttgtttttttaagaactaaggctgctttaaggatatagtagaagtgttttgactcctctacagacgattttatgatcaaatagttgtatacaaaaattaatctagcatttcgtacaatcgcttgttttgtagattcgtttaggttttcgtttatttttgaaacaaacaagcttaaatttaggtcttcaatataatctttttgctgtttgtattctactgaacgaatatcgttgttttcgtcttcaattccttctactaatgcgaagaatgtagttttgccttgaggatttcttgctttgttcgctaaaaatcttttaaacagctttattacatcataagttctcattagcgaaacagtattttttgtttcgttgtaattaaaatcatttctattaaacctttcttcctctgctgccaaagtaaaaaagctttctattacttcttcttctgatttatctttattggtgtagtgtatataaaagcacaacgaaaaattatctagttcgtaaggataggtattgattaactcaaaagacatctttgcctccttcaaaagaaagcgttaatttaaacaaattagtcgagtttatagcagcttcttttgctttgtatttgtaagaaagataagatatcacttcaaattctttagcttcgaagtaaaagactttaggcagtccttgtgcgatatatttgtttaggttttcgctaaatagcgaaatagaaaattcttttattggcgtttctatctttatcagctcatgttcgtcaaggtatgcgcattctgcaacacatgtcatttcaaagtaaaagaacctttctttaataaactttttatcttttgagaaatagtctttcgcaaaattttcaaacatatcacgaattttttcttctccttttagaataaacaagtttctttcttcatcaaagtcgatgagattttctcttagttctatgtagttgtcaatagcgccaattttttcatatcctccattaattcttgaataaaacttagagaaatgttcaacctttgcgcttttaagcgaaaaaggaacaaagttattaatttcaatcgctttgcttttttcctctttcatgctcacctccttctcagaacagctcatctcttactagtcttactgatatgtctacagaacgcttctttcttgatagctcattatctaaaaactcaacgtagatcgtcatgtaaaatatagcttttggaaagtctttgtttttgctcaaaatgtcgtataaagtgtttcttatttcattcgttatgtcaaacttaaaaatttcttctaaagagtaagacttttcgtagtcgtgaattatttcttgctttttgctaggttcaggataaatcataaagaaagacaaaacaacagaaaatattcctgctttttttgcctttgcgattataaagtcatctcttagttctcttctaatgatttcaaattcgttaagaaattcttcataattttcaaagtctttttcttcgttaagatgtcttagcgctattgtagcgctttcaaatttagcgaaaggcaaacctaacatgttcgcctcctttcaaaacaagtcttcgtgcatatagaagaagtttttgctgagatgaaatattaaattagaattttcaaaaaacatatctacatcaatgaaaatgctttcgcttcctgttactaaagcgccaaagcgaagcattgaataagatgctttttcaagaaattctttgatttcttcgtaattctttaatgcgaggttttctttataagacgctttaggaaacatttctttgtaatagcatctaatctcaaccttatatttttcgatgtcaaattctcgcatgagattgaaaaatttgtctttaaaagacaacgcttttctgctattagtaaagactaaaccttcttcatttttcttaagaacattttttgttcttttaagaacgactttttcttcaaaatttcttctgtagtcgtctgtcttatcaataacaaagaagcttatttttactttttcaagcttgtaaggaatttcttctttcacaaagaagtcaagtttcattttgcacctcctcttgcagaggggattatacaacgggacaggacgcatgtcaaggcctcaggagttaattaaaagacatttaatttcactagagttaaaaaggatcatttctcgctatacgaattgtcatttctagatttgtttttacatctgaatcgtagttgtcttttattgactcgtagattaagtaaaacgaaaaagaaatctcataaaagctaggaatttctttcatagttttcttaacaatctttttcaaggttgacttaatatttagcgaactgaaattctcaatgttgtagtgaacaggctcaacctgatagaaatctctttcttgttgacctgacaacaaatctttagcataaaagtaaacagaataaaaatgataagcattaaaatgcaagtttgctatttcttctttttgtttcaaagacttaaagatatttagttcttttattatccttttgtgaagcagtctaaaagcgctgataaattcagaaaagtttgaagtgtttagatgatagttctcaacagatgtttctttgtcttttctagcgaaagacaactttgcgctatttactttcgcatagctagtcaacacaacgcacctcctctcaagctctaggataagcgctataggacgctttgtcaagagtctagtgtcgtcaactaagcgaagcgaaataaggctttatcttgcgtgaaaatcttatttaaactttaattcgctaaaacacgcttaaaaacttgtactttaaggatgtgttaaaagaaaacactaacgaaaattcatctatgtcaacataaagatcctttcctgaagtcaaaatgaaattgttaaggtttagtttaagcgcttcttttatcgctagcgaagaaaaacttttaagaatttctttgctttcttctagtttgcttagtttgtgattttgctttaagaagactttgaaatttggatcctcataaaatgcagaaactttaaaatcgtaatgctcaatattaaaaagttctaaggcgttgaaaaaatgttttctaagttctaaagcttttgtgctattaataaacagcagatcttctttatcttttctaaatttctttttgtttggcgtaaatattaatttttcttttcgaactaattttctagaatagctaattccataactgacttctttttcgatgtaaaaataaaggctaactctagtgattttgtaaggcatttcttctttgacaaaaagctcaaacttcatcttccacctccttaggaatgtccttcctggtggttctctagaaaggaaaattgctaacaacttctactgaacacgatatggactgttttttgtaaatttccttatttttaaaataagctgcataaatcaacaaattaaattgcgcctttgaaaaaaatccacaggtttttaaagctttatataagagattttcaatgctgtttgttatatcaaggtcaaaaacttcctctagtttgtaagattctccagagatatattctccaaagaggtctaagtcatcaatgaggagtcctttctctattggtaaatagataaggtcagaaaaagagatggcatttatctctaaattatttcctttagcaaaaagatattcatcttttactttgttaatatggtttttaagctccattaaaaattcttcatagttgcgaaaacttttggcttcttctaaagcaaaatctttttttatcttcttagaatattctgttttgacataagcgattttgaattctgtgaaggctaacctcacgctacacacctcccctctgctaccccaactataagcctcacaaggccctatgtcaagggggggttagtgtgtttttactagaagaagcacggcgaggcttttattgtgagtgaaatcttgtttaaacatttttgggtggtacactattatccaagtctggaaggacaaacaaacaacgctagaaaacctcattacccttcttgtattcaaacttcactacgaagagtttttctgtgctttcaacttctttgtttgctagtagtccctgtagaagcgtaggaaacttgatgatagttgcattaaagcgaaagtagtaagtaatgctcaccattccttctcttaaagcttttattgtgatagattttccaaataggtttatcattgaagcgaattcgttttcattaaagtttgcaattatgttgttttctttacaaatgtaaatcggcagagaaactagtttgtaaggtgagaacattaagtaggagaactcaaaagatgagtctacttttactagtccagactttgaagataggtcttttttaaattcctctaagtagtatttgacttcttcaaaaaagttgtaaaaatcaaaataatctttgtatagtgattgttttaatgcttttttattttttgcctccttgtagagaattgtgataaaatctaaggagtaaggaagaaagctctcaaacatgtctttatcctctcgcttgcgataatatcgcctaaatcataggcaggtcaagaggttggtacactattataggaatctggaagatactcactaacagcgaaagaataaagaaatactatctctagataaggataaagaagttcaaagtatttgtatcttcctctagcaaacgatgctactatttcgacaccttcaagattttcgtttaattcttcttctgtattattcaagaagttttctttttctttaattaaagcgttatagacattttcaactgttgtgtttcttcctaggtttacttctttgatttcaggaatgtagctcaaggtcttttctttcatgttctttatttgtttgtgataaatcttgacgaaagaaagcgttaaatagaggtcaagagagaagtttgcgtttagaactttgtctaaagataaagtttttttgatgaatgtctttatgataaaagacagcttttcttcacaaaattcttttttgcgagtatctaattcaacatagatcaaggttctttttgatgtatttcttatttccagctttgatagctttcttatttccagagaagagccgctatcaaagtgtattgttgctttttctagtgtgatgttcgtcttcatgtttctttacctcagaataagacattaaactcaaaagaaacttctatgcgcaaatcttgtgaaatagtcgctttattagtttttaactttgcgaaagataaaaagaagacattgagcgttttttcttctttaatagcatatttatagcgtttattaatttcacgctgcattagactaaaaatcttttgttcactaaattcttctaagctaataaacttctttcctaagcgctttcttagctctttgtaaggcatatcgaaataataatcgcttgatagcaaagatacgactgcaaaatgtcctaatttgaaaaaaggcgaattaaagtatctttttaaaatctcaaaactccttgagaaattagtaaacgcttgatcaaagaattcgcttctttcttcttcaaaaaactcagttttcctagttgaaagaaggctatcgttaatcaaatattcttttataaacctcaaacttttaggtttttgcagcgaaaaaagctcttcgattttcatgctctcacctaaaagacttcttttctttctagagtagcttctacttctagaaagactttatcaaggtttcttgttagatagcctagttctatgaagattactattgcagtaatttctttttcgttcctaaacgcttcattgtaaatttgacttatgcttccttcaataaattttaggactgactttttattaacgaaagaagtatttttaacgcccaaatcttcaaatttaggtgttccgttttttgtatttgtgattagtgtatagtgtatttttaaaatgtcttgcacaacatcgccttcagaattttttgtttgctgatagtattttgaaatgaaacctttaaggtacttataaaagtctttagctttttcttctttaagtatctttttaaacgaaaaattattttcttcatcttttgagtttttaaagtattcaaaggcaaacttcactttcacgaattcaaaaggcgctagcatagcttcatgctagcacgacaagtcaatcgtgtcaacttggtacactattactaaagtctggaagatgtactatgtcgtagtagtatacaacctctagagatgcaacatgatcatccttataaggtgatcctttagtgaagattattgaaatgataatctgatcctttttgtctatgttttttagctttaacagctttgttagcaattcgtttctttctttgaggattgtcttaagcacttcttctggttttatatcgtatccaagaggtttttctttagtatcaaggtcttctttcctcatgttgtgattgactgctaaatagaaatcttcgtcttcatatacttcaagaagtttttcaatttctattgtttgttttataaacttaagcattagttgaattgctttttcttctagttctttttcttcgtgatctttgttgaaggtaaatttgtatttattttctgctgcttttaaaagtccttttgacgcagcaaaagactcctgtaagtaaaaaagtgcgatttcgctgagcttcagtttcatctcttctcctttcaaaaaacgccgtgcaaagagtagtgcattttcaagaacaatttttttactatcaaagatgtttctgtctttagcgcaacaaagaacatagattctaatgataggttttcgttatttaacaaagcttctctgtatttcttgtagaagcattcttctgctaaacgagaaaatttttcttcgctaaattcttcaattctataaacattttcaggaaatctatctagcagacttttttctctgccataaatctcaaaatattcttcagaaaaacttcctaaagaaaaaactacaaaatagcctttgtgaaaatactttgacaacacaaaccttttgacgatgtttaactgcttcaaaaatacagcattaaagctttcaatttcttcttctggaaaattgtagtaatatttctgctttctagctttaacaaattttttcttcacgaagtgctcagtcgacacttctacgaaaaatattttgtgtgttttaatcaagtcttcaatgcgcataacacctcaaaagctaggaagaacaatgtcgtttatttttatcgtgtaaggaggctttaaagaaaagatgtgttctatttctaaaacccaaactttttcgctttgttttatttcaaaaactattcttggcgctatagatatcgtgtaatcgttaaagtttgtaatattttttcttatttcatttagtgttttttctgtgctttcaatcatttcataagctttctctttgagaaaaaacgaaagcttctttttcttgtctgacaaaatttcgtcttgtttaatgttttttatgtcctcgaagtaaaaccttgtttttcctaaaacatagcctctataattgacgaatatgtaaatagagattgtctttacatcctgctttagcgatttcaatacaaagctatcaagcgttttaataagtttgtctttttcattcacagaaaaaacaacatttgatgcattcgtgtagctaaagaagggtttgtatattcctttctttgtgattaagcaaagcctttctaatctcacttttctcctccacagaagaaatcatagaacacaaacctaaggctgtcaagcgttggtacactattaataagtctggaagatgtaaatgactgacacttgaagactaggtgtctatatgctagtatgtagctatgcatcctcaggaaggttggctttttgatgagtttgtcttcttgagaagagcgttttctcattgttcatatgttgacagcaataacatagttcgtgattatcaggaagaaattgaaagcttagaaaaactagaggctattgttgaattagcaaaggaaaaattaaaagaatatcataaagaattaaaggagctaattaggtttggattaagaatagaagaaaattttcatttaatcgctatagcaaacttttataagaggtcattaagacacaagtacgatagaaagttaacgcttgacgaagtaatgtctttttctattagagatgcgatagacgaaactatatcgcacttcttgagattttgtgaaattgaagatttatctttgattttgagcttgaagtttccttatttgtctgtttacaagcctatgttaatttctagagcttatgattgttttatgatgattccttatacttcacagctttttcacattagcgctgaatttctcgttgataaaaatttgcttgttttctaaaaaaattcttctatttcagcgattaaagaataatcgaacaaatttggttcgcttaaaatcttcactatctctctagtgtgtttgtcaaatattttagtatctatttgttttgtgtttgtcgaataaagatctgatgaaatttcttcatcgaaaacatttaatcgaaaatcaaacttgaaccgaaaggaaatttttgacattaagccaaaatttttaagaaaatacatcgttgctttttctaactctttttttagagataaggaaggaagttcagaaagcttgtattcttttttcaagtcaaatcctttgtcttctccatagaaggctaaagcgctaaattctaaagattcaaaagtaacgacatttaatttgttgttgctgctttgatttcttactactcgttttatattatttaattcacgattaaacatcttagctctatctttagcgagattaaagaagttttttatttcttcaaacacatcacagtagtttggaaaaaagtagctaaattttacttcgctaaactcgcaagaattttttgcaggcaacttcttatctgctttcttttctaggtagcttatgtagacgaagcctctcgtgaaatttagtatgtcaaaggcgctgaaggacactttaaacacctccctcctacctctctagtatagaggctaggcgcaaagctgccaagcctttatttttagggtaagccttatttaaaagtttaaacatttgctagataaaaatcttttatgttcattatctcttaaacgctaggatgaggagttggtacactattgtataagtctggaaggttgaataaatgctgcccaagatctaagactttgtatttcagtttaaagacagaactgtagggagttttgtgtcttgatctttcgtgagaagataagctaacaacaagatcaaatctagttaaaggaattccttttcttagactaaagaattcaattagttcttctattgaatcctcgatgaagaaataaaacgcattttttattctcttttcatttggcattactagcctcgcaaattgaatttgcttagacatttctaaatctagcttgcattctttgttttttcctagtgaatagacatttatgctttttgagatcgttaggtcaaagtctttcttaattattgcttttgacaatactttttctataagcctctctaaagagtttttatttttcgctgatatttctgctaaataatgaaacttaaactcttcaaggttcttttcattgaaatactcgtagtttatttcgatttgattaagaaaaaaggtttgcttgttgattttgataagttctttcatcttcagatctcaattttttttctttcaaaatacactaaaaaccaaatgacatacttttcaggaaaaggtattgttttatttttaaagcttgcgacaattctttgcgcaaaagatatcttcaaggtcttgttttcgtatatgcagtctttaaacatgctattttcgattatcttttctattctttcagaaatcatttttgataggtctacttcgctgacatcttcaatgaattctttgtttgaacctagaaaatttgttaaattttttattctctttttcttttgtccttgtacttcaatttcaacatcattagcgatgacgacatctaaattctcttttttgatttctttgaatgttttagcgagaaatttttttgcagcttcaaagtcttctgaaacaaactttctttggttgaataattcgtttgtaaggccgctaaaatcaattcttctataaatgctaactgcacaatttttcattaaaaatctttgttcgcctaagtctttgttataaaacaaaagctcttttctcatcctagacctcctcttatgtgtaatctttaaagacttcttcatctagcgctctttcaaactcttctttagtgagcgttataatctttccttcctcaaagaagtgaatgcgcttgaagtctaattcatttaatatgcttgcgcaatacaagcaaggtctagtattcttaagaaggttttttctgtagccgaaaacaaaaatatcactaggataaaagttttcttttctagccttgtgaaaaaagtctaactcagcgtgaagcgctgaaaagtgaggatagtcgtatttaacttgcttgctgatgatgtatttattttcgccaattagaatttcttttctatttacgccaacgcaatacatcttcactttgccttttggtatattcgcatctctttctagcaaaatattcattagacgaagtaaacgctcaatcttcatgtgcgtcataataacacacgcttgacagaatgtctagcttgctttatagtaggagttgcaatgaggggaggtgtttaccttgctgaagttaggcgcactagacctaggctcaagaagacagatgattgtaaaaggagtcgtagaagacgatgtactgatagttaaaagctacaactattcttacattgaagaggaagaaaagaaagaaaagattagaaagttagacgagtttttagagaacaatgttttaaatgaagaccttgatacaatcattttagaaagaagatttaaagacaaaagaaaaaataaaatactaacaaaaaaagaaggatacaaaactaacatagatgcgtttatcactttaagcgaatacgctaagaacaaacttttgaaaagcaatataaagttgctaacagtagaagctagtaatattgaagcttttaaaataacgagaagaaaaggaaacttttcttcaccaggctggaagcaaattcttactggaatcaataagcctaacgacaaagaaattgaaaaagaaataaacagtcttgtaaataaaggaaaaatcatatacgaagtaaacattgaagatttacttgcgcaaaaagttaaattttacatagacttcattgacgctgtaggaatgctttatgcatacattaaaaaaccagaaatttatgcgttagaagaacaaaatgataacaaagaacctgaagcagacttatacgaagaaattacaaaggagttgtcaaaaaaacttcttggcccaatagtgtcttgagtgaaataaaactctttttgtgtttaatgaaattaaaaattttttagtgttcctttttatctcattgctaataacaaaagcgtttatttctttgtagtacttataagcgaacctagcgtcctcattaaagtctgcaacgaaaagaattaagtcttctagataataagaattcacgatgtagctttttatcgtcttgtagtcttcctttgtctttatgtgatagaagtcaaaaacttcttcaacttcaatgaattttttatacgccttatacaaaggcaaaggattttgcaggaagtaaaaaaacaaatatctagaaaaagtttctaaatagttagtgctaaagcgatatctttcaaactctgagatttttcttatagacttgtattgcttaacaaaagcgaactctttcagtatcttcttttgtattggacttatgttaaaaataatcctatcgatgaagtaaacgcttctagcgtttcttttaaacgcttcgcttgctcttagaattggaaacgatagtttagtgttcactctttattcactatagcacaagacctttatatcgtcctgcacgcaaaaattaagattttaaaagtgaaaccttgatttttgggcaaaattgggggttttattttcttatgtcgattttcctataggcgaaacaaaattatagggcgacataaaatcaaaatctttgatttttgctaaaaattgaaagttttatttttttataagattttcctataagcgaaataaaactttaggtggtgtgaaaataaaaatctttgaattttgcgcaaaattgaaggttttaaaatttcataaggttttgctataggtgaaataaaaaactgaaagaacaaaaaactaaaatctttgattttttggcgaaattaaaagttttaaaattttgtaaggttttataggggattaaataaaacataaacaaaatttccagcagtaggaactttcatatagccattagctttcatgatttcgctttcagatgaattttcgtcaaagaattttatttcgtatttattttgctcgtaataaccttttaatttatgtttttgaaaaagttctctaggataaagctctcttccgttcttcgtataaaagtatccaggctttgttattccaattcttttaaatccaagtttctcgtaaaccttgccactagaatacataaggtctgcataagagattaagttttttggtgaaaagtgttttatcatttttcctaatgcgccaataacaatcgtgtttttctttgaggcaaacctaactagctcccaagagaaattctcgttgtatctaggttttgcaaaagaagctgctgctacgatttcgtcatcataatacagaccaaaagcatacttaaaacttgcgcttccagaaatatgattttcttcaaaaaattcgtttgcagtagtgtagtcaacttcttcaatttttgttttcctagcaaatatttttctttcaaagagtcctagtttcgctttaattagcgatttgacgatatctagtttttggtaaataataggttcgtagaagaacagaacatcaattcctctatcaagaaacagcagctttttcttttgcaggtgattgaaaggtttcttttcctctgagtgccaataagctccgttgagttctattgctagattttttgccggaataaaaaagtctaattctagttcatctctatacctaaattcataagaaaaaggaatgttctttaaaacttcgttttcaaaaaattcttcaagcgtgcttttcttaatcctacaactaggacaagaagttatgttatattgaagtagaacatttaaataatattcaaaaacatttccacaagagcaaagtaaaggaactctagattttatattgtagtcgtcatatagaatctttaagttcgccttagatgtaaactcttctattttttgtctagcttttaatgtgatttcatttttctgcttaagtttttcttcttttttatctactcccttctttttctcctcaattaaaaagaaaaacctttcctttttctttagataaacatctcgtagcgttgtgtacttcaagcgccaaatttcttggtttttgtaaatgagagaaaattctaaattagacaaattcaaaacagcctcaattgaaaaattcttttctgcttcttttacaagatgtgaaatttgcggcaaatgaataaaaaatttcatactagtcaaaaattcaaaaaacgctttctcaaaagaaatttcttcaaaactctcaacaagtttataatgcctcttcgtttctaaattatagactaaatctttttgggccttttgagcgtttgtagcctttgtataaagcgaaggatacggaataagaaacttgttgtctttataaaagacaattacgccgtctttagaaaatcttttctcgaagttttctacaaaagaaagcatcttgtcaaactttttcttcgcttgaagaacttttttgttttcaatgtggttaggaaggatgatcttcacagcatcagtataacaaaaaaatttggtggggcgtgtaggactcgaacctacaaccaatcggttatgagccgaccgctctcaccgcttgagctaacgccccacgattattatttggcgaggggtgcaggacttgaacctgcaacctagggattagaaatcccttgctctgtcctgttgagctaacccctcaaacacaagtctagtatagcacaccctcacaagtctgtcaagcctgtgtgtcaggctccaaaacaacctcttcaacgagttctcctactagatatgtataatactttgaaaattctttataaccaggtgcaaacacttttaatgtcatatttactctagccttatcttcaattacaataacttcaccctcttgaacattgtattcaacaaggttttctccttcttgtctttgcattgaacatctagcgaaaacaggattgccttgctggtctttaatgattagcgttagcttaaaagtagtttttatttgcccaccaataactccataaataccagcttctaacatcttacctatagtgcattcatcaacgccaaggtttttattagtaagctgcatcaagataaaaacaggttctccttctatagttatactacctgtttctaatgtataccacttaccgtctttataaaaaccatcagcgtactcaattgttactcttaaagactctgttgcgaaatctctttctatgtgaaggcttttgtgtcttctctctatttttgtttgaacttctcctacttctaaagccataattatattctataacaaagtcgtcttgtggtatgatgtgtgtatggaaatttactacgagctaaaagataaatttgattttggttatttcattaagataaattttgatgcgagaaatttgttttttgttagcgcaacaaattttgaggaaaaattttgcgaaataaaaaatactctacttcttaatgatgttcaaatttcttataaactcctctaaaggaactgaagaaatattgccttctacaataatcacatgtgttcctcttactccagaagatacttgtaaagaaggcgttgtagtagaagaaatagaaacactagaagcagtattagttgaaggaatgttttcaatatttctacgcatctcgtcaattatttttttagactctgggcttaaattgagatacttgtaaagaaaatcagttcctccttctatcgcctcagaagttaaattaataattccttttcctaaaggctcactaagctttagcgtaatagcagctaaagtagtgatgtttttatttagacttacaactgcttgagagttttctttaaaagtgtcaataagaagttggaaagaagaatttaacacatctaactgcctagcaaaaacttcatctggtgtctttgtagttggcattccttttcctagctctgtttctacaattctcctagctctcgtaatatcgccagtaatagcagacgcaattagagttctaaacacctcaggagaaatattagtaagacctaagacttgctgcattataggaattaaaaagttttctgcttgcgctgtaaatcttcctccagtaaattgactaactaatccagtttctatagaaagtcttcttagaacctgaattaaattatcaggtgtagcacctctttgtaagtctaaaggtgtcaaacctgcacgaataccaatacctaaagctataggatttccacttaagccgcttctaataaattcgttaaaagtagaaacaccttgttgaactatctcaggatttctttcagccaatctagcgatttgatagtttgaaaagtttcgcaaaacagtttgcgctgttaaaaaagtattaaagtaatcataaggattcaaaccttgcctaatagcgatagaagttgaggcgctaatcaaagcttcactaaaagctttagtaaacgctgaatattcacttcctaataaaaaggctcctcttatagctcttctttcatctattaacgctcttctagaagtagctacaagatttacagcctctgcaatagaagtaccaaacagattagctactctagcgctgtttaataaagcagaagaaaaattatctacgcttctacttaagccgccaatagaagcgaacaacctagctgatgtacctagatattcttctgttgtaaatccttgattagcaggaaatactcttcctccaaaaataaattgcctagaaacattagctacatctcctcctaattgctcacgaatttgctgaggaagaagtcttatttgtgtaaagcggctataaaaagtatcataagctttgtttacgctctgcaaataaaaatcagtaagaaaagctgctgaacctgctagaacgcctgcacctacgctagctgctgatgtgagtatgtttcttcctactagattgttagcagacaaagaagctgctaaacctaaagaacctacagaaactgctgctgcgcttgttgcgatagaaaattgatttaaattttttgtggtttcagagatgtattgcttaacttcttctttctctttaggaggaaaaatcttatctaataagaaaccagttcctacacctgctgctacgccgagtataggattaaaaagtcctacaagcgaacctactgcaaaagaagctcctgtttttaaaacattttcgatattaaaagttcttcttaatccttcgcctaaatttccagattctaaagtatttagaatagcgtctaaacctataccaaataacgcacctgttgcgcctactcttaaacctctagaaaaatttgtaggagttctatcagtagtaggtctattcgtaggaacaggtgtaggagttctattagtaggaggaggagttgttataggagttctgcttaacgaaggaaagtttctagctaataaattatctactgcgctaaatacatttgtgcctatgagaaaaccttgtctaagcgaagaagcaaagatatctacaaaaggatttcttgttgttgaaaaaatataatctaattctcttatgctgaaatttttgaaggagtttaaaagttctcttagttctttattcgcttcatttctaatctttaattcttctgcatatagctgagtaagagattttagatagttaaatctctcttcttgaagcctagcaacttcttctggtgattgcgctttttgtaagctttcatttactctgtttaattgcgcaataaaatttgacctagcgatgcttattgcgtttaaagattcgttgtaaaactcactaagcgttctagatagaaatatttctctttcgcttttaaaaattctttcagatatagaaagctgatttcttgttgatttcgctagctcaaaaagacttctaaattctgagaacgctctttgcgcacctgtttgtcttaggtcaaagtagttagaaagaaaatctctatctaaatcgcctgaagcttctaaatagtatccagttataggaaacttttctatagctcttttataaaatgcgcctaaagcgatgtcttgtatagatttttcctgagttctaatatcttgaagttttaaatcagcaacaaatttttctacagcctttctaaattctcctagatttactgaagctatattaagcgctttaggaagttgttgtgataattcttcaacaaaatttcttcctgtagcttgagagaggagatttaagttttctatttgctgctttaatgatgaaagaaaattttgcagctcatttaaatctctagcataaggcaaggatatgcctcttgatcttctaactgatgaaacgctgtattctaattgctgtaaagtttcaattagctgttgtgtatttctagatgcagcttttagtgttgctgataaaatttgcgaaaatgttcctgtttctgagcctagaagttcttctattgagcctttagattttattagatttcttagctcacctaaaatttcttttgtgttttcatctctgtcttcgggcatattcttcttttttattatgctagataagctttgatatttagtattactggcaaattaaacactctttctattgcgccaactagacttacttttagtgcgaaaggatagaagtcttgataatataacctttctctaatttctattgaacttcctttaaacgctataaactcatctatactcttaccttgaaactcaattatttctccatagggtgtataagataagtgaattttatttttgccgaaaaaatttgcaacaactacttctgattttatgttttttagcagatacaaatcaaatttatcctggtgaaatttatagtctatgtattgactaatttttatcttataaacataaaattcgctctctaattcgttaacgaaatagaaaaacctatattgaaaatttcccaaaattgaatctaattcgttaaatgtaaaagtgagtttatatgtttcttctgctaggtggccgcctagagaattttttctttctgagtttcttatctttagtggaaaaataagattacttgaaaacataatcttttatttcttattatcgcacagcgaaacaaactttcattggtgtcaaacttcttctagtgctaaagctaggacaaacgctttgtgtcattgggctatatatcttattttgaccaaaactctttagaaaatttaaaggctcaatgtagccataatacttcgcataagagcctccataaaagatttcgtccgaggtagcttgaacatatgaattattttttccgaaaaaattccagggcccacttggatccataaaagaagatatgtagtacttgtaatctatacaatattgcgttaatgatgttgtattttttagtgtgtcatacaaaattgctgatatataagctaaatttaggcctttgtctttatacatgtttcttggatagtttttgggaaaatttgagtagatattttttaatgtagtttcaaagtgaagatgagatgtgctattgcatttgtattctcctattgaagctattctttctcccgccttgactttttttccaatgtttacttctggaacaacatgaccataaagcgcatagaaaactttatcttgacatctatgttctattgccacaaatccagaacaatcgcaagaagcactacaaaatcgtctactagcattgatcacaatgccgtcagttactgcatatacaggaagtcctaaatcttgatttcctgaaccgtagtttatatcaacgcctggatgaaatgaaatatatttaaaatttgctttattaatcaactttgctctgtctgataaaatttttcctctagcgaagtttaatttttgcgggtttgtgtttactatgaattccaaaagaagtattctattttctatagatgacaaatcttgtggcaaatttgggtaattttgtttgaaggaagaatagtttaattcgtaagattgtattaaagaggttatatcaaataaaattttttgttcttggtgcgtaaaaaatttttgattgggatacggttcaccatagtttgtggctatggttattggtgataatgagcagttgattggaaagtaaaccttttgcgccataactttaccaagcagtcattactggtctgtaataaggagataatagttctcttaatctttctttgtattcgttataaagattttcgtaaagtagaccattgtttccaaaggatgcgttaaagccttctataccgaaagacctaagtcctgcagttaggtttgcagaaatggctaagatgaattgcattgcagctcttatcgctactgcttctagtaaatcgtatggagtgtctgtgtagccaaattcgtaatcaatcactatgacttgaggaattcttggtgcgattggaccatattgcgaaccccaaagtgggtcttgagaggttgtttgtgataaaattctagccattacagcaggaaatatgtgtatttctccagttctataaaagtaaaattcatttggtttatacaacctaaaaaagagatttttcccttcgtatccaggaggcgctgtaaacgctaaagccaaagaataaacagttattattggggcataataagtaataattagtggtgtgccttttccgtcttgtctttctgcatcaccaattttatttggggcaacacgaatatttgaaacccattggtttaattgatcgtttgtcaaaagctcaaaaggaagagaacctggaacataaaaactaggattgttaccataaagatttttttgagccgctgttaaatcttcccatttaggaaattgatagaaattttcatttatggcatccttgtatttagctctgttttcgtaagcttcttttggagttttaggaagatttgtaaatctagctggtgtaagagaagttctcaatctgttctctatgtattgtgcagacctttctatttctctctctaaatactgctgaacttcaggagtaatttctggtaataatacaccctcaactttgtctagcaatatttgaaatatgtcttgttttactttttctgtaagcatagtcttatattagagtgtatagatgataaattattgctattaggcccaaaatcattaatagcgccaaaacatgagcaaccaaaggttttgtttttgattttctataaaatctttcagaaagcgtaactccagttagctttatagatatgtattctcctatacctagagctgtaagaaccagaagagtaaatattgaccattcttttaatccagccaaataaaaagcaacagtccaaatcatgaatgttcctaatactattagcataatttcattttatacaggaacttttgttttttcgctttttgttttacaatataaatgtgcagacacttaaaacatggtatcaaaataagatgtcagtttatagagaaacaaacaaaacaattagagatatacaaaacttgaggacattttactatctaatgaaagcagaacacggagaatatgctataaaggattggaacaccttatttcgaaaatttttaaaagaacaaggacttttgtgatagaattaagtggaggaagttatgaagaaggtaaaagtaaagtttaatccaagaacaactcttagagttgcctacaaaggtcatatttacagacttagcacagaagatgaaggacaatttgttgagttaccaatttcaacaatgggcaagaaggaagcaaaagagcttttagagcatctaaaaaatataggaattttatcctcttacgacacagaagttgttgaagataaggaagaagtcaatgttgtgccaaacgatgatgtcttaatgaaagaaatggataatttaactgaagaacaggatactgaaaaaagagttgaagaggaacaaaatactgaagagcctgcggttgagaaagatacaacagaaaagccaaaaagaaaaagaggaaagaaaagcaaataattagcttgttatgattcaaatatcgctttctcaagcaaacccactaacaacaacattatctggtagtacttttgatgttaataatgtaaattattataatgcaggtgtaataaaagaagaagattcagggagaacaaaaatttttatttatggtgaagatttacaacttgttttaagtccaaagtttatctttctaaaaattagtgcagataaatttttgactcaccaagatttttgctcatcttattctgttgatatttgtgatgggtttactataaattatcttcacggcgctgaacatatttcgttaatttacataaggggaaacattctgaatatattaaggtttttgtttagtttgacgcatccaatattcacactaaacacaacttttaatgaaaactacatacacgcaaaagtggcatttgaagaagggccgataaagataagaacagaaaacattgacgctggtagtccttataaaaaatattgtttttgaaaatttaaaataaaaagcgatgaaaacaaagtttgaaatattaaactttttggaaaacacattcttagcaatacaaaataaaataaatgcgataaagcccggtagcgtaataagatctattttttactctgttgctagcgctatagaagatgtttacacgctcattgatgaggtaaaacaaaattcatacatacatacagctacaggtgaatatttggaccaattaatctatggtttttcaaaattagaaagaataagaggacgaagagcttttggatatgtagttcttagccctattggttttaattttgactccattcaaagcatagacttgttgtctaatatagttttctcaaaatatgatataaaaaataacctagttttttctccccccctctctgccaagtttagtatttcaacagcatatggaatttttaactatcttttactacccccgattaaattatcacttcaatacgaagacgatttttattatgaaataaatcccaacacaggaaagcaaagaatagccgaaatttttgctgaaactataaaagctaactttcttcaaaatccgacaaagaggataaaatacatagttctacctataatttcggaagactttggaaaggaaaagaacttactaccatcatcaatagatgttgttttacaatttggcacagcgaaattttctttaagtaacaatttcgaatacatgcttttaacagatgaaacttctgtagatttagattcagtatcttattatgaagacacacttctcgttttgggcgaatcttcggagattaaaggaggatcttcagaagaaacagacgatgaatacagaaacaggtattggaactttttaaactctttatctaaaggaacaatttcctctattgaaaatgccctttctcaagtaatacagggtgcgaaagtaaaggctattaactctcccaatcctggagtaatagatgtttacatttattcagacactcaaataataagcaattcgcttgttttactttctcaagaaattgtagacgaagttaagcctgctggttctataataaatataaggtttcctaaaactgtttacataaatcttcttgttgacagtaaaaactccattacaaaaaacgacattgaaagtcttagattgaagtttagagattcgttttctgaattaaacattggagattctttttcttacaacaagttttgggaatttttaagggaccaaaactcctcgtttgcaaatccaatgtttggcttatcgttaaatcgtgaactttttgagctttataaaaacaatattaaaaagtttgtttgtttaaactactacgaaagccacacatactttacaacactaccagatttttgtagtggcccatcggagttcacttatagtgattatttgagaattatttcagaaggaaaaatagctttatatatttacgataaaaacaacaattacaagtcttacataacaaacaacaactattcttcacagttaaatatacaagcacctagatatccattaaaaaaagttatttcaagagtgcttggaggagaagaaatcctacatccaagcataagaaacgcaataaaagcaagatgcagaaatatttctgcggacatatgtttaaaagaaatatctagaaatggagatttaaacgcttctgaaatccaaagcagaatagctatacctatttttgatttagacgaggtgtatgcaaactacttcaaaataaagctcatgacactaccagtaaggtcaacaaatgacatagaactttgtgaaaataaaatatcagactattcatactgttccggaaattcttatgaatttaagattttgtatttcttgttaaacgatgttcagtatatagaatataaagacttagacgaaatacaatatgcagatggactaatccgcttacaaacatctagtcaacttaataaagactttatagcaaaatataatgtagctgtaaggagcgaacaatgaaaaagtttaggcctagttttgcaaaattcataagattttccgatttgagttcgttaaacgaagaaaacacatcctttttattaaagctaataaagactacttttggcaaaggttattattctgacaaaaacataaaccaggttgtttcctttgatgttcaaactgtaggaataacagaatactttaactttaggataaaaacaaacgaaaccttttcatttttagacgaaaatggattttccgaatctgttggcggaaatttgaatttttatagtgttatttttgacgaaaaaaatcctttaaatggttctaccgaaaaccctattagaataccctatggtttgtttccagtaaatactgttgtagggtacagtaaagaaaacattccaattctttatgtaaatggaacaccacaattcagtagcgatttttcaacatctaaagattcagaatattcaaaaataagcttttctagaattcgtggaggatatatttatccaatacccaaggataaaaacggaaagtttaatacaaaggtggttactggtacaacctttttggactttaaacaggatttttgcgatgatgatttaaattgttatttggattttgggtttttaaatacagtaagcacaaataaatatcttgtactaagagttacatcactaggattggaaacatattatcatatacctaaaaacctaccaatttataacaacaatttatactatttaacttctgcagataaaccaaatgttgttattatcaacttttctgagttcgctttaaacgacaacgatactgtagaattctatttcatagaaaattccccaccatcaataaccacaacaagcgaaacttttataaatcagtacaacataaatgtagacaatatccttgtagacacaaattcgtcagcaattgttttggcactagatgaacaaagcgggacatttataaacctttccaatttaaatgcccagctagcagcaaacatttatacaataaatacttcctacggaaaaatacaagtaaatacttcttatctccctagcccacaaagtaaggtctttgtggtgaactactactatgctcccccaataaagatttacccaaatctagattacctaaataacatatcactctatggtacattcggtagctcaatatcaataaattcaaaaccgaaaacaattcaaatttacgcaaaaaatacacatattcctttttacggaacaacatactatgattcctcttcaaacatagtatctgaaccttatgtatacgatatacttccatacttcttgctttctgagtccacaataaacgattctcaatatacttatataggctctgtaaccgtaaacacagacaacagcacaaacttctctccttctagcaacatacaagttccttaccaaaatcttataaacttagctcaacaaatcaatcctcctcccaaatggaggacattaaatataaataaatcagaattctcatctggtacaaacacaacaatcgttattccaatactgcccgttgtaacagaaaataataaggtaacctcaataaaagcgattcatttaaaacttagagtttatgtatacgatataaacaacggtcaagttgatataccatttgacttaaaactctccttgagagatggtaatactataaaaaacacaataaccttaggatcttctttatctacagcaacctctcaagtgtaccaatatggaaacaataatttgtttagtttaacaagtctaatgtcccataattttaattccaataacgatgtagctccaatttattatttagagttttatgcatcttcaacaaattattttgtcgtttttgattacgcaattatctactttgagatatactaaaaaatagactatggctatagaacacttgaaacaatcagaagtttatacctctgaaacaacaaacgactctgtagacttaactacctttgctcctctaaacatacaagacgacttaaatgtaataagaacgatattaaaaagtattattggtggaacaaattttaaagatattgcaaacatctatacgctgaaaaacctatatacttggagaaacacacattttacttcttttggattaaaagcaacatctatagatgttagtgttgttgctgatgtagacaatttcagatttgttttgtctaataactcctattggcaaaaactaaaaagttttgctcaacaatatttaaatacgcaaaacaaagttctttggtctggtatagcttatacgactactgaattagataacgctataagaacacttccagttattacttctcttcaaacaaatgttagcagtttacaaacatcagttaactctcttcaaactaatcaatccattttctttcaagaaatttatggagcagtagtttcttcttcctctggaaaccagcctatattaaataggatttctgccttagaatcacaagttagcagtttaagtggcttacttaattcaagcaattacattaatgtaataagccaagaaatttatggctattttcctcctccaggtgcgccattaggggcagattctatacttacaagactaaacacagtagaaaatgacatagattctttgcaaaacaatattagtcaagttccacaaattcaaacagatttacaaaatctacaaaacaaagttggttactttgactacaacacttacaccaatacaataatcagcataaatacaaatataaacttcctagattttgtttctcaatttggagtaaaagaaaaaatattattggactttgaaaaattaaaagtattagatttagataggacgattaaaaatttcattgaatctcaaccagctgcaaatattatttcacttttaaacgcattaaaaagtaacgatatagcagaagttttcattccctataggtatatacatttggtaaatagttctaatataaccttgattgacaaaataaaagaattgttagagtccttaaactactccggtaatttagataagtttaccaaaataacaatccttataattttgaaccgttctactacacaagatgaaagtgtgctctactctcaattacctcaacttatcgccaaacttcttccctatgtagatgtagcccttcaaatagaagaaaattttgatgtttggatagattctacttattcatctacaattactgtaccttttaatgcctttgctaatcaagtggaaaaagctattaaatacatatacgaaaaagaaaaatctgtaaaagtgtttacaaataacccaaaatttgtgctgtcaaggttggggctatacaatttagaaattttttctccggttcaattttattggcctgtaaatattaatggattcaatgtaaaaattgataaagttacattgtttgataacgcatttgatgtaagctacttttgtacacaatttcctcccaatatagtggatagttcaacagataatactgtatgtgatttaattttcagtccatcattccaaggagttacatttgatgtatcttttgacacaagtacaaataattacacttcgccatcagacattgaaaagctttttgctgcagcatcagatagtattaaggtgggagttcttacaaattatacatccaattataaagtgttttctcccatttccatagatctttctgatccttccaatattcaagtttacttttctagtgtaaaatcaggattttttaagctctatagaaaactgaagatatttaataaagatactaacaacttcccaacaacagaaactggtaagaacacttcagtaaacgaattttatgattactacttgttagacacaataaattcttctgctataatctacgaatcggggataaaagatgcattaatttttgctacatctttaggagagaattttgaaatagaactatttattaatggttatgatatttacaatcaatcgtacaactacatattaaaactttcctatgatgcatctacgggagatttagctggatacgacagttcaaataacgcaataactgggttttctaagaacataggtactagcggtttaggtatttttaaggtgtttcttataaacaacagtacagatttagatatttatttgggaagtatatctgatattacacctccaacatcccccaaatacacaataacaaacatgaattttgatggatcaatcaatattattttgaactttacaggaactacagacttgaaagtaaaagtactgtcaaacgatgtataagttaaagataaacaccgaactaggaacttttgaggggatagacacaaaggaagtttatttgccgatttttaacataattgatgaagataatccttatcaggaacttgaacttttaattgaaactactgaaccgcaaagtttagattatatatacccccacacatcgttattaatgtttaatatttctccttccataactttggaaaacgacttctctccattaacattagaagtttcaattcaaaactcagaaaaccttctcgttattaacgacaaaagagcgtttgatgggtatagtgttgttggagtaatcttttcgtctgattacgatttaattgtaaactttgaaaattctgacatcttaagaagaaaatttattcttgttccatataagttatcagcaaatagcgtttactcaataaaaataaaaatctacagaagcacagaagaagctcaagcttcaattataagaaacaaattttacagatatgaaaaaattatatacgacttttcaaaatcctactctggtgctctggaaacaataaataatatatctctagcagagttttacgaaacctacggaagagaaagaagttttttggtgataaacaaagaagccctgcttaataaagttttttatcccaaagactttttatatacgcatctagcaacaaactctaccattgttcttgatgacggctctttagattcgtttattcttaactacgaaaaaaattttttatccgattacagaattgaggcaagcgctgcctcaaaatttccttatctaaaattctccaacacgataaacacattagtaggaacctatgaaaatatatcaatcaacacagaaaactatctaaaagaagtttctaaccttgtttttgcggaaattttaaaaccttatattctcaactctggatttttcccaataaatactgaacataacataaaattattagcaagtttcttggaaagaattcttcaacaacttattacattaactaccgttaaagaaacaatagtaaactatacgattttttctggagatttgttgccgcaagaaaaacaaaaactttggttttctattttatatcttgagtcggttttctttatagacttcgtaaacagccttttgcataaaggatatataagccaaataccaacagaggttttgtctttaaaggatgttctagattcttattttcatacctttttagatttttcttactttttgaataacctaagcctaatagaaaaagatactttagatacagattttataatgtctttgttctttattgtttatttgtatcatttatcaggaaaaaatatatttttctctggctcatcgttagttaaatatttggaaattttttacgattattatatgaaagtttcagaatcattgttttttgtaataacaaatctaaatccaaatagattcataaacgactcattagtttccataaaaacagtatcaaacaaagctttgttgttgtctttatattcgtctttgtttggtagagaagaattctatatagacctactaaatagactaagcgcctatggtgcaagcaatataaatatcgaagaaaacacagaatactatacaatttcaatagaactttcttctttatcctctggaacaagacagaccttgtatggctcaaataccgttgttttatatccaggaatcggaaatgacgaaaatttttatggtaaaaatgttgttttagcaaacccatctctcaacgaatattacatattcttgcccttctctattttttctacagaaataaaaggaaacaaactgattttaaaggttagtaaaagaaactacatagttaatgaagaacaaaaattagttccaaggtttattacactagatctttctgcaaatatgataaatttatacacaaaaataaatgcgctaattatagataacaactactttatttctagtggaggaaataatcaaattgaggctatcgcaaatacatttgtttatgaatatttgaaattcttactttctggaaatttaaacaacaaagtttctcaaaacacaacaaatataaaaattattttgacaaacaccttttgaaaagtttatattaaaatgtagctatggccgaaagaacttttaagctacattatccacaaaatacgctaatagatccgatgggatcaattatcattaacgtaaacacaacagtaaacccgcaattcgaatgggcatatataaattctttaaacggcgttggtccagttgacccctcaaaagaagtaaatatttccttattaggttttaaagatacaacatcctacaaaataaggtacttagaaaacataactagacacaacaacacatcactaaccgtaggttcagcggcttgtttaacaagtccagatgctgtatcttgtcagggttatggtccttatatagttttcggtaagcggtattatatagattttatcgttttgtattttgtaaacagaagcgtagtaccgcaagggggcaatgaagccgattattatttatctagcccaatattttcagtagatacttctagatgtacaccagattacggaatctttattgcttcaccaaaacccaaaacaccaaccaatagctctgatccatacgtagaagcaacagctggtactggtgatagaaaaatgtttactacactaccttcttatactaatgaactaggattttttaatcaaagtataaaatacagaataaccgctttaacaagcacaaattccaatagctatatggatgatgttttgttaactccaggaaatattgtgcaagcgttgcaaaatacaattaattatggacaaactagcattgtgtctgtaaaagaagacaatacaccaatagtgttaaggcctcaagctagcaacgctcccttatttaaccctcaatcatatttaccttttatcttgttcagatatataagtgtagatgccccaacaaaaagagtatttctttccttctcttttgctcctaacgcatagacaattatgaacaccatctacatagattttataaacagattctttccctctattgattttagaggtgcggtaaacatacttaattctgatttccccgatggaaatttagcggtttacaacataataaatagagagtatccaacaattagagattgtaatagaactccaacaatgatctttcatggaaacaacgagcaaataaattttagaatttcatatggaattgttgtttcttctacaaaagaaataaatgttagtgcagaaggaatgattgatgataagcaactaataaagaagctttttgataatttgcctaactattttgcctctttggaggaacaagaggatgaaaattagcaatatattgattgacaataacaaaacttttcctatagtaatttctccagaagtaaataatgttgaagaaatgagaaataaatactatttttatggactaaataagtattttggtgtgttaccaatttccctttataatgaaaaaggacttacttcactatttgagtttctttcaaacacttcgaaaactttagtagatttagcatattctttggatgaattagaaagcaaatattttaaaagttcaaaaataacaaagggagttattgaagaaacaagaagatttttttatccagtatataacgatgatgtgttttctataaaaccagagctttgggacacgctgtataagtttttatacctgttttacaaatctgttttatatgacggcctaaggatatacatgtcttacactttgacaaaagtaaacaagttcttttcgttttcaatattaaaaacagaatgttctaacagtagtgaagtagaaattaaatccttttttgaaaaaataagaagagccttttataaaactataacaccagatacaaatgatgccttaagaagttatcttctttctttgacatcggacgctttttatatagaagatcctattttgcccaacaaggaaacagacttaaacttttatgacgcttatacaaatgcattgaggggagctacgataaaacagctaaaattagaagaactttaccaaagctttaaccaagagtttgattcgttttatagaggaaagttatctgagattttttcccaaaataatattttttaccaagaacttgttagtattgtaggaggaagtttagcagatgaataccttaagttcctcatctcctaagttaatatttagtcctcctataccgggcacttcaaagcctgaagaaaaacaattatcttttagtcctgagcaggtatcaataaaagaaagggcctcgctaaaggaatttaacaacaccaagcttacagctaaagatgttacacaagcctttatactttattgggaaccatattacacaaacggctttattaacattccttaccgtgatgtatatgcgaaatttctaagagaatacggaaacaacatagaaccagaaatagtttacagatatcgtgggctggatgtcctcttaatgaatgaaacagatttcaacaactttgaaaacaatgaaatagctgtgtatttgccaatttcagtaggaagatatttggtattagagaagtttgagcttcctaggacaataataagcgcacaaaaagtttcccaagaaggaaaatttgtttctgtgtttacagaaaactttcccagattttcaatgacactaaaagctgtaatctttggtagcgtgcatataaaggcaaagcagttttttgatatcgtaataaaacatatttcaaacaacaagcccggtagattgattctttatgatatatttggaaatgtaaagatatctcaagctgacatttttctaactttgcagtataacaaaacacctttttatcgccttttacctacaaacgtttcttattcaagaacttcatctgaaaacacatacttaaacatagagattactggtacagtattagaaatacacaatgaaagatatgtttagttcagaaacttttagaacatatgcagttgtagtaattgaatctctttggccttctagggaagattatagaggagaaggagaaaatatagtttttagtagcggcgctaaaaattatgaattactgaaattttctcctcttcaaagcaaatattctggttattacaaaataataccgttaaatattcttagcgttaacacatcgttttccaaggagggtggaaattttaatgtttcgtttacggctaacaatctactatttctatcagatagagaacttccagaaagattgaaaaaaatactagaatcaacttcaaccagattagaaaacttccctatagaccaatttccctttttagaaaaggttttagttggaaatgacaagttaagggttggttacataacaaactacaatgttatatctcaatttataagagaacttgatactgtttcaatctatttggtgaatataaaagaaagtctttcaaacgatttgcttcctttatatatcgggaaaaaacctggaagaacaaagcaatctgaggcggctcaattagtagacaaaaagaatgaaattctaatttctctcggtatatcaaaaaatgaattggtttttttagatagtgtattgagaagtaaagactttaaaaacatgttattttactacagaaagtttagttatcctctcataattaatagatttttcaaggcgttaaacattcaactgttttctcttaattctgagtttagaaacaaaatttatcaacaagaattgaataattcagaaacagaagacatagaaaatttcataaatttgtttgggcgaacaatttttgattactactttaaagatagaaatgctagggaacagattaagtcttgtattgaatcttattataaagcgaattctgttttaagtaaactaatttcatggactgcctttgaaactttgcgattaaagctaaatgaatctttaggcgtttctccaggactttgcttttctagaaactcaaaggtagaagaagaaagaaaatttaaaacaatactgaacgcaataattattgacaactatttgtatgaatatgctaacaaagtcttaggctccgaaagccaagaagaaatagagtttggtataaactttataaacaacattcttggcaataatttgttcagctttgaagattacttgttaggtatacacagccttttgtctatattcgtaaaagattttaataacacagaatcaaatacggaagaaataaaatttgatgatattcaagaagagctaactattaataattccggtgatgcgcttagccttgtctttaggggacacataacagacatttcaagaagtgtatcaattagtgaaaattcatctgatatagttatttcaataaaaggaagggatcttggttatcctttaaggaaccacgaagtttatgcagattatacttcaataaagtttgcggcacaacctctagaaaactactcggtaaaaattttaactccagtagaagctgtaataaaaataatgaactcttttgccccacaaaaaataaacattctggaactagacagagaaaccttcttaacatccgttatttcttctaggggatttgataccttctataacgctggtacacaaaatatattgataaatcagggaaatgtcgttgcgccgtcttatgatgccaaatttgaagaattgtcaatttttacgccaatacattatgttagtttagattttatacaaatggtaaaaaatgcctttgatactgaaggaattcttcaaaaatttttggcgaatgtgaatgttgatatttctgatgggccattgtttgatgtactaaaaagagttgtatcctcaaactctgcatataggatttatgttgatcacctaggttatttaaaaattgagtatgaaccagcacatatatcttttccattttcactaagtcttaatagggaaatagacgactactacacattctccatagaacactcttcaagcgaaagtaatgtaactactttcgtagaagtgataccaaaatcatttggtacgagcagtccaagctctatgggtattgcatcgttgtatgggagaagcatacctccaacaattcaagagctttattcaacagtagaatacatagatagcaacaaagataagtttgtagaatttctcgctgaggcagtaaagttattggatggcagccttaagaatcacattataaaaagagatggcgaatatgaaagggaggtttttaacaaccttacaaagttaaatacctttataaatcaacaaaacgataacgatttgctttccgcccttaggggtcttactaaaaaggaatctgcacctcagcaaatatcttctgtagatatagttaaaacaaccacctataaggaagatttttgtacgggggaaagaatagctaacacctcttattcacttcaagaaactactaaaactatacaaacacaagttgaagtaccaattagtttgtttaaaccagaagttctatcaaatatattcaatgttggaagaatagacgaaaagttcttctatacaaatgtagactacgaatactataaactttttggaaaatcttttgctacattaaacgaatattatgcagacaaaacttctctattaagtggtatatgtacaaaacttaacgctatagcaagaacaaagtttaacctatcctcttctgctagtgtttgctttgaagaaaacttagatttcttggcgagtttaatggttcttggcgcaaaatttgaactttacggaaaaacagataaagaaaacgctactttaggaaatttaattacaaacataattaactccataagaatcaaatcttctgaaaacgtcttcaagattaacttgtcgaaggttgatgagaataaaaatcaaacaataagttatcttaaagacttttacgcatatttcatacccttttcagatatatctaatgttgtaaaaaacgaaaacaatatactcaaaaatctttcaccagacctgtttatatatggtctaaggagaaaaactttttctgacgcttttatatctgtagtggactcattaagaaatgaagaaagaatttctgccaagcgtgctgaaattataaggaagttaaattctaagccaattaacacggcgaaagtttctgttttgggacacaattactttgttggatatactactctactgataaacgaaaaattacctcctttaaatgatgcctttatttctcagaaaactttaaacgacataaacgatgatgctaaatttatcaaagataaggaaaacctaattgtttacatcaacgaaaaggttttaacaagaagattttcagaaatctacaataaattattgccgcaaatacctggttataatcctccttcaaagattgatgaagaaacagtaataaactactttacagaaggtattaagtttgttttaagttttaactatgagtatgtgccttatcaatattgggtacctctttggggagtttatacaaaagaaagcctggaaaacataaacctcggagattatgtaaaaattttgcttactgagcttttgtatcattccccagaaaaacaacaagattcttctgaatactctgacctgttattcaggtttatatcaaacaaccaacaatatatgcacatatatcaagaatatgtaaagccgcaaaattacttagtagcccaaggacatatagaaaatgttgagcactcttggtctttaggaagtgtatacacaacaagtgtcgggcttaactatttacttccagctatttatacatatcttccgctaggaaaaaagaaaataattataggatatgtagttatagactctcctctttccaagttcaacgaatctggtcagtataacaagttcttgaaagatgaggagtttttcaagtactttagacttttgaaacgactgtatttaaacttctatgaaaatgaattaaatttcgtaagaaataatgcaatactgagtttgctataagttgttgtgatagaataatgaggatatgagagaaaaacacaaaacaatgttgaactttgaagagggcgttcaaagaaatgaattttcttttggcgcttataaattagtcgttaaagttccaactctttttgaggagtctaaaatttttcttaagaggtttgagatttttaaaaagtttttgaacgaggatgaagattttagaaaatattttagtgatgtaactgtgaaggaactaaagataaatgatgaaggcaaaccagaaatagttgatgtttcctttgatcccaaaaaacaagaccacattgaacatgtatttaataattacctaggagaggaatatagggaacttatcaatagattagcctatcttgatgtcgttgtagaaaaaatttatagaggagaggaagagcttagtggaaagttttctgatctgttcttttttcaaccaacaagtggtattcatttaagagagatatttaatctctctgaggaaatcatcaattggatcaactcagtaaacgaggttgacccttttttgtcaaaaaactaatatctgaagatattcaactaaatgctataataaatgcttacttctcttcattttcagttccagcgattgactcttcaataaaggcagatataaaatccttagcagaaggagggggtattagtcagcccctatttcttccccttctaaaaccctatgaaaaatattttcctcttaacactctaacgaaaggaaagctaaatttaattcttggaatagaattttactttggaagacaaaaaactttagcattatatcttttatcaacaaaaaacaaaaaactcaaagaattagttgaagcagcgtctaagttgaagattctaaaagacaaaagccttgggctccacgaagaaatagcagaaaaaggtttatcaagactagatgaaatattaacagaaattgaaaaactagacataaaagtaaaaacacttttagcagaggtttcttctgacaagtctttacaaagcgagttgggagttggaggagaagaattgaaaactatgattgaagaatttatagcctaagatcaaacaattcttctgtaaaagaatttttggcgaactttttatttacagcgtctatataaaaggttctaaaaccatcctttacgacagactcttcgtatatttctatgtgttctttcaatttttctacaaatacactatcaactgatatggtaactatttcgtttttagtttcatctttattgttcctgggtgaatatagaaactttattctatcaagatatttagcaatagaagctacatctgaatcaacatttaaatattttcccctaccaacccttttatagcccaaaagaagaattcctaaatcaatacttgaaatactaggaaattcttcggataaaaatttaaaacttttgtatttggcctcatatacctttcttatgatctttttcagactttgttgatctttgaagtgtattccttcaattaagtgaacttttattctaaactttatgtcaaatgctggactatctaaatcaagcaaatctagcagactgtaattttttattttattcttttcttttcgtataatcttttgtattttagatgaaaacggatacaatctaaaattgtaaaagttttccacattatttaacaaaatttttacttcactatactccttacctggtgttggactgaaaccaacacctatggaaaacgaaaattctgcaccatttgaatttaagtagtactgcaagtaattttctttattgcttttaacttcttcaaaaagcctaagaagatttctgtaactaaatatgtcttgtgcaaaattcaaattcttggtggttagagaaaagaacaaccttctctttattgtttcaagaaacacatcaaatatatagggatattgtgttggttctcctccaccaactactacctctaacacatcattttcattaagaaaatctagaactttttttatgtattcaaaatcagcatgttttccagtaatcttagagttttggtagcaaaaagtgcaaccataatcacaatagtcagttacttttaggtcaacgcttattgcctggtttttgatgttttgcaggttcatcttagatctccacctcctagttcgtaaatcgcataaatgttttcttgcggatcatataaaactagctcatttccttttctggaagatattgagaaattaacattcttcgcttttcctaaggcattaacctccctatgaaaaacattagtttctaggttattttgtctttctgtgatgctgtctataacaatgtctttaaaaatagcttttctatttcttttgtatggcttagaatttccataaaatttagtaattgccacttcatttgttaaaccttttcccaaaaatgaagtttcttcctcttcgtatatcagaagtagtgtttttactcttttctttaattctctataattgtccaaaatcgcttcttcgtcaaaatcatatgtttttctatctagtacaggattaaacagtattttcaaatagttggcgtagatgtagagaggaaaatttagtgtggcaaacttggcaataaatagatttccttctttcttcatttcctccaaataaatatttacaaattcattttcaggaagaagaacatctcctatttttctaagtatcttgtatatcctatttttcacagaattcttatctacctttttaaacttgtgataaaagtcagatatttcaataaatgattcaactgctaaaacaagcagaagaaagttccttatcttttcattttcattgaccaaataaacaacgaaatcttcttcaagtctatttacataattaaaggaagcgccttttaattctttcttaaatatttcgtagtttgaaagctttttgatgaagctactagatttctcatcttcctttatgaacaaaagcaaatcgccataaaacacatcatcaaaagaagaatctttatctgaaggtataactagcatagaagcggaagaggaatttgtcatgctggtaagtcttacttcttttagccttctcatgctcttcttcacctcctagaaaagctctaccttttcagcatctaaagttatcgctttgtgagccttcaaaaattctactacttggtatatatcttcaaagcgcttagtaagtttaacatcgtcagagggtattggaatgaatattcctttaacatttaaacttttaatgctaaattcgccaaaaatgtagagatttatattcaaaaacacctcatttatcaacgaaaagctaagcaaatttaaaaataccaaagagtgaagagtaattatctcaacatctttacctccatctaatttttctggtaagtacttaattgctataaaattttttgctatcaatgaaaacaaatcataatctagtacatctaccgccaaaacaaaatctgtggattgtatattgttaaacttttcttttgcaaaattgtttgaaaattttaccgctgaaatccagctttcgtaaaattctaaaaacggatttttctttccgctaattgtagaaacaatgtggcttgaagttatatctaatctaaacttataaaacccaaaatttgtgtttttaactttaacatctttttcattaattgcatctattgctacctctaaaaatttttttaatttttctctatcgtctttcagaacgcctggcactctcacaccacaaccttaccacaccgtgcctttggtgtcaaggcgctgatggaagatgttaaaataaagctatgacaaacactttaaacttgctagcaaaggaagttgccaatagattgggatttctttacttgaacacaaaagatttacatcacaaacttgaacctgttaaattttttgtttcaagatatttagaagacagagtaatagatgaagaagatatagagttcttaacttctagctacgaagactttccttcatcaaaccttttgagaactgaaagcacttttttaattcaaagcttttctgatttggctggttttgcagaaaacatagagttgtcattgtctaacattgcctcgccctcatctcttataaggatagatgactattcaaaaataataactgttgatgacattgaaataatgttctatgctatagaggattacgagcatgttcttttgccctattttttgtttcttcttcttgtaggtatcaataatgttgaagacgttgtaggtagtaagttagaaccatcttctataaataaagaagactttagaaagttctcaaaagaaacaaaacaaaaaaccaatcttcttgcaaaattattgtgaaggacagttcaaaaaattgaaataaaatgaagtagagatggaaattatagccaaagaatattacaaattgggagagaaccttctttttgttattaggggatttactaatgatgtaagcctaacaatacgaacaataaaatctggaaaatatatttttcgtaacacttttggttggacaacagccgatacacaaaccccaacaacttttcttttttctgatgcattagacgataacggaactaagtattttattatagaaataccaaaggaaagtttattaaaaaacgacatctacaagttatttgttgatgatggtaacacacaaacagaaaagtttgtttttgttggagattctttcacttccccccccaaaataaatgtttacggcaatttatatgatgcacttggtagaccagtttcatctacaacgcttacatttagcgttcttaatcctgttggttactttgataactcaccaattaccagtatgaatgcttctacaacaacagatgaaaatggatacttttcgatactattgaacaaaaattacaactatgtagttgttgttccagaactaaattaccgaaaagttgtaaagctttctgagcttcctgatagtgttacttctgtagaacttgtctttggagaggtaagcaatctatgctaaaggtgttaaaaattgagttatctaacctgtttacaaaacctttggagggggcaaaaatagaaatttatccctatccaacaaaggtctattatttgcaagacaacagaagaatctatttgcctagcgttattgcttatctaacagcaaaccaatggggaaacgctgaaactcagttagtacccaacgatttgcttagaccttctcccaacttttatgtagtaaaagttatgtataaaggaagaatttactatttcgtagcaaaaataactagtgatatgaatgatattgtttatctacatgatgtagttataaataaggaaaaagaatgtgaaaaccagcaaaaccttccttatagggtgcttggcaataattactatatttaaataaaaacaaaaagcaaaccttaaaattaaataggggaggcatattatggctattgaactacctcaatattcagaaactatagaaaccaaacaatttacaactcagtctgaaattatcagacttccgtacgtatttcatgctattggtattacaaacaaaggaagtgtaccagtaaaggtttacttatcagaatctaatccaaacgcttatgttgttgtagacgctaattcttacaaggaggtttctgcatcctctaactatataaagtttgtttacgatactacaggaacatatgacataacatttgaaatctttagccttttttctgagggatttttagcaccaagatttccaaacctttatgttcaagacataaacggaaattatgttttgtacaatcctctaaatactgacggcttctttacagaaacatactcttatggcgactcattagcagttaatagaccttacaactccgaaacgatgttagaagtagactattggtttaatggtcaaaagttagcttctacagacggcacattttctagcgatttctttacaacattaatacaaaactttataacctctgtatttccctattctgatgtaaatgatgttttgcgccctctcccaggtggacaaactacagatatcataaacaaaatcaaagattattggcagcaaatagatagaccaattaacgatgaatatgtaggcggttgggcaaaacttgagtcctttgacagaaaacttagtttacttagctttgttttaagctctggtgataagatagagtttccgtttactgtattgggttttaatatagaaaaccttgatgactcaaatagcgctactgtaaggatttacaacgacagctcttacacagaccaaaccatttctccttcttctagttatattggaaaccttgaaccagcaacaaagtttgaggtattaagtggattagtaagaatttctttcagcggttacgctgttaaaatgctttcttttgctccaataattaatatttactaccctattggatttgatgaaactagcggtttaccaaacgatgcctatacactcttccaaaacaggctaacagaatttgaaacttggctaacaaactctaaattttctggtagagttttcaaatataagtgggtacagcatgtaccagaaacaagaaatgtttgggatgccgactattatcaaataacttttgccgattctaaaattcctcttccctttgatgagttaaggctttacttaagagattggtatacatattctcaacttgaaaaacaaataatttctagagtttgggacgaaaattattctgttttggggcaatatggaaaacctaaagatataaatgaagttattaataacgacaacattatttatgttttgggacaaaatttctctggatatgctgctggtacggcaagaagagatgcaaaaagagaatttttaagaagagtgttgcttgaaggctatctaggaaactacgccggaactttaacttattccataaattaacgacttaatgttgataaaatgaagttatgcctaaggaatatgaacattgcaaagaaagttatctaaaaaagggagtttcagaaaaaaaggcgaaagcaatttgcgctggaatgtattataatagacatggtataacagtaaacgaagccaaaaagagggggttaaaaagcatgaaacttttatatttaggtagaacaaaagaggctctaaaatcattaattttagctaatggagaagaactaaaagactttgttatcgtaaaagtgcccgattctctaaggaagaatgaagtagttaaatctatctatccaaaggtagaaaatgaagagtatgtttatgtggctgttcataagtctgttgttgatgtaactgttgaaggaaaggttgttctttctcttgaaaagctcctttctaaagttgaagaaaacgaaacttctaagcaaaacgaggacaatgttgctcatgtggaagctggtaaggaagttgttaaggaaggaaagctagaaaaaagctctggagaaagcgggcaaactggttctctaaaagaagaaattcgtgaagaggtcgtagtaactaaatctaagcacagcgtagaagaagatgatagtaatgttccttttctagatgatgttattgaaaaactaaagagaaagaatattctcacaaaagaagaaagggctgcgctagttaacgctcttagagtcattgaggaatttatgcaggacaagcttactgacgaacaaaagaccaatctttctactgcaattagcgatgtagatatggcgctttcagaagaaagtaataagagtgaagacaaatcagaagacaaagcattagaagctacctctgctacagtagaggcactagaaaaagaggatcttgaatctgaggttgttcctgtaacaaaaagcctttctgaacacgaaaagtttttgagcttaaagtctattgatggcacaaatcctgtttttgaaagcaagcatgatgctaatagatatgctagattgtacaaccttcacaatgaagataagaaggctgtcgtacttccagcttcaatgaacgataagaggaagtacggtactaaagctaaatatgtagttgtttttgaatgatacaatttaaaaaaactaagcagggcctgttttggtcctgctttctttgttataatagcaatatgactgagcaagaacttaatcaagttatttttaaaaaatttgaagaactaagggatgctataaaaaccgaattttacaactctatagatgaaaaatatgtagagcaaataagaacaaacaaaactttttataattcagaagaattagctaatcttgtcagcacaaaactaaaaaatcaatttgaacaaatttttcaggaagcagaaaacgaattcaagtcttttaaattctatctatttgatgatgcggttgggagagaggacttgaattttaatcctgggctttgtaaaacatctaaagttcatataattttagaaggccaatttaaaacagtagattgcggctttttagtattagaaaactctcaaatccttaacaaatttattgaatctttcataagaagaacctttggcgcactaatcgtaagaaggctaaatgaaatttctgcaaaaatagtggaagaaaatgctataatatctaccgatagttatgattcagcttaagttactagatcaagactttgaagtagaggtagacgacagatatctaattctttactcaaatgacgatttttgctcttctccaaaaacaatgagatttgttggatttgaaatttacacatttgacaatgttttttgcggaataaagattagattagaaaaattagatgacagaaagctagaatcaataattcttgttgatgaatataattacgaaatagatttatcttcatttgtttccagttacatttaggtaccaaaagaaaggtagaccacgccagtaggaacatttgcgtagtctgaactagtatatctattttttagatataaatagttagagcttctgaacttaatgaagcgtcatataaaaatagctattgtgcgattttcagaagttaaaaaactatacctaattaacgctccacctaagcattaaatcgctttatttggtaaaaattttttccgtat

SEQ ID NO: 172 - DNA, 74 bp, YS40 BACTERIOPHAGE
  misc_feature: MetAUG tRNA Polymerase Nucleic Acid Sequence Thermus_031217 (120026bp - 120099bp, complementary)
ggggcgttagctcaagcggtgagagcggtcggctcataaccgattggttgtaggttcgagtcctacacgcccca

SEQ ID NO: 173 - DNA, 74 bp, YS40 BACTERIOPHAGE
  misc_feature: ArgAGA tRNA polymerase nucleic acid sequence Thermus_031217 (120115bp - 120188bp, complementary)
gaggggttagctcaacaggacagagcaagggatttctaatccctaggttgcaggttcaagtcctgcacccctcg

SEQ ID NO: 174 - DNA, 73 bp, YS40 BACTERIOPHAGE
  misc_feature: ThrACA tRNA Polymerase Nucleic Acid Sequence Thermus_031217 (52068bp - 52140bp, direct)
ggcctcgtagctcaacaggaagagcactcgccttgtaagcggggggttgtgggttcaaatcccaccggggcct